(19) United States
(12) Patent Application Publication
(10) Pub. No.: US 2005/0037370 A1
(43) Pub. Date: Feb. 17, 2005

(54) Oligomeric compounds having modified bases for binding to adenine and guanine and their use in gene modulation

(76) Inventors: Brenda F. Baker, Carlsbad, CA (US); Anne B. Eldrup, Ridgefield, CT (US); Muthiah Manoharan, Weston, MA (US); Balkrishen Bhat, Carlsbad, CA (US); Richard Griffey, Vista, CA (US); Eric E. Swayze, Carlsbad, CA (US); Stanley T. Crooke, Carlsbad, CA (US); Thazha P. Prakash, Carlsbad, CA (US); Kallanthottathil G. Rajeev, Cambridge, MA (US)

Correspondence Address: WOODCOCK WASHBURN LLP, ONE LIBERTY PLACE - 46TH FLOOR, PHILADELPHIA, PA, 19103, US

(21) Appl. No.: 10/700,884

(22) Filed: Nov. 4, 2003

Related U.S. Application Data
(63) Continuation-in-part of application No. 10/635,380, filed on Aug. 6, 2003.
(63) Continuation-in-part of application No. 10/078,949, filed on Feb. 20, 2002.
Application No. 10/078,949 is a continuation of application No. 09/479,783, filed on Jan. 7, 2000.
Application No. 09/479,783 is a division of application No. 08/870,608, filed on Jun. 6, 1997, now Pat. No. 6,107,094.
Application No. 08/870,608 is a continuation-in-part of application No. 08/659,440, filed on Jun. 6, 1996, now Pat. No. 5,898,031.
(60) Provisional application No. 60/423,760, filed on Nov. 5, 2002.

(51) Int. Cl. C12Q 1/68; A61K 48/00

(52) U.S. Cl. 435/6; 435/375; 514/44

(57) ABSTRACT

Oligomer compositions comprising first and second oligomers are provided wherein at least a portion of the first oligomer is capable of hybridizing with at least a portion of the second oligomer, at least a portion of the first oligomer is complementary to and capble of hybridizing to a selected target nucleic acid, and at least one of the first or second oligomers has a modified base for binding to an adenine or guanine base in the opposite strand. Oligonucleotide/protein compositions are also provided comprising an oligomer complementary to and capable of hybridizing to a selected target nucleic acid and at least one protein comprising at least a portion of an RNA-induced silencing complex (RISC), wherein the oligomer has a modified base for binding to an adenine or guanine base in the opposite strand.

### CROSS-REFERENCE TO RELATED APPLICATIONS

The present application is a continuation in part of U.S. Ser. No. 10/635,380 filed Aug. 6, 2003 and a continuation in part of 10/078,949 filed Feb. 20, 2002 which is a continuation of 09/479,783 filed Jan. 7, 2000, which is a divisional of U.S. Ser. No. 08/870,608 filed Jun. 6, 1997 which was issued as U.S. Pat. No. 6,107,094 on Aug. 22, 2002, which is a continuation-in part of U.S. Ser. No. 08/659,440 filed Jun. 6, 1996 which was issued as U.S. Pat. No. 5,898,031 on Apr. 27, 1999, each of which is incorporated herein by reference in its entirety. The present applicaton also claims benefit to U.S. Provisional Application Ser. No. 60/423,760 filed Nov. 5, 2002, which is incorporated herein by reference in its entirety.

### FIELD OF THE INVENTION

The present invention provides modified oligomers that modulate gene expression via a RNA interference pathway. The oligomers of the invention include one or more modifications thereon resulting in differences in various physical properties and attributes compared to wild type nucleic acids. The modified oligomers are used alone or in compositions to modulate the targeted nucleic acids. In preferred embodiments of the invention, the modified oligomers contain at least one adenine (A) and guanine (G) modified binding base.

### BACKGROUND OF THE INVENTION

In many species, introduction of double-stranded RNA (dsRNA) induces potent and specific gene silencing. This phenomenon occurs in both plants and animals and has roles in viral defense and transposon silencing mechanisms. This phenomenon was originally described more than a decade ago by researchers working with the petunia flower. While trying to deepen the purple color of these flowers, Jorgensen et al. introduced a pigment-producing gene under the control of a powerful promoter. Instead of the expected deep purple color, many of the flowers appeared variegated or even white. Jorgensen named the observed phenomenon “cosuppression”, since the expression of both the introduced gene and the homologous endogenous gene was suppressed (Napoli et al., Plant Cell, 1990, 2, 279-289; Jorgensen et al., Plant Mol. Biol., 1996, 31, 957-973).

Cosuppression has since been found to occur in many species of plants, fungi, and has been particularly well characterized in Neurospora crassa, where it is known as “quelling” (Cogoni and Macino, Genes Dev. 2000, 10, 638-643; Guru, Nature, 2000, 404, 804-808).

The first evidence that dsRNA could lead to gene silencing in animals came from work in the nematode, Caenorhabditis elegans. In 1995, researchers Guo and Kemphues were attempting to use antisense RNA to shut down expression of the par-1 gene in order to assess its function. As expected, injection of the antisense RNA disrupted expression of par-1, but quizzically, injection of the sense-strand control also disrupted expression (Guo and Kempheus, Cell, 1995, 81, 611-620). This result was a puzzle until Fire et al. injected dsRNA (a mixture of both sense and antisense strands) into C. elegans. This injection resulted in much more efficient silencing than injection of either the sense or the antisense strands alone. Injection of just a few molecules of dsRNA per cell was sufficient to completely silence the homologous gene's expression. Furthermore, injection of dsRNA into the gut of the worm caused gene silencing not only throughout the worm, but also in first generation offspring (Fire et al., Nature, 1998, 391, 806-811).

The potency of this phenomenon led Timmons and Fire to explore the limits of the dsRNA effects by feeding nematodes bacteria that had been engineered to express dsRNA homologous to the C. elegans unc-22 gene. Surprisingly, these worms developed an unc-22 null-like phenotype (Timmons and Fire, Nature 1998, 395, 854; Timmons et al., Gene, 2001, 263, 103-112). Further work showed that soaking worms in dsRNA was also able to induce silencing (Tabara et al., Science, 1998, 282, 430-431). PCT publication WO 01/48183 discloses methods of inhibiting expression of a target gene in a nematode worm involving feeding to the worm a food organism which is capable of producing a double-stranded RNA structure having a nucleotide sequence substantially identical to a portion of the target gene following ingestion of the food organism by the nematode, or by introducing a DNA capable of producing the double-stranded RNA structure (Bogaert et al., 2001).

The posttranscriptional gene silencing defined in Caenorhabditis elegans resulting from exposure to double-stranded RNA (dsRNA) has since been designated as RNA interference (RNAi). This term has come to generalize all forms of gene silencing involving dsRNA leading to the sequence-specific reduction of endogenous targeted mRNA levels; unlike co-suppression, in which transgenic DNA leads to silencing of both the transgene and the endogenous gene.

Introduction of exogenous double-stranded RNA (dsRNA) into Caenorhabditis elegans has been shown to specifically and potently disrupt the activity of genes containing homologous sequences. Montgomery et al. suggests that the primary interference affects of dsRNA are post-transcriptional. This conclusion being derived from examination of the primary DNA sequence after dsRNA-mediated interference and a finding of no evidence of alterations, followed by studies involving alteration of an upstream operon having no effect on the activity of its downstream gene. These results argue against an effect on initiation or elongation of transcription. Finally using in situ hybridization they observed that dsRNA-mediated interference produced a substantial, although not complete, reduction in accumulation of nascent transcripts in the nucleus, while cytoplasmic accumulation of transcripts was virtually eliminated. These results indicate that the endogenous mRNA is the primary target for interference and suggest a mechanism that degrades the targeted mRNA before translation can occur. It was also found that this mechanism is not dependent on the SMG system, an mRNA surveillance system in C. elegans responsible for targeting and destroying aberrant messages. The authors further suggest a model of how dsRNA might function as a catalytic mechanism to target homologous mRNAs for degradation. (Montgomery et al., Proc. Natl. Acad. Sci. USA, 1998, 95, 15502-15507).

Recently, the development of a cell-free system from syncytial blastoderm Drosophila embryos, which recapitulates many of the features of RNAi, has been reported. The interference observed in this reaction is sequence specific, is promoted by dsRNA but not single-stranded RNA, functions by specific mRNA degradation, and requires a minimum length of dsRNA. Furthermore, preincubation of dsRNA potentiates its activity demonstrating that RNAi can be mediated by sequence-specific processes in soluble reactions (Tuschl et al., Genes Dev., 1999, 13, 3191-3197).

In subsequent experiments, Tuschl et al, using the Drosophila in vitro system, demonstrated that 21- and 22-nt RNA fragments are the sequence-specific mediators of RNAi. These fragments, which they termed short interfering RNAs (siRNAs), were shown to be generated by an RNase III-like processing reaction from long dsRNA. They also showed that chemically synthesized siRNA duplexes with overhanging 3′ ends mediate efficient target RNA cleavage in the Drosophila lysate, and that the cleavage site is located near the center of the region spanned by the guiding siRNA. In addition, they suggest that the direction of dsRNA processing determines whether sense or antisense target RNA can be cleaved by the siRNA-protein complex (Elbashir et al., Genes Dev., 2001, 15, 188-200). Further characterization of the suppression of expression of endogenous and heterologous genes caused by the 21-23 nucleotide siRNAs have been investigated in several mammalian cell lines, including human embryonic kidney (293) and HeLa cells (Elbashir et al., Nature, 2001, 411, 494-498).

The Drosophila embryo extract system has been exploited, using green fluorescent protein and luciferase tagged siRNAs, to demonstrate that siRNAs can serve as primers to transform the target mRNA into dsRNA. The nascent dsRNA is degraded to eliminate the incorporated target mRNA while generating new siRNAs in a cycle of dsRNA synthesis and degradation. Evidence is also presented that mRNA-dependent siRNA incorporation to form dsRNA is carried out by an RNA-dependent RNA polymerase activity (RdRP) (Lipardi et al., Cell, 2001, 107, 297-307).

The involvement of an RNA-directed RNA polymerase and siRNA primers as reported by Lipardi et al. (Lipardi et al., Cell, 2001, 107, 297-307) is one of the many intriguing features of gene silencing by RNA interference. This suggests an apparent catalytic nature to the phenomenon. New biochemical and genetic evidence reported by Nishikura et al. also shows that an RNA-directed RNA polymerase chain reaction, primed by siRNA, amplifies the interference caused by a small amount of “trigger” dsRNA (Nishikura, Cell, 2001, 107, 415-418).

Investigating the role of “trigger” RNA amplification during RNA interference (RNAi) in Caenorhabditis elegans, Sijen et al revealed a substantial fraction of siRNAs that cannot derive directly from input dsRNA. Instead, a population of siRNAs (termed secondary siRNAs) appeared to derive from the action of the previously reported cellular RNA-directed RNA polymerase (RdRP) on mRNAs that are being targeted by the RNAi mechanism. The distribution of secondary siRNAs exhibited a distinct polarity (5′-3′; on the antisense strand), suggesting a cyclic amplification process in which RdRP is primed by existing siRNAs. This amplification mechanism substantially augmented the potency of RNAi-based surveillance, while ensuring that the RNAi machinery will focus on expressed mRNAs (Sijen et al., Cell, 2001, 107, 465-476).

Most recently, Tijsterman et al. have shown that, in fact, single-stranded RNA oligomers of antisense polarity can be potent inducers of gene silencing. As is the case for co-suppression, they showed that antisense RNAs act independently of the RNAi genes rde-1 and rde-4 but require the mutator/RNAi gene mut-7 and a putative DEAD box RNA helicase, mut-14. According to the authors, their data favor the hypothesis that gene silencing is accomplished by RNA primer extension using the mRNA as template, leading to dsRNA that is subsequently degraded suggesting that single-stranded RNA oligomers are ultimately responsible for the RNAi phenomenon (Tijsterman et al., Science, 2002, 295, 694-697).

Several recent publications have described the structural requirements for the dsRNA trigger required for RNAi activity. Recent reports have indicated that ideal dsRNA sequences are 21 nt in length containing 2 nt 3′-end overhangs (Elbashir et al, EMBO (2001), 20, 6877-6887, Sabine Brantl, Biochimica et Biophysica Acta, 2002, 1575, 15-25.) In this system, substitution of the 4 nucleosides from the 3′-end with 2′-deoxynucleosides has been demonstrated to not affect activity. On the other hand, substitution with 2′-deoxynucleosides or 2′-OMe-nucleosides throughout the sequence (sense or antisense) was shown to be deleterious to RNAi activity.

Investigation of the structural requirements for RNA silencing in C. elegans has demonstrated modification of the internucleotide linkage (phosphorothioate) to not interfere with activity (Parrish et al., Molecular Cell, 2000, 6, 1077-1087.) It was also shown by Parrish et al., that chemical modification like 2′-amino or 5-iodouridine are well tolerated in the sense strand but not the antisense strand of the dsRNA suggesting differing roles for the 2 strands in RNAi. Base modification such as guanine to inosine (where one hydrogen bond is lost) has been demonstrated to decrease RNAi activity independently of the position of the modification (sense or antisense). Some “position independent” loss of activity has been observed following the introduction of mismatches in the dsRNA trigger. Some types of modifications, for example introduction of sterically demanding bases such as 5-iodoU, have been shown to be deleterious to RNAi activity when positioned in the antisense strand, whereas modifications positioned in the sense strand were shown to be less detrimental to RNAi activity. As was the case for the 21 nt dsRNA sequences, RNA-DNA heteroduplexes did not serve as triggers for RNAi. However, dsRNA containing 2′-F-2′-deoxynucleosides appeared to be efficient in triggering RNAi response independent of the position (sense or antisense) of the 2′-F-2′-deoxynucleosides.

In one study the reduction of gene expression was studied using electroporated dsRNA and a 25mer morpholino oligomer in post implantation mouse embryos (Mellitzer et al., Mehanisms of Development, 2002, 118, 57-63). The morpholino oligomer did show activity but was not as effective as the dsRNA.

A number of PCT applications have recently been published that relate to the RNAi phenomenon. These include: PCT publication WO 00/44895; PCT publication WO 00/49035; PCT publication WO 00/63364; PCT publication WO 01/36641; PCT publication WO 01/36646; PCT publication WO 99/32619; PCT publication WO 00/44914; PCT publication WO 01/29058; and PCT publication WO 01/75164.

U.S. Pat. Nos. 5,898,031 and 6,107,094, each of which is commonly owned with this application and each of which is herein incorporated by reference, describe certain oligonucleotide having RNA like properties. When hybridized with RNA, these oligonucleotides serve as substrates for a dsRNase enzyme with resultant cleavage of the RNA by the enzyme.

In another recently published paper (Martinez et al., Cell, 2002, 110, 563-574) it was shown that single stranded as well as double stranded siRNA resides in the RNA-induced silencing complex (RISC) together with elF2C1 and elf2C2 (human GERp950) Argonaute proteins. The activity of 5′-phosphorylated single stranded siRNA was comparable to the double stranded siRNA in the system studied. In a related study, the inclusion of a 5′-phosphate moiety was shown to enhance activity of siRNA's in vivo in Drosophilia embryos (Boutla, et al., Curr. Biol., 2001, 11, 1776-1780). In another study, it was reported that the 5′-phosphate was required for siRNA function in human HeLa cells (Schwarz et al., Molecular Cell, 2002, 10, 537-548).

In yet another recently published paper (Chiu et al., Molecular Cell, 2002, 10, 549-561) it was shown that the 5′-hydroxyl group of the siRNA is essential as it is phosphorylated for activity while the 3′-hydroxyl group is not essential and tolerates substitute groups such as biotin. It was further shown that bulge structures in one or both of the sense or antisense strands either abolished or severely lowered the activity relative to the unmodified siRNA duplex. Also shown was severe lowering of activity when psoralen was used to cross link an siRNA duplex.

Like the RNAse H pathway, the RNA interference pathway for modulation of gene expression is an effective means for modulating the levels of specific gene products and, thus, would be useful in a number of therapeutic, diagnostic, and research applications involving gene silencing. The present invention therefore provides oligomeric compounds useful for modulating gene expression pathways, including those relying on mechanisms of action such as RNA interference and dsRNA enzymes, as well as antisense and non-antisense mechanisms. One having skill in the art, once armed with this disclosure will be able, without undue experimentation, to identify preferred oligonucleotide compounds for these uses.

### SUMMARY OF THE INVENTION

In certain aspects, the invention relates to oligomer compositions comprising a first oligomer and a second oligomer in which at least a portion of the first oligomer is capable of hybridizing with at least a portion of the second oligomer, and at least a portion of the first oligomer is complementary to and capable of hybridizing to a selected target nucleic acid. At least one of the first or second oligomers includes at least one A and G modified binding base.

In certain other embodiments, the invention is directed to oligonucleotide/protein compositions comprising an oligomer complementary to and capable of hybridizing to a selected target nucleic acid, and at least one protein comprising at least a portion of a RNA-induced silencing complex (RISC). The oligomer includes at least one A and G modified binding base.

In other aspects, the invention relates to oligomers having at least a first region and a second region where the first region of the oligomer is complementary to and is capable of hybridizing with the second region of the oligomer, and at least a portion of the oligomer is complementary to and is capable of hybridizing to a selected target nucleic acid. The oligomer further includes at least one A and G modified binding base.

Also provided by the present invention are pharmaceutical compositions comprising any of the above compositions or oligomeric compounds and a pharmaceutically acceptable carrier.

Methods for modulating the expression of a target nucleic acid in a cell are also provided, wherein the methods comprise contacting the cell with any of the above compositions or oligomeric compounds.

Methods of treating or preventing a disease or condition associated with a target nucleic acid are also provided, wherein the methods comprise administering to a patient having or predisposed to the disease or condition a therapeutically effective amount of any of the above compositions or oligomeric compounds.

### DETAILED DESCRIPTION OF THE INVENTION

The present invention provides oligomeric compounds useful in the modulation of gene expression. Although not intending to be bound by theory, oligomeric compounds of the invention are believed to modulate gene expression by hybridizing to a nucleic acid target resulting in loss of normal function of the target nucleic acid. As used herein, the term “target nucleic acid” or “nucleic acid target” is used for convenience to encompass any nucleic acid capable of being targeted including without limitation DNA, RNA (including pre-mRNA and mRNA or portions thereof) transcribed from such DNA, and also cDNA derived from such RNA. In a preferred embodiment of this invention modulation of gene expression is effected via modulation of a RNA associated with the particular gene RNA.

The invention provides for modulation of a target nucleic acid that is a messenger RNA. The messenger RNA is degraded by the RNA interference mechanism as well as other mechanisms in which double stranded RNA/RNA structures are recognized and degraded, cleaved or otherwise rendered inoperable.

The functions of RNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. In the context of the present invention, “modulation” and “modulation of expression” mean either an increase (stimulation) or a decrease (inhibition) in the amount or levels of a nucleic acid molecule encoding the gene, e.g., DNA or RNA. Inhibition is often the preferred form of modulation of expression and mRNA is often a preferred target nucleic acid.

Compounds of the Invention

In certain aspects, the invention relates to oligomeric compounds that comprise at least one nucleotide containing a modified base. These modified bases are bases that will bind or hybridize to either an “A” base, i.e., an adenine base on an adenosine nucleotide, or a “G” bases, i.e., a guanine base on a guanosine nucleotide. Since these modified bases will bind to either an A base or a G base, for the purposes of this specification and the claims attached hereto the modified bases of the invention are identified as “A and G modified binding bases. Binding is meant in a Watson/Crick, Hoogsteen or reverse Hoogsteen like sense wherein one or more hydrogen bonds are formed between two bases forming a pair of complementary bases.

Excluded from the definition of A and G modified binding bases are the three natural pyrimidine bases T (thymine), U (uracil) and C (cytosine). While the T, U and C bases bind to the A and G bases via hydrogen bonds in Watson/Crick type binding, they are not modified but exist in their natural form. Thus they are not A and G modified binding bases.

For the purposes of this specification and the claims attached thereto, A and G modified binding bases include synthetic or natural modified pyrimidine bases, extended pyrimidine bases, pyrimidine bases that are joined to sugar moieties in nucleotides via a carbon atom, i.e., C-pyrimidine base, six membered heterocyclic rings having 1, 2 or 3 nitrogen atoms in the ring and certain bases known in the art as universal bases.

Modified pyrimidine bases include 3-deaza pyrimidines, 1-deaza-pyrimidines, 5-aza-pyrimidines, 6-aza-pyrimidines, 3-deaza-5-aza-pyrimidines, 3-deaza-6-aza-pyrimidines, 1-deaza-5-aza-pyrimidines, 1-deaza-6-aza-pyrimidines, 5,6-diaza-pyrimidines, 2-substituted-pyrimidines, 4-substituted-pyrimidines, 3-N-substituted-pyrimidines, 5-substituted-pyrimidines, 6-substituted-pyrimidines, 5,6-disubstituted-pyrimidines or combinations thereof. These and other modified pyrimidine bases have been described in the art and identified in greater detail below.

Extended pyrimidines include ring systems having two or three rings in the system that include a pyrimidine or a modified pyrimidine as one of the rings of the ring system. Extended pyrimidines also include multiple ring systems wherein a pryimidine ring is covalently bonded to a further single ring or to multiple rings via a covalent bond between the pryimidine ring and the other ring or multiple ring or via a a linker extending from the pyrimidine ring to the other ring or multiple rings. These ring systems may also include one or more linear side groups that extend from the ring system much like a tail. One such extended pyrimidine includes a ring system having a “tail” is known in the art as a “G clamp.” It comprisese three rings, one of which is a pryimidine ring, that has a linear side chain that terminates in with an amino group. This “extended pyrimidine” is capable of forming four hydrogen bonds to a guanidine ring on an opposing stand. These and other extended pyrimidine bases have been described in the art and identified in greater detail below.

Pyrimidine bases that are joined to sugar via a carbon atom in the pyrimidine ring (as opposed to the N-1 nitrogen atom) are known in the art as C-pyrimidines. They include pyrimidines jointed to ribo sugar via the C-5 carbon atom of the pryimidine ring. Various C-pyrimidine bases have been described in the art and are identified in greater detail below.

Six-membered heterocyclic rings having 1, 2 or 3 nitrogen atoms in the ring include 1,3,5-triazole, i.e., 5-aza-pyrimidines, 1,3,6-triazole, i.e., 5-aza-pyrimidine, 1,4-diazole, i.e., 3-deaza-4-aza-pyrimidines as well as other 6 membered ring nitrogen contain ring systems. Various six membered heterocyclic rings having 1, 2 or 3 nitrogen atoms have been described in the art and are identified in greater detail below.

Certain bases are known in the art as universal bases. While they can bind to a base in an opposing strand in, as for instance, an opposing base of a Watson/Crick base pair, their scaffold or core ring systems is not a pyrimdine ring. Various universal bases have been described in the art and are identified in greater detail below.

Preferred compounds that comprise A and G modified binding bases include, but are not limited to, boronated pyrimidine bases; C-2 and C-4 modified pyrimidine bases, 3-deazauracil and 3-deazacytosine, pryimidine bases containing C4 sutstituted with a reactive group that is derivatizable with a detectable label; C5 and C6 modified or C5/C6 bismodified wherein the modifications include halo, alkyl, aza, amino, cationic moieties, detectable labels or other modifications. Further preferred compounds that comprise A and G modified binding bases include tricyclic modified pyrimidine bases and pyrimidines that include polycyclic aromatic groups.

Hybridization

In the context of this invention, “hybridization” means the pairing of complementary strands of oligomeric compounds. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases that pair through the formation of hydrogen bonds. Hybridization can occur under varying circumstances.

An oligomeric compound of the invention is believed to specifically hybridize to the target nucleic acid and interfere with its normal function to cause a loss of activity. There is preferably a sufficient degree of complementarity to avoid non-specific binding of the oligomeric compound to non-target nucleic acid sequences under conditions in which specific binding is desired, i.e., under physiological conditions in the case of in vivo assays or therapeutic treatment, and under conditions in which assays are performed in the case of in vitro assays.

In the context of the present invention the phrase “stringent hybridization conditions” or “stringent conditions” refers to conditions under which an oligomeric compound of the invention will hybridize to its target sequence, but to a minimal number of other sequences. Stringent conditions are sequence-dependent and will vary with different circumstances and in the context of this invention; “stringent conditions” under which oligomeric compounds hybridize to a target sequence are determined by the nature and composition of the oligomeric compounds and the assays in which they are being investigated.

“Complementary,” as used herein, refers to the capacity for precise pairing of two nucleobases regardless of where the two are located. For example, if a nucleobase at a certain position of an oligomeric compound is capable of hydrogen bonding with a nucleobase at a certain position of a target nucleic acid, then the position of hydrogen bonding between the oligonucleotide and the target nucleic acid is considered to be a complementary position. The oligomeric compound and the target nucleic acid are complementary to each other when a sufficient number of complementary positions in each molecule are occupied by nucleobases that can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

It is understood in the art that the sequence of the oligomeric compound need not be 100% complementary to that of its target nucleic acid to be specifically hybridizable. Moreover, an oligomeric compound may hybridize over one or more segments such that intervening or adjacent segments are not involved in the hybridization event (e.g., a loop structure or hairpin structure). It is preferred that the oligomeric compounds of the present invention comprise at least 70% sequence complementarity to a target region within the target nucleic acid, more preferably that they comprise 90% sequence complementarity and even more preferably comprise 95% sequence complementarity to the target region within the target nucleic acid sequence to which they are targeted. For example, an oligomeric compound in which 18 of 20 nucleobases of the oligomeric compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an oligomeric compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an oligomeric compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

Targets of the invention

“Targeting” an oligomeric compound to a particular nucleic acid molecule, in the context of this invention, can be a multistep process. The process usually begins with the identification of a target nucleic acid whose function is to be modulated. This target nucleic acid may be, for example, a mRNA transcribed from a cellular gene whose expression is associated with a particular disorder or disease state, or a nucleic acid molecule from an infectious agent.

The targeting process usually also includes determination of at least one target region, segment, or site within the target nucleic acid for the interaction to occur such that the desired effect, e.g., modulation of expression, will result. Within the context of the present invention, the term “region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the present invention, are defined as positions within a target nucleic acid. The terms region, segment, and site can also be used to describe an oligomeric compound of the invention such as for example a gapped oligomeric compound having 3 separate segments.

Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA transcribed from a gene encoding a nucleic acid target, regardless of the sequence(s) of such codons. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively).

The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with the antisense oligomeric compounds of the present invention.

The open reading frame (ORF) or “coding region,” which is known in the art to refer to the region between the translation initiation codon and the translation termination codon, is also a region which may be targeted effectively. Within the context of the present invention, a preferred region is the intragenic region encompassing the translation initiation or termination codon of the open reading frame (ORF) of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. It is also preferred to target the 5′ cap region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. Targeting splice sites, i.e., intron-exon junctions or exon-intron junctions, may also be particularly useful in situations where aberrant splicing is implicated in disease, or where an overproduction of a particular splice product is implicated in disease. Aberrant fusion junctions due to rearrangements or deletions are also preferred target sites. mRNA transcripts produced via the process of splicing of two (or more) mRNAs from different gene sources are known as “fusion transcripts”. It is also known that introns can be effectively targeted using oligomeric compounds targeted to, for example, pre-mRNA.

It is also known in the art that alternative RNA transcripts can be produced from the same genomic region of DNA. These alternative transcripts are generally known as “variants”. More specifically, “pre-mRNA variants” are transcripts produced from the same genomic DNA that differ from other transcripts produced from the same genomic DNA in either their start or stop position and contain both intronic and exonic sequences.

Upon excision of one or more exon or intron regions, or portions thereof during splicing, pre-mRNA variants produce smaller “mRNA variants”. Consequently, mRNA variants are processed pre-mRNA variants and each unique pre-mRNA variant must always produce a unique mRNA variant as a result of splicing. These mRNA variants are also known as “alternative splice variants”. If no splicing of the pre-mRNA variant occurs then the pre-mRNA variant is identical to the mRNA variant.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. Within the context of the invention, the types of variants described herein are also preferred target nucleic acids.

The locations on the target nucleic acid to which preferred compounds and compositions of the invention hybridize are herein below referred to as “preferred target segments.” As used herein the term “preferred target segment” is defined as at least an 8-nucleobase portion of a target region to which an active antisense oligomeric compound is targeted. While not wishing to be bound by theory, it is presently believed that these target segments represent portions of the target nucleic acid that are accessible for hybridization.

Once one or more target regions, segments or sites have been identified, oligomeric compounds are chosen which are sufficiently complementary to the target, i.e., hybridize sufficiently well and with sufficient specificity, to give the desired effect.

In accordance with an embodiment of the this invention, a series of nucleic acid duplexes comprising the antisense strand oligomeric compounds of the present invention and their respective complement sense strand compounds can be designed for a specific target or targets. The ends of the strands may be modified by the addition of one or more natural or modified nucleobases to form an overhang. The sense strand of the duplex is designed and synthesized as the complement of the antisense strand and may also contain modifications or additions to either terminus. For example, in one embodiment, both strands of the duplex would be complementary over the central nucleobases, each having overhangs at one or both termini.

For the purposes of describing an embodiment of this invention, the combination of an antisense strand and a sense strand, each of can be of a specified length, for example from 18 to 29 nucleotides (or nucleosidic bases) long, is identified as a complementary pair of siRNA oligonucleotides. This complementary pair of siRNA oligonucleotides can include additional nucleotides on either of their 5′ or 3′ ends. Further they can include other molecules or molecular structures on their 3′ or 5′ ends such as a phosphate group on the 5′ end. A preferred group of compounds of the invention include a phosphate group on the 5′ end of the antisense strand compound. Other preferred compounds also include a phosphate group on the 5′ end of the sense strand compound. Even further preferred compounds would include additional nucleotides such as a two base overhang on the 3′ end.

For example, a preferred siRNA complementary pair of oligonucleotides comprise an antisense strand oligomeric compound having the sequence CGAGAGGCGGACGGGACCG (SEQ ID NO:1) and having a two-nucleobase overhang of deoxythymidine(dT) and its complement sense strand. These oligonucleotides would have the following structure:

| | | |
|---|---|---|
| 5′ cgagaggcggacgggaccgTT 3′ | Antisense Strand | (SEQ ID NO: 2) |
| \|\|\|\|\|\|\|\|\|\|\|\|\|\|\|\|\|\|\| | | |
| 3′ TTgctctccgcctgccctggc 5′ | Complement Strand | (SEQ ID NO: 3) |

In an additional embodiment of the invention, a single oligonucleotide having both the antisense portion as a first region in the oligonucleotide and the sense portion as a second region in the oligonucleotide is selected. The first and second regions are linked together by either a nucleotide linker (a string of one or more nucleotides that are linked together in a sequence) or by a non-nucleotide linker region or by a combination of both a nucleotide and non-nucleotide structure. In each of these structures, the oligonucleotide, when folded back on itself, would be complementary at least between the first region, the antisense portion, and the second region, the sense portion. Thus the oligonucleotide would have a palindrome within it structure wherein the first region, the antisense portion in the 5′ to 3′ direction, is complementary to the second region, the sense portion in the 3′ to 5′ direction.

In a further embodiment, the invention includes oligonucleotide/protein compositions. Such compositions have both an oligonucleotide component and a protein component. The oligonucleotide component comprises at least one oligonucleotide, either the antisense or the sense oligonucleotide but preferably the antisense oligonucleotide (the oligonucleotide that is antisense to the target nucleic acid). The oligonucleotide component can also comprise both the antisense and the sense strand oligonucleotides. The protein component of the composition comprises at least one protein that forms a portion of the RNA-induced silencing complex, i.e., the RISC complex.

RISC is a ribonucleoprotein complex that contains an oligonucleotide component and proteins of the Argonaute family of proteins, among others. While we do not wish to be bound by theory, the Argonaute proteins make up a highly conserved family whose members have been implicated in RNA interference and the regulation of related phenomena. Members of this family have been shown to possess the canonical PAZ and Piwi domains, thought to be a region of protein-protein interaction. Other proteins containing these domains have been shown to effect target cleavage, including the RNAse, Dicer. The Argonaute family of proteins includes, but depending on species, are not necessary limited to, elF2C1 and elF2C2. elF2C2 is also known as human GERp95. While we do not wish to be bound by theory, at least the antisense oligonucleotide strand is bound to the protein component of the RISC complex. Additionally, the complex might also include the sense strand oligonucleotide. Carmell et al, Genes and Development 2002, 16, 2733-2742.

Also, while we do not wish to be bound by theory, it is further believe that the RISC complex may interact with one or more of the translation machinery components. Translation machinery components include but are not limited to proteins that effect or aid in the translation of an RNA into protein including the ribosomes or polyribosome complex. Therefore, in a further embodiment of the invention, the oligonucleotide component of the invention is associated with a RISC protein component and further associates with the translation machinery of a cell. Such interaction with the translation machinery of the cell would include interaction with structural and enzymatic proteins of the translation machinery including but not limited to the polyribosome and ribosomal subunits.

In a further embodiment of the invention, the oligonucleotide of the invention is associated with cellular factors such as transporters or chaperones. These cellular factors can be protein, lipid or carbohydrate based and can have structural or enzymatic functions that may or may not require the complexation of one or more metal ions.

Furthermore, the oligonucleotide of the invention itself may have one or more moieties which are bound to the oligonucleotide which facilitate the active or passive transport, localization or compartmentalization of the oligonucleotide. Cellular localization includes, but is not limited to, localization to within the nucleus, the nucleolus or the cytoplasm. Compartmentalization includes, but is not limited to, any directed movement of the oligonucleotides of the invention to a cellular compartment including the nucleus, nucleolus, mitochondrion, or imbedding into a cellular membrane surrounding a compartment or the cell itself.

In a further embodiment of the invention, the oligonucleotide of the invention is associated with cellular factors that affect gene expression, more specifically those involved in RNA modifications. These modifications include, but are not limited to posttrascriptional modifications such as methylation. Furthermore, the oligonucleotide of the invention itself may have one or more moieties which are bound to the oligonucleotide which facilitate the posttranscriptional modification.

The oligomeric compounds of the invention may be used in the form of single-stranded, double-stranded, circular or hairpin oligomeric compounds and may contain structural elements such as internal or terminal bulges or loops. Once introduced to a system, the oligomeric compounds of the invention may interact with or elicit the action of one or more enzymes or may interact with one or more structural proteins to effect modification of the target nucleic acid.

One non-limiting example of such an interaction is the RISC complex. Use of the RISC complex to effect cleavage of RNA targets thereby greatly enhances the efficiency of oligonucleotide-mediated inhibition of gene expression. Similar roles have been postulated for other ribonucleases such as those in the RNase III and ribonuclease L family of enzymes.

Preferred forms of oligomeric compound of the invention include a single-stranded antisense oligonucleotide that binds in a RISC complex, a double stranded antisense/sense pair of oligonucleotide or a single strand oligonucleotide that includes both an antisense portion and a sense portion. Each of these compounds or compositions is used to induce potent and specific modulation of gene function. Such specific modulation of gene function has been shown in many species by the introduction of double-stranded structures, such as double-stranded RNA (dsRNA) molecules and has been shown to induce potent and specific antisense-mediated reduction of the function of a gene or its associated gene products. This phenomenon occurs in both plants and animals and is believed to have an evolutionary connection to viral defense and transposon silencing.

The compounds and compositions of the invention are used to modulate the expression of a target nucleic acid. “Modulators” are those oligomeric compounds that decrease or increase the expression of a nucleic acid molecule encoding a target and which comprise at least an 8-nucleobase portion that is complementary to a preferred target segment. The screening method comprises the steps of contacting a preferred target segment of a nucleic acid molecule encoding a target with one or more candidate modulators, and selecting for one or more candidate modulators which decrease or increase the expression of a nucleic acid molecule encoding a target. Once it is shown that the candidate modulator or modulators are capable of modulating (e.g. either decreasing or increasing) the expression of a nucleic acid molecule encoding a target, the modulator may then be employed in further investigative studies of the function of a target, or for use as a research, diagnostic, or therapeutic agent in accordance with the present invention.

Oligomeric Compounds

In the context of the present invention, the term “oligomeric compound” or oligomer refers to a polymeric structure capable of hybridizing a region of a nucleic acid molecule. This term includes oligonucleotides, oligonucleosides, oligonucleotide analogs, oligonucleotide mimetics and combinations of these. Oligomeric compounds are routinely prepared linearly but can be joined or otherwise prepared to be circular, and may also include branching. Oligomeric compounds can hybridized to form double stranded compounds that can be blunt ended or may include overhangs. In general an oligomeric compound comprises a backbone of linked momeric subunits where each linked momeric subunit is directly or indirectly attached to a heterocyclic base moiety. The linkages joining the monomeric subunits, the sugar moieties or surrogates and the heterocyclic base moieties can be independently modified giving rise to a plurality of motifs for the resulting oligomeric compounds including hemimers, gapmers and chimeras.

As is known in the art, a nucleoside is a base-sugar combination. The base portion of the nucleoside is normally a heterocyclic base moiety. The two most common classes of such heterocyclic bases are purines and pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. The respective ends of this linear polymeric structure can be joined to form a circular structure by hybridization or by formation of a covalent bond, however, open linear structures are generally preferred. Within the oligonucleotide structure, the phosphate groups are commonly referred to as forming the internucleoside linkages of the oligonucleotide. The normal internucleoside linkage of RNA and DNA is a 3′ to 5′ phosphodiester linkage.

In the context of this invention, the term “oligonucleotide” refers to an oligomer or polymer of ribonucleic acid (RNA) or deoxyribonucleic acid (DNA). This term includes oligonucleotides composed of naturally-occurring nucleobases, sugars and covalent internucleoside linkages. The term “oligonucleotide analog” refers to oligonucleotides that have one or more non-naturally occurring portions which function in a similar manner to oligonulceotides. Such non-naturally occurring oligonucleotides are often preferred over the naturally occurring forms because of desirable properties such as, for example, enhanced cellular uptake, enhanced affinity for nucleic acid target and increased stability in the presence of nucleases.

In the context of this invention, the term “oligonucleoside” refers to nucleosides that are joined by internucleoside linkages that do not have phosphorus atoms. Internucleoside linkages of this type include short chain alkyl, cycloalkyl, mixed heteroatom alkyl, mixed heteroatom cycloalkyl, one or more short chain heteroatomic and one or more short chain heterocyclic. These internucleoside linkages include but are not limited to siloxane, sulfide, sulfoxide, sulfone, acetal, formacetal, thioformacetal, methylene formacetal, thioformacetal, alkeneyl, sulfamate; methyleneimino, methylenehydrazino, sulfonate, sulfonamide, amide and others having mixed N, O, S and CH₂ component parts.

In addition to the modifications described above, the nucleosides of the oligomeric compounds of the invention can have a variety of other modifications so long as these other modifications either alone or in combination with other nucleosides enhance one or more of the desired properties described above. Thus, for nucleotides that are incorporated into oligonucleotides of the invention, these nucleotides can have sugar portions that correspond to naturally-occurring sugars or modified sugars. Representative modified sugars include carbocyclic or acyclic sugars, sugars having substituent groups at one or more of their 2′, 3′ or 4′ positions and sugars having substituents in place of one or more hydrogen atoms of the sugar. Additional nucleosides amenable to the present invention having altered base moieties and or altered sugar moieties are disclosed in U.S. Pat. No. 3,687,808 and PCT application PCT/US89/02323.

Altered base moieties or altered sugar moieties also include other modifications consistent with the spirit of this invention. Such oligonucleotides are best described as being structurally distinguishable from, yet functionally interchangeable with, naturally occurring or synthetic wild type oligonucleotides. All such oligonucleotides are comprehended by this invention so long as they function effectively to mimic the structure of a desired RNA or DNA strand. A class of representative base modifications include tricyclic cytosine analog, termed “G clamp” (Lin, et al., J. Am. Chem. Soc. 1998, 120, 8531). This analog makes four hydrogen bonds to a complementary guanine (G) within a helix by simultaneously recognizing the Watson-Crick and Hoogsteen faces of the targeted G. This G clamp modification when incorporated into phosphorothioate oligonucleotides, dramatically enhances antisense potencies in cell culture. The oligonucleotides of the invention also can include phenoxazine-substituted bases of the type disclosed by Flanagan, et al., Nat. Biotechnol. 1999, 17(1), 48-52.

The oligomeric compounds in accordance with this invention preferably comprise from about 8 to about 80 nucleobases (i.e. from about 8 to about 80 linked nucleosides). One of ordinary skill in the art will appreciate that the invention embodies oligomeric compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length.

In one preferred embodiment, the oligomeric compounds of the invention are 12 to 50 nucleobases in length. One having ordinary skill in the art will appreciate that this embodies oligomeric compounds of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobases in length.

In another preferred embodiment, the oligomeric compounds of the invention are 15 to 30 nucleobases in length. One having ordinary skill in the art will appreciate that this embodies oligomeric compounds of 15, 16, 17, 18, 19, 20, 21, 22, 23, 24, 25, 26, 27, 28, 29, or 30 nucleobases in length.

Particularly preferred oligomeric compounds are oligonucleotides from about 15 to about 30 nucleobases, even more preferably those comprising from about 21 to about 24 nucleobases.

General Oligomer Synthesis

Oligomerization of modified and unmodified nucleosides is performed according to literature procedures for DNA-like compounds (Protocols for Oligonucleotides and Analogs, Ed. Agrawal (1993), Humana Press) and/or RNA like compounds (Scaringe, Methods (2001), 23, 206-217. Gait et al., Applications of Chemically synthesized RNA in RNA:Protein Interactions, Ed. Smith (1998), 1-36. Gallo et al., Tetrahedron (2001), 57, 5707-5713) synthesis as appropriate. In addition specific protocols for the synthesis of oligomeric compounds of the invention are illustrated in the examples below.

RNA oligomers can be synthesized by methods disclosed herein or purchased from various RNA synthesis companies such as for example Dharmacon Research Inc., (Lafayette, Colo.).

Irrespective of the particular protocol used, the oligomeric compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed.

For double stranded structures of the invention, once synthesized, the complementary strands preferably are annealed. The single strands are aliquoted and diluted to a concentration of 50 uM. Once diluted, 30 uL of each strand is combined with 15 uL of a 5× solution of annealing buffer. The final concentration of the buffer is 100 mM potassium acetate, 30 mM HEPES-KOH pH 7.4, and 2 mM magnesium acetate. The final volume is 75 uL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA compound is 20 uM. This solution can be stored frozen (−20° C.) and freeze-thawed up to 5 times.

Once prepared, the desired synthetic duplexes are evaluated for their ability to modulate target expression. When cells reach 80% confluency, they are treated with synthetic duplexes comprising at least one oligomeric compound of the invention. For cells grown in 96-well plates, wells are washed once with 200 μL OPTI-MEM-1 reduced-serum medium (Gibco BRL) and then treated with 130 μL of OPTI-MEM-1 containing 12 μg/mL LIPOFECTIN (Gibco BRL) and the desired dsRNA compound at a final concentration of 200 nM. After 5 hours of treatment, the medium is replaced with fresh medium. Cells are harvested 16 hours after treatment, at which time RNA is isolated and target reduction measured by RT-PCR.

Oligomer and Monomer Modifications

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally preferred. In addition, linear compounds may have internal nucleobase complementarity and may therefore fold in a manner as to produce a fully or partially double-stranded compound. Within oligonucleotides, the phosphate groups are commonly referred to as forming the internucleoside linkage or in conjunction with the sugar ring the backbone of the oligonucleotide. The normal internucleoside linkage that makes up the backbone of RNA and DNA is a 3′ to 5′ phosphodiester linkage.

Modified Internucleoside Linkages

Specific examples of preferred antisense oligomeric compounds useful in this invention include oligonucleotides containing modified e.g. non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides.

In the C. elegans system, modification of the internucleotide linkage (phosphorothioate) did not significantly interfere with RNAi activity. Based on this observation, it is suggested that certain preferred oligomeric compounds of the invention can also have one or more modified internucleoside linkages. A preferred phosphorus containing modified internucleoside linkage is the phosphorothioate internucleoside linkage.

Preferred modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

In more preferred embodiments of the invention, oligomeric compounds have one or more phosphorothioate and/or heteroatom internucleoside linkages, in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester internucleotide linkage is represented as —O—P(═O)(OH)—O—CH₂—]. The MMI type internucleoside linkages are disclosed in the above referenced U.S. Pat. No. 5,489,677. Preferred amide internucleoside linkages are disclosed in the above referenced U.S. Pat. No. 5,602,240.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

Oligomer Mimetics

Another preferred group of oligomeric compounds amenable to the present invention includes oligonucleotide mimetics. The term mimetic as it is applied to oligonucleotides is intended to include oligomeric compounds wherein only the furanose ring or both the furanose ring and the internucleotide linkage are replaced with novel groups, replacement of only the furanose ring is also referred to in the art as being a sugar surrogate. The heterocyclic base moiety or a modified heterocyclic base moiety is maintained for hybridization with an appropriate target nucleic acid. One such oligomeric compound, an oligonucleotide mimetic that has been shown to have excellent hybridization properties, is referred to as a peptide nucleic acid (PNA). In PNA oligomeric compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA oligomeric compounds include, but are not limited to, U.S. Pat. Nos. 5,539,082; 5,714,331; and 5,719,262, each of which is herein incorporated by reference. Further teaching of PNA oligomeric compounds can be found in Nielsen et al., Science, 1991, 254, 1497-1500.

One oligonucleotide mimetic that has been reported to have excellent hybridization properties is peptide nucleic acids (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

PNA has been modified to incorporate numerous modifications since the basic PNA structure was first prepared. The basic structure is shown below: 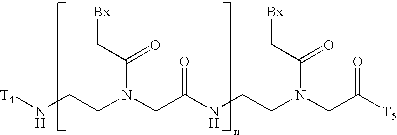
wherein
Bx is a heterocyclic base moiety;
T₄ is hydrogen, an amino protecting group, —C(O)R₅, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, a reporter group, a conjugate group, a D or L α-amino acid linked via the α-carboxyl group or optionally through the co-carboxyl group when the amino acid is aspartic acid or glutamic acid or a peptide derived from D, L or mixed D and L amino acids linked through a carboxyl group, wherein the substituent groups are selected from hydroxyl, amino, alkoxy, carboxy, benzyl, phenyl, nitro, thiol, thioalkoxy, halogen, alkyl, aryl, alkenyl and alkynyl;
T₅ is —OH, —N(Z₁)Z₂, R₅, D or L α-amino acid linked via the α-amino group or optionally through the (ω-amino group when the amino acid is lysine or ornithine or a peptide derived from D, L or mixed D and L amino acids linked through an amino group, a chemical functional group, a reporter group or a conjugate group;
Z₁ is hydrogen, C₁-C₆ alkyl, or an amino protecting group;
Z₂ is hydrogen, C₁-C₆ alkyl, an amino protecting group, —C(═O)—(CH₂)ₙ-J-Z₃, a D or L α-amino acid linked via the α-carboxyl group or optionally through the co-carboxyl group when the amino acid is aspartic acid or glutamic acid or a peptide derived from D, L or mixed D and L amino acids linked through a carboxyl group;
Z₃ is hydrogen, an amino protecting group, —C₁-C₆ alkyl, —C(═O)—CH₃, benzyl, benzoyl, or —(CH₂)ₙ—N(H)Z₁;
each J is O, S or NH;
R₅ is a carbonyl protecting group; and
n is from 2 to about 50.

Another class of oligonucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. A preferred class of linking groups have been selected to give a non-ionic oligomeric compound. The non-ionic morpholino-based oligomeric compounds are less likely to have undesired interactions with cellular proteins. Morpholino-based oligomeric compounds are non-ionic mimics of oligonucleotides which are less likely to form undesired interactions with cellular proteins (Dwaine A. Braasch and David R. Corey, Biochemistry, 2002, 41(14), 4503-4510). Morpholino-based oligomeric compounds are disclosed in U.S. Pat. No. 5,034,506, issued Jul. 23, 1991. The morpholino class of oligomeric compounds have been prepared having a variety of different linking groups joining the monomeric subunits.

Morpholino nucleic acids have been prepared having a variety of different linking groups (L₂) joining the monomeric subunits. The basic formula is shown below: 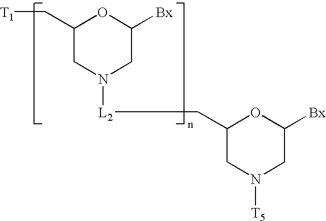
wherein
T₁ is hydroxyl or a protected hydroxyl;
T₅ is hydrogen or a phosphate or phosphate derivative;
L₂ is a linking group; and
n is from 2 to about 50.

A further class of oligonucleotide mimetic is referred to as cyclohexenyl nucleic acids (CeNA). The furanose ring normally present in an DNA/RNA molecule is replaced with a cyclohenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation. Furthermore the incorporation of CeNA into a sequence targeting RNA was stable to serum and able to activate E. Coli RNase resulting in cleavage of the target RNA strand.

The general formula of CeNA is shown below: 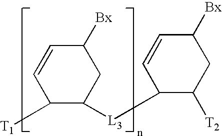
wherein
each Bx is a heterocyclic base moiety;
T₁ is hydroxyl or a protected hydroxyl; and
T2 is hydroxyl or a protected hydroxyl.

Another class of oligonucleotide mimetic (anhydrohexitol nucleic acid) can be prepared from one or more anhydrohexitol nucleosides (see, Wouters and Herdewijn, Bioorg. Med. Chem. Lett., 1999, 9, 1563-1566) and would have the general formula:

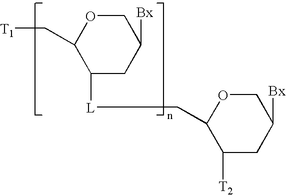

A further preferred modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage is preferably a methylene (—CH₂—)ₙ group bridging the 2′ oxygen atom and the 4′ carbon atom wherein n is 1 or 2 (Singh et al., Chem. Commun., 1998, 4,455-456). LNA and LNA analogs display very high duplex thermal stabilities with complementary DNA and RNA (Tm=+3 to +10 C), stability towards 3′-exonucleolytic degradation and good solubility properties. The basic structure of LNA showing the bicyclic ring system is shown below:

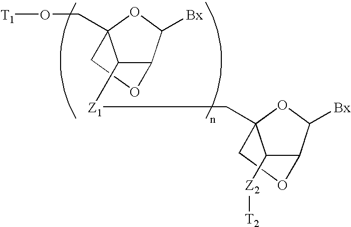

The conformations of LNAs determined by 2D NMR spectroscopy have shown that the locked orientation of the LNA nucleotides, both in single-stranded LNA and in duplexes, constrains the phosphate backbone in such a way as to introduce a higher population of the N-type conformation (Petersen et al., J. Mol. Recognit., 2000, 13, 44-53). These conformations are associated with improved stacking of the nucleobases (Wengel et al., Nucleosides Nucleotides, 1999, 18, 1365-1370).

LNA has been shown to form exceedingly stable LNA:LNA duplexes (Koshkin et al., J. Am. Chem. Soc., 1998, 120, 13252-13253). LNA:LNA hybridization was shown to be the most thermally stable nucleic acid type duplex system, and the RNA-mimicking character of LNA was established at the duplex level. Introduction of 3 LNA monomers (T or A) significantly increased melting points (Tm=+15/+11) toward DNA complements. The universality of LNA-mediated hybridization has been stressed by the formation of exceedingly stable LNA:LNA duplexes. The RNA-mimicking of LNA was reflected with regard to the N-type conformational restriction of the monomers and to the secondary structure of the LNA:RNA duplex.

LNAs also form duplexes with complementary DNA, RNA or LNA with high thermal affinities. Circular dichroism (CD) spectra show that duplexes involving fully modified LNA (esp. LNA:RNA) structurally resemble an A-form RNA:RNA duplex. Nuclear magnetic resonance (NMR) examination of an LNA:DNA duplex confirmed the 3′-endo conformation of an LNA monomer. Recognition of double-stranded DNA has also been demonstrated suggesting strand invasion by LNA. Studies of mismatched sequences show that LNAs obey the Watson-Crick base pairing rules with generally improved selectivity compared to the corresponding unmodified reference strands.

Novel types of LNA-oligomeric compounds, as well as the LNAs, are useful in a wide range of diagnostic and therapeutic applications. Among these are antisense applications, PCR applications, strand-displacement oligomers, substrates for nucleic acid polymerases and generally as nucleotide based drugs.

Potent and nontoxic antisense oligonucleotides containing LNAs have been described (Wahlestedt et al., Proc. Natl. Acad. Sci. U.S.A., 2000, 97, 5633-5638.) The authors have demonstrated that LNAs confer several desired properties to antisense agents. LNA/DNA copolymers were not degraded readily in blood serum and cell extracts. LNA/DNA copolymers exhibited potent antisense activity in assay systems as disparate as G-protein-coupled receptor signaling in living rat brain and detection of reporter genes in Escherichia coli. Lipofectin-mediated efficient delivery of LNA into living human breast cancer cells has also been accomplished.

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

The first analogs of LNA, phosphorothioate-LNA and 2′-thio-LNAs, have also been prepared (Kumar et al., Bioorg. Med. Chem. Lett., 1998, 8, 2219-2222). Preparation of locked nucleoside analogs containing oligodeoxyribonucleotide duplexes as substrates for nucleic acid polymerases has also been described (Wengel et al., PCT International Application WO 98-DK393 19980914). Furthermore, synthesis of 2′-amino-LNA, a novel conformationally restricted high-affinity oligonucleotide analog with a handle has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

Further oligonucleotide mimetics have been prepared to incude bicyclic and tricyclic nucleoside analogs having the formulas (amidite monomers shown): 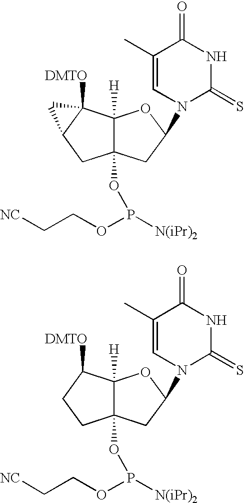
(see Steffens et al., Helv. Chim. Acta, 1997, 80, 2426-2439; Steffens et al., J. Am. Chem. Soc., 1999, 121, 3249-3255; and Renneberg et al., J. Am. Chem. Soc., 2002, 124, 5993-6002). These modified nucleoside analogs have been oligomerized using the phosphoramidite approach and the resulting oligomeric compounds containing tricyclic nucleoside analogs have shown increased thermal stabilities (Tm's) when hybridized to DNA, RNA and itself. Oligomeric compounds containing bicyclic nucleoside analogs have shown thermal stabilities approaching that of DNA duplexes.

Another class of oligonucleotide mimetic is referred to as phosphonomonoester nucleic acids incorporate a phosphorus group in a backbone the backbone. This class of olignucleotide mimetic is reported to have useful physical and biological and pharmacological properties in the areas of inhibiting gene expression (antisense oligonucleotides, ribozymes, sense oligonucleotides and triplex-forming oligonucleotides), as probes for the detection of nucleic acids and as auxiliaries for use in molecular biology.

The general formula (for definitions of variables see: U.S. Pat. Nos. 5,874,553 and 6,127,346 herein incorporated by reference in their entirety) is shown below.

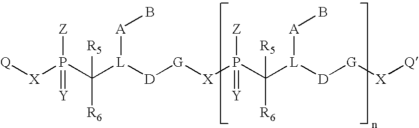

Another oligonucleotide mimetic has been reported wherein the furanosyl ring has been replaced by a cyclobutyl moiety.

Modified Sugars

Oligomeric compounds of the invention may also contain one or more substituted sugar moieties. Preferred oligomeric compounds comprise a sugar substituent group selected from: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other preferred oligonucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, poly-alkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other preferred sugar substituent groups include methoxy (—O—CH₃), aminopropoxy (—OCH₂CH₂CH₂NH₂), allyl(—CH₂—CH═CH₂), —O-allyl(—O—CH₂—CH═CH₂) and fluoro (F). 2′-Sugar substituent groups may be in the arabino (up) position or ribo (down) position. A preferred 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

Further representative sugar substituent groups include groups of formula Iₐ or IIₐ: 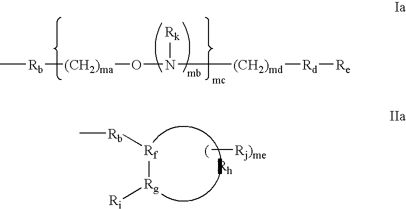
wherein:
R_{b} is O, S or NH;
R_{d} is a single bond, O, S or C(═O);
Rₑ is C₁-C₁₀ alkyl, N(Rₖ)(Rₘ), N(Rₖ)(Rₙ), N═C(Rₚ)(R_{q}), N═C(R)(R) or has formula IIIₐ; 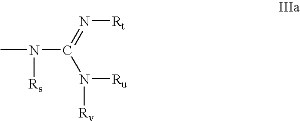
Rₚ and R_{q} are each independently hydrogen or C₁-C₁₀ alkyl;
Rᵣ is —Rₓ—R_{y};
each Rₛ, Rₜ, & and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;
or optionally, Rᵤ and Rᵥ, together form a phthalimido moiety with the nitrogen atom to which they are attached;
each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;
Rₖ is hydrogen, a nitrogen protecting group or —Rₓ—R_{y};
Rₚ is hydrogen, a nitrogen protecting group or —Rₓ—R_{y};
Rₓ is a bond or a linking moiety;
R_{y} is a chemical functional group, a conjugate group or a solid support medium;
each Rₘ and Rₙ is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein the substituent groups are selected from hydroxyl, amino, alkoxy, carboxy, benzyl, phenyl, nitro, thiol, thioalkoxy, halogen, alkyl, aryl, alkenyl, alkynyl; NH₃⁺, N(Rᵤ)(Rᵥ), guanidino and acyl where said acyl is an acid amide or an ester;
or Rₘ and Rₙ, together, are a nitrogen protecting group, are joined in a ring structure that optionally includes an additional heteroatom selected from N and O or are a chemical functional group;
Rᵢ is OR_{z}, SR_{z}, or N(R_{z})₂;
each R_{z} is, independently, H, C₁-C₈ alkyl, C₁-C₈ haloalkyl, C(═NH)N(H)Rᵤ, C(═O)N(H)Rᵤ or OC(═O)N(H)Rᵤ;
R_{f}, R_{g} and Rₕ comprise a ring system having from about 4 to about 7 carbon atoms or having from about 3 to about 6 carbon atoms and 1 or 2 heteroatoms wherein said heteroatoms are selected from oxygen, nitrogen and sulfur and wherein said ring system is aliphatic, unsaturated aliphatic, aromatic, or saturated or unsaturated heterocyclic;
Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ)ORₖ, halo, SRₖ or CN;
mₐ is 1 to about 10;
each mb is, independently, 0 or 1;
mc is 0 or an integer from 1 to 10;
md is an integer from 1 to 10;
me is from 0, 1 or 2; and
provided that when mc is 0, md is greater than 1.

Representative substituents groups of Formula I are disclosed in U.S. patent application Ser. No. 09/130,973, filed Aug. 7, 1998, entitled “Capped 2′-Oxyethoxy Oligonucleotides,” hereby incorporated by reference in its entirety.

Representative cyclic substituent groups of Formula II are disclosed in U.S. patent application Ser. No. 09/123,108, filed Jul. 27, 1998, entitled “RNA Targeted 2′-Oligomeric compounds that are Conformationally Preorganized,” hereby incorporated by reference in its entirety.

Particularly preferred sugar substituent groups include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10.

Representative guanidino substituent groups that are shown in formula III and IV are disclosed in co-owned U.S. patent application Ser. No. 09/349,040, entitled “Functionalized Oligomers”, filed Jul. 7, 1999, hereby incorporated by reference in its entirety.

Representative acetamido substituent groups are disclosed in U.S. Pat. No. 6,147,200 which is hereby incorporated by reference in its entirety.

Representative dimethylaminoethyloxyethyl substituent groups are disclosed in International Patent Application PCT/US99/17895, entitled “2′-O-Dimethylaminoethyloxyethyl-Oligomeric compounds”, filed Aug. 6, 1999, hereby incorporated by reference in its entirety.

Modified Nucleobases/Naturally Occurring Nucleobases

Oligomeric compounds may also include nucleobase (often referred to in the art simply as “base” or “heterocyclic base moiety”) modifications or substitutions. As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl(—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties may also include those in which the pyrimidine base is replaced with an A and G modified binding base, such as those described below.

Boronated pyrimidine bases. In certain embodiments, the invention relates to oligonucleotides comprising at least one boronated pyrimidine base wherein the boron-containing substituent on the pyrimidine base is selected from the group consisting of —BH₂CN, —BH₃, and —BH₂COOR, wherein R is C1 to C₁₋₈ alkyl. Preferably, R is C1 to C9 alkyl, and most preferably R is C1 to C4 alkyl. Such boronated pyrimidine bases are described, for example, in U.S. Pat. No. 5,130,302, hereby incorporated by reference in its entirety. Synthesis of oligonucleotides containing such modified pyrimidine bases is described in Example 228.

C-2 and C-4 modified A and G modified binding bases. In certain other aspects, the invention relates to oligonucleotides comprising at least one nucleotide containing a C-2 and C-4 modified A and G modified binding base of one of the following structures as described, for example, in U.S. Pat. No. 6,060,592, hereby incorporated by reference in its entirety: 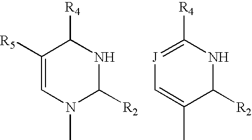
wherein: J is N or CH; R₅ is H or CH₃; one of R₂ and R₄ is ═O, ═NH, or ═NH₂+ or the tautomeric form —OH, —NH₂, —NH₃+; and the other of R₂ and R₄ is Q, ═C(R_{A})-Q, C(R_{A})(R_{B})—C(R_{C})(R_{D})-Q, C(R_{A})═C(R_{C})-Q or C≡C-Q; R_{A}, R_{B}, R_{C} and R_{D}, independently, are H, SH, OH, NH₂, or C₁-C₂₀ alkyl, or one of (R_{A})(R_{B}) or (R_{C})(R_{D}) is ═O; Q is halogen, hydrogen, C₁-C₂₀ alkyl, C₁-C₂₀ alkylamine, C₁-C₂₀ alkyl-N-phthalimide, C₁-C₂₀ alkylimidazole, C₁-C₂₀ alkylbis-imidazole, imidazole, bis-imidazole, amine, N-phthalimide, C₂-C₂₀ alkenyl, C₂-C₂₀ alkynyl, hydroxyl, thiol, keto, carboxyl, nitrates, nitro, nitroso, nitrile, trifluoromethyl, trifluoromethoxy, O-alkyl, S-alkyl, NH-alkyl, N-dialkyl, O-aralkyl, S-aralkyl, NH-aralkyl, azido, hydrazino, hydroxylamino, isocyanato, sulfoxide, sulfone, sulfide, disulfide, silyl, O-(hydroxyl protecting group), a leaving group, a heterocycle, an intercalator, a reporter molecule, a conjugate, a polyamine, a polyamide, a polyethylene glycol, a polyether, a group that enhances the pharmacodynamic properties of oligonucleotides, a group that enhances the pharmacokinetic properties of oligonucleotides, a RNA cleaving moiety or a depurination enhancing group. Synthesis of oligonucleotides containing such A and G modified binding bases is described in Example 229.

A wide variety of protecting groups can be employed in the methods of the invention. See, e.g., Beaucage, et al., Tetrahedron 1992, 12, 2223, hereby incorporated herein by reference in its entirety. In general, protecting groups render chemical functionality inert to specific reaction conditions, and can be appended to and removed from such functionality in a molecule without substantially damaging the remainder of the molecule. Representative hydroxyl protecting groups include t-butyldimethylsilyl (TBDMS), t-butyldiphenylsilyl (TBDPS), dimethoxytrityl (DMTr), monomethoxytrityl (MMTr), and other hydroxyl protecting groups as outlined in the above-noted Beaucage reference.

Leaving groups according to the invention are chemical functional groups that can be displaced from carbon atoms by nucleophilic substitution. Representative leaving groups include, but are not limited to halogen, alkylsulfonyl, substituted alkylsulfonyl, arylsulfonyl, substituted arylsulfonyl, hetercyclcosulfonyl or trichloroacetimidate groups. Preferred leaving groups include chloro, fluoro, bromo, iodo, p-(2,4-dinitroanilino)benzenesulfonyl, benzenesulfonyl, methylsulfonyl (mesylate), p-methylbenzenesulfonyl (tosylate), p-bromobenzenesulfonyl, trifluoromethylsulfonyl (triflate), trichloroacetimidate, acyloxy, 2,2,2-trifluoroethanesulfonyl, imidazolesulfonyl, and 2,4,6-trichlorophenyl groups.

Heterocycles according to the invention are functional groups that include atoms other than carbon in their cyclic backbone.

Intercalators according to the invention generally include non-carcinogenic, polycyclic aromatic hydrocarbons or heterocyclic moieties capable of intercalating between base pairs formed by a hybrid oligonucleotide/RNA target sequence duplex. Intercalators can include naphthalene, anthracene, phenanthrene, benzonaphthalene, fluorene, carbazole, acridine, pyrene, anthraquinone, quinoline, phenylquinoline, xanthene or 2,7-diazaanthracene groups. Other intercalators believed to be useful are described by Denny, Anti-Cancer Drug Design 1989, 4, 241, hereby incorporated herein by reference in its entirety. Another intercalator is the ligand 6-[[[9-[[6-(4-nitrobenzamido)hexyl]amino]acridin-4-yl]carbonyl]-amino]hexa noylpentafluorophenyl ester.

Reporter molecules are those compounds that have physical or chemical properties that allow them to be identified in gels, fluids, whole cellular systems, broken cellular systems and the like utilizing physical properties such as spectroscopy, radioactivity, colorimetric assays, fluorescence, and specific binding. Particularly useful reporter molecules include biotin and fluorescein dyes. Particularly useful as reporter molecules are biotin, fluorescein dyes, alkaline phosphates, and horseradish peroxidase.

The term “depurination enhancing moiety” includes chemical moieties that are capable of enhancing the rate of depurination of a purine-containing nucleic acid species. Depurination enhancing moieties enhance the rate of removal, break down, and/or loss of adenine and guanine nucleobases from adenosine and guanosine nucleotides. They also enhance the rate of the removal, break down, and/or loss of other purine-containing nucleotides such as 7-methylguanosine, 3-methylguanosine, wyosine, inosine, 2-aminoadenosine, and other “minor” or synthetic nucleotides. Preferred depurination enhancing moieties are sulfur-containing compounds, including sulfur-containing heterocycles and both cyclic and alicyclic sulfonium compounds. Specific examples include but are not limited to thiophene, thianthrene, isothiazole, alkyl sulfonium salts, thiophenium salts, 1,3-thiazolium salts, 1,2-oxathiolanium salts, alkyl 1,4-dithianium salts, alkyl thiazolium salts, thioniabicyclo[2,2,1]heptane salts and 3aH-1,6-dithia-6a-thioniapentalene salts. Anions for such salts include halide anions and other anions.

Conjugates are functional groups that improve the uptake of the compounds of the invention. Representative conjugates include steroid molecules, reporter molecules, non-aromatic lipophilic molecules, reporter enzymes, peptides, proteins, water soluble vitamins, and lipid soluble vitamins, as disclosed by U.S. patent application Ser. No. 782,374, filed Oct. 24, 1991, and PCT Application US92/09196, filed Oct. 23, 1992, the disclosures of which are incorporated herein by reference. Representative conjugates also are disclosed by Goodchild, Bioconjugate Chemistry 1990, 1, 165, herby incorporated herein by reference in its entirety.

1,2,6 optionally modified pyrimidine bases. In certain other embodiments, the invention relates to oligonucleotides comprising at least one nucleotide containing a modified pyrimidine base of the following structure as described, for example, in U.S. Pat. Nos. 6,174,998 and 6,320,035, hereby incorporated by reference in their entireties: 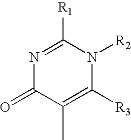
in which R₁, R₂, and R₃ can be same or different and are hydrogen, halogen, hydroxy, thio or substituted thio, amino or substituted amino, carboxy, lower alkyl, lower alkenyl, lower alkinyl, aryl, lower alkyloxy, aryloxy, aralkyl, aralkyloxy or a reporter group. Synthesis of oligonucleotides containing such modified pyrimidine bases is described in Example 230.

C2 modified pyrimidine bases. In certain other embodiments, the invention relates to oligonucleotides comprising at least one nucleotide containing one of the following modified pyrimidine bases: 2-fluoropyridine-3-yl, pyridin-2-one-3-yl, pyridin-2-(4-nitrophenylethyl)-one-3-yl, 2-bromopyridine-5-yl, pyridin-2-one-5-yl, 2-aminopyridine-5-yl, or pyridin-2-(4-nitrophenylethyl)-one-5-yl. Such modified bases are described, for example, in U.S. Pat. No. 6,248,878, hereby incorporated by reference in its entirety. Synthesis of oligonucleotides containing such modified pyrimidine bases is described in Example 231.

3-deazauracil and 3-deazacytosine. In certain other embodiments, the invention relates to oligonucleotides comprising at least one nucleotide containing a 3-deazauracil or 3-deazacytosine analogue of one of the following structures as described, for example, in U.S. Pat. No. 5,134,066, hereby incorporated by reference in its entirety: 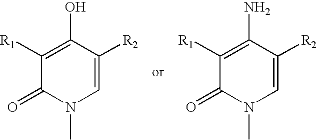
wherein R₁ and R₂, independently, are C₁-C₅ alkyl, C₂-C₅ alkenyl, halo or hydrogen. Synthesis of oligonucleotides containing such modified pyrimidine bases is described in Example 232.

A and G modified binding bases containing C4 substituted with a reactive group derivatizable with a detectable label. In certain other embodiments, the invention relates to oligonucleotides comprising at least one nucleotide containing an A and G modified binding base of the following structure as described, for example, in U.S. Pat. No. 6,268,132, hereby incorporated by reference in its entirety: 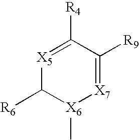
wherein X₅ is N, O, C, S, or Si; X₆ is N or CH, and at least one of X₅ and X₆ is N, and wherein X₇ is —CH—; R₄ is a reactive group derivatizable with a detectable label wherein said reactive group is selected from the group consisting of NH₂, SH, ═O, and optionally, a linking moiety selected from the group consisting of an amide, a thioether, a disulfide, a combination of an amide a thioether or a disulfide, R₁-(CH₂)ₓ—R₂ and R₁—R₂—(CH₂)ₓ—R₃ wherein x is an integer from 1 to 25 inclusive, and R₁, R₂, and R₃ are H, OH, alkyl, acyl, amide, thioether, or disulfide, and wherein said detectable label is selected from the group consisting of radioisotopes, fluorescent or chemiluminescent reporter molecules, antibodies, haptens, biotin, photobiotin, digoxigenin, fluorescent aliphatic amino groups, avidin, enzymes, and acridinium; R₆ is H, NH₂, SH, or ═O; R₉ is hydrogen, methyl, bromine, fluorine, or iodine, alkyl or aromatic substituents, or an optional linking moiety selected from the group consisting of an amide, a thioether, a disulfide linkage, and a combination thereof. Synthesis of oligonucleotides containing such A and G modified binding bases is described in Example 233.

5-substituted cytosine or uracil. In certain aspects, the invention relates to oligonucleotides comprising at least one nucleotide that contains a 5-substituted cytosine or uracil base as described, for example, in U.S. Pat. No. 5,484,908, hereby incorporated by reference in its entirety. In preferred embodiments, the 5-substituted cytosine or uracil is a base of one of the following formulas: 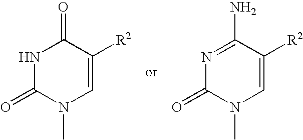
wherein R₂ is selected from the group consisting of propynyl (—C≡C—CH₃), propenyl (—CH═CH—CH₃), 3-buten-1-ynyl (—C≡C—CH═CH₂), 3-methyl-1-butynyl (—C≡C—CH(CH₃)₂), 3,3-dimethyl-1-butynyl (—C≡C—C(CH₃)₃), phenyl, m-pyridinyl, p-pyridinyl and o-pyridinyl. Synthesis of oligonucleotides containing such modified pyrimidine bases is described in Example 234.

5-substituted cytosine or uracil optionally modified at C2 and C4. In certain other embodiments, the 5-substituted cytosine or uracil is a base of one of the following formulas, as described, for example, in U.S. Pat. Nos. 5,645,985 and 6,380,368, hereby incorporated by reference in their entireties: 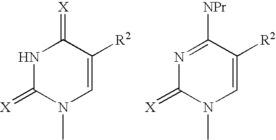
wherein each X is independently O or S; R² is a group comprising at least one pi bond connected to a carbon atom attached to the base; and Pr is (H)₂ or a protecting group. In preferred embodiments, R₂ is selected from the group consisting of vinyl, 1-butenyl, 1-pentenyl, 1-hexenyl, 1-heptenyl, 1-octenyl, 1,3-pentadiynyl, 1-propynyl, 1-butynyl, 1-pentynyl, 3-methyl-1-butynyl, 3,3-dimethyl-1-butynyl, 3-buten-1-ynyl, bromovinyl, 1-hexynyl, 1-heptynyl, 1-octynyl, —C≡C-Z wherein Z is C₁₋₁₀ alkyl or C₁₋₁₀ haloalkyl, a 5-heteroaromatic group, or a 5-(1-alkynyl)-heteroaromatic group; wherein the 5-heteroaromatic group and the 5-(1-alkynyl)-heteroaromatic group are optionally substituted on a ring carbon by oxygen or C₁₋₄ alkyl or are substituted on a ring nitrogen by C₁₋₄ alkyl. Synthesis of oligonucleotides containing such modified pyrimidine bases is described in Example 235.

C5 or C6 modified pyrimidine bases. In certain other aspects, the invention relates to oligonucleotides comprising at least one nucleotide containing a substituted pyrimidine base analogue as described, for example, in U.S. Pat. No. 5,614,617, hereby incorporated by reference in its entirety. Such substitutions may occur at the 5 or 6 position of the pyrimidine ring by substituting a heteroatom for a carbon atom of the pyrimidine ring at these positions. In the alternative, a substituent group can be added to the 5 and 6 positions of the pyrimidine ring.

Substituent groups can be methyl, hydroxyl, alkoxy, alcohol, ester, benzyl, phenyl, nitro, thiol, thioalkoxy, halogen, halocarbon, fused carbon rings or heteroatom containing rings. In accordance with some preferred embodiments of the invention, substitutions of the pyrimidine ring may be aza at the 5 or 6 or both the 5 and 6 position. In accordance with other preferred embodiments of the invention, substituent groups added to the 5 or 6 positions may be one or more of nitro-, methyl-, bromo-, iodo-, chloro-, fluoro-, trifluoro-, trifluoromethyl-2,4-dinitrophenyl-, mercapto-, or methylmercapto-groups. Other preferred substituents are ethers, thioethers, alcohols and thioalcohols such as HS—C—, MeS—C—, OH—C—, MeO—C—, HOCH₂—C—, and cyclopentyl, cyclohexyl and imidazo rings fused to the pyrimidine ring via the 5 and 6 positions of the pyrimidine ring.

Accordingly, some preferred embodiments of this invention may incorporate a modified pyrimidine base or bases having the following structure: 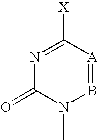
wherein X is OH or NH₂, and A and B may be the same or different and are: C-lower alkyl, N, C—CF₃, C—F, C—Cl, C—Br, C—I, C-halocarbon including C-fluorocarbon, C—NO₂, C—OCF₃, C—SH, C—SCH₃, C—OH, C—O-lower alkyl, C—CH₂OH, C—CH₂SH, C—CH₂SCH₃, C—CH₂OCH₃, C—NH₂, C—CH₂ NH₂, C-alkyl-NH₂, C-benzyl, C-aryl, C-substituted aryl, C-substituted benzyl; or one of A and B are as above and the other is C—H; or together A and B are part of a carbocyclic or heterocyclic ring fused to the pyrimidine ring through A and B. It is preferred that one or both of A and B be C-lower alkyl, C—O-lower alkyl, C—OH, C-phenyl, C-benzyl, C-nitro, C-thiol, C-halocarbon, or C-halogen. In accordance with other preferred embodiments, at least one of A and B is C-halogen or C-halocarbon including C-fluorocarbon, especially C-trifluoromethyl. Other fluorocarbons include C—C(CF₃)₃, C—CF₂—CF₃ and C—CF₂—CF₂—CF₃. Halogens includes fluorine, bromine, chlorine and iodine.

In accordance with other preferred embodiments, one or both of A and B are nitrogen atoms. It is still more preferred that A be nitrogen. In other embodiments, A is C—CH₃ or C—CF₃ and B is nitrogen or A is C—Br and B is nitrogen. Synthesis of oligonucleotides containing such modified pyrimidine bases is described in Example 236.

C5 and C6 alkyl-, aza-, or halo-modified pyrimidine bases. In certain other embodiments, the invention relates to oligonucleotides comprising at least one nucleotide containing one of the following modified pyrimidine bases: 5-alkylcytidine such as, for example, 5-methylcytidine; 5-alkyluridine such as, for example, ribothymidine; 5-halouridine such as, for example, bromouridine; 6-azapyrimidine; or 6-alkyluridine. Such modified bases are described, for example, in U.S. Pat. No. 5,672,511, hereby incorporated by reference in its entirety. Synthesis of oligonucleotides containing such modified pyrimidine bases is described in Example 237.

5-fluorouracil. In certain other embodiments, the invention relates to oligonucleotides comprising at least one 5-fluorouracil base as described, for example, in U.S. Pat. No. 5,457,187, hereby incorporated herein by reference in its entirety. Synthesis of oligonucleotides containing such modified pyrimidine bases is described in Example 238.

C5 halo- or alkyl-substituted pyrimidine bases. In certain other embodiments, the invention relates to oligonucleotides comprising at least one nucleotide containing a modified pyrimidine base of the following structure as described, for example, in U.S. Pat. No. 6,166,197, hereby incorporated by reference in its entirety: 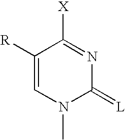
wherein X is hydroxyl or amino; R is halo or C₁-C₆ alkyl or substituted C₁-C₆ alkyl wherein said substitution is halo, amino, hydroxyl, thiol, ether or thioether; and L is oxygen or sulfur. Synthesis of oligonucleotides containing such modified pyrimidine bases is described in Example 239.

C5-amino modified pyrimidine bases. In certain other aspects, the invention relates to oligonucleotides comprising at least one nucleotide containing a modified pyrimidine base of the following structure as described, for example, in U.S. Pat. No. 5,552,540, hereby incorporated by reference in its entirety: 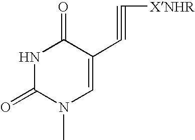
wherein X′ is a C₁₋₁₅ alkyl group which may be branched or unbranched; R is an amino protecting group, a fluorophore, other non-radioactive detectable marker, or the group Y′NHA, where Y′ is an alkyl (C₁₋₄₀) carbonyl group which may be branched or unbranched, and A is an amino protecting group or a fluorophore or other non-radioactive detectable marker. Synthesis of oligonucleotides containing such modified pyrimidine bases is described in Example 240.

Any amino protecting group may be employed. For example, amino protecting groups may be selected from acyl, particularly organic acyl, for example, substituted or unsubstituted aliphatic hydrocarbonoxycarbonyl such as alkoxycarbonyl (e.g. methoxycarbonyl, ethoxycarbonyl, propoxycarbonyl, butoxycarbonyl, t-butoxycarbonyl, 5-pentoxycarbonyl), haloalkoxycarbonyl (e.g. chloromethoxycarbonyl, tribromoethoxycarbonyl, trichloroethorycarbonyl), an alkane- or arene-sulfonylalkoxycarbonyl (e.g. 2-(mesyl)ethoxycarbonyl, 2-(p-toluenesulonyl)ethoxycarbonyl), an alkylthio- or arylthioalkoxycarbonyl (e.g. 2-(ethylthio)ethoxycarbonyl, 2-(p-tolylthio)ethoxycarbonyl), substituted or unsubstituted alkanoyl such as halo(lower)alkanoyl (e.g. formyl, trifluoroacetyl), a monocyclic or fused cyclic-alicyclic oxycarbonyl (e.g. cyclohexyloxycarbonyl, adamantyloxycarbonyl, isobornyloxycarbonyl), substituted or unsubstituted alkenyloxycarbonyl (e.g. allyoxycarbonyl), substituted or unsubstituted alkynyloxycarbonyl (e.g. 1,1-dimethylpropargyloxycarbonyl), substituted or unsubstituted aryloxycarbonyl (e.g. phenoxycarbonyl, p-methylphenoxycarbonyl), substituted or unsubstituted aralkoxycarbonyl (e.g. benzyloxycarbonyl, p-nitrobenzyloxycarbonyl, p-phenylazobenzyloxycarbonyl, p-(p-methoxyphenylazo)benzyloxycarbonyl, p-chlorobenzyloxycarbonyl, p-bromobenzyloxycarbonyl, α-naphthylmethoxycarbonyl, p-biphenylisopropoxycarbonyl, fluorenymethoxycarbonyl), substituted or unsubstituted arenesulfonyl (e.g. benzenesulfonyl, p-toluenesulfonyl), substituted or unsubstituted dialkylphosphoryl (e.g. dimethylphosphoryl), substituted or unsubstituted diaralkylphosphoryl (e.g. O,O-dibenzylphosphoryl), substituted or unsubstituted aryloxyalkanoyl (e.g. phenoxyacetyl, p-chlorophenoxyacetyl, 2-nitrophenoxyacetyl, 2-methyl-2-(2-nitrophenoxy)propyonyl), substituted or unsubstituted aryl such as phenyl, tolyl, substituted or unsubstituted aralkyl such as benzyl, diphenylmethyl, trityl or nitrobenzyl.

The term “fluorophore” refers to a moiety which in itself is capable of fluoresence or which confers fluoresence on another moiety. As used in this specification the term “fluorophore” also refers to a fluorophore precursor which contains one or more groups which suppress fluoresence, but which is capable of fluoresence once these groups are removed. (For example, diisobutyryl 6-carboxy fluorescein is non-fluorescent. Treatment with ammonia removes the diisobutyryl groups to give fluorescent 6-carboxy fluorescein). Examples of fluorophores or fluorophore precursors include: fluoroscein-5-isothiocyanate acyl (for example: diisobutyryl, acetyl or dipivaloyl)-5-and/or 6-carboxy-fluorescein pentafluorophenyl ester, 6-(diaryl-5 and/or 6-carbonyl-fluorescein)aminohexanoic acid pentafluorophenyl ester, Texas Red (Trademark of Molecular Probes, Inc.), tetramethylrhodamine-5 (and 6) isothiocyanate (hereinafter referred to as rhodamine), eosin-5-isothiocyanate, erythrosin-5-isothiocyanate, 4-chloro-7-nitrobenz-2-oxa-1,3-diazole, 4-fluoro-7-nitrobenz-2-oxa-1,3-diazole, 3-(7-nitrobenz-2-oxa-1,3-diazol-4-yl)methylaminopropionitrile, 6-(7-nitrobenz-2-oxa-1,3-diazol-4-yl)aminohexanoic acid, succinimidyl 12-(N-methyl-N-(7-nitrobenz-2-oxa-1,3-diazol-4-yl)aminododecanoate, 7-diethylamino-3-(4′-isothiocyanatophenyl)-4-methylcoumarin (CP), 7-hydroxycoumarin-4-acetic acid, 7-dimethylaminocoumarin-4-acetic acid, succinimidyl 7-dimethylaminocoumarin-4-acetate, 7-methoxycoumarin-4-acetic acid, 4-acetamido-4′-isothiocyanatostilbene-2-2′-disulfonic acid (SITS), 9-chloroacridine, succinimidyl 3-(9-carbazole)propionate, succinimidyl 1-pyrenebutyrate, succinimidyl 1-pyrenenonanoate, p-nitrophenyl 1-pyrenebutyrate, 9-anthracenepropionic acid, succinimidyl anthracene-9-propionate, 2-anthracenesulfonyl chloride.

Preferably, the fluorophores or fluorogenic substances have the following spectroscopic properties: (i) an excitation maximum coinciding with one of the strong emmission lines of the commercially used high pressure mercury lamps; (ii) an emmission maximum in the visible part of the spectrum.

Non-radioactive detectable markers include entities which may be detected directly by their physical properties, such as electron dense materials which can be detected under a microscope; or entities which may be detected indirectly by their chemical or biochemical properties, such as by the reaction of the detectabler marker with a suitable substrate(s) to produce a detectable signal, such as colour. Examples of non-radioactive detectable markers which may be detected directly include colloidal compounds such as colloidal gold and silver, and ferritin. Examples of non-radioactive detectable markers which may be detected indirectly include biotin, avidin and enzymes such as β-galactosidase, urease, peroxidase and alkaline phosphatase.

Pyrimidine bases containing C5 substituted with a cationic moiety. In certain other aspects, the invention relates to oligonucleotides comprising at least one nucleotide containing a modified pyrimidine base of one of the following structures as described, for example, in U.S. Pat. No. 5,596,091, hereby incorporated by reference in its entirety: 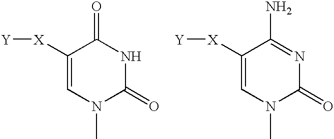
wherein X is a linking group which is C₁-C₁₀ alkyl, C₁-C₁₀ unsaturated alkyl, dialkyl ether or dialkylthioether; Y is a cationic moiety which is —(NH₃)⁺, —(NH₂R¹)⁺, —(NHR¹R²)⁺, —(NR¹R²R³)⁺, dialkylsulfonium or trialkylphosphonium; and R¹, R², and R³ are each independently lower alkyl having from one to ten carbon atoms. Preferred linking groups for X are C₁-C₁₀ alkyl and C₁-C₁₀ unsaturated alkyl. Particularly preferred linking groups for X are C₃-C₆ alkyl and C₃-C₆ unsaturated alkyl. Preferred groups for Y are —(NH₃)⁺, —(NH₂R¹)⁺, —(NHR¹R²)⁺, —(NR¹R²R³)⁺, with —(NH₃)⁺ being particularly preferred. Synthesis of oligonucleotides containing such modified pyrimidine bases is described in Example 241.

A and G modified binding bases for forming non-standard base pairs. In certain other embodiments, the invention relates to oligonucleotides comprising at least one nucleotide containing an A and G modified binding base of one the following structures as described, for example, in U.S. Pat. Nos. 5,432,272, 6,001,983 and 6,037,120, hereby incorporated by reference in their entireties: 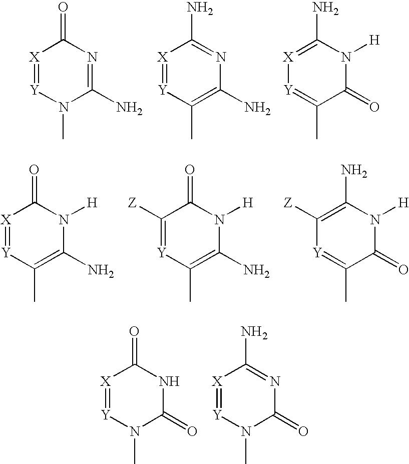
wherein X is selected from the group consisting of a nitrogen atom and a carbon atom bearing a substituent Z; Z is either a hydrogen, an unfunctionalized lower alkyl chain, or a lower alkyl chain bearing an amino, carboxyl, hydroxy, thiol, aryl, indole, or imidazoyl group; and Y is selected from the group consisting of N and CH. Synthesis of oligonucleotides containing such A and G modified binding bases is described in Example 242.

A and G modified binding universal bases. In certain other embodiments, the invention relates to oligonucleotides comprising at least one nucleotide containing an A and G modified binding universal base of the following structure as described, for example, in U.S. Pat. No. 5,681,947, hereby incorporated by reference in its entirety: 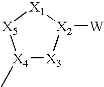
wherein the foregoing structure has least two double bonds in one of its possible tautomeric forms; X₁, X₃ and X₅ are each members of the group consisting of N, O, C, S and Se; X₂ and X₄ are each members of the group consisting of N and C; and W is a member of the group consisting of F, Cl, Br, I, O, S, OH, SH, NH₂, NO₂, C(O)H, C(O)NHOH, C(S)NHOH, NO, C(NOCH₃)NH₂, OCH₃, SCH₃, SeCH₃, ONH₂, NHOCH₃, N₃, CN, C(O)NH₂, C(NOH)NH₂, CSNH₂ and CO₂H. Synthesis of oligonucleotides containing such A and G modified binding universal bases is described in Example 243.

A and G modified binding bases containing a polycyclic aromatic group. In certain other aspects, the invention relates to oligonucleotides comprising at least one nucleotide containing an A and G modified binding base of the following structure as described, for example, in U.S. Pat. No. 5,175,273, hereby incorporated by reference in its entirety: 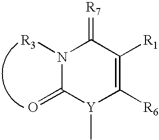
wherein R₃ is a polycyclic aromatic group; Y is C or N; R₇ is N or ═C(R₁)—; and R₁ and Rr are independently selected from the group consisting of H, halogen, C₁-C₁₀-alkyl, saturated or unsaturated cycloalkyl, C₁-C₁₀-alkylcarbonyloxy, hydroxy-C₁-C₁₀-alkyl, heterocycle (N,O, or S), and nitro. Synthesis of oligonucleotides containing such A and G modified binding bases is described in Example 244.

Tricyclic A and G modified binding bases optionally containing a detectable label. In other embodiments, the invention relates to oligonucleotides comprising at least one nucleotide containing an A and G modified binding base of the following structure as described, for example, in U.S. Pat. Nos. 6,007,992; 6,028,183; and 6,414,127, hereby incorporated by reference in their entireties: 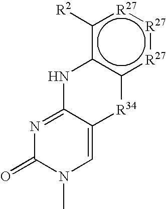
wherein R₂ is A(Z)_{X1}, wherein A is a spacer and Z independently is a label bonding group optionally bonded to a detectable label; R²⁷ is independently —CH═, —N═, —C(C₁₋₈ alkyl)=or —C(halogen)=, but no adjacent R²⁷ are both —N═, or two adjacent R²⁷ are taken together to form a ring having the structure, 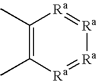
where each R^{a} is, independently, —CH═, —N═, —C(C₁₋₈ alkyl)=or —C(halogen)=, but no adjacent R^{a} are both —N═; R³⁴ is —O—, —S— or —N(CH₃)—; and X1 is 1, 2 or 3. Synthesis of oligonucleotides containing such A and G modified binding bases is described in Example 245.

Spacer A typically contains a backbone chain of 2, 3, 4, 5, 6, 7, 8, 9, 10, 11, 12, 13, 14, 15 or 16 carbon atoms, any 1, 2 or 3 of which are optionally replaced with N, O or S atoms, usually 1 N, O or S atom. The backbone chain refers to the atoms that connect the Z group(s) to the ring carbon atom at the R₂ binding site on the polycycle. The number of spacer backbone atoms does not include terminal Z group atoms. R₂ does not include protected amine as described in U.S. Pat. No. 5,502,177, hereby incorporated by reference in its entirety.

The spacer A backbone is linear or one or more backbone atoms are substituted, which results in branching. Ordinarily, when 1 Z group is present then A will contain a linear backbone of 2 to 8, usually 2 to 4 atoms. The backbone generally is carbon only, bonded by saturated or unsaturated bonds. If unsaturated bonds are present, the backbone generally will contain 1 to 2 double or triple bonds. Preferably, the backbone is saturated. If a heteroatom is present in the backbone it typically will be O or S. Preferably the heteroatom is 0, and preferably only 1 O is present in the backbone chain. Heteroatoms are used to replace any of the backbone carbon atoms, but preferably are used to replace the carbon atom alpha (adjacent) to the polycyclic ring. Usually the atom in the spacer chain that is bonded to the polycyclic substructure is unsubstituted, e.g., —O—, —S—, —NH— or —CH₂—, and, in general, the next 1, 2 or 3 atoms in the spacer are unsubstituted carbon.

The spacer A backbone is optionally substituted independently with 1, 2 or 3 of the following: C₁-C₈ alkyl, —OR₅, ═O, —NO₂, —N₃, —COOR₅, —N(R₅)₂, or —CN groups, C₁-C₈ alkyl substituted with —OH, ═O, —NO₂, —N₃, —COOR₅, —N(R₅)₂, or —CN groups, or any of the foregoing in which —CH₂— is replaced with —O—, —NH— or —N(C₁-C₈ alkyl), wherein R₅ is H or a protecting group. Certain of these groups may function as Z sites for linking to detectable labels, but need not be used for that purpose unless desired. In some embodiments these substituents are useful in increasing the lipophilicity of the compounds of this invention.

Group Z detectable labels include all of the conventional assayable substances used heretofore in labeling oligonucleotides or proteins. Examples are well known and include fluorescent moieties such as fluorescein, chemiluminescent substances, radioisotopes, chromogens, or enzymes such as horseradish peroxidase. For the purposes herein, the residue of any bifunctional or multifunctional agent used to crosslink the Z group(s) to the A backbone is defined to be part of the Z group, and the residue of the detectable label is considered also to represent part of Z.

Group Z also encompasses substituents that are not detectable by conventional diagnostic means used in clinical chemistry settings (e.g., UV or visible light absorption or emission, scintillation or gamma counting, or the like) but which are nonetheless capable of reacting with a crosslinking agent or a detectable label to form a covalent bond. In this regard, the Z groups function as intermediates in the synthesis of the labelled reagent. Typical Z groups useful for this purpose include —NH₂, —CHO, —SH, —CO₂Y or OY, where Y is H, 2-hydroxypyridine, N-hydroxysuccinimide, p-nitrophenyl, acylimidazole, maleimide, trifluoroacetate, an imido, a sulfonate, an imine 1,2-cyclohexanedione, glyoxal or an alpha-halo ketone. Suitable spacers, reactive groups and detectable labels have been described, e.g., U.S. Pat. Nos. 5,668,266, 5,659,022, 5,646,261, 5,629,153, 5,525,465 and 5,260,433, WO 88/10264, WO 97/31008, EP 063 879 B1, Urdea “NAR” 16:4937-4956 (1988), Prober “Science” 238:336-341 (1987), each of which is hereby incorporated by reference in its entirety.

Z also is a hydrogen bond donor moiety or a moiety, when taken together with the influence of spacer A, has a net positive charge of at least about +0.5 at pH 6-8 in aqueous solutions. Such Z groups are designated R_{2D}. In these embodiments, R_{2D} is covalently linked to a short spacer A having a backbone (otherwise described above) of 2, 3, 4, 5 or 6 atoms, designated R_{2C}.

The R_{2C} short spacer chain backbone atoms are C atoms and optionally one or two atoms independently selected from the group consisting of O, N or S atoms. R_{2C} short spacer chain backbones include unbranched and branched alkyl that optionally contain one or two independently selected O, N or S atoms. Usually R₂C is unbranched, i.e. the backbone has no hydrocarbon substituents. Any branching, if present, will usually consist of a C₁-C₃ alkyl group, usually a methyl or ethyl group, or C₁-C₃ alkyl substituted with —OH, ═O—O(C₁-C₃ alkyl), —CN, N₃ or 1, 2, 3 or 4 halogen atoms.

Tricyclic modified pyrimidine bases. In certain other aspects, the invention relates to oligonucleotides comprising at least one nucleotide containing a base analogue of the following structure, as described, for example, in U.S. Pat. Nos. 5,502,177; 5,763,588; and 6,005,096 hereby incorporated by reference in their entireties: 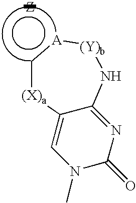
wherein a and b are 0 or 1, and the total of a and b is 0 or 1; A is N or C; X is S, O, —C(O)—, NH or NCH₂R₆; Y is —C(O)—; Z is taken together with A to form an aryl or heteroaryl ring structure comprising 5 or 6 ring atoms wherein the heteroaryl ring comprises a single O ring heteroatom, a single N ring heteroatom, a single S ring heteroatom, a single 0 and a single N ring heteroatom separated by a carbon atom, a single S and a single N ring heteroatom separated by a carbon atom, 2 N ring heteroatoms separated by a carbon atom, or 3 N ring heteroatoms at least two of which are separated by a carbon atom, and wherein at least 1 nonbridging ring carbon atom is substituted with R₆ or ═O; R₃ is a protecting group or H; R₆ is independently H, C₁-C₆ alkyl, C₂-C₆ alkenyl, C₂-C₆ alkynyl, NO₂, N(R₃)₂, C—N or halo, or R₆ is taken together with an adjacent R₆ to complete a ring containing 5 or 6 ring atoms. Synthesis of oligonucleotides containing such modified pyrimidine bases is described in Example 246.

Non-heterocyclic A and G modified binding bases. In other aspects, the invention relates to oligonucleotides comprising at least one nucleotide containing a non-heterocyclic A and G modified binding base. Such nucleotides contain the following structure: —O—Rₘ—O—Rₙ wherein Rₘ is C₁ to C₁₆ alkylene or an oxyethylene oligomer —(CH₂CH₂O)_{z}— where z is an integer in the range of 1 to 16 inclusive, and & is selected from the group consisting of: 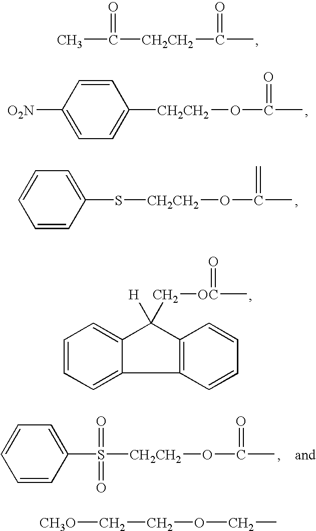
Such non-heterocyclic A and G modified binding bases are described, for example, in U.S. Pat. No. 5,367,066, hereby incorporated by reference in its entirety. Synthesis of oligonucleotides containing such non-heterocyclic A and G modified binding bases is described in Example 247.
Conjugates

A further preferred substitution that can be appended to the oligomeric compounds of the invention involves the linkage of one or more moieties or conjugates which enhance the activity, cellular distribution or cellular uptake of the resulting oligomeric compounds. In one embodiment such modified oligomeric compounds are prepared by covalently attaching conjugate groups to functional groups such as hydroxyl or amino groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Nanoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.

The oligomeric compounds of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

Chimeric Oligomeric Compounds

It is not necessary for all positions in an oligomeric compound to be uniformly modified, and in fact more than one of the aforementioned modifications may be incorporated in a single oligomeric compound or even at a single monomeric subunit such as a nucleoside within a oligomeric compound. The present invention also includes oligomeric compounds which are chimeric oligomeric compounds. “Chimeric” oligomeric compounds or “chimeras,” in the context of this invention, are oligomeric compounds that contain two or more chemically distinct regions, each made up of at least one monomer unit, i.e., a nucleotide in the case of a nucleic acid based oligomer.

Chimeric oligomeric compounds typically contain at least one region modified so as to confer increased resistance to nuclease degradation, increased cellular uptake, and/or increased binding affinity for the target nucleic acid. An additional region of the oligomeric compound may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of inhibition of gene expression. Consequently, comparable results can often be obtained with shorter oligomeric compounds when chimeras are used, compared to for example phosphorothioate deoxyoligonucleotides hybridizing to the same target region. Cleavage of the RNA target can be routinely detected by gel electrophoresis and, if necessary, associated nucleic acid hybridization techniques known in the art.

Chimeric oligomeric compounds of the invention may be formed as composite structures of two or more oligonucleotides, oligonucleotide analogs, oligonucleosides and/or oligonucleotide mimetics as described above. Such oligomeric compounds have also been referred to in the art as hybrids hemimers, gapmers or inverted gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

3′-endo Modifications

In one aspect of the present invention oligomeric compounds include nucleosides synthetically modified to induce a 3′-endo sugar conformation. A nucleoside can incorporate synthetic modifications of the heterocyclic base, the sugar moiety or both to induce a desired 3′-endo sugar conformation. These modified nucleosides are used to mimic RNA like nucleosides so that particular properties of an oligomeric compound can be enhanced while maintaining the desirable 3′-endo conformational geometry. There is an apparent preference for an RNA type duplex (A form helix, predominantly 3′-endo) as a requirement (e.g. trigger) of RNA interference which is supported in part by the fact that duplexes composed of 2′-deoxy-2′-F-nucleosides appears efficient in triggering RNAi response in the C. elegans system. Properties that are enhanced by using more stable 3′-endo nucleosides include but aren't limited to modulation of pharmacokinetic properties through modification of protein binding, protein off-rate, absorption and clearance; modulation of nuclease stability as well as chemical stability; modulation of the binding affinity and specificity of the oligomer (affinity and specificity for enzymes as well as for complementary sequences); and increasing efficacy of RNA cleavage. The present invention provides oligomeric triggers of RNAi having one or more nucleosides modified in such a way as to favor a C3′-endo type conformation.

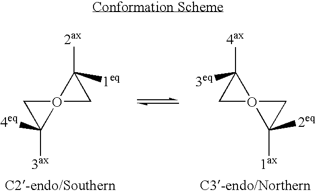

Nucleoside conformation is influenced by various factors including substitution at the 2′, 3′ or 4′-positions of the pentofuranosyl sugar. Electronegative substituents generally prefer the axial positions, while sterically demanding substituents generally prefer the equatorial positions (Principles of Nucleic Acid Structure, Wolfgang Sanger, 1984, Springer-Verlag.) Modification of the 2′ position to favor the 3′-endo conformation can be achieved while maintaining the 2′-OH as a recognition element, as illustrated in FIG. 2, below (Gallo et al., Tetrahedron (2001), 57, 5707-5713. Harry-O'kuru et al., J. Org. Chem., (1997), 62(6), 1754-1759 and Tang et al., J. Org. Chem. (1999), 64, 747-754.) Alternatively, preference for the 3′-endo conformation can be achieved by deletion of the 2′-OH as exemplified by 2′deoxy-2′F-nucleosides (Kawasaki et al., J. Med. Chem. (1993), 36, 831-841), which adopts the 3′-endo conformation positioning the electronegative fluorine atom in the axial position. Other modifications of the ribose ring, for example substitution at the 4′-position to give 4′-F modified nucleosides (Guillerm et al., Bioorganic and Medicinal Chemistry Letters (1995), 5, 1455-1460 and Owen et al., J. Org. Chem. (1976), 41, 3010-3017), or for example modification to yield methanocarba nucleoside analogs (Jacobson et al., J. Med. Chem. Lett. (2000), 43, 2196-2203 and Lee et al., Bioorganic and Medicinal Chemistry Letters (2001), 11, 1333-1337) also induce preference for the 3′-endo conformation. Along similar lines, oligomeric triggers of RNAi response might be composed of one or more nucleosides modified in such a way that conformation is locked into a C3′-endo type conformation, i.e. Locked Nucleic Acid (LNA, Singh et al, Chem. Commun. (1998), 4, 455-456), and ethylene bridged Nucleic Acids (ENA, Morita et al, Bioorganic & Medicinal Chemistry Letters (2002), 12, 73-76.) Examples of modified nucleosides amenable to the present invention are shown below in Table I. These examples are meant to be representative and not exhaustive.

TABLE I

| | | |
|---|---|---|
| 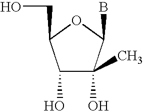 | 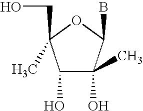 | 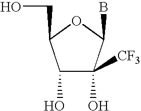 |
| 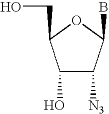 | 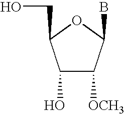 | 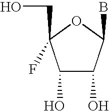 |
| 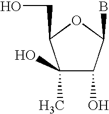 | 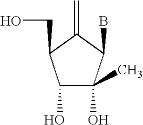 | 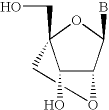 |
| 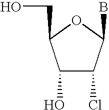 | 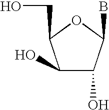 | 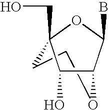 |
| 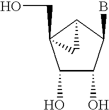 | 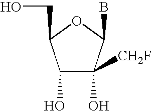 | 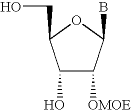 |
| 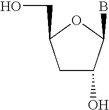 | 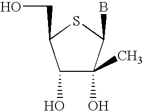 | 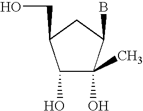 |
| 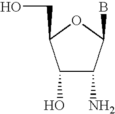 | | |

The preferred conformation of modified nucleosides and their oligomers can be estimated by various methods such as molecular dynamics calculations, nuclear magnetic resonance spectroscopy and CD measurements. Hence, modifications predicted to induce RNA like conformations, A-form duplex geometry in an oligomeric context, are selected for use in the modified oligoncleotides of the present invention. The synthesis of numerous of the modified nucleosides amenable to the present invention are known in the art (see for example, Chemistry of Nucleosides and Nucleotides Vol 1-3, ed. Leroy B. Townsend, 1988, Plenum press., and the examples section below.)

In one aspect, the present invention is directed to oligonucleotides that are prepared having enhanced properties compared to native RNA against nucleic acid targets. A target is identified and an oligonucleotide is selected having an effective length and sequence that is complementary to a portion of the target sequence. Each nucleoside of the selected sequence is scrutinized for possible enhancing modifications. A preferred modification would be the replacement of one or more RNA nucleosides with nucleosides that have the same 3′-endo conformational geometry. Such modifications can enhance chemical and nuclease stability relative to native RNA while at the same time being much cheaper and easier to synthesize and/or incorporate into an oligonulceotide. The selected sequence can be further divided into regions and the nucleosides of each region evaluated for enhancing modifications that can be the result of a chimeric configuration. Consideration is also given to the 5′ and 3′-termini as there are often advantageous modifications that can be made to one or more of the terminal nucleosides. The oligomeric compounds of the present invention include at least one 5′-modified phosphate group on a single strand or on at least one 5′-position of a double stranded sequence or sequences. Further modifications are also considered such as internucleoside linkages, conjugate groups, substitute sugars or bases, substitution of one or more nucleosides with nucleoside mimetics and any other modification that can enhance the selected sequence for its intended target.

The terms used to describe the conformational geometry of homoduplex nucleic acids are “A Form” for RNA and “B Form” for DNA. The respective conformational geometry for RNA and DNA duplexes was determined from X-ray diffraction analysis of nucleic acid fibers (Arnott and Hukins, Biochem. Biophys. Res. Comm., 1970, 47, 1504.) In general, RNA:RNA duplexes are more stable and have higher melting temperatures (Tm's) than DNA:DNA duplexes (Sanger et al., Principles of Nucleic Acid Structure, 1984, Springer-Verlag; New York, N.Y.; Lesnik et al., Biochemistry, 1995, 34, 10807-10815; Conte et al., Nucleic Acids Res., 1997, 25, 2627-2634). The increased stability of RNA has been attributed to several structural features, most notably the improved base stacking interactions that result from an A-form geometry (Searle et al., Nucleic Acids Res., 1993, 21, 2051-2056). The presence of the 2′ hydroxyl in RNA biases the sugar toward a C3′ endo pucker, i.e., also designated as Northern pucker, which causes the duplex to favor the A-form geometry. In addition, the 2′ hydroxyl groups of RNA can form a network of water mediated hydrogen bonds that help stabilize the RNA duplex (Egli et al., Biochemistry, 1996, 35, 8489-8494). On the other hand, deoxy nucleic acids prefer a C2′ endo sugar pucker, i.e., also known as Southern pucker, which is thought to impart a less stable B-form geometry (Sanger, W. (1984) Principles of Nucleic Acid Structure, Springer-Verlag, New York, N.Y.). As used herein, B-form geometry is inclusive of both C2′-endo pucker and 04′-endo pucker. This is consistent with Berger, et. al., Nucleic Acids Research, 1998, 26, 2473-2480, who pointed out that in considering the furanose conformations which give rise to B-form duplexes consideration should also be given to a 04′-endo pucker contribution.

DNA:RNA hybrid duplexes, however, are usually less stable than pure RNA:RNA duplexes, and depending on their sequence may be either more or less stable than DNA:DNA duplexes (Searle et al., Nucleic Acids Res., 1993, 21, 2051-2056). The structure of a hybrid duplex is intermediate between A- and B-form geometries, which may result in poor stacking interactions (Lane et al., Eur. J. Biochem., 1993, 215, 297-306; Fedoroff et al., J. Mol. Biol., 1993, 233, 509-523; Gonzalez et al., Biochemistry, 1995, 34, 4969-4982; Horton et al., J. Mol. Biol., 1996, 264, 521-533). The stability of the duplex formed between a target RNA and a synthetic sequence is central to therapies such as but not limited to antisense and RNA interference as these mechanisms require the binding of a synthetic oligonucleotide strand to an RNA target strand. In the case of antisense, effective inhibition of the mRNA requires that the antisense DNA have a very high binding affinity with the mRNA. Otherwise the desired interaction between the synthetic oligonucleotide strand and target mRNA strand will occur infrequently, resulting in decreased efficacy.

One routinely used method of modifying the sugar puckering is the substitution of the sugar at the 2′-position with a substituent group that influences the sugar geometry. The influence on ring conformation is dependant on the nature of the substituent at the 2′-position. A number of different substituents have been studied to determine their sugar puckering effect. For example, 2′-halogens have been studied showing that the 2′-fluoro derivative exhibits the largest population (65%) of the C3′-endo form, and the 2′-iodo exhibits the lowest population (7%). The populations of adenosine (2′-OH) versus deoxyadenosine (2′-H) are 36% and 19%, respectively. Furthermore, the effect of the 2′-fluoro group of adenosine dimers (2′-deoxy-2′-fluoroadenosine-2′-deoxy-2′-fluoro-adenosine) is further correlated to the stabilization of the stacked conformation.

As expected, the relative duplex stability can be enhanced by replacement of 2′-OH groups with 2′-F groups thereby increasing the C3′-endo population. It is assumed that the highly polar nature of the 2′-F bond and the extreme preference for C3′-endo puckering may stabilize the stacked conformation in an A-form duplex. Data from UV hypochromicity, circular dichroism, and ¹H NMR also indicate that the degree of stacking decreases as the electronegativity of the halo substituent decreases. Furthermore, steric bulk at the 2′-position of the sugar moiety is better accommodated in an A-form duplex than a B-form duplex. Thus, a 2′-substituent on the 3′-terminus of a dinucleoside monophosphate is thought to exert a number of effects on the stacking conformation: steric repulsion, furanose puckering preference, electrostatic repulsion, hydrophobic attraction, and hydrogen bonding capabilities. These substituent effects are thought to be determined by the molecular size, electronegativity, and hydrophobicity of the substituent. Melting temperatures of complementary strands is also increased with the 2′-substituted adenosine diphosphates. It is not clear whether the 3′-endo preference of the conformation or the presence of the substituent is responsible for the increased binding. However, greater overlap of adjacent bases (stacking) can be achieved with the 3′-endo conformation.

One synthetic 2′-modification that imparts increased nuclease resistance and a very high binding affinity to nucleotides is the 2-methoxyethoxy (2′-MOE, 2′-OCH₂CH₂OCH₃) side chain (Baker et al., J. Biol. Chem., 1997, 272, 11944-12000). One of the immediate advantages of the 2′-MOE substitution is the improvement in binding affinity, which is greater than many similar 2′ modifications such as O-methyl, O-propyl, and O-aminopropyl. Oligonucleotides having the 2′-O-methoxyethyl substituent also have been shown to be antisense inhibitors of gene expression with promising features for in vivo use (Martin, P., Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926). Relative to DNA, the oligonucleotides having the 2′-MOE modification displayed improved RNA affinity and higher nuclease resistance. Chimeric oligonucleotides having 2′-MOE substituents in the wing nucleosides and an internal region of deoxy-phosphorothioate nucleotides (also termed a gapped oligonucleotide or gapmer) have shown effective reduction in the growth of tumors in animal models at low doses. 2′-MOE substituted oligonucleotides have also shown outstanding promise as antisense agents in several disease states. One such MOE substituted oligonucleotide is presently being investigated in clinical trials for the treatment of CMV retinitis.

Chemistries Defined

Unless otherwise defined herein, alkyl means C₁-C₁₂, preferably C₁-C₈, and more preferably C₁-C₆, straight or (where possible) branched chain aliphatic hydrocarbyl.

Unless otherwise defined herein, heteroalkyl means C₁-C₁₂, preferably C₁-C₈, and more preferably C₁-C₆, straight or (where possible) branched chain aliphatic hydrocarbyl containing at least one, and preferably about 1 to about 3, hetero atoms in the chain, including the terminal portion of the chain. Preferred heteroatoms include N, O and S.

Unless otherwise defined herein, cycloalkyl means C₃-C₁₂, preferably C₃-C₈, and more preferably C₃-C₆, aliphatic hydrocarbyl ring.

Unless otherwise defined herein, alkenyl means C₂-C₁₂, preferably C₂-C₈, and more preferably C₂-C₆ alkenyl, which may be straight or (where possible) branched hydrocarbyl moiety, which contains at least one carbon-carbon double bond.

Unless otherwise defined herein, alkynyl means C₂-C₁₂, preferably C₂-C₈, and more preferably C₂-C₆ alkynyl, which may be straight or (where possible) branched hydrocarbyl moiety, which contains at least one carbon-carbon triple bond.

Unless otherwise defined herein, heterocycloalkyl means a ring moiety containing at least three ring members, at least one of which is carbon, and of which 1, 2 or three ring members are other than carbon. Preferably the number of carbon atoms varies from 1 to about 12, preferably 1 to about 6, and the total number of ring members varies from three to about 15, preferably from about 3 to about 8. Preferred ring heteroatoms are N, O and S. Preferred heterocycloalkyl groups include morpholino, thiomorpholino, piperidinyl, piperazinyl, homopiperidinyl, homopiperazinyl, homomorpholino, homothiomorpholino, pyrrolodinyl, tetrahydrooxazolyl, tetrahydroimidazolyl, tetrahydrothiazolyl, tetrahydroisoxazolyl, tetrahydropyrrazolyl, furanyl, pyranyl, and tetrahydroisothiazolyl.

Unless otherwise defined herein, aryl means any hydrocarbon ring structure containing at least one aryl ring. Preferred aryl rings have about 6 to about 20 ring carbons. Especially preferred aryl rings include phenyl, napthyl, anthracenyl, and phenanthrenyl.

Unless otherwise defined herein, hetaryl means a ring moiety containing at least one fully unsaturated ring, the ring consisting of carbon and non-carbon atoms. Preferably the ring system contains about 1 to about 4 rings. Preferably the number of carbon atoms varies from 1 to about 12, preferably 1 to about 6, and the total number of ring members varies from three to about 15, preferably from about 3 to about 8. Preferred ring heteroatoms are N, O and S. Preferred hetaryl moieties include pyrazolyl, thiophenyl, pyridyl, imidazolyl, tetrazolyl, pyridyl, pyrimidinyl, purinyl, quinazolinyl, quinoxalinyl, benzimidazolyl, benzothiophenyl, etc.

Unless otherwise defined herein, where a moiety is defined as a compound moiety, such as hetarylalkyl (hetaryl and alkyl), aralkyl (aryl and alkyl), etc., each of the sub-moieties is as defined herein.

Unless otherwise defined herein, an electron withdrawing group is a group, such as the cyano or isocyanato group that draws electronic charge away from the carbon to which it is attached. Other electron withdrawing groups of note include those whose electronegativities exceed that of carbon, for example halogen, nitro, or phenyl substituted in the ortho- or para-position with one or more cyano, isothiocyanato, nitro or halo groups.

Unless otherwise defined herein, the terms halogen and halo have their ordinary meanings. Preferred halo (halogen) substituents are Cl, Br, and I. The aforementioned optional substituents are, unless otherwise herein defined, suitable substituents depending upon desired properties. Included are halogens (Cl, Br, I), alkyl, alkenyl, and alkynyl moieties, NO₂, NH₃ (substituted and unsubstituted), acid moieties (e.g. —CO₂H, —OSO₃H₂, etc.), heterocycloalkyl moieties, hetaryl moieties, aryl moieties, etc. In all the preceding formulae, the squiggle (˜) indicates a bond to an oxygen or sulfur of the 5′-phosphate.

Phosphate protecting groups include those described in U.S. Pat. No. 5,760,209, U.S. Pat. No. 5,614,621, U.S. Pat. No. 6,051,699, U.S. Pat. No. 6,020,475, U.S. Pat. No. 6,326,478, U.S. Pat. No. 6,169,177, U.S. Pat. No. 6,121,437, U.S. Pat. No. 6,465,628 each of which is expressly incorporated herein by reference in its entirety.

Screening, Target Validation and Drug Discovery

For use in screening and target validation, the compounds and compositions of the invention are used to modulate the expression of a selected protein. “Modulators” are those oligomeric compounds and compositions that decrease or increase the expression of a nucleic acid molecule encoding a protein and which comprise at least an 8-nucleobase portion which is complementary to a preferred target segment. The screening method comprises the steps of contacting a preferred target segment of a nucleic acid molecule encoding a protein with one or more candidate modulators, and selecting for one or more candidate modulators which decrease or increase the expression of a nucleic acid molecule encoding a protein. Once it is shown that the candidate modulator or modulators are capable of modulating (e.g. either decreasing or increasing) the expression of a nucleic acid molecule encoding a peptide, the modulator may then be employed in further investigative studies of the function of the peptide, or for use as a research, diagnostic, or therapeutic agent in accordance with the present invention.

The conduction such screening and target validation studies, oligomeric compounds of invention can be used combined with their respective complementary strand oligomeric compound to form stabilized double-stranded (duplexed) oligonucleotides. Double stranded oligonucleotide moieties have been shown to modulate target expression and regulate translation as well as RNA processing via an antisense mechanism. Moreover, the double-stranded moieties may be subject to chemical modifications (Fire et al., Nature, 1998, 391, 806-811; Timmons and Fire, Nature 1998, 395, 854; Timmons et al., Gene, 2001, 263, 103-112; Tabara et al., Science, 1998, 282, 430-431; Montgomery et al., Proc. Natl. Acad. Sci. USA, 1998, 95, 15502-15507; Tuschl et al., Genes Dev., 1999, 13, 3191-3197; Elbashir et al., Nature, 2001, 411, 494-498; Elbashir et al., Genes Dev. 2001, 15, 188-200; Nishikura et al., Cell (2001), 107, 415-416; and Bass et al., Cell (2000), 101, 235-238.) For example, such double-stranded moieties have been shown to inhibit the target by the classical hybridization of antisense strand of the duplex to the target, thereby triggering enzymatic degradation of the target (Tijsterman et al., Science, 2002, 295, 694-697).

For use in drug discovery and target validation, oligomeric compounds of the present invention are used to elucidate relationships that exist between proteins and a disease state, phenotype, or condition. These methods include detecting or modulating a target peptide comprising contacting a sample, tissue, cell, or organism with the oligomeric compounds and compositions of the present invention, measuring the nucleic acid or protein level of the target and/or a related phenotypic or chemical endpoint at some time after treatment, and optionally comparing the measured value to a non-treated sample or sample treated with a further oligomeric compound of the invention. These methods can also be performed in parallel or in combination with other experiments to determine the function of unknown genes for the process of target validation or to determine the validity of a particular gene product as a target for treatment or prevention of a disease or disorder.

Kits, Research Reagents, Diagnostics, and Therapeutics

The oligomeric compounds and compositions of the present invention can additionally be utilized for diagnostics, therapeutics, prophylaxis and as research reagents and kits. Such uses allows for those of ordinary skill to elucidate the function of particular genes or to distinguish between functions of various members of a biological pathway.

For use in kits and diagnostics, the oligomeric compounds and compositions of the present invention, either alone or in combination with other compounds or therapeutics, can be used as tools in differential and/or combinatorial analyses to elucidate expression patterns of a portion or the entire complement of genes expressed within cells and tissues.

As one non-limiting example, expression patterns within cells or tissues treated with one or more compounds or compositions of the invention are compared to control cells or tissues not treated with the compounds or compositions and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds that affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression)(Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (To, Comb. Chem. High Throughput Screen, 2000, 3, 235-41).

The compounds and compositions of the invention are useful for research and diagnostics, because these compounds and compositions hybridize to nucleic acids encoding proteins. Hybridization of the compounds and compositions of the invention with a nucleic acid can be detected by means known in the art. Such means may include conjugation of an enzyme to the compound or composition, radiolabelling or any other suitable detection means. Kits using such detection means for detecting the level of selected proteins in a sample may also be prepared.

The specificity and sensitivity of compounds and compositions can also be harnessed by those of skill in the art for therapeutic uses. Antisense oligomeric compounds have been employed as therapeutic moieties in the treatment of disease states in animals, including humans. Antisense oligonucleotide drugs, including ribozymes, have been safely and effectively administered to humans and numerous clinical trials are presently underway. It is thus established that oligomeric compounds can be useful therapeutic modalities that can be configured to be useful in treatment regimes for the treatment of cells, tissues and animals, especially humans.

For therapeutics, an animal, preferably a human, suspected of having a disease or disorder that can be treated by modulating the expression of a selected protein is treated by administering the compounds and compositions. For example, in one non-limiting embodiment, the methods comprise the step of administering to the animal in need of treatment, a therapeutically effective amount of a protein inhibitor. The protein inhibitors of the present invention effectively inhibit the activity of the protein or inhibit the expression of the protein. In one embodiment, the activity or expression of a protein in an animal is inhibited by about 10%. Preferably, the activity or expression of a protein in an animal is inhibited by about 30%. More preferably, the activity or expression of a protein in an animal is inhibited by 50% or more.

For example, the reduction of the expression of a protein may be measured in serum, adipose tissue, liver or any other body fluid, tissue or organ of the animal. Preferably, the cells contained within the fluids, tissues or organs being analyzed contain a nucleic acid molecule encoding a protein and/or the protein itself.

The compounds and compositions of the invention can be utilized in pharmaceutical compositions by adding an effective amount of the compound or composition to a suitable pharmaceutically acceptable diluent or carrier. Use of the oligomeric compounds and methods of the invention may also be useful prophylactically.

Formulations

The compounds and compositions of the invention may also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The compounds and compositions of the invention encompass any pharmaceutically acceptable salts, esters, or salts of such esters, or any other compound which, upon administration to an animal, including a human, is capable of providing (directly or indirectly) the biologically active metabolite or residue thereof. Accordingly, for example, the disclosure is also drawn to prodrugs and pharmaceutically acceptable salts of the oligomeric compounds of the invention, pharmaceutically acceptable salts of such prodrugs, and other bioequivalents. The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed in WO 93/24510 to Gosselin et al., published Dec. 9, 1993 or in WO 94/26764 and U.S. Pat. No. 5,770,713 to Imbach et al.

The term “pharmaceutically acceptable salts” refers to physiologically and pharmaceutically acceptable salts of the compounds and compositions of the invention: i.e., salts that retain the desired biological activity of the parent compound and do not impart undesired toxicological effects thereto. For oligonucleotides, preferred examples of pharmaceutically acceptable salts and their uses are further described in U.S. Pat. No. 6,287,860, which is incorporated herein in its entirety.

The present invention also includes pharmaceutical compositions and formulations that include the compounds and compositions of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compounds and compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions of the present invention include, but are not limited to, solutions, emulsions, foams and liposome-containing formulations. The pharmaceutical compositions and formulations of the present invention may comprise one or more penetration enhancers, carriers, excipients or other active or inactive ingredients.

Emulsions are typically heterogenous systems of one liquid dispersed in another in the form of droplets usually exceeding 0.1 μm in diameter. Emulsions may contain additional components in addition to the dispersed phases, and the active drug that may be present as a solution in either the aqueous phase, oily phase or itself as a separate phase. Microemulsions are included as an embodiment of the present invention. Emulsions and their uses are well known in the art and are further described in U.S. Pat. No. 6,287,860, which is incorporated herein in its entirety.

Formulations of the present invention include liposomal formulations. As used in the present invention, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein in its entirety.

The pharmaceutical formulations and compositions of the present invention may also include surfactants. The use of surfactants in drug products, formulations and in emulsions is well known in the art. Surfactants and their uses are further described in U.S. Pat. No. 6,287,860, which is incorporated herein in its entirety.

In one embodiment, the present invention employs various penetration enhancers to effect the efficient delivery of nucleic acids, particularly oligonucleotides. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein in its entirety.

One of skill in the art will recognize that formulations are routinely designed according to their intended use, i.e. route of administration.

Preferred formulations for topical administration include those in which the oligonucleotides of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

For topical or other administration, compounds and compositions of the invention may be encapsulated within liposomes or may form complexes thereto, in particular to cationic liposomes. Alternatively, they may be complexed to lipids, in particular to cationic lipids. Preferred fatty acids and esters, pharmaceutically acceptable salts thereof, and their uses are further described in U.S. Pat. No. 6,287,860, which is incorporated herein in its entirety. Topical formulations are described in detail in U.S. patent application Ser. No. 09/315,298 filed on May 20, 1999, which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides of the invention are administered in conjunction with one or more penetration enhancers surfactants and chelators. Preferred surfactants include fatty acids and/or esters or salts thereof, bile acids and/or salts thereof. Preferred bile acids/salts and fatty acids and their uses are further described in U.S. Pat. No. 6,287,860, which is incorporated herein in its entirety. Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Compounds and compositions of the invention may be delivered orally, in granular form including sprayed dried particles, or complexed to form micro or nanoparticles. Complexing agents and their uses are further described in U.S. Pat. No. 6,287,860, which is incorporated herein in its entirety. Certain oral formulations for oligonucleotides and their preparation are described in detail in U.S. application Ser. Nos. 09/108,673 (filed Jul. 1, 1998), 09/315,298 (filed May 20, 1999) and 10/071,822, filed Feb. 8, 2002, each of which is incorporated herein by reference in their entirety.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Certain embodiments of the invention provide pharmaceutical compositions containing one or more of the compounds and compositions of the invention and one or more other chemotherapeutic agents that function by a non-antisense mechanism. Examples of such chemotherapeutic agents include but are not limited to cancer chemotherapeutic drugs such as daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). When used with the oligomeric compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Combinations of compounds and compositions of the invention and other drugs are also within the scope of this invention. Two or more combined compounds such as two oligomeric compounds or one oligomeric compound combined with further compounds may be used together or sequentially.

In another related embodiment, compositions of the invention may contain one or more of the compounds and compositions of the invention targeted to a first nucleic acid and one or more additional compounds such as antisense oligomeric compounds targeted to a second nucleic acid target. Numerous examples of antisense oligomeric compounds are known in the art. Alternatively, compositions of the invention may contain two or more oligomeric compounds and compositions targeted to different regions of the same nucleic acid target. Two or more combined compounds may be used together or sequentially

Dosing

The formulation of therapeutic compounds and compositions of the invention and their subsequent administration (dosing) is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 ug to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 ug to 100 g per kg of body weight, once or more daily, to once every 20 years.

While the present invention has been described with specificity in accordance with certain of its preferred embodiments, the following examples serve only to illustrate the invention and are not intended to limit the same.

The entire disclosure of each patent, patent application, and publication cited or described in this document is hereby incorporated by reference.

### EXAMPLE 1

Scheme 1 is the synthetic scheme for monomers and intermediates described in Examples 1-12 and 120. 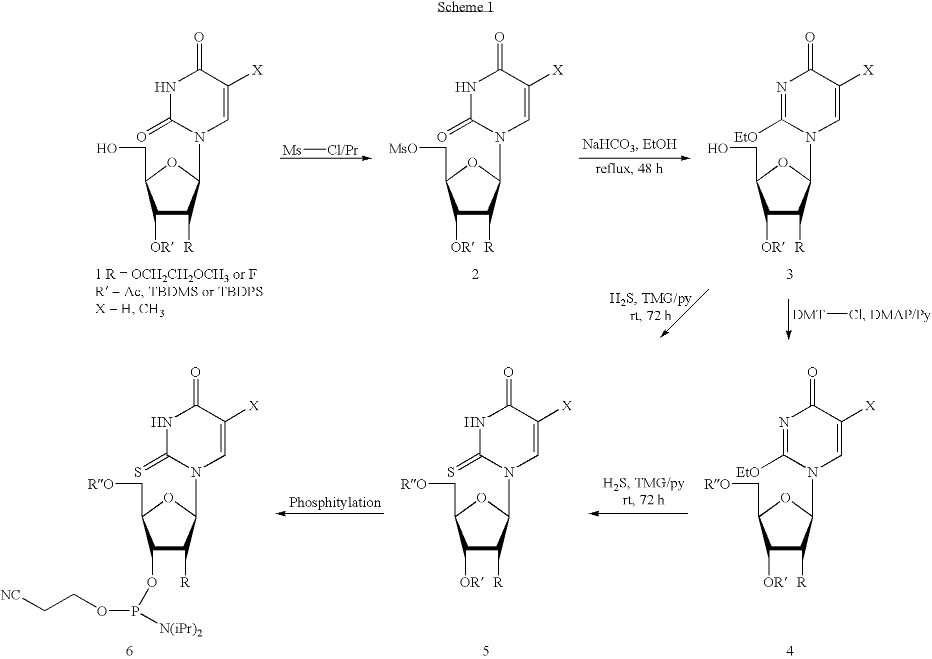
Compound 3 (R=OCH₂CH₂OCH₃, R′=H, X=CH₃, Scheme 1).

Compound 2 (R=OCH₂CH₂OCH₃, R′=OAc, X=CH₃) was prepared from 2′-O-(2-methoxy)ethyl-3′-O— thymidine (prepared as reported, Martin P. Helvetica Chimica Acta, 1995, 78, 486-504) and methanesulfonyl chloride according to standard procedure. Compound 2 (10.0 g, 22.94 mmol) after drying over P₂O₅ under vacuum was refluxed in absolute ethanol (100 mL) in the presence of anhydrous sodium bicarbonate (4.82 g, 57.37 mmol, 2.5 molar eq.) under argon for 30 h. Progress of the reaction was monitored by TLC. After cooling to room temperature, reaction mixture was diluted with ethyl acetate and the precipitated sodium salt was removed by filtration. Filtrate was concentrated to a white solid and was purified by silica gel column chromatography: eluent, 4% MeOH in DCM, to obtain compound 3 (5.95 g, 75.4%) as a white solid. ¹H NMR (200 MHz, DMSO-d₆): δ 7.93-7.92 (d, 1H), 5.79-5.77 (d, 1H, J=4.40 Hz), 5.23-5.18 (t, 1H, exchangeable with D₂O), 5.09-5.06 (d, 1H, exchangeable with D₂O), 4.40-4.28 (m, 2H), 4.15-4.06 (m, 1H), 4.01-3.96 (t, 1H), 3.90-3.84 (m, 1H), 3.75-3.53 (m, 4H), 3.45-3.40 (t, 2H), 3.32 (s, 1H exchangeable with D₂O), 3.20 (s, 3H), 1.78-1.77 (d, 3H), 1.33-1.25 (t, 3H). ¹³C NMR (50 MHz, DMSO-d₆): δ 170.2, 154.6, 133.8, 115.8, 87.6, 85.1, 82.0, 71.2, 69.2, 68.0, 64.2, 60.1, 58.1, 14.0, 13.4. FAB-Glycerol MS: Calc. for C₁₅H₂₄N₂O₇ 344.16, Found 345 (MH⁺).

### EXAMPLE 2

Compound 4 (R=OCH₂CH₂OCH₃, R′=H, R″=DMT, X=CH₃, Scheme 1). Compound 3 (5.60 g, 16.279 mmol), after drying over P₂O₅ under vacuum, was reacted with DMT-Cl (6.06 g, 17.88 mmol, 1.1 molar eq.) in the presence of DMAP (0.20 g, 1.64 mmol) in anhydrous pyridine under argon atmosphere at ambient temperature for 4 h. Removed pyridine from the reaction mixture and the residue suspended in ethyl acetate (50 mL) was washed with saturated sodium bicarbonate solution (20 mL) and water (20 mL). The organic phase was evaporated to dryness and the residue loaded on a silica gel column was eluted out with 4% MeOH in DCM to obtain compound 4 (9.5 g, 90.33%) as a white foam. ¹H NMR (200 MHz, DMSO-d₆): δ 7.61(s, 1H), 7.41-7.24 (m, 9H), 6.91-6.87 (d, 4H), 5.80-5.78 (d, 1H, J=4.0 Hz), 5.23-5.20 (d, 1H, exchangeable with D₂O), 4.39-4.21 (m, 3H), 4.15-4.11 (m, 1H), 4.02 (bm, 1H), 3.76-3.49 (m, 8H), 3.49-3.44 (t, 2H), 3.31-3.21 (m, 5H), 1.38 (s, 3H), 1.32-1.25 (t, 3H). ¹³C NMR (50 MHz, DMSO-d₆): δ 170.1, 158.2, 154.6, 144.6, 135.3, 135.1, 133.3, 129.8, 127.9, 127.7, 126.8, 116.0, 113.3, 88.2, 86.0, 83.1, 81.8, 71.4, 69.5, 68.6, 64.3, 62.8, 58.2, 55.1, 14.0, 12.8. FAB MS: Calc. for C₃₆H₄₂N₂O₉ 646.29, Found 647 (MH⁺).

### EXAMPLE 3

Compound 5 (R=OCH₂CH₂OCH₃, R′=H, R″=DMT, X=CH₃, Scheme 1). Compound 4 (6.0 g, 9.28 mmol, R=OCH₂CH₂OCH₃, R′=H, R″=DMT, X=CH₃, Example 1) after thorough drying over P₂O₅ under vacuum was placed in a 250 mL round bottom flask (RB) under argon atmosphere and cooled over a freezing bath. A solution of anhydrous 1,1,3,3-tetramethylguanidine (TMG, 11.7 mL, 93.25 mmol) in pyridine (100 mL) was flushed with argon and cooled to 0° C. over a freezing bath. After cooling the pyridine solution was saturated with hydrogen sulfide for 45 min by maintaining the temperature of the bath below 0° C. The solution was then transferred into the pre-cooled flask containing compound 5 under argon pressure. Temperature of the flask was slowly brought up to room temperature and stored for 72 h. H₂S was gently flushed out into a chlorox bath and then pyridine was removed from the reaction mixture under vacuum. Residue suspended in ethyl acetate was subjected to water wash followed by standard workup. The desired product was purified by column chromatography using ethyl acetate and hexane (1:1) as eluent to yield compound 4 as a white foamy solid (4.03 g, 66.2%). ¹H NMR (200 MHz, DMSO-d₆): δ 12.64 (bs, 1H, exchangeable with D₂O), 7.61 (s, 1H), 7.38-7.20 (m, 9H), 6.89-6.84 (d, 4H), 6.61-6.59 (d, 1H, J=3.4 Hz), 5.13-5.10 (d, 1H, exchangeable with D₂O), 4.28-4.20 (m, 1H), 4.08-3.96 (m, 2H), 3.90-3.69 (m, 8H), 3.51-3.46 (t, 2H), 3.30-3.20 (bm, accounted for 14H, 5H+water from the solvent), 1.31 (s, 3H). ¹³C NMR (50 MHz, DMSO-d₆): δ 174.8, 160.6, 158.2, 144.6, 136.0, 135.3, 135.0, 129.8, 128.0, 127.7, 126.9, 115.3, 113.3, 91.5, 86.0, 82.8, 82.1, 71.4, 69.9, 68.7, 62.5, 58.2, 55.1, 11.9. FAB-NBA MS: Calc. for C₃₄H₃₈N₂O₈S 634.23, Found 635 (MH⁺). FAB-NBA/LiCl M.⁷Li⁺ 641. HRMS: Calc. for C₃₄H₃₈N₂O₈S. ⁷Li 641.250893, Found 641.252500.

### EXAMPLE 4

Compound 3 (R=OCH₂CH₂OCH₃, R′=—Si[TBDP], R″=OH, X=CH₃, Scheme 1). Compound 2 (R=OCH₂CH₂OCH₃, R′=OSiTBDP, X=CH₃) was prepared from 2′-O-(2-methoxy)ethyl-3′-O-(t-butyldiphenyl)silyl-thymidine and methanesulfonyl chloride according to standard procedure. Compound 2 (4.7 g, 7.44 mmol) was refluxed with anhydrous sodium bicarbonate (950 mg, 11.31 mmol, 1.52 molar eq.) in absolute ethanol under argon for 48 h, followed by standard workup and purification (silica gel column chromatography: eluent 2% MeOH in DCM) as reported in Example 1, to obtain compound 3 (3.0 g, 69.3%) as a white foam. ¹H NMR (200 MHz, DMSO-d₆): δ 7.80 (s, 1H), 7.70-7.65 (m, 4H), 7.61-7.57 (m, 6H), 5.90-5.88 (d, 1H, J=5.0 Hz), 5.22-5.17 (t, 1H, exchangeable with D₂O), 4.33-4.23 (m, 3H), 3.99-3.97 (bm, 1H), 3.68-3.55 (m, 2H), 3.42-3.17 (m, 5H), 3.17 (s, 3H), 1.74-1.73 (d, 3H), 1.29-1.22 (t, 3H), 1.04 (s, 9H). ¹³C NMR (50 MHz, DMSO-d₆): δ 170.0, 154.5, 135.5, 135.3, 133.5, 132.9, 132.8, 130.0, 129.9, 127.8, 127.7, 115.8,m 86.9, 85.0, 81.3, 71.1, 70.6, 69.0, 64.2, 59.8, 58.2, 26.7, 18.9, 13.9, 13.4. FAB-NBA MS Calc. for C₃₁H₄₂N₂O₇Si 582.28, Found 583 (MH⁺).

### EXAMPLE 5

Compound 5 (R=OCH₂CH₂OCH₃, R′=TBDPS, R″=H, X=CH₃, Scheme 1). Compound 5 (as specified) was prepared from compound 3 (0.4 g, 0.69 mmol, from Example 4) and TMG-H₂S as described in Example 3. Yield 0.25 g, 63.8%. ¹H NMR (200 MHz, DMSO-d₆): δ 12.59 (s, 1H, exchangeable with D₂O), 7.97 (s, 1H), 7.71-7.57 (m, 4H), 7.48-7.32 (bm, 6H), 6.81-6.79 (d, 1H, J=4.4 Hz), 5.29 (bt, 1H), 4.32-4.27 (bt, 1H), 4.01 (bs, 1H), 3.79-3.59 (bm, 2H), 3.38-3.20 (m, 5H), 3.16 (s, 3H), 1.76 (s, 3H), 1.04 (s, 9H).

### EXAMPLE 6

Compound 3 (R=OCH₂CH₂OCH₃, R′=TBDPS X=H, Scheme 1). Compound 2 (R=OCH₂CH₂OCH₃, R′=TBDPS, X═H) was prepared from 2′-O-(2-methoxy)ethyl-3′-O-(t-butyldiphenyl)silyl-uridine and methanesulfonyl chloride according to standard procedure. Compound 2 (4.186 g, 6.77 mmol) was refluxed with anhydrous sodium bicarbonate (1.14 g, 13.57 mmol, 2 molar eq.) in absolute ethanol under argon for 60 h, followed by standard workup and purification (silica gel column chromatography: eluent 5% MeOH in DCM) as reported in Example 1, to obtain compound 3 (2.2 g, 57.2%) as a white foam. ¹H NMR (200 MHz, DMSO-d₆): δ 7.95-7.91 (d, 1H, J=7.6 Hz), 7.70-7.56 (m, 4H), 7.50-7.36 (m, 6H), 5.89-5.87 (d, 1H, J=4.4 Hz), 5.82-5.78 (d, 1H, J=7.8 Hz), 5.20-5.15 (t, 1H, exchangeable with D₂O), 4.36-4.23 (m, 3H), 4.00-3.98 (bm, 1H), 3.64-3.57 (m, 2H), 3.43-3.21 (m, 22H, accounts for 5H and water present in the solvent), 3.13 (s, 3H), 1.29-1.22 (t, 3H), 1.04 (s, 9H). ¹³C NMR (50 MHz, DMSO-d₆): δ 169.5, 154.8, 137.8, 135.5, 135.3, 132.9, 132.8, 130.0, 129.9, 127.9, 127.7, 107.9, 87.2, 85.0, 81.5, 71.1, 70.5, 69.0, 64.3, 59.7, 58.2, 26.7, 19.0, 13.8.

### EXAMPLE 7

Compound 5 (R=OCH₂CH₂OCH₃, R′=TBDPS, R″=H, X=H, Scheme 1). Compound 5 (as specified) was obtained from compound 3 (2.15 g, 3.79 mmol, from Example 6) as described in Example 3. White solid, 1.60 g (76.0% yield). ¹H NMR (200 MHz, DMSO-d₆): δ 12.64 (s, 1H exchangeable with D₂O), 8.06-8.02 (d, 1H, J=8.2 Hz), 7.71-7.58 (m, 4H), 7.50-7.36 (m, 6H), 6.84-6.82 (d, 1H, J=4.6 Hz), 6.00-5.94 (dd, 1H, J′=8.2, J″=1.8 Hz), 5.26-5.22 (t, 1H, exchangeable with D₂O), 4.34-4.29 (t, 1H), 4.00-3.96 (bm, 1H), 3.66-3.54 (m, 3H, accounts for 2H and water from the solvent), 3.34-3.17 (m, 5H), 3.15 (s, 3H), 1.04 (s, 9H). ¹³C NMR (50 MHz, DMSO-d₆): δ 176.2, 159.3, 140.6, 135.5, 135.4, 132.9, 132.8, 130.0, 129.9, 127.8, 127.7, 106.8, 90.0, 85.0, 81.6, 71.2, 70.9, 69.4, 59.6, 58.2, 26.7, 18.9. FAB-NBA MS Calc. for C₂₈H₃₆N₂O₆SiS: 556, Found: 557 (MH⁺).

### EXAMPLE 8

Compound 6 (R=OCH₂CH₂OCH₃, X=CH₃, R″=DMT, Scheme 1). Compound 5 (0.33 g, 0.52 mmol) from Example 3 was dried over anhydrous P₂O₅ under vacuum along with tetrazole diisopropylammonium salt (0.09 g, 0.53 mmol) overnight and then suspended in anhydrous MeCN (5 mL) under argon atmosphere. 2-Cyanoethyl tetraisopropylphosphrodiamidite (0.33 mL, 1.04 mmol) was added into the suspension at ambient temperature and stirred for 6 h. Removed MeCN from the reaction mixture, residue in ethyl acetate (20 mL) was washed with saturated sodium bicarbonate followed by standard workup. Compound 6 was purified by column chromatography, eluent: ethyl acetate/hexane (1:1) to yield 0.41 g (94.4% yield). ³¹P NMR (80.95 MHz, CDCl₃): δ 151.6, 150.74. HRMS Calc. for C₄₃H₅₆N₄O₉PS 835.350565, Found 835.351090.

### EXAMPLE 9

Compound 2 (R=F, R′=Ac, X=Me, Scheme 1): Compound 1 (R=F, X=Me, 750 mg, 2.48 mmol, prepared as reported, Condington, J. F. et. al. J. Org. Chem. 1964, 29, 558-564) was treated with methanesulfonylchloride (0.4 mL, 5.16 mmol) in pyridine-dichoromethane (1:1, 5 mL) at −20° C. bath temperature for 1 hour. Solvents were removed from the reaction mixture and the residue, suspended in water (10 mL), was extracted with ethylacetate (25 mL), washed with saturated NaHCO₃ solution (10 mL) and brine (10 mL). The product extracted was purified by flash column chromatography to obtain the desired compound 2 as a white foam, eluent: 5% MeOH in dicholoromethane; yield: 930 mg, (98.5%). ¹H NMR (200 MHz, DMSO-d₆): δ 11.47 (s, exchangeable with D₂O), 7.57 (s, 1H), 5.96-5.84 (dd, 1H, H1′, J′=2.20, J″=21.80 Hz), 5.65-5.62 (m, 0.5H), 5.37-5.21 (m, 1.5H), 4.54-4.34 (m, 3H), 3.20 (s, 3H), 2.11 (s, 3H), 1.77 (s, 3H).

### EXAMPLE 10

Compound 4 (R=F, R′=H, R″=DMT, X=Me, Scheme 1): Compound 2 (900 mg, 2.37 mmol) obtained from Example 9 was mixed with anhydrous NaHCO₃ (500 mg, 5.95 mmol) and dried over P₂O₅ under vacuum overnight. The mixture was then suspended in absolute ethanol (200 proof, 10 mL) and refluxed as reported in Example 1 to obtain the 2-O-ethyl derivative, which was subsequently reacted with DMT-Cl (800 mg, 2.36 mmol) in the presence catalytic amount of DMAP (30 mg, 0.25 mmol) in anhydrous pyridine as reported in Example 2 to obtain the desired compound 4. The product was purified by flash chromatography; eluent: DCM/EtOAc (1:4); yield: 400 mg (28.6%). ¹H NMR (200 MHz, CDCl₃): δ 7.64-7.64 (d, 1H), 7.43-7.15 (m, 9H), 6.87-6.81 (m, 4H), 6.11-6.02 (dd, 1H, H1′, J′=2.50 and J″=15.30 Hz), 5.21-5.17 (m, 0.5H, H2′), 4.95-4.91 (m, 0.5H, H2′), 4.60-4.46 (m, 3H), 4.18-4.14 (m, 1H), 3.79-3.65 (m, 6H), 3.65-3.43 (m, 2H), 1.51 (s, 3H), 1.38-1.29 (t, 3H).

### EXAMPLE 11

Compound 5 (R=F, R′=H, R″=DMT, X=Me, Scheme 1): Compound 4 (300 mg, 0.51 mmol) obtained from Example 10 was taken in a 25 mL RB and dried over P₂O₅ under vacuum overnight, sealed the flask under argon and cooled over an ice bath under argon pressure. Anhydrous pyridine (10 mL) was placed on an ice bath under argon atmosphere and after cooling the dry H₂S gas was bubbled through the solvent for 30 min. The pyridine-H₂S solution was then transferred into the flask containing compound 4 under cold. The reaction mixture sealed and placed on 60° C. oil bath for 72 h. Removed pyridine and the residue taken in EtOAc (25 mL) was washed with water and bicarbonate solution. After evaporation of EtOAc, the residue was subjected to flash column chromatography to obtain the desired 2-thio-2′-fluoro nucleoside 5 as a white solid. Eluent: Hexane:EtOAc 3:1; yield: 140 mg (47.6 5). ¹H NMR (200 MHz, CDCl₃+DMSO-d₆+D₂O): δ 7.95 (s, 1H), 7.39-7.27 (m, 9H), 6.87-6.83 (m, 4H), 6.71-6.63 (d, 1H, H1′, J=15.60 Hz), 5.28 (bs, 0.5, H2′), 5.01 (bs, 0.5H, H2′), 4.60-4.42 (bm, 1H), 4.24-4.19 (bm, 1H), 3.80 (s, 6H), 3.69-3.47 (m, 2H), 1.27 (s, 3H).

### EXAMPLE 12

Compound 6 (R=F, R″=DMT, X=Me, Scheme 1): Compound 5 from Example 11 is phosphitylated as reported in Example 8 to obtain the desired phosophoramidite 6.

### EXAMPLE 13

Schemes 2a is the synthetic scheme for monomers and intermediates described in Examples 13-24 and 27.

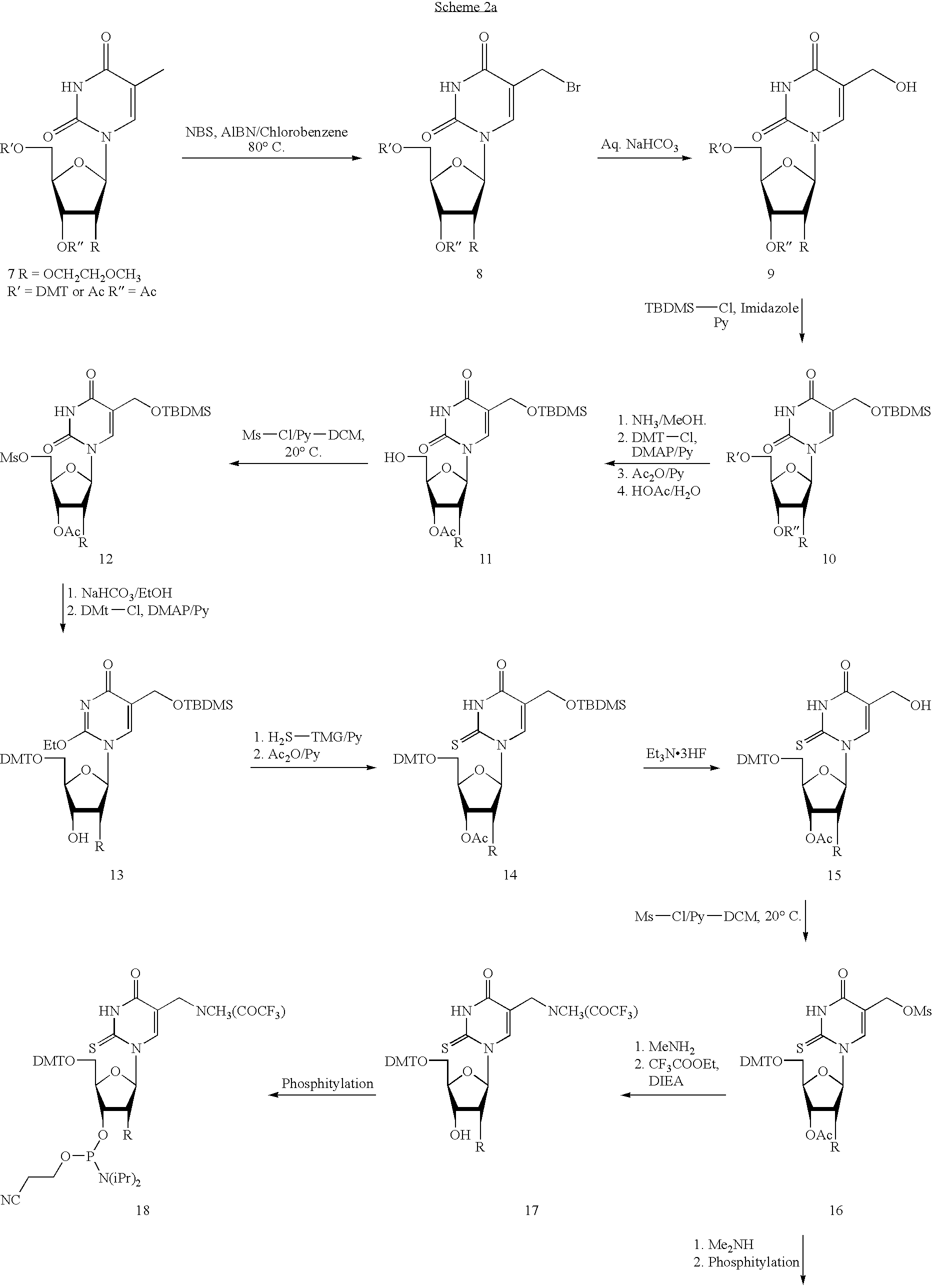

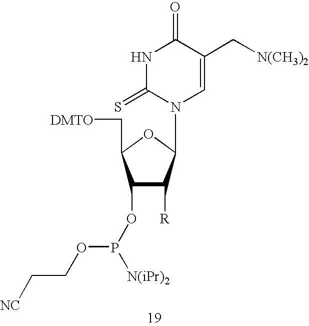

Compound 8 (R′, R″=Ac, R=OCH₂CH₂OCH₃, Scheme 2a): Compound 7 (16.5 g, 41.25 mmol, R′, R″=Ac, R=OCH₂CH₂OCH₃, Example 2a) was co-evaporated with chlorobenzene and subsequently redissolved in chlorobenzene (200 mL). The solution was thoroughly deoxygenated by gentle flushing of anhydrous argon through the solution for 10 min. Finally powdered NBS (11.02 g, 61.91 mmol, 1.5 mol eq.) was added into the solution under argon. The reaction mixture was again flushed with argon for 5 min and then placed over a pre-heated oil bath of 80° C. under constant stirring. AIBN (100 mg, 0.6089; 1 mol %) was added into the hot reaction mixture, the pale golden yellow reaction mixture turned to brown after the addition of AIBN and the brown coloration disappeared after ten min. The stirring was continued for 30 minute and the mixture turned to brown again. TLC after 15 min and after 30 min of addition of AIBN showed about 60% product formation. The reaction mixture was cooled to room temperature and the precipitated succinimide was filtered off, washed with chlorobenzene. The filtrate after concentration under vacuum was directly loaded on column of silica gel and the bromo derivative 8 was eluted out with ethyl acetate/hexane (1:1) to obtain 11.05 g (55.6%) as a white solid. ¹H NMR (200 MHz, DMSO-d₆): δ 11.96 (s, 0.2H, exchangeable with D₂O, minor rotamer), 11.71 (s, 0.6H, exchangeable with D₂O, major rotamer), 8.03 (s, 0.75H, major rotamer), 7.96 (s, 0.25H, minor rotamer), 5.83-5.78 (m, 1H), 5.17-5.10 (m, 1H), 4.38-4.19 (m, 6H), 3.66-3.48 (m, 3H), 3.40-3.33 (m, 2H), 3.19-3.16 (m, 3H), 2.08-2.04 (m, 6H). ¹H NMR (200 MHz, DMSO-d₆+D₂O; after 2 h): δ 7.52 (s, 1H), 5.88-5.85 (d, 1H, J=6.2 Hz), 5.19-5.15 (m, 1H), 4.31-4.15 (m, 6H), 3.57-3.51 (m, 2H), 3.33-3.30 (m, 2H), 3.15-3.14 (m, 3H), 2.09-2.07 (m, 6H).

### EXAMPLE 14

Compound 9 (R′, R″=Ac, R=OCH₂CH₂OCH₃, Scheme 2a): Compound 8 (4.2 g, 8.77 mmol) in 20 mL ethyl acetate was mixed with 5 mL of 10% aq. NaHCO₃ and stirred at ambient temperature for 3 h. After 3 h, the hydroxy compound formed was repeatedly extracted from the aqueous layer with EtOAc (6×25 mL) as the product was soluble in both aqueous and organic phase. Evaporated the organic layer and the residue obtained was subjected to silica gel column chromatography due to mild contamination of succinimide from the NBS reaction (Scheme 9). Eluent: 4% MeOH in DCM; Compound 9: 2.49 g (68.3%, white foam). ¹H NMR (200 MHz, DMSO-d₆): δ 11.47 (s, 1H, exchangeable with D₂O), 7.53 (s, 1H), 5.90-5.87 (d, 1H, J=6.4 Hz), 5.21-5.18 (m, 1H), 5.13-5.08 (t, 1H, exchangeable with D₂O), 4.33-4.16 (m, 6H), 3.59-3.52 (m, 2H), 3.37-3.31 (m, 2H), 3.17-3.16 (m, 3H), 2.09-2.07 (m, 6H). ¹³C NMR (50 MHz, DMSO-d₆): δ 170.8, 170.2, 162.7, 150.7, 136.2, 115.2, 87.0, 79.6, 79.0, 71.7, 70.9, 70.0, 63.6, 58.4, 56.0, 20.81, 20.79.

### EXAMPLE 15

Compound 10 (R′, R″=Ac, R=OCH₂CH₂OCH₃, Scheme 2a): Compound 9 (2.3 g, 5.53 mmol), TBDMS-CL (1.25 g, 8.29 mmol) and imidazole (1.13 g, 16.6 mmol) were stirred in anhydrous pyridine at ambient temperature for overnight. Removed pyridine from the reaction mixture followed by standard workup. The residue obtained was passed through a column of silica gel to remove excess TBDMS-Cl to obtain compound 10 as a white foam (2.35 g, 80.2%). ¹H NMR (200 MHz, CDCl₃): δ 8.72 (s, 1H, exchangeable with D₂O), 7.45-7.44 (d, 1H), 5.90-5.88 (d, 1H, J=4 Hz), 5.04-4.98 (t, 1H, J′=5.8, J″=6.0 Hz), 4.50-4.49 (m, 2H), 4.41-4.24 (m, 4H), 3.77-3.67 (m, 2H), 3.50-3.45 (t, 2H, J′=4.6, J″=4.4 Hz), 3.31 (s, 3H), 2.15-2.12 (d, 6H), 0.91 (s, 9H), 0.11 (s, 6H).

### EXAMPLE 16

Compound 11 (R=OCH₂CH₂OCH₃, Scheme 2a): Compound 10 (2.2 g, 4.15 mmol) was subjected to methanolic ammonia treatment at ambient temperature for 4 h. Progress of the deacetylation was monitored by TLC and after complete deprotection, ammonia and methanol were removed under vacuum. The residue was repeatedly evaporated with DCM and then dried over anhydrous P₂O₅ under vacuum. The anhydrous residue was then treated with DMT-Cl (1.68 g, 4.96 mmol) and DMAP (120 mg, 0.98 mmol) as reported in Example 2. Acetic anhydride (1 mL, excess) was added into the reaction mixture after overnight treatment with DMT-Cl in pyridine to acetylate 3′-hydroxyl function of the sugar moiety. The reaction mixture was stirred for 4 h. Methanol was added into reaction to quench excess anhydride. Removed pyridine, the residue in ethyl acetate (30 mL) was washed with saturated NaHCO₃ solution. After evaporating ethyl acetate, the solid obtained was dissolved in 80% aqueous acetic acid and stirred at ambient temperature for 4 h. Acetic acid was removed from the reaction mixture under vacuum and the residue in ethyl acetate (40 mL) was washed with water and aqueous bicarbonate solution. Compound 11 was then purified by silica gel column chromatography. Eluent: 4% methanol in DCM, 1.25 g (61.7%, white foam, hygroscopic). ¹H NMR (200 MHz, DMSO-d₆): δ 11.47 (s, 1H, exchangeable with D₂O), 7.80 (s, 1H), 5.92-5.88 (d, 1H, J=6.8 Hz), 5.27-5.18 (m, 2H) [Note: After D₂O exchange: δ 5.24-5.20, m, 1H], 4.33 (s, 2H), 4.24-4.18 (t, 1H), 4.07=4.05 (m, 1H), 3.60-3.49 (m, 4H), 3.34-3.31 (m, 2H), 3.15 (s, 3H), 2.09 (s, 3H), 0.86 (s, 9H), 0.07 (s, 6H).

### EXAMPLE 17

Compound 12 (Scheme 2a): Compound 11 (1.1 g, 2.25 mmol) was taken in 10 mL of anhydrous DCM-Pyridine (1:1) and stirred at −20° C. Methanesulfonyl chloride (0.5 mL, 6.46 mmol) was added into the stirring solution drop wise and the stirring was continued for 2 h at −20° C. Removed pyridine from the reaction mixture under diminished pressure and standard workup in ethyl acetate was followed. The sulfonate 12 was passed through a column of silica gel; eluent DCM/EtOAc (3:2), to obtain the desired product as a white foam, yield 1.28 g (quantitative). ¹H NMR (200 MHz, CDCl₃): δ 9.18 (s, 1H, exchangeable with D₂O), 7.36 (s, 1H), 5.80-5.78 (d, 1H, H1′, J=4.40 Hz), 5.17-5.11 (t, 1H), 4.51-4.38 (m, 6H), 3.73-3.68 (m, 2H), 3.49-3.45 (m, 2H), 3.31 (s, 3H), 3.07 (s, 3H), 2.16 (s, 3H), 0.93 (s, 9H), 0.13 (s, 6H). ¹³C NMR (200 MHz, CDCl₃): 170.1, 162.1, 150.0, 137.3, 115.0, 91.5, 79.6, 79.3, 72.0, 71.0, 70.5, 67.6, 58.9, 58.0, 37.7, 25.9, 20.6, 18.4.

### EXAMPLE 18

Compound 13 (Scheme 2a): Compound 12 (1.25 g, 2.2 mmol) was mixed with anhydrous NaHCO₃ (470 mg, 5.59 mmol) and dried over P₂O₅ under vacuum overnight. The mixture was then suspended in absolute ethanol (200 proof, 10 mL) and refluxed as reported in Example 1 to obtain the 2-O-ethyl derivative, which was subsequently reacted with DMT-Cl (750 mg, 2.21 mmol) in the presence catalytic amount of DMAP (27 mg, 0.22 mmol) in anhydrous pyridine as reported in Example 2 to obtain the desired compound 13. The product was purified by flash chromatography; eluent: EtOAc; yield: 1.02 g (59.5%). ¹H NMR (200 MHz, DMSO-d₆): δ 7.46-7.20 (m, 10H), 6.88-6.83 (d, 4H), 5.83-5.82 (d, 1H, H1′, J=1.80 Hz), 5.25 (bs, 1H, exchangeable with D₂O), 4.38-4.16 (m, 3H), 4.02-3.92 (bm, 4H), 3.72-3.65 (m, 8H), 3.47-3.42 (m, 2H), 3.31-3.19 (m, 7H, became 5H after D₂O exchange, the additional 2H could be due to the presence of water from DMSO-d₆ or from the compound), 1.31-1.24 (t, 3H), 0.73 (s, 9H), −0.07 (s, 3H), −0.10 (s, 3H). ¹³C NMR (200 MHz, DMSO-d₆): δ 168.9, 158.3, 155.0, 144.8, 135.6, 135.4, 134.3, 129.9, 128.1, 127.8, 127.0, 119.5, 113.4, 88.9, 86.0, 82.9, 81.7, 71.6, 69.7, 69.1, 64.9, 63.5, 58.7, 58.4, 55.2, 25.9, 18.1, 14.1, −5.3, −5.4.

### EXAMPLE 19

Compound 14 (Scheme 2a): Compound 13 (950 mg, 1.22 mmol) was reacted with H₂S in the presence of TMG (1.54 mL, 12.27 mmol) in anhydrous pyridine as reported in Example 3 to obtain the corresponding 2-thio derivative. The 2-thioderivative after workup was purified by flash column chromatography. Eluent: 30% EtOAc in hexane, yield: 760 mg (81.3%, white solid). ¹H NMR (200 MHz, DMSO-d₆): δ 12.78 (s, 1H, exchangeable with D₂O), 7.54 (s, 1H), 7.42-7.24 (m, 9H), 6.89-6.85 (d, 4H), 6.68 (s, 1H, H1′), 5.22-5.20 (d, exchangeable with D₂O), 4.16-3.96 (m, 4H), 3.86-3.72 (m, 8H), 3.51-3.45 (m, 2H), 3.31-3.15 (m, 6H), 0.75 (s, 9H), −0.06-−0.10 (m, 6H). Acetylation of the compound thus obtained with acetic anhydride in pyridine yields the desired product 14.

### EXAMPLE 20

Compound 15 (Scheme 2a): Treatment of compound 14 with triethylamine trihyrdofluoride in THF yields compound 15.

### EXAMPLE 21

Compound 16 (Scheme 2a): Compound 15 is reacted with methanesulfonyl chloride as reported in Example 17 to obtain compound 16.

### EXAMPLE 22

Compound 17 (Scheme 2a): Compound 16 is stirred with methylamine at low temperature and subsequently treated with ethyl trifluoroacetate in the presence of DIEA to obtain compound 17.

### EXAMPLE 23

Compound 18 (Scheme 2a): Phosphitylation of compound 17 under the conditions as reported in Example 8 yields the phosphoramidite 18.

### EXAMPLE 24

Compound 19 (Scheme 2a): Treatment of compound 16 with dimethylamine followed phosphitylation as reported in Example 8 to obtain the required amidite 19.

### EXAMPLE 25

Scheme 2b is the synthetic scheme for monomers and intermediates described in Examples 25 and 26.

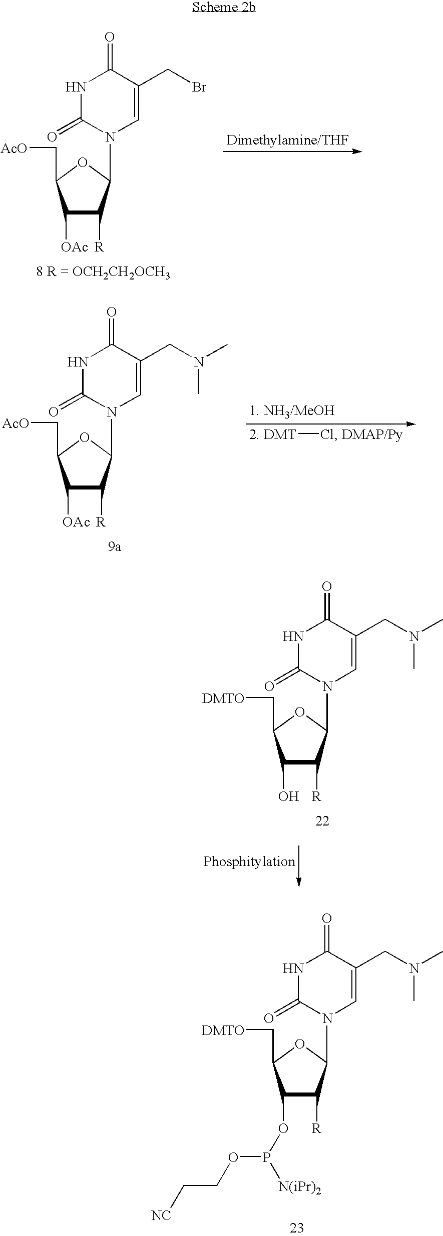

Compound 22 (Scheme 2b): Compound 8 (R′, R″=acetyl, Example 2a, 5.2 g, 10.86 mmol, purity about 90%) was treated with 2M dimethylamine in anhydrous THF (30 mL) for 10 minute at ambient temperature. Removed excess amine and THF in vacuo, and the residue were extracted into ethyl acetate. Removed the solvent in vacuo and dried under vacuum overnight to obtain compound 9a. ¹H NMR (200 MHz, DMSO-d,+D₂O): δ 7.53 (s, 1H), 5.83-5.80 (d, 1H, H1′, J=6.20 Hz), 5.18-5.14 (m, 1H), 4.33-4.19 (m, 4H), 3.56-3.51 (m, 2H), 3.35-3.15 (m, 2H), 3.15-3.12 (m, 5H), 2.14 (s, 6H), 2.06-2.05 (d, 6H).

Compound 9a was treated with methanolic ammonia for 4 h at ambient temperature to remove the acetyl protection. After ammonia treatment the residue was dried over P₂O₅ under vacuum overnight. The dried residue was treated with DMT-Cl (3.4 g, 10.03 mmol) and DMAP (25 mg, 0.20 mmol) in anhydrous pyridine under argon atmosphere to obtain compound 22. After removing pyridine from the reaction mixture the product was extracted into ethyl acetate (50 mL). The separation of aqueous and organic phase took longer time due to the presence of tertiary amino moiety in the product. The aqueous layer was re extracted with dichloromethane (50 mL) and combined the organic phase, evaporated to dryness in vacuo. The product was purified by flash column chromatography to obtain compound 22 as a yellowish solid. Eleunt: EtOAc/MeOH (1:1); isolated yield: 1.3 g (18.1%). ¹H NMR (200 MHz, DMSO-d₆): δ 11.52 (bs, exchangeable with D₂O), 7.54 (s, 1H), 7.41-7.15 (bm, 9H), 6.89-6.85 (d, 4H), 5.84-5.82 (bd, 1H, H1′, J=4.00 Hz), 5.16-5.13 (d, 1H, exchangeable with D₂O), 4.17-4.00 (bm, 3H), 3.72 (bs, 8H), 3.49-3.45 (bm, 2H), 3.22-3.03 (bm, 6H), 2.86-2.84 (bd, 1H), 2.04 (s, 6H). ¹³C NMR (50 MHz, DMSO-d₆): δ 163.9, 158.8, 150.8, 145.1, 140.6, 136.0, 135.9, 130.5, 128.6, 128.4, 127.7, 113.9, 109.1, 87.9, 86.5, 83.4, 81.7, 71.9, 70.0, 69.3, 63.7, 58.8, 55.7, 55.2, 53.9, 44.2.

### EXAMPLE 26

Compound 23 (Scheme 2b): Compound 23 was prepared from compound 22 (1.2 g, 1.82 mmol), 2-cyanoethyl tetraisopropylphosphorodiamidite (1 mL, 3.15 mmol) and tetrazole diisopropylammonium salt (310 mg, 1.81 mmol) as reported in Example 8. Due to the presence of the dimethylaminomethyl moiety in the amidite, standard chromatographic purification was not successful. So the amidite was initially precipitated from dichloromethane-hexane and subsequently purified by flash column chromatography using 30% acetone in dichloromethane as eluent under anhydrous condition to obtain the pure phosphoramidite 23 as a pale yellow solid. Isolate yield 1.16 g (77.0%). ³¹P NMR (80.96 MHz, CDCl₃): δ 151.36, 151.14.

### EXAMPLE 27

Compound 8 (R=O(CH₂)₂OCH₃, R′=Ac, R″=DMT, Scheme 2a): 5-Me-2′-O-MOE-3′-O-acetyl-5′-O-DMT-U (7) is reacted with NBS under free radical conditions as reported in Example 13 to obtain the corresponding bromo compound 8.

### EXAMPLE 28

Scheme 3 is the synthetic scheme for monomers and intermediates described in Examples 28-30.

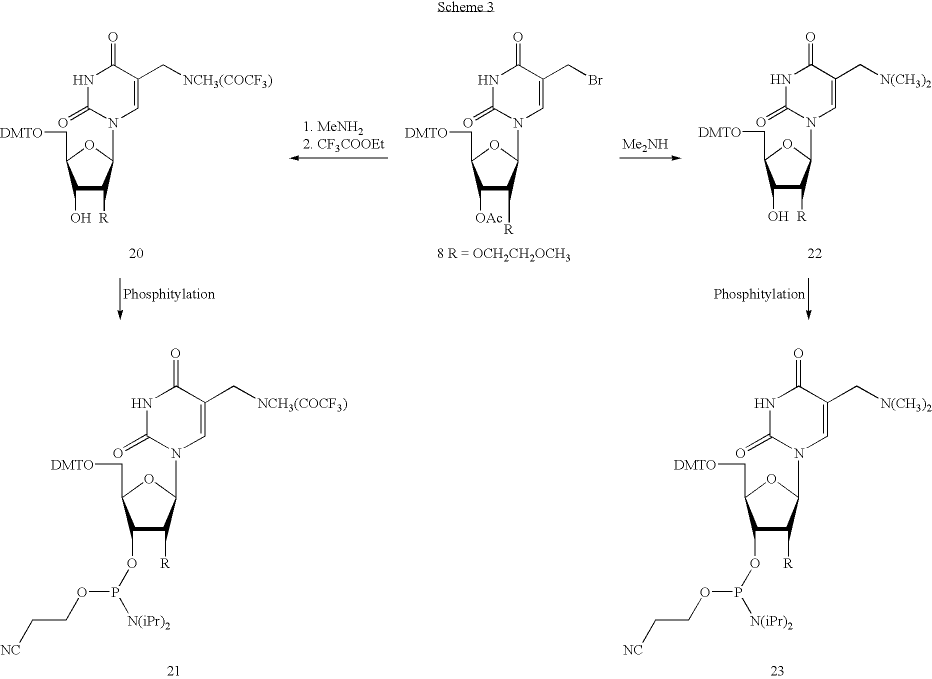

Compound 20 (R=O(CH₂)₂OCH₃, Scheme 3): Reaction of compound 8 from Example 25 with anhydrous methylamine in THF followed by ethyl trifluoroacetate in the presence of DIEA gives compound 20.

### EXAMPLE 29

Compound 21 (R=O(CH₂)₂OCH₃, Scheme 3): Phosphitylation of compound 20 under the conditions reported in Example 8 yields compound 21.

### EXAMPLE 30

Compound 23 (R=O(CH₂)₂OCH₃, Scheme 3): Treatment of the bromo compound 8 with dimethylamine followed by phosphitylation as reported in Examples 25 and 26 yields compound 23.

### EXAMPLE 31

Scheme 4 is the synthetic scheme for monomers and intermediates described in Examples 31-34. 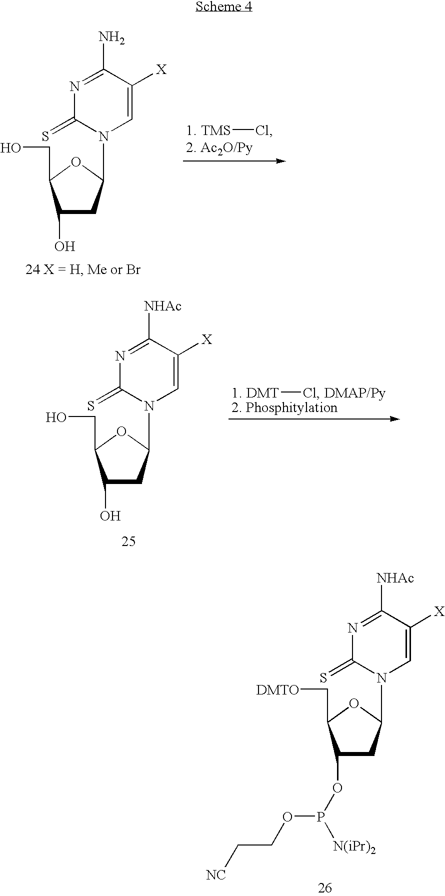
NH₂ NHAC NHAC

HO SXN 1. TM HO So N 1DMTr. DMAPPy DMTO Ski 2. AcO/Py 2. PhosphibyatIn H OH OH 70 24 X═H, Me or Br 25°-P 26 N(iPr2 NC Scheme 4

Compound 25 (X=Me, Scheme 4): Compound 24 is prepared from 5-methyl-2-thiocytosine and 1-chloro-3,5-di-O-p-toluyil-2-deoxyribofuranose as reported in the literature (Bretner et. al., Nucleosides and Nuceotides, 1995, 14, 657-660). Transient protection of the sugar hydroxyl functions of compound 24 with TMS-Cl and subsequent reaction of the silylated derivative with acetic anhydride in pyridine gives the N-acylated derivative 25.

### EXAMPLE 32

Compound 26 (X=Me, Scheme 4): Reaction of compound 25 with DMT-Cl in the presence of DMAP as reported in Example 2 gives the corresponding 5′-O-DMT protected nucleoside. The 3′-hydroxyl of which is phosphitylated under the conditions reported in Example 8 to obtain the phosphoramidite 26.

### EXAMPLE 33

Compound 26 (X=H, Scheme 4): Phosophoramidite 26 of 2-mercapto-2′-deoxycytidine is prepared from 2-thiocytosine and 1-chloro-3,5-di-O-p-toluyil-2-deoxyribofuranose as reported in Examples 31 and 32.

### EXAMPLE 34

Compound 26 (X=Br, Scheme 4): Phosphoramidite of 5-Bromo-2-thiocytidine 26 is prepared from corresponding 5-bromo-2-mercaptocytosine and 1-chloro-3,5-di-O-p-toluyil-2-deoxyribofuranose as reported in Examples 31 and 32.

### EXAMPLE 35

Scheme 5 is the synthetic scheme for monomers and intermediates described in Examples 35-41.

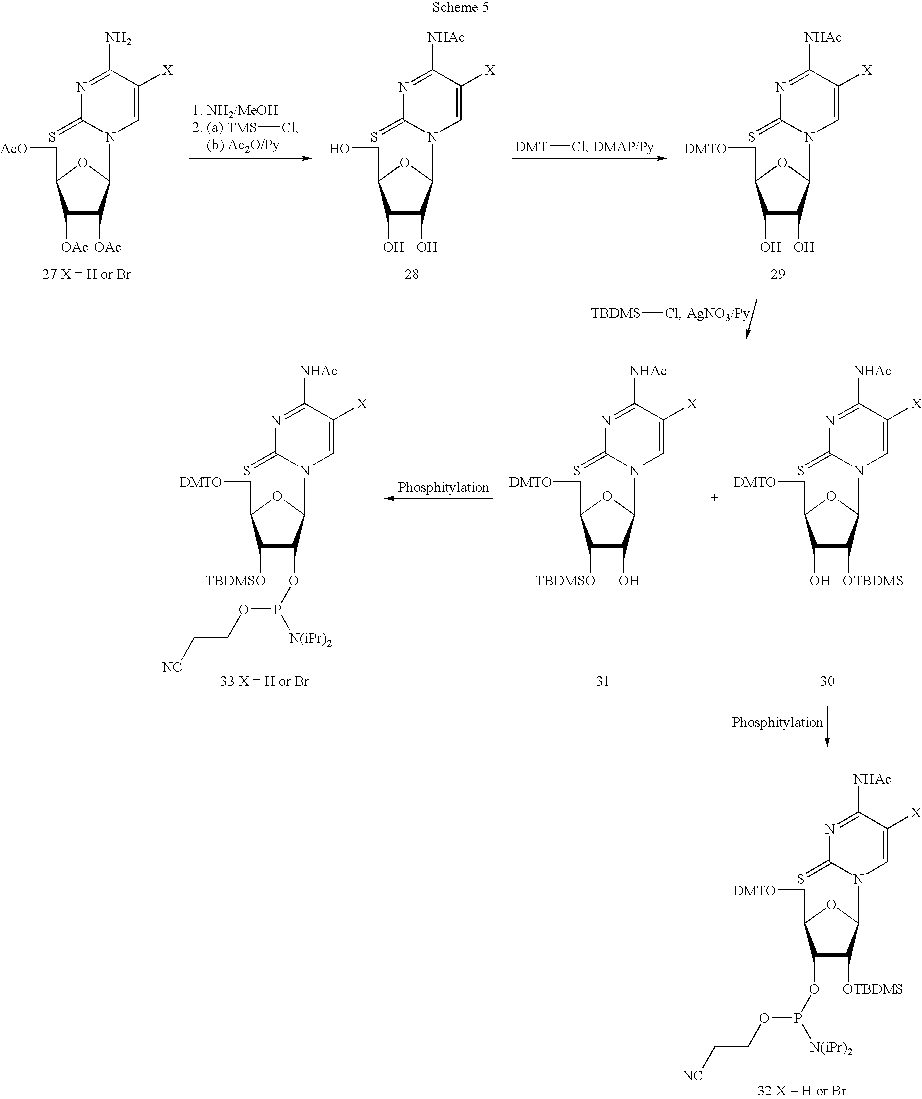

Compound 28 (X=H, Scheme 5): Compound 25 as defined is obtained from 2-mercapto-cytosine and 1,2,3,5-tetra-O-acetyl-p-D-ribofuranose as reported in the literature (Rajeev and Broom, Org. Lett., 2000, 2, 3595-3598). Compound 27 is stirred with methanolic ammonia at 0° C. to deblock the acetyl protection from the sugar moiety of compound 27. After thorough drying of the unprotected nucleoside the hydroxyl functions are transiently protected as its triemethylsilyl derivative by treatment with TMS-Cl. The sugar-protected nucleoside thus obtained is reacted with acetic anhydride in pyridine to obtain compound 28.

### EXAMPLE 36

Compound 29 (X=H, Scheme 5): Compound 28 is reacted with DMT-Cl as reported in Example 2 to obtain compound 29.

### EXAMPLE 37

Compound 30 and 31 (X=H, Scheme 5): Reaction of compound 29 with TBDMS-C1 in THF-pyridine in the presence of AgNO₃ yields mostly the 2′-O-TBDMS derivative 30 along with its 3′-O-TBDMS derivative 31 (Milicki et. al., Tetrahedron, 1999, 55, 6603-6622). Both the isomers are separated by silica gel column chromatography.

### EXAMPLE 38

Compound 32 (X=H, Scheme 5): Phosphitylation of compound 30 under the conditions reported in Example 8 yields the phosphoramidite 32.

### EXAMPLE 39

Compound 33 (X=H, Scheme 5): Compound 31 is phosphitylated as reported in Example 8 to obtain compound 33.

### EXAMPLE 40

Compound 32 (X=Br, Scheme 5): The 5-bromo-2-thio derivative of cytidine phosphoramidite is prepared from the corresponding 5-bromo-2-thiocytosine and 1,2,3,5-tetra-O-acetyl-p-D-ribofuranose as reported in Examples 35, 36, 37 and 38.

### EXAMPLE 41

Compound 33 (X=Br, Scheme 5): The desired phosphoramidite 33 is obtained from corresponding 5-halo/H-2-mercaptocytosine and 1,2,3,5-tetra-O-acetyl-o-D-ribofuranose as reported in Examples 35, 36, 37 and 38.

### EXAMPLE 42

Scheme 6 is the synthetic scheme for monomers and intermediates described in Examples 42-47.

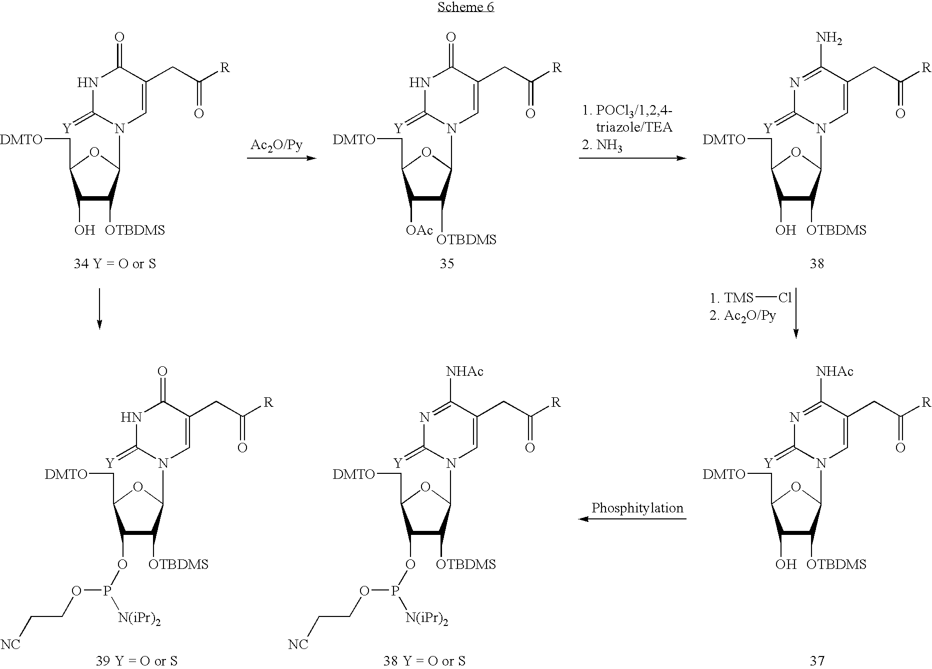

Compound 35 (R=OCH₃, Y=S, Scheme 6): Compound 34 is prepared according to the literature procedure (Bajji and Davis, Org. Lett., 2000, 2, 3865-3868). Treatment of compound 34 with acetic anhydride gives compound 35.

### EXAMPLE 43

Compound 36 (R=NH₂, Y=S, Scheme 6): Compound 35 in anhydrous acetonitrile is added dropwise into a cold stirring mixture of POCl₃, TEA and 1,2,4-triazole in anhydrous acetonitrile at −20° C. After the addition of compound 35, the reaction mixture is stirred at −20° C. for 3 h. Acetonitrile is removed from the reaction and the residue is extracted with EtOAc, washed with water and bicarbonate solution. After evaporation of EtOAc, the residue is treated with ammonia to obtain compound 36 (Shigeta et. al., Antiviral Chem., 1999, 10, 195-209).

### EXAMPLE 44

Compound 37 (R=NH₂, Y=S, Scheme 6): The free 3′-hydroxyl group of compound 36 is transiently protected using TMS-Cl and then treated with acetic anhydride in pyridine to obtain compound 37.

### EXAMPLE 45

Compound 38 (R=NH₂, Y=S, Scheme 6): Phosphitylation of compound 37 under the conditions reported in Example 8 yields compound 38.

### EXAMPLE 46

Compound 38 (R=NH₂, Y=O, Scheme 6): Phosphoramidite 38 of the cytidine derivative as defined is synthesized from the corresponding cytidine precursor 34 (Y=0) as reported in Examples 42, 43, 44 and 45. Compound 34 is obtained according to literature procedure (Bajji and Davis, Org. Lett., 2000, 2, 3865-3868).

### EXAMPLE 47

Phosphoramidite 39 (R=OMe, Y=O or S, Scheme 6): The phosphoramidite is obtained from compound 34 as reported by Bajji and Davis (Or. Lett., 2000, 2, 3865-3868).

### EXAMPLE 48

Scheme 7 is the synthetic scheme for monomers and intermediates described in Examples 48-53.

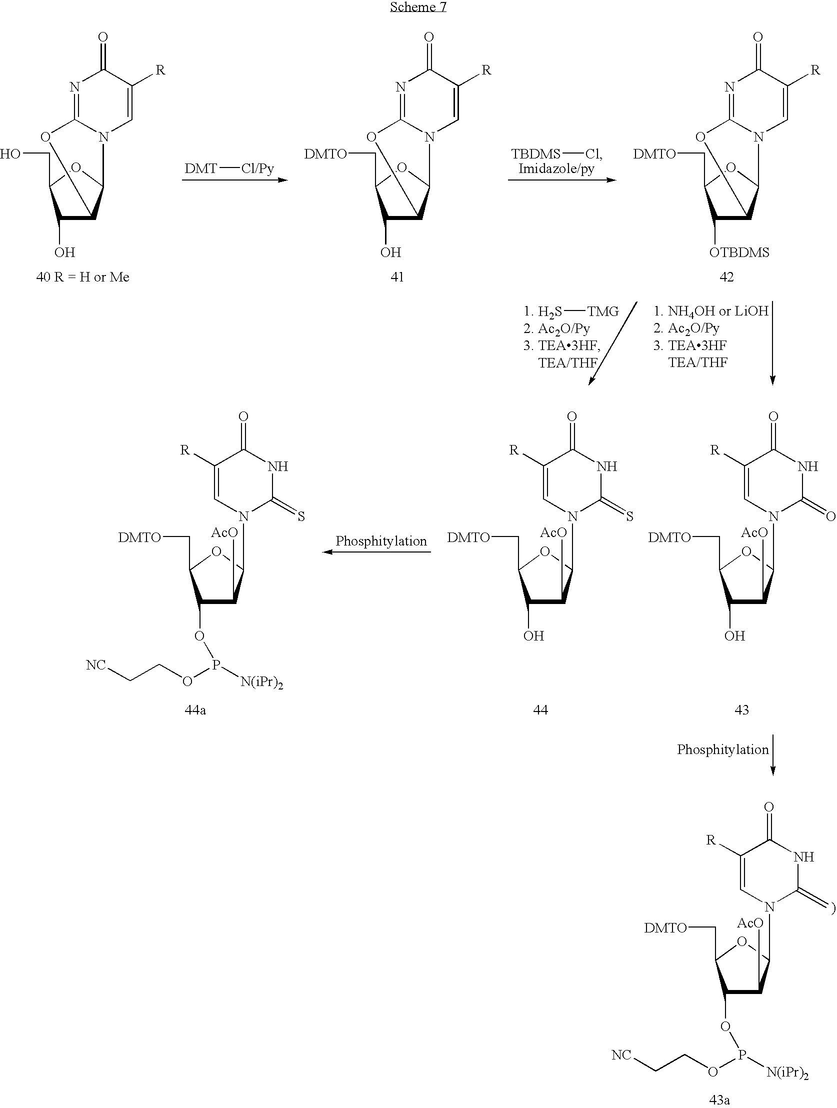

Compound 41 (R=Me, Scheme 7): 2,2′-anhydrouridne 40 is prepared from 5-methyluridine according to the literature procedure (Sebasta et. al., Tetrahedron, 1996, 52, 14385-14402). Reaction of compound 40 with DMT-Cl in the presence of DMAP in pyridine yields compound 41 (McGee et. al., J. Org. Chem., 1996, 61, 781-785).

### EXAMPLE 49

Compound 42 (R=Me, Scheme 7): Silylation of compound 41 with TBDMS-Cl in the presence of imidazole in pyridine yields compound 42.

### EXAMPLE 50

Compound 43 (R=Me, Scheme 7): Treatment of the 2,2′-anhydro nucleoside derivative 42 with ammonium hydroxide (Gazz. Chim. Ital., 1990, 120, 661-2) or with LiOH (Collect. Czech. Chem. Commun., 1990, 55, 1801-11) yields the corresponding arabino nucleoside. The arabino nucleoside thus obtained is treated with acetic anhydride in pyridine and subsequent treatment with triethylamine trihydrogenfluoride yields compound 43.

### EXAMPLE 51

Compound 43a (R=Me, Scheme 7): Phosphitylation of compound 43 as reported in Example 8 yields the phosphoramidate 43a.

### EXAMPLE 52

Compound 44 (R=Me, Scheme 7): Treatment of compound 42 with hydrogen sulfide in the presence of TMG in pyridine yields the 2-mercapto arabino nucleoside (Jpn. Kokai Tokkyo Koho, 093019931, 25 Nov. 1997, Heisei). The arabino nucleoside thus obtained is treated with acetic anhydride in pyridine and subsequent treatment with triethylamine trihydrogenfluoride yields compound 44.

### EXAMPLE 53

Compound 43a (R=Me, Example 7): Phsophitylation of compound 44 as reported in Example 8 yields the phosphoramidate 44a.

### EXAMPLE 54

Scheme 8 is the synthetic scheme for monomers and intermediates described in Examples 54-62.

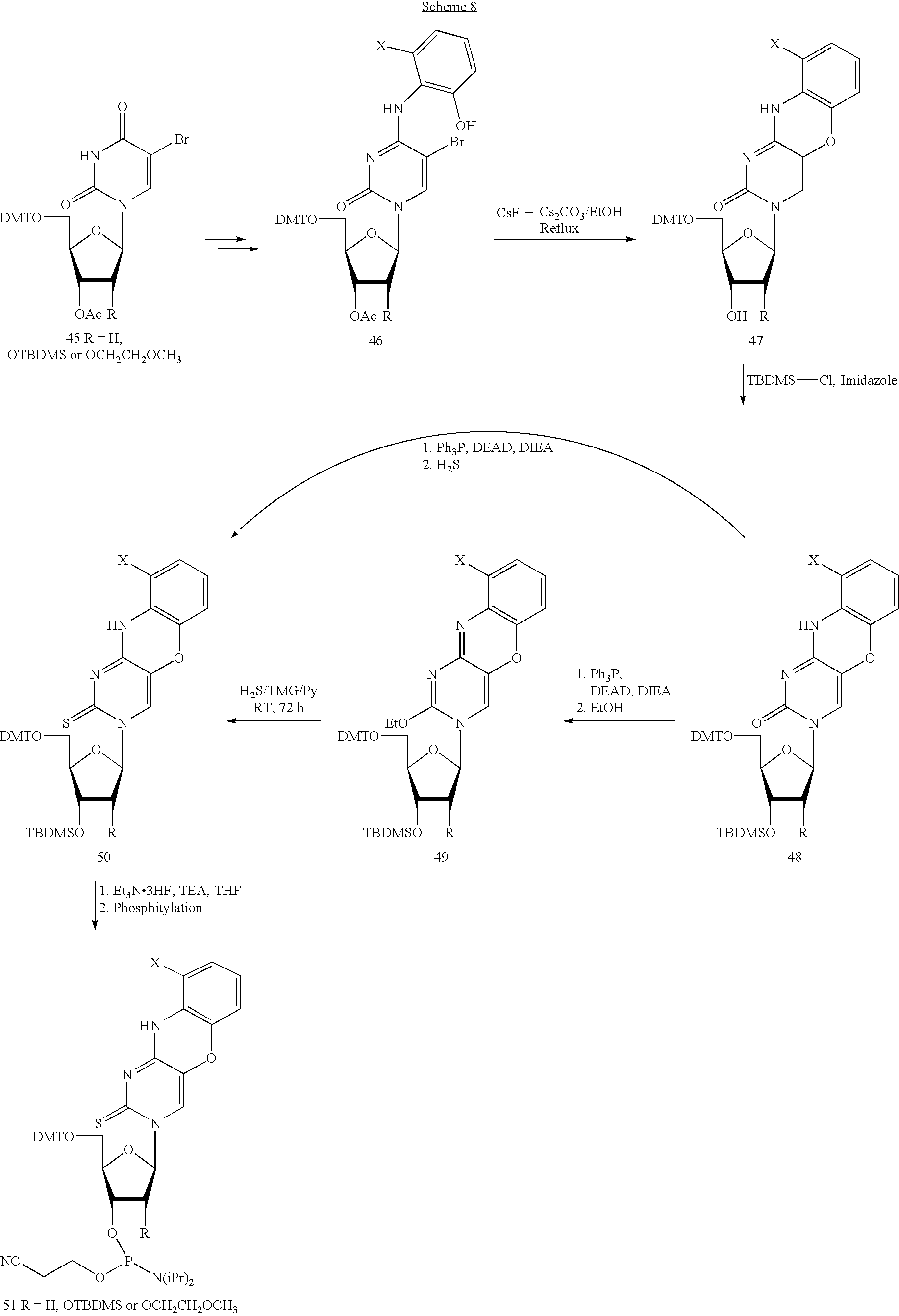

Compound 46 (Scheme 8). Cytidine derivative 46 with desired combination of R(H or OTBDMS or O(CH₂)₂OCH₃) and X (H or O-alkylamino) is synthesized from the corresponding 5-bromo-3′-O—Ac-5′-O-DMT-dU (45) according to the literature procedure by Lin and Matteucci (J. Am. Chem. Soc., 1998, 120, 8531-8532).

### EXAMPLE 55

Compound 47 (R=H, X=H, Scheme 8). Compound 46 after thorough drying over P₂O₅ is refluxed in absolute ethanol in the presence of 10 molar excess of CsF and 2 molar excess of Cs₂CO₃ to obtain compound 47.

### EXAMPLE 56

Compound 48 (R=H, X=H, Scheme 8). Silylation of compound 47 with TBDMS-Cl as reported in Example 15 yields compound 48.

### EXAMPLE 57

Compound 49 (R=H, X=H, Scheme 8). Reaction of compound 48 (1 mmol) with ethanol (1 mmol) under Mitsunobu alkylation condition (Ph₃P and DEAD 1 mmol each) in presence of DIEA in acetonitrile yields compound 49.

### EXAMPLE 58

Compound 50 (R=H, X=H, Scheme 8). Compound 49 (1 mmol) after thorough drying over P₂O₅ under vacuum is taken in a reaction flask under argon. TMG (10 mmol) in anhydrous pyridine, placed on a freezing bath, is saturated with anhydrous H₂S for 45 min. After 45 min, the resulting solution is transferred into the precooled pressure reactor containing compound 49 under argon and is sealed. The sealed vessel is then brought to ambient temperature and is stored at ambient temperature for 3 days. Bubbles off the H₂S into a chlorox bath and removes pyridine from the reaction mixture under vacuum. The residue after standard work up and purification yields compound 50.

### EXAMPLE 59

Compound 50 (R=H, X=H, Scheme 8). Compound 48 is treated with Ph₃P and DEAD in acetonitrile in the presence of DIEA under anhydrous condition and under argon for 1 h. After one hour, anhydrous H₂S gas is passed through the reaction mixture for 10 minute and the mixture is allowed to stir at ambient temperature for overnight to obtain compound 50 in one step from 47.

### EXAMPLE 60

Compound 51 (R=H, X=H, Scheme 8). Compound 50 is treated with TBAF or triethylamine trihydrofluoride in THF to remove the 3′-OTBDMS group. The resulting 3′-OH group is subjected to phosphitylation under the conditions described in Example 8 to obtain compound 51.

### EXAMPLE 61

Compound 51 (R=OTBDMS or O(CH₂)₂OCH₃, X=H, Scheme 8). The ribonucleoside or the 2′-O-MOE phosphoramidite 51 is prepared from the corresponding nucleoside precursor 46 as reported in Examples 56-60.

### EXAMPLE 62

Compound 51 (R=H or OTBDMS or O(CH₂)₂OCH₃, X=O(CH₂)₂NH₂, Scheme 8). The desired 2-mercapto ‘G-clamp’ (Lin and Matteucci, J. Am. Chem. Soc., 1998, 120, 8531-8532) phosphoramidite 51 is synthesized from the appropriate precursor 46 as reported in Examples 56-60.

### EXAMPLE 63

Scheme 9 is the synthetic scheme for monomers and intermediates described in Examples 63 and 64.

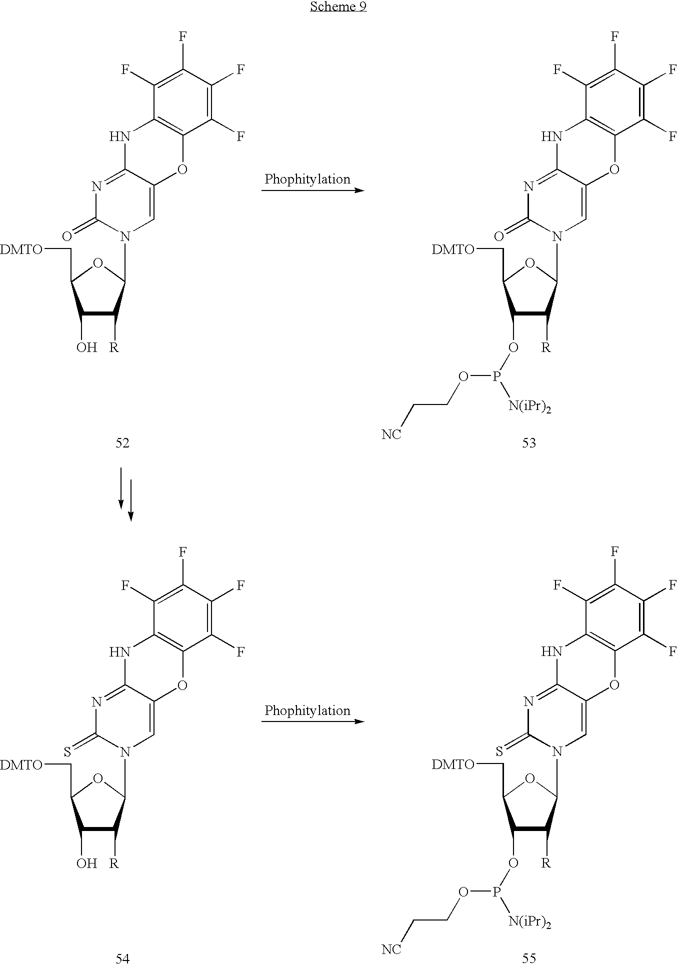

Compound 53 (R=H, X=H, Scheme 9). Compound 52 and the desired phosphoramidite are prepared according to the reported procedure in the literature (Wang et. al., Tetrahedron Lett., 1998, 39, 8385-8388).

### EXAMPLE 64

Compound 55 (R=H, X=H, Scheme 9). Compound 52 is obtained according to the literature procedure (Wang et. al., Tetrahedron Lett., 1998, 39, 8385-8388). The 2-thio analogue 55 of compound 52 is synthesized from compound 52 as reported in Examples 56-60.

### EXAMPLE 65

Scheme 10 is the synthetic scheme for monomers and intermediates described in Examples 65-72.

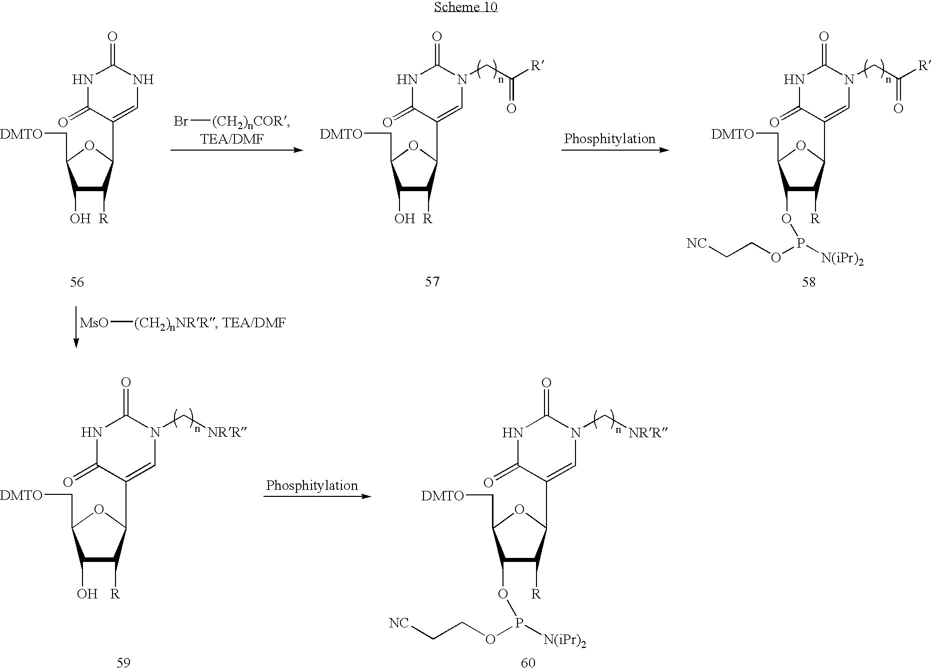

Compound 57 (R=H, R′=OEt, n=1, Scheme 10): Pseudouridine derivative 56 is prepared according to reported procedure (Grohar and Chow, Tetrahedron Let., 1999, 40, 2049-2052). Compound 56 is stirred with one equivalent of ethylbromoacetate in anhydrous DMF in the presence of triethylamine to obtain compound 57.

### EXAMPLE 66

Compound 58 (R=H, R′=OEt, n=1, Scheme 10): Phosphitylation of compound 57 under the conditions reported in Example 8 yields the phosphoramidate 58.

### EXAMPLE 67

Compound 58 (R=H, R′=NH₂, n=1, Scheme 10): Compound 57 upon treatment with ammonia under anhydrous condition yields the corresponding amide, which is then subjected to phosphitylation as reported in Example 8 to obtain compound 58.

### EXAMPLE 68

Compound 59 (R=H, R′, R″=Me, n=2, Scheme 10). Compound 56 is stirred with [2-(dimethylamino)ethyl]methanesulfonate in the presence of triethylamine in anhydrous DMF to obtain compound 59.

### EXAMPLE 69

Compound 60 (R=H, R′, =Me, n=2, Scheme 10). Phosphitylation of compound 59 as reported in Example 8 yields compound 60.

### EXAMPLE 70

Compound 58 (R=OTBDMS, R′=OEt, Scheme 10): Compound 56, where R=OTBDMS is prepared according to literature procedure (Gasparotto et. al., Nucleic Acids Res., 1992, 20, 5159-5166). The desired phosphoramidate 58 is obtained from compound 56 by following the procedures reported in Examples 65 and 66.

### EXAMPLE 71

Compound 58 (R=OTBDMS, R′=NH₂, n=1, Scheme 10): Compound 56, where R=OTBDMS is prepared according to literature procedure (Gasparotto et. al., Nucleic Acids Res., 1992, 20, 5159-5166). The desired phosphoramidate 58 is obtained from compound 56 by following the procedures reported in Examples 65 and 67.

### EXAMPLE 72

Compound 60 (R=OTBDMS, R′, R″=Me, n=2, Scheme 10): Compound 56, where R=OTBDMS is prepared according to literature procedure (Gasparotto et. al., Nucleic Acids Res., 1992, 20, 5159-5166). The desired phosphoramidate 58 is obtained from compound 56 by following the procedures reported in Examples 68 and 69.

### EXAMPLE 73

Scheme 11 is the synthetic scheme for monomers and intermediates described in Examples 73-78.

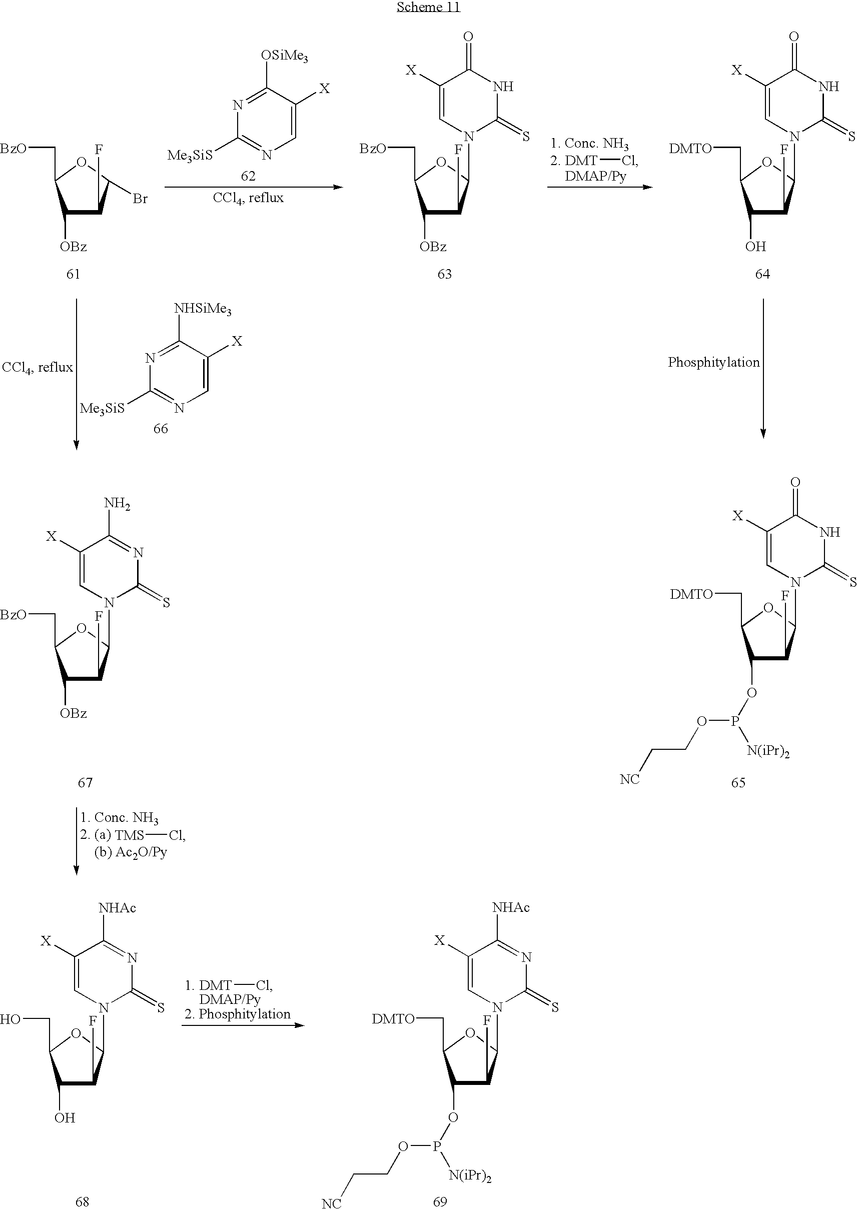

Compound 63 (X=Me, Scheme 11). Compound 61 is obtained from 1,3,5-tri-O-benzoyl-α-D-ribofuranose according to the reported procedure (Wilds and Damha, Nucleic Acids Res., 2000, 28, 3625-3635). A mixture of compound 61 (1 mmol) and 2-S-(trimethylsilyl)-4-O-(trimethylsilyl)thymine (62, 1.2 mmol) in CCl₄ is allowed to reflux for 72 h as reported in the literature (Wilds and Damha, Nucleic Acids Res., 2000, 28, 3625-3635). The reaction is quenched with methanol and solid formed is filtered. Evaporation of the solution followed by flash column chromatography yields compound 63.

### EXAMPLE 74

Compound 64 (X=Me, Scheme 11). Compound 63 is stirred with concentrated ammonia at ambient temperature to deprotect benzoyl groups from 3′ and 5′ hydroxyl groups. This after thorough drying over P₂O₅ is reacted with DMT-Cl in pyridine in the presence of DMAP to obtain compound 64.

### EXAMPLE 75

Compound 65 (X=Me, Scheme 11). Phosphitylation of compound 64 as reported in Example 8 yields the phosphoramidite 65.

### EXAMPLE 76

Compound 67 (X=Me, Scheme 11). A mixture of compounds 61 (1 mmol) and 5-methyl-2-S-(trimethylsilyl)-4-N-(trimethylsilyl)cytosine (66, 1.2 mmol) in CCl₄ is allowed to reflux for 72 h. The reaction is quenched with methanol and solid formed is filtered. Evaporation of the solution followed by flash column chromatography yields compound 67 (Wilds and Damha, Nucleic Acids Res., 2000, 28, 3625-3635).

### EXAMPLE 77

Compound 68 (X=Me, Scheme 11). Compound 67 is stirred with concentrated aqueous ammonia to remove the benzoate. The product thus obtained is transiently protected with trimethylsilyl chloride in anhydrous pyridine and subsequently reacted with acetic anhydride to obtain compound 68.

### EXAMPLE 78

Compound 69 (X=Me, Scheme 11). The phosphoramidite 69 is prepared from compound 68 in two steps as reported in Examples 74 and 75.

### EXAMPLE 79

Scheme 12 is the synthetic scheme for monomers and intermediates described in Examples 79-82.

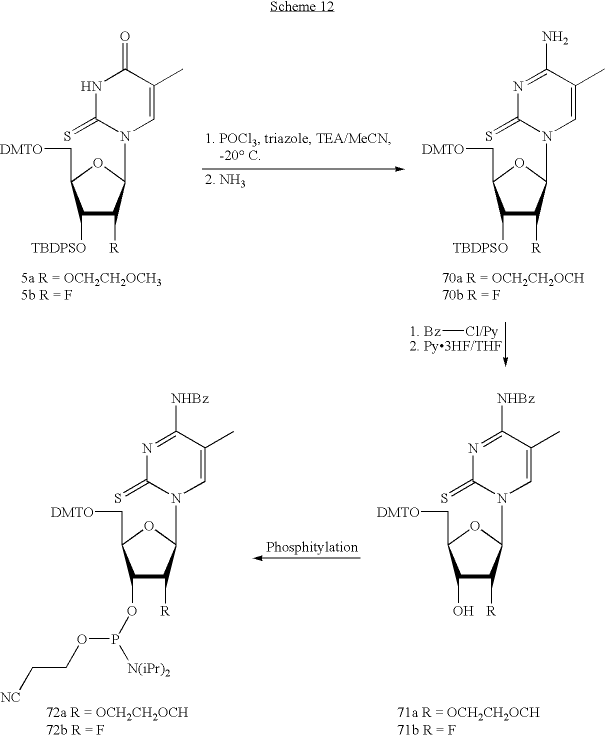

Compound 70a (R=OCH₂CH₂OCH₃, Scheme 12): Compound 5 (1.75 g, 3.07 mmol, obtained from Example 5, Example 1) was treated with DMT-Cl (1.35 g, 3.98 mmol) in the presence of DMAP (50 mg, 0.41 mmol) in anhydrous pyridine as reported in Example 2, to obtain compound 5a (as specified in Example 5). Compound 5a was purified by flash column chromatography; eluent: Hexane/EtOAc (3:1); yield: 2.6 g, (97.1%). ¹H NMR, 6 (DMSO-d₆): 7.97 (bs, 1H, exchangeable with D₂O), 7.62-7.59 (m, 2H), 7.47-7.10 (m, 17H), 6.99-6.97 (d, 1H, H1′, J=3.00 Hz), 6.86-6.80 (m, 4H), 4.24-4.10 (m, 2H), 4.06-3.97 (m, 1H), 3.72 (s, 6H), 3.64-3.60 (t, 1H), 3.31-3.20 (m, 4H), 3.17 (s, 3H), 3.02-2.95 (m, 1H), 1.36 (s, 3H), 0.91 (s, 9H).

Compound 5a (2.35 g, 2.69 mmol) was mixed with triazole (1.9 g, 27.53 mmol) and dried overnight over anhydrous P₂O₅ under vacuum. The mixture was suspended in anhydrous CH₃CN under argon and stirred at −20° C. TEA (3.8 mL, 27.26 mmol) was added into the stirring suspension and the stirring was continued for 20 minutes. While maintaining the bath temperature at −20° C., POCl₃ (0.75 mL, 8.06 mmol) was added into the reaction mixture drop-wise. The addition was completed in 20 min and the mixture was allowed stir at −20° C. for 2 h. Removed CH₃CN from the reaction mixture at low temperature under vacuum and the triazolide formed was extracted into ethylacetate, washed with water and saturated sodium bicarbonate solution. Evaporation of ethyl acetate gave a yellow solid. The solid thus obtained was dissolved in THF (10 mL), aqueous ammonia (10 mL) was added into the THF solution and stirred at ambient temperature for 40 min. Removed THF and ammonia from the reaction mixture and the residue in EtOAc (30 mL) was washed with water and sodium bicarbonate solution followed by evaporation of solvent to dryness. The cytidine derivative 70a was finally purified to obtain as a pale yellowish white solid by flash column chromatography; eluent: 3% MeOH in dichloromethane; yield: 2.25 g, (95.9%). ¹H NMR, 5 (CDCl₃-d₆): 8.29-8.26 (d, 2H), 7.82 (s, 1H), 7.827.18 (m, 22H), 6.82-6.73 (m, 5H), 4.32-4.27 (m, 2H), 4.09-4.00 (m, 1H), 3.79 (bs, 7H), 3.55-3.35 (m, 4H), 3.30 (s, 3H), 3.10-3.06 (m, 1H), 1.42 (s, 3H), 0.99 (s, 9H).

### EXAMPLE 80

Compound 71a (Scheme 12): Compound 70a (1.9 g, 2.18 mmol) was dissolved into a mixture of pyridine-dichloromethane (1:1, 10 mL) and stirred at −20° C. under argon. Benzoyl chloride (0.4 mL, 3.45 mmol) was added drop-wise into the stirring solution. The stirring was continued at −20° C. bath temperature for 1 h. Methanol was added into the reaction to quench excess benzoyl chloride. Removed pyridine and dichloromethane in vacuo. The residue was taken in EtOAc (30 mL) and washed with sodium bicarbonate solution followed by standard workup. The N4-benzoylated product 70a was purified by flash column chromatography; eluent: 20% EtOAc in Heaxane; yield: 1.41 g (66.4%, yellowish white solid).

¹H NMR, δ (CDCl₃-d₆): 8.29-8.26 (d, 2H), 7.82 (s, 1H), 7.827.18 (m, 22H), 6.82-6.73 (m, 5H), 4.32-4.27 (m, 2H), 4.09-4.00 (m, 1H), 3.79 (bs, 7H), 3.55-3.35 (m, 4H), 3.30 (s, 3H), 3.10-3.06 (m, 1H), 1.42 (s, 3H), 0.99 (s, 9H).

The compound thus obtained (1.34 g, 1.38 mmol) was dissolved in anhydrous THF (10 mL) under argon and stirred at ambient temperature. To the stirring solution TEA (0.45 mL, 3.23 mmol) was added followed by triethylamine trihydrofluoride (0.85 mL, 5.21 mmol). The reaction mixture was allowed to stir overnight under argon. THF was removed from the reaction mixture and the residue taken in EtOAc (30 mL) was washed with saturated sodium bicarbonate (20 mL) and water (10 mL). Organic phase was evaporated to a solid mass. The desired N⁴-benzoylated product 71a was finally purified by flash silica gel column chromatography; eluent: 40% EtOAc in hexane; yield: 900 mg (88.9%, yellowish white solid.:

¹H NMR, δ (CDCl₃-d₆+D₂O): 8.30-8.27 (d, 2H), 8.13 (s, 1H), 7.53-7.26 (m, 12H), 6.88-6.84 (m, 4H), 6.49 (s, 1H), 4.53-4.46 (m, 1H), 4.32-4.26 (bm, 1H), 4.17-4.10 (m, 2H), 3.98-3.89 (bm, 1H), 3.80 (s, 6H), 3.65-3.47 (m, 4H), 3.40-3.39 (m, 3H), 1.46 (s, 3H). ¹³C NMR, 6 (CDCl₃): 179.5, 170.9, 158.8, 158.7, 156.1, 144.3, 136.9, 135.3, 135.2, 132.5, 130.2, 129.9, 128.2, 128.1, 128.0, 117.3, 113.3, 93.3, 86.9, 83.6, 83.4, 71.7, 71.6, 68.6, 61.2, 58.9, 55.3, 13.0.

### EXAMPLE 81

Compound 72a (Scheme 12): Treatment of compound 71a (850 mg, 1.15 mmol) with 2-cyanoethyl tetraisopropylphosphorodiamidite (750 μL, 2.36 mmol) and tetrazole diisopropylammonium salt (200 mg, 1.17 mmol) as reported in Example 8 to obtain compound 72a. The amidite thus formed was purified by flash silica gel column chromatography; eluent: 20% EtOAc in Hexane; yield: 790 mg (73.1%). ³¹P NMR, δ (CDCl₃-d₆): 151.71, 150.74

### EXAMPLE 82

Compound 72b (R=F, Scheme 12): Compound 5, where R=F, R′=H and =DMT, obtained from Example 11 is silylated with TBDMS-Cl in the presence of imidazole in anhydrous pyridine to obtain compound 5b. The desired phosphoramidate 72b is prepared from compound 5b as reported in Examples 79 (appropriate parts of the experimental procedure), 80 and 81.

### EXAMPLE 83

Scheme 13 is the synthetic scheme for monomers and intermediates described in Examples 83-90.

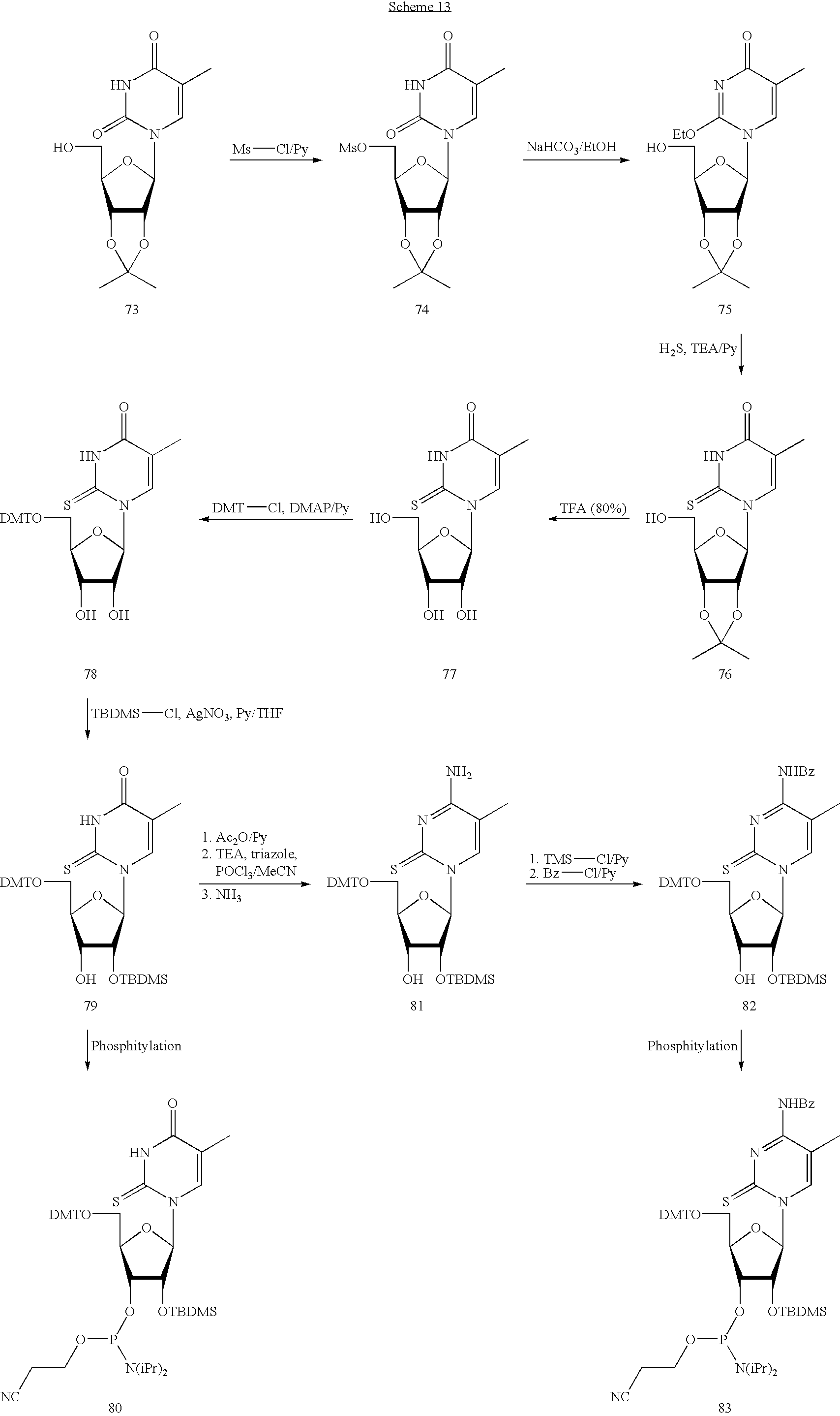

Compound 74 (Scheme 13): Compound 73 was prepared according to the literature procedure (Kumar and Walker, Tetrahedron, 1990, 46, 3101-10). Compound 73 (42.5 g, 142.62 mmol) was dissolved in pyridine-dichloromethane (1:1, 150 mL) and stirred at −20° C. under argon. Methanesulfonyl chloride (22 mL, 284.24 mmol) was added drop-wise into the stirring solution, the addition was completed in 10 min and the mixture was allowed to stir for 1 h at −20° C. Removed pyridine and dichloromethane in vacuo and the residue suspended in EtOAc (400 mL) was washed with water and saturated sodium bicarbonate solution. After removal of the ethyl acetate in vacuo, the residue was redissolved in dichloromethane (200 mL) and treated with activated charcoal, filtered through a column of celite and evaporated to a white solid, yield: 52.16 g (97.3%). ¹H NMR (200 MHz, DMSO-d₆): δ 11.40 (s, 1H, exchangeable with D₂O), 7.55 (s, 1H), 5.81 (s, 1H), 5.06-5.03 (m, 1H), 4.84-4.79 (m, 1H), 4.43-4.23 (m, 3H), 3.19 (s, 3H), 1.76 (s, 3H), 1.49 (s, 3H), 1.29 (s, 3H). ¹³C NMR (50 MHz, DMSO-d₆): δ 163.8, 150.3, 138.4, 113.5, 109.7, 92.2, 83.8, 83.3, 80.4, 69.4, 36.7, 26.9, 25.1, 11.9.

### EXAMPLE 84

Compound 75 (Scheme 13): Compound 74 (47.5 g, 126.33 mmol) and NaHCO₃ (21.23 g, 252.71 mmol) were mixed in a 200 ML RB and dried over P₂O₅ under vacuum overnight. Absolute ethanol (200 proof, 200 mL) was added into the mixture under argon atmosphere and refluxed for 48 h under argon. The reaction mixture was cooled to room temperature and filtered through a sintered funnel, the solid residue was thoroughly washed with methanol, combined the washing and concentrated to 50 mL. Compound 75 was precipitated from the solution by adding diethyl ether (200 mL) in to the methanolic solution. The precipitate was filtered and dried over P₂O₅ under vacuum overnight to obtain a white solid, 28.54 g (69.3%). ¹H NMR (200 MHz, DMSO-d₆): δ 7.71 (s, 1H), 5.81-5.80 (d, 1H, H1′, J=2.60 Hz), 5.16 (s, 1H, exchangeable with D₂O), 4.92-4.87 (m, 1H), 4.77-4.72 (m, 1H), 4.39-4.28 (q, 2H), 4.11-4.09 (bm, 1H), 3.59 (bs, 2H), 1.78 (s, 3H), 1.49 (s, 3H), 1.33-1.26 (m, 6H). ¹³C NMR (50 MHz, DMSO-d₆): δ 170.8, 154.8, 135.2, 116.0, 113.4, 92.0, 86.3, 84.2, 80.3, 64.7, 61.2, 27.2, 25.4, 14.1, 13.5.

### EXAMPLE 85

Compound 76 (Scheme 13): Compound 75 (6.15 g, 18.87 mmol) was dried over P₂O₅ under vacuum overnight and was treated with H₂S and triethylamine in anhydrous pyridine as reported in Example 3. After removing H₂S and pyridine the product was precipitated out from water, filtered, washed with water and diethyl ether to obtain the desired compound 76 as a white solid, 5.49 g (92.7%). ¹H NMR (200 MHz, DMSO-d₆): δ12.65 (s, 1H, exchangeable with D₂O), 7.88 (s, 1H), 6.90-6.89 (d, 1H, H1′, J=1.40 Hz), 5.34-5.29 (t, 1H, exchangeable with D₂O), 4.80 (bm, 2H), 4.10-4.09 (m, 1H), 3.69-3.62 (m, 2H), 1.81 (s, 3H), 1.50 (s, 3H), 1.28 (s, 3H). ¹³C NMR, (50 MHz, DMSO-d₆): δ 175.2, 160.7, 137.4, 115.8, 113.6, 92.7, 86.1, 84.4, 79.5, 27.3, 25.5, 12.6.

### EXAMPLE 86

Compound 77 (Scheme 13): Compound 76 (5.1 g, 16.24 mmol) was stirred in 80% trifluoroacetic acid (60 mL) for 6 h. After removing the acid and water from the reaction, the residue was thoroughly washed with ethyl acetate followed by drying under vacuum over P₂O₅ to obtain compound 77 as a white solid, yield 3.85 g (86.5%). ¹H NMR (200 MHz, DMSO-d₆): δ 12.55 (s, 1H, exchangeable with D₂O), 8.11-8.10 (d, 1H, H6 J=1.20 Hz), 6.55-6.53 (d, 1H, H1′, J=3.40 Hz), 4.06-3.55 (m, 5H), 1.80-1.79 (d, 3H). ¹³C NMR, (50 MHz, DMSO-d₆): δ 175.1, 160.6, 137.1, 114.8, 92.5, 84.5, 74.4, 68.8, 59.8, 12.5.

### EXAMPLE 87

Compound 78 (Scheme 13): Compound 77 (3.5 g, 12.77 mmol) was treated with DMT-Cl (4.76 g, 14.05 mmol) in the presence on DMAP (350 mg, 2.86 mmol) in anhydrous pyridine as reported in Example 2 to obtain the desired compound. The compound 78 was purified by flash silica gel column chromatography; eluent: 4% methanol in dichloromethane; yield: 4.37 g, 59.4 g. ¹H NMR (200 MHz, CDCl₃): δ 7.93-7.92 (d, 1H, J=1 Hz), 7.42-7.19 (in, 9H), 6.87-6.81 (m, 4H), 6.48-6.47 (d, 1H, H1′, J=2 Hz), 4.49-4.41 (m, 2H), 4.26-4.23 (m, 1H), 3.79 (s, 6H), 3.63-3.40 (m, 2H), 1.45-1.44 (d, 3H, J=0.4 Hz).

### EXAMPLE 88

Compound 79 (Scheme 13): Compound 79 is obtained from compound 78 and TBDMS-Cl as reported in Example 37.

### EXAMPLE 89

Compound 80 (Scheme 13): Phosphitylation of compound 79 as reported in Example 8 yields the desired phosphoramidate 80.

### EXAMPLE 90

Compound 83 (Scheme 13): Compound 83 is obtained from compound 79 as reported in Examples 79 (appropriate parts of experimental procedure), 80 and 81.

### EXAMPLE 91

Scheme 14a is the synthetic scheme for monomers and intermediates described in Examples 91-104.

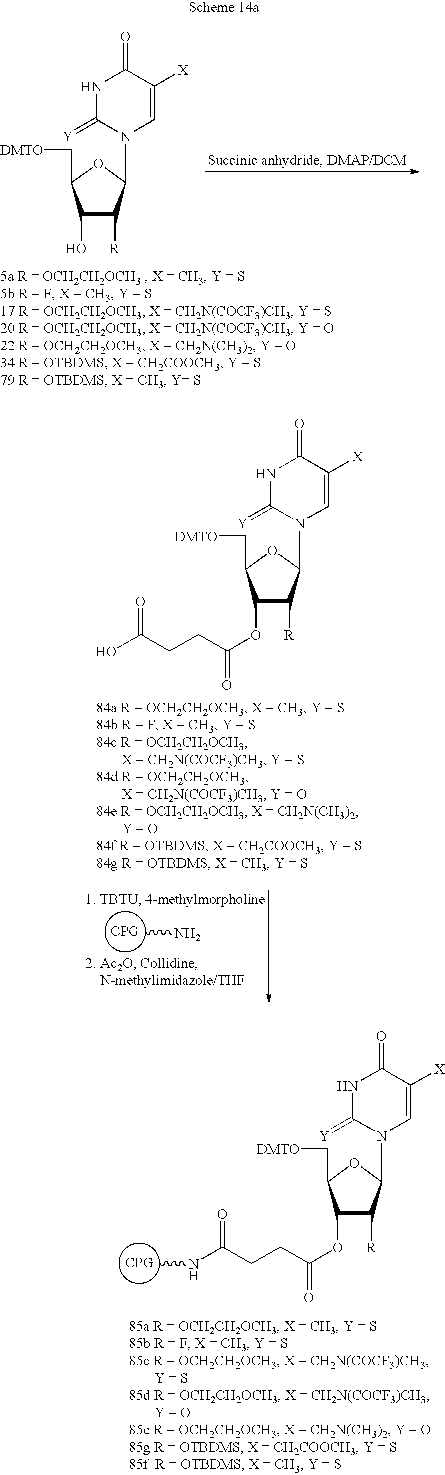

Compound 84a (R=OCH₂CH₂OCH₃, Scheme 14a): Compound 5a (1 mmol) is mixed with succinic anhydride (2 mmol) and dimethlyaminopyridine (1 mmol), and is dried over P₂O₅ in vacuo overnight. Dichloromethne (0.9 mL) is added into the mixture and stirs at ambient temperature for 8 h. The reaction mixture is diluted with excess dichloromethane and the organic layer is subjected ice cold aqueous citric acid wash (10% solution) and brine. The organic phase is dried over anhydrous Na₂SO₄ and concentrated to dryness to yield the succinic acid derivative 84a.

### EXAMPLE 92

Compound 85a (R=OCH₂CH₂OCH₃, Scheme 14a): Compound 84a (1 mmol) is dried over P₂O₅ under vacuum overnight. Anhydrous DMF is added into the dried 84a and mixed with 2-(1H-benzotriazole-1-yl)-1,1,3,3-tetramethyluronium tetrafluoroborate (TBTU, 1 mmol) and 4-methylmorpholine (2 mmol) with vortexing to give a clear solution. Calculated amount of CPG (118.9 μmol/g, particle size 80/120, mean pore diameter 569 Å) is added into the clear solution and allows to shake on a shaker at ambient temperature for 18 h. An aliquot of the support is withdrawn and washed with DMF, CH₃CN and diethylether, and dries in vacuo. Loading capacity is determined by following standard procedure. Functionalized CPG is then washed with DMF, CH₃CN, diethylether and dried in vacuo. Unfunctionalized sites on the CPG are capped with acetic anhydride/collidine/N-methylimidazole in THF (2 mL Cap A and 2 mL Cap B solutions from Perspective Biosystems Inc.) and allows to shake on a shaker for 2 h. The CPG is filtered, washed with CH₃CN followed by diethlether, and dries in vacuo. The final loading capacity of 85a is determined after capping.

### EXAMPLE 93

Compound 85b (Scheme 14a): The desired solid support 85b is obtained from its corresponding precursor 5b as reported in Examples 91 and 92.

### EXAMPLE 94

Compound 85c (Scheme 14a): The desired solid support 85c is obtained from its corresponding precursor 17 as reported in Examples 91 and 92.

### EXAMPLE 95

Compound 85d (Scheme 14a): The desired solid support 85d is obtained from its corresponding precursor 20 as described in Examples 91 and 92.

### EXAMPLE 96

Compound 85e (Scheme 14a): The desired solid support 85e is obtained from its corresponding precursor 22 as described in Examples 91 and 92.

### EXAMPLE 97

Compound 85g (Scheme 14a): The desired solid support 85f is obtained from its corresponding precursor 34 as described in Examples 91 and 92.

### EXAMPLE 98

Compound 85f (Scheme 14a): The desired solid support 85f is obtained from its corresponding precursor 79 as described in Examples 91 and 92.

### EXAMPLE 99

Scheme 14b is the synthetic scheme for monomers and intermediates described in Examples 99-104.

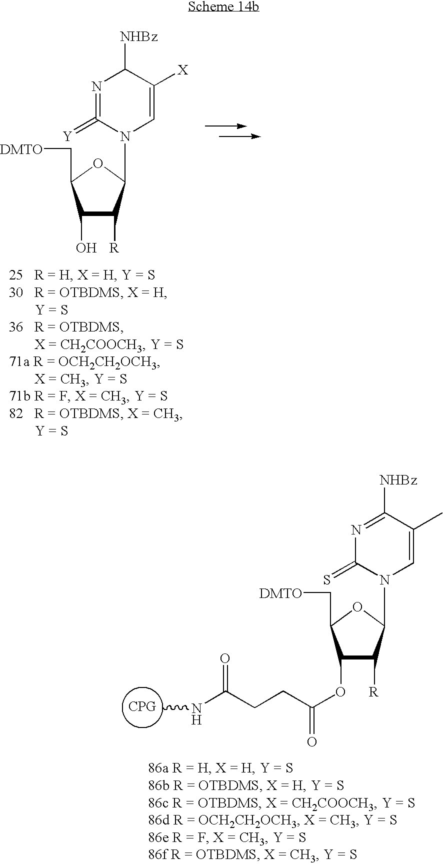

Compound 86a (Scheme 14b): The desired solid support 86a is obtained from its corresponding precursor 25 as described in Examples 91 and 92.

### EXAMPLE 100

Compound 86b (Scheme 14b): The desired solid support 86b is obtained from its corresponding precursor 30 as described in Examples 91 and 92.

### EXAMPLE 101

Compound 86c (Scheme 14b): The desired solid support 86c is obtained from its corresponding precursor 36 as described in Examples 91 and 92.

### EXAMPLE 102

Compound 86d (Scheme 14b): The desired solid support 86d is obtained from its corresponding precursor 71a as described in Examples 91 and 92.

### EXAMPLE 103

Compound 86e (Scheme 14b): The desired solid support 86e is obtained from its corresponding precursor 71b as described in Examples 91 and 92.

### EXAMPLE 104

Compound 86f (Scheme 14b): The desired solid support 86f is obtained from its corresponding precursor 82 as described in Examples 91 and 92.

### EXAMPLE 105

Scheme 14c is the synthetic scheme for monomers and intermediates described in Examples 105-107.

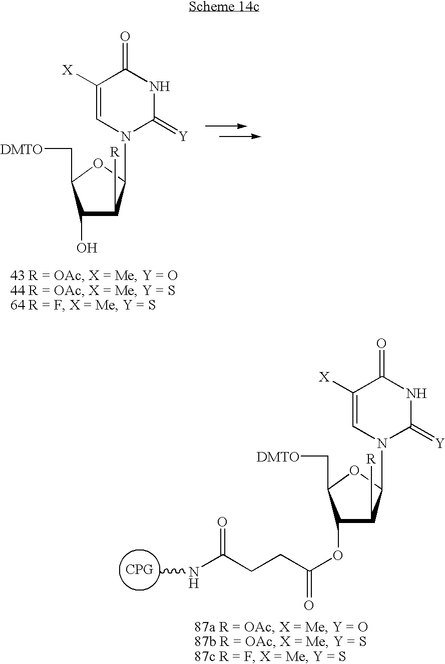

Compound 87a (Scheme 14c): The desired solid support 87a is obtained from its corresponding precursor 43 as described in Examples 91 and 92.

### EXAMPLE 106

Compound 87b (Scheme 14c): The desired solid support 87b is obtained from its corresponding precursor 44 as described in Examples 91 and 92.

### EXAMPLE 107

Compound 87c (Scheme 14c): The desired solid support 87c is obtained from its corresponding precursor 64 as described in Examples 91 and 92.

### EXAMPLE 108

Scheme 15 is the synthetic scheme for monomers and intermediates described in Examples 108-119 and 121-124.

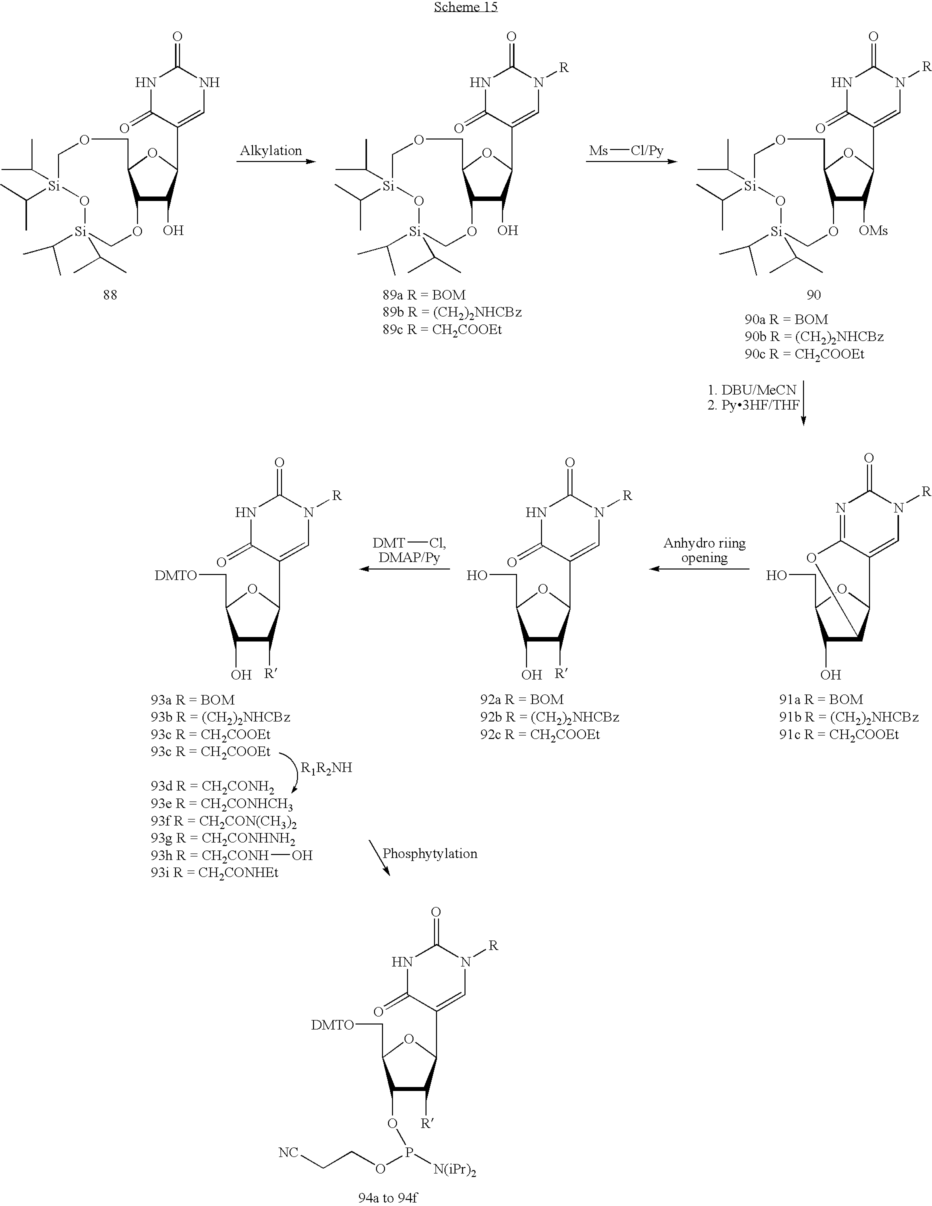

Compound 89a (R=BOM, Scheme 15): Compound 88 is prepared according to the literature procedure (Nucleosides Nucleotides, 1985, 4, 613-24). Compound 88 (1 mmol) is stirred with BOM-C1 in dichloromethane in the presence of TEA to obtain compound 89a.

### EXAMPLE 109

Compound 90a (R=BOM, Scheme 15): Compound 89a is stirred in pyridine with methanesulfonyl chloride at 0° C. for 1 hr to obtain compound 90a.

### EXAMPLE 110

Compound 91a (R=BOM, Scheme 15): Compound 90a is treated with DBU in MeCN to obtain the corresponding sugar protected anhydro derivative. Treatment of the protected nucleoside thus obtained with pyridinium trihydrogen fluoride in anhydrous THF yields compound 91a.

### EXAMPLE 111

Compound 92a (R=BOM, R′=OCH₂CH₂OCH₃, Scheme 15): The compound 91a (1 mmol) with 2 equivalent of (CH₃OCH₂CH₂O)₃B in the presence of PTSA yields 2′-O-metohxyethyl-pseudouriidne 92a.

### EXAMPLE 112

Compound 93a (R=BOM, R′=OCH₂CH₂OCH₃, Scheme 15): Compound 92a is stirred with DMT-Cl in anhydrous pyridine in the presence of DMAP as described in Example 2 to obtain compound 93a.

### EXAMPLE 113

Compound 94a (R=H R′=OCH₂CH₂OCH₃, Scheme 15): Catalytic reduction of compound 93a followed by basic hydrolysis gives the corresponding N1 deprotected nucleoside (Macor et. al., Tetrahedron Let., 1977, 38, 1673). Phosphitylation of compound, obtained from the reductive hydrolysis, as described in Example 8 yields compound 94a.

### EXAMPLE 114

Compound 89b (R=CH₂CH₂NHCbz, Scheme 15): Compound 88 is stirred with N-carbobenzyloxyethanolamine-O-mesylate [(CBz)HNCH₂CH₂OSO₂Me] in the presence of base to obtain compound 89b. The mesylate is prepared from N-carbobenzyloxyethanolamine according to standard procedure.

### EXAMPLE 115

Compound 92b (R=CH₂CH₂NHCbz, R′=OCH₂CH₂OCH₃, Scheme 15): Compound 92b as defined is obtained from compound 89b as described in Examples 109,110 and 111.

### EXAMPLE 116

Compound 93b (R=(CH₂)₂NHCOCF₃, R′=OCH₂CH₂OCH₃, Scheme 15): 5′-hydroxyl function of compound 92b is protected as its DMT derivative as described in Example 2. Compound thus obtained is treated with 10 molar excess of ammonium formate in the presence of 10% activated Pd—C in EtOAc for 10 min. The side chain free amino group thus formed is stirred with ethyltrifluoroacete in the presence of TEA in dichloromethane to obtain compound 93b.

### EXAMPLE 117

Compound 94b (R=(CH₂)₂NHCOCF₃, R′=OCH₂CH₂OCH₃, Scheme 15): Phosphitylation of compound 93b with 2-Cyanoethyl tetraisopropylphosphrodiamidite as reported in Example 8 yields compound 94b.

### EXAMPLE 118

Compound 89c (R=CH₂CO₂Et, Scheme 15): Compound 88 is stirred with ethylbromoacetate in the presence of DIEA in DCM to obtain compound 89c.

### EXAMPLE 119

Compound 93c (R=CH₂CO₂Et, R′=OCH₂CH₂OCH₃, Scheme 15): Compound 93c as defined is obtained from compound 89c according to the procedure reported in Examples 109 to 112.

### EXAMPLE 120

Compound 94c (R=CH₂CO₂Et, R′=OCH₂CH₂OCH₃, Scheme 1): Compound 93c from Example 119 is phosphitylated as described in Example 8 to obtain the phosphoramidite 94c.

### EXAMPLE 121

Compound 93d to 93i (R=CH₂COY, R′=OCH₂CH₂OCH₃, Scheme 15): Compound 93c obtained from Example 119 is treated with:
(a) ammonia to obtain compound 93d (Y=NH₂);
(b) methylamine to obtain compound 93e (Y=NHMe);
(c) dimethylamine to obtain compound 93f (Y=NMe₂);
(d) hydrazine to obtain compound 93g (Y=NH—NH₂);
(e) hydroxylamine to obtain compound 93h (Y=NH—OH);
(f) ethylamine to obtain compound 93i (Y=NHEt).

### EXAMPLE 122

Compound 94d (R=CH₂CONH₂, R′=OCH₂CH₂OCH₃, Scheme 15): Phosphitylation of compound 93d as described in Example 8 yields the phosphoramidate 94d.

### EXAMPLE 123

Compound 94e (R=CH₂CONHCH₃, R′=OCH₂CH₂OCH₃, Scheme 15): Phosphitylation of compound 93e as described in Example 8 yields the phosphoramidate 94e.

### EXAMPLE 124

Compound 94f (R=CH₂CON(CH₃)₂, R′=OCH₂CH₂OCH₃, Scheme 15): Phosphitylation of compound 93f as described in Example 8 yields the phosphoramidate 94e.

### EXAMPLE 125

Scheme 16 is the synthetic scheme for monomers and intermediates described in Example 125.

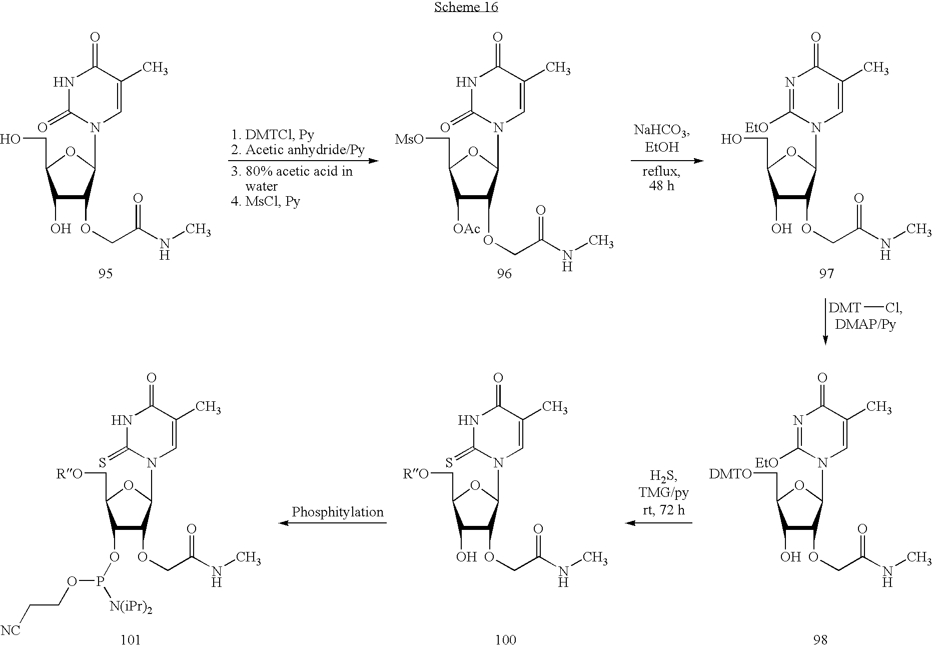

Compound 101 (Scheme 16): Compound 95 is prepared according to the procedure described in the literature (U.S. Pat. No. 6,147,200). Tritylation at 5′-O-position of compound 95 with DMT-Cl in pyridine at room temperature, then acetylation at 3′-O-positon with acetic anhydride in pyridine yields 5′-O-DMT-3′-O-acetyl derivative. Detritylation with 80% acetic acid followed by treatment with methanesulfonyl chloride in pyridine yields compound 96. Compound 101 is prepared from compound 96 according to the procedure described for the synthesis of compound 6 from compound 2 in Example 1.

### EXAMPLE 126

Scheme 17 is the synthetic scheme for monomers and intermediates described in Example 126.

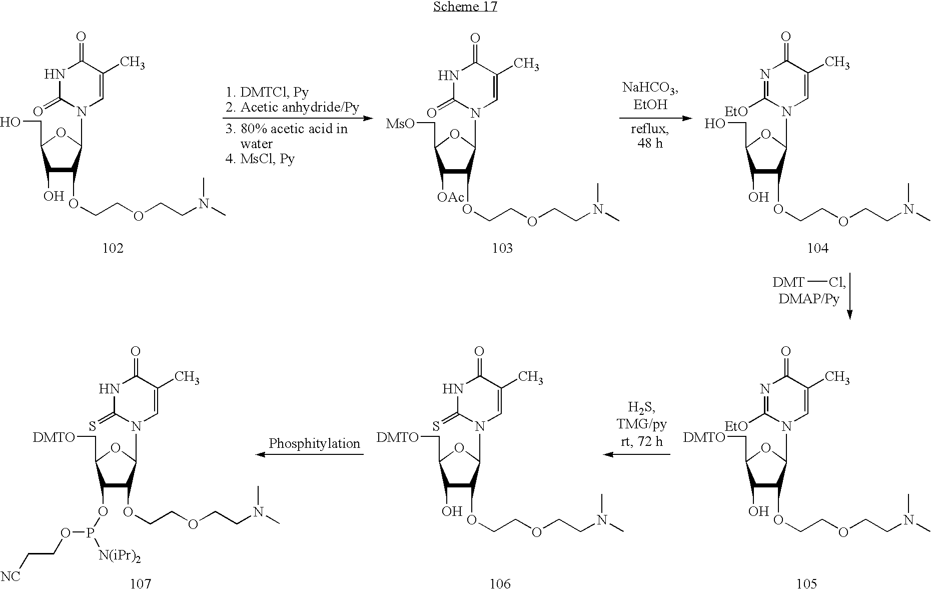

Compound 107 (Scheme 17): Compound 102 is prepared according to the procedure described in the literature (U.S. Pat. No. 6,043,352). Tritylation at 5′-O— position of compound 102 with DMT-Cl in pyridine at room temperature, followed by acetylation at 3′-O-positon with acetic anhydride in pyridine yields 5′-O-DMT-3′-O-acetyl derivative. Detritylation with 80% acetic acid followed by treatment with methanesulfonyl chloride in pyridine yields compound 103. Compound 107 is prepared from compound 103 according to the procedure described for the synthesis of compound 6 from compound 2 in Example 1.

### EXAMPLE 127

Scheme 18 is the synthetic scheme for monomers and intermediates described in Example 127.

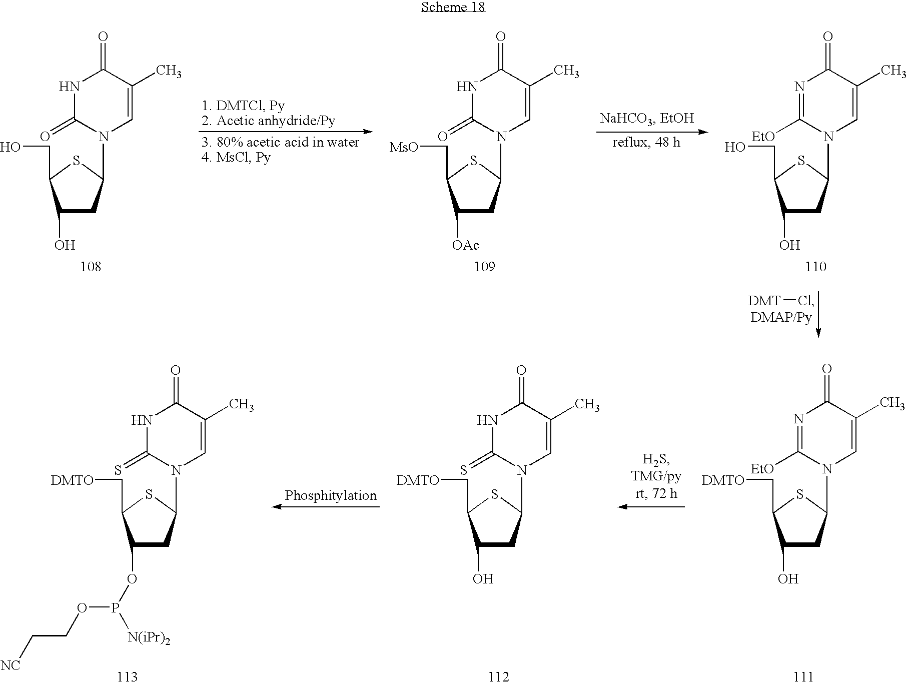

Compound 113 (Scheme 18): Compound 108 is prepared according to the procedure reported (Secrist, J, A. et al. J. Med. Chem. 1991, 56, 2361-2366, Tiwari, K. N. et. al. Nucleosides, Nucleotides 1995, 14, 675-686). Tritylation at 5′-O— position of compound 102 with DMT-CL in pyridine at room temperature, then acetylation at 3′-O-positon with acetic anhydride in pyridine yields 5′-O-DMT-3′-O-acetyl derivative. Detritylation with 80% acetic acid followed by treatment with methanesulfonyl chloride in pyridine yields compound 109. Compound 113 is prepared from compound 109 according to the procedure described for the synthesis of compound 6 from compound 2 in Example 1.

### EXAMPLE 128

Scheme 19 is the synthetic scheme for monomers and intermediates described in Example 128.

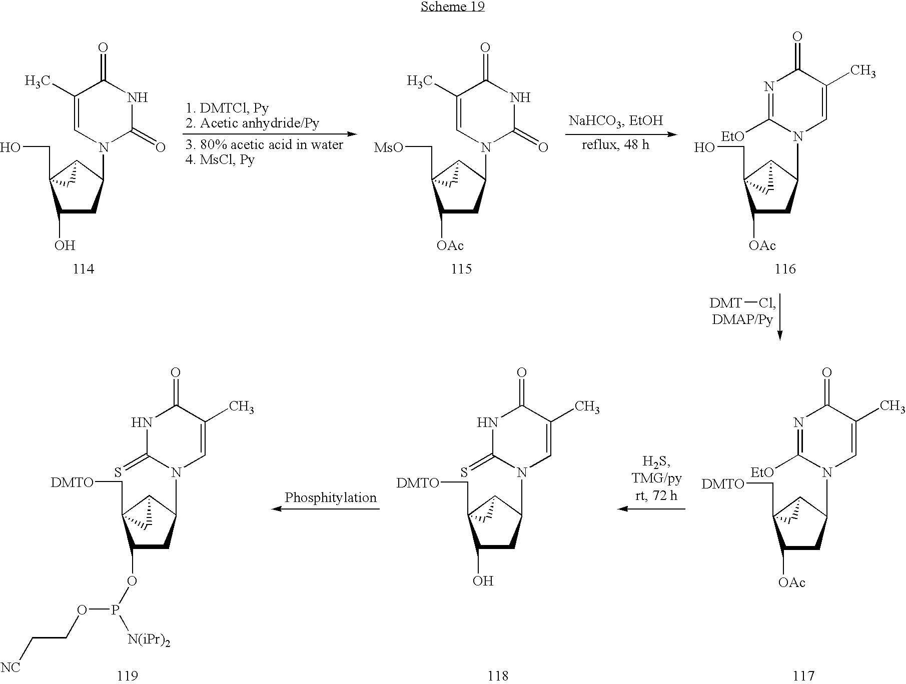

Compound 119 (Scheme 19): Compound 114 is prepared according to the procedure reported (Ezzitouni, A. et. al. J. Org. Chem. 1997, 62, 4870-4873). Tritylation at 5′-O-position of compound 114 with DMT-Cl in pyridine at rt, then acetylation at 3′-O-positon with acetic anhydride in pyridine yields 5′-O-DMT-3′-O-acetyl derivative. Detritylation with 80% acetic acid followed by treatment with methanesulfonyl chloride in pyridine yield compound 115. Compound 119 is prepared from compound 115 according to the procedure described for the synthesis of compound 6 from compound 2 in Example 1.

### EXAMPLE 129

Scheme 20 is the synthetic scheme for monomers and intermediates described in Example 129.

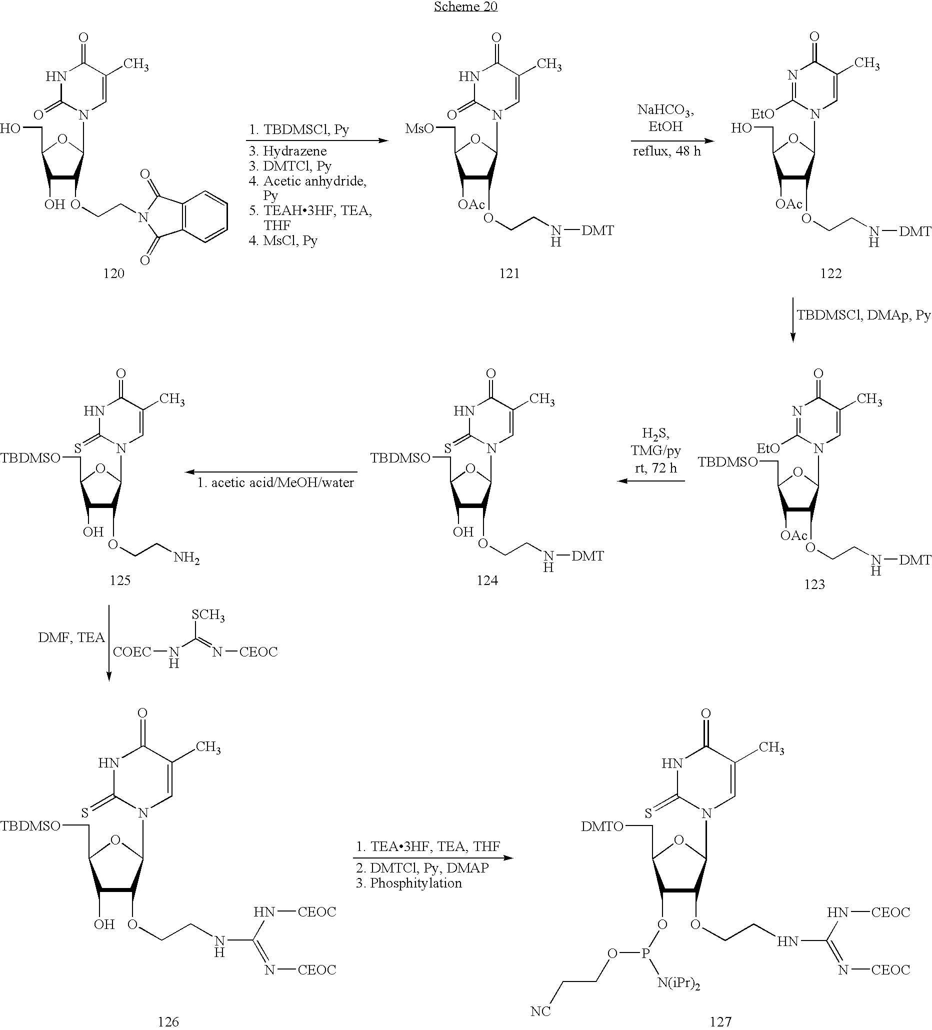

Compound 127 (Scheme 20): Compound 120 is prepared according to the procedure reported (Manoharan M. et. al. J. Org. Chem. 1999, 64, 6468-6472). Silylation of compound 120 with TBDMS-Cl yield 5′-O-TBDMS derivative which on refluxing with hydrazine with methanol give 2′-O-[2-(amino)ethyl derivative, then amino group at 2′ side chain is protected with DMT group by reacting with DMT-Cl in pyridine then acetylation of 3′ hydroxyl group with acetic anhydride in pyridine yield 5′-O-TBDMS-3′-O-acetyl-2′-O-[2-(DMT-amino)ethyl-5-methyl uridine. This is then desilylated with triethylamine trihydofluoride and triethylamine in THF, then treatment with methanesulfonyl chloride in pyridine yields 121. Compound 121 is refluxed in ethanol in presence of NaHCO₃ to yield compound 122, which is subsequently treated with TBDMS-Cl in pyridine to get compound 123. A saturated solution of H₂S in pyridine and tetramethyl guanidine is added to compound 123 and keep at room temperature to get 124. Compound 124 is treated with acetic acid in water to get compound 125. The compound 125 on treatment with N,N′-bis-CEOC-2-methyl-2-thiopseudourea (prepared as reported in U.S. patent application Ser. No. 09/612,531, filed Jul. 7, 2000, the specification of which is incorporated herein by reference) in DMF and TEA at room temperature to yield compound 126. Desilylation of compound 126 with TEA.3HF and TEA in THF, then tritylation at 5′-position followed by phosphitylation at 3′-postion yields compound 127.

### EXAMPLE 130

Scheme 21 is the synthetic scheme for monomers and intermediates described in Examples 130-132.

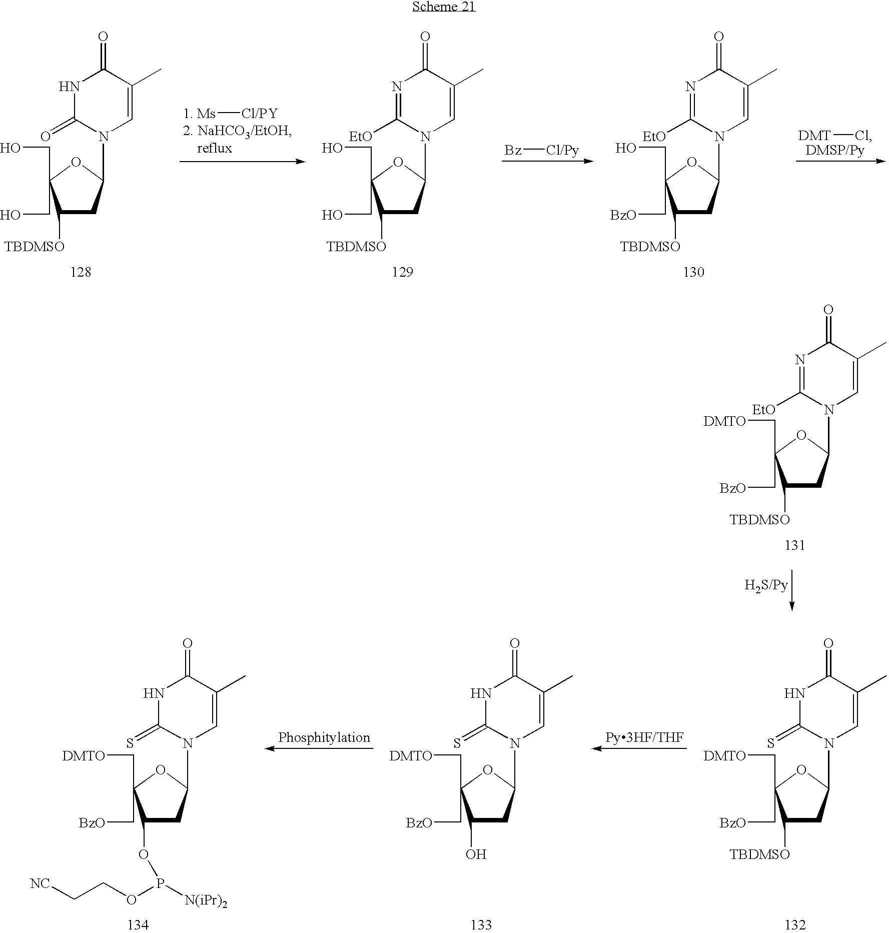

Compound 129 (Scheme 21): Compound 128 is prepared according to the literature procedure (Thrane et. al., Tetrahedron, 1995, 51, 10389-10402). Mesylation of compound 128 with mehtanesulfonyl chloride and subsequent treatment with NaHCO₃ in absolute ethanol as described in Example 1 yields compound 129.

### EXAMPLE 131

Compound 130 (Scheme 21): Benzoylation of compound 129 with benzoyl chloride in pyridine as reported in the literature yields compound 130 (Thrane et. al., Tetrahedron, 1995, 51, 10389-10402).

### EXAMPLE 132

Compound 134 (Scheme 21): Compound 134 is prepared from compound 130 as described in Example 2, 3 and 8 for the synthesis of compound 6 from compound 3 (Scheme 1).

### EXAMPLE 133

Scheme 22 is the synthetic scheme for monomers and intermediates described in Examples 133-136.

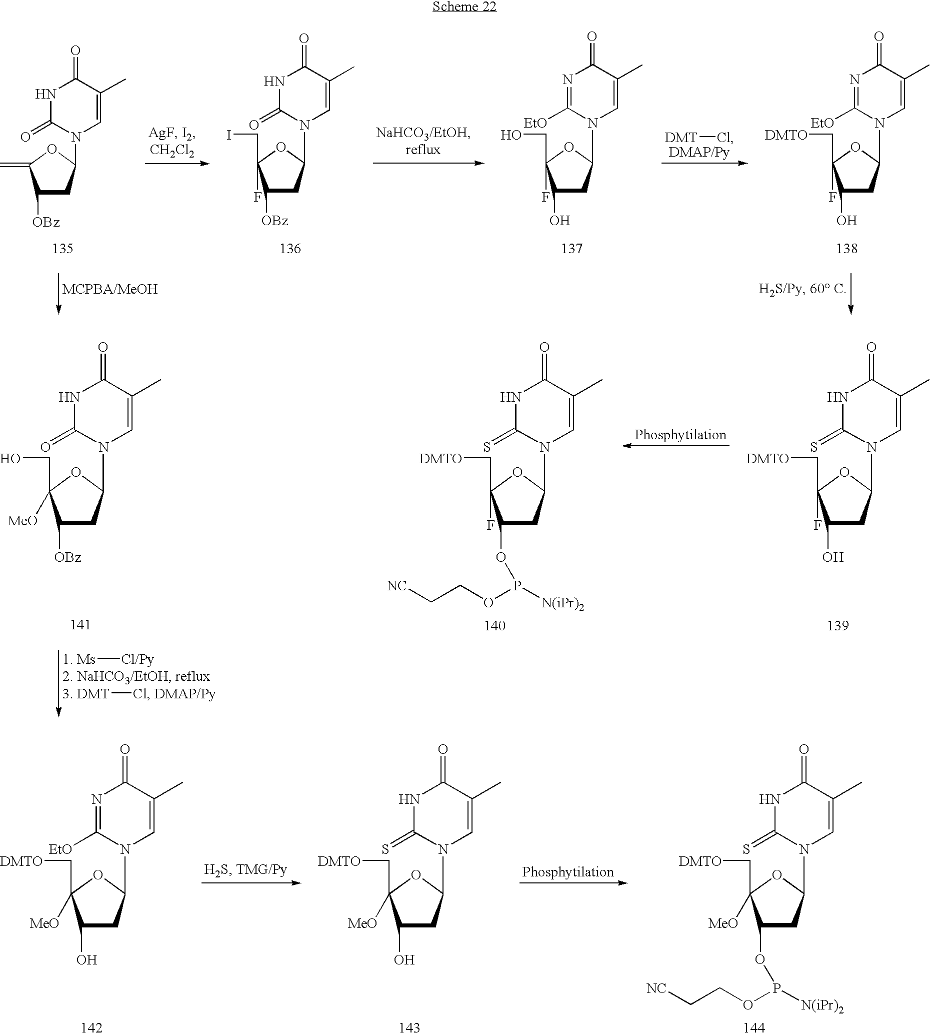

Compound 136 (Scheme 22): Compounds 135 is prepared according to the literature reports (Han et. al., Bull. Korean Chem. Soc., 2000, 21, 321-327). Compound 136 is obtained from compound 135 according to literature procedure (Guillerm et. al., Bioorg. Med. Chem. Lett., 1995, 5, 1455-1460).

### EXAMPLE 134

Compound 140 (Scheme 22): Compound 140 is prepared from compound 136 as described in Examples 10, 11 and 12 for the synthesis of 5′-O-DMT-2′-deoxy-2′-fluoro-2-thio-5-methyluridine 3′-phosphoramidite (6, Example 1).

### EXAMPLE 135

Compound 141 (Scheme 22): Compounds 135 is prepared according to the literature reports (Han et. al., Bull. Korean Chem. Soc., 2000, 21, 321-327). Compound 141 is obtained from compound 135 according to the procedure reported in the literature (Maag et. al., J. Med. Chem., 1992, 35, 1440-1451).

### EXAMPLE 136

Compound 144 (Scheme 22): The desired phosphoramidate 144 is prepared from compound 140 as described in Examples 1, 2, 3 and 8 for the synthesis of compound 6 from compound 1 (Scheme 1)

### EXAMPLE 137

Scheme 23 is the synthetic scheme for monomers and intermediates described in Examples 137-139.

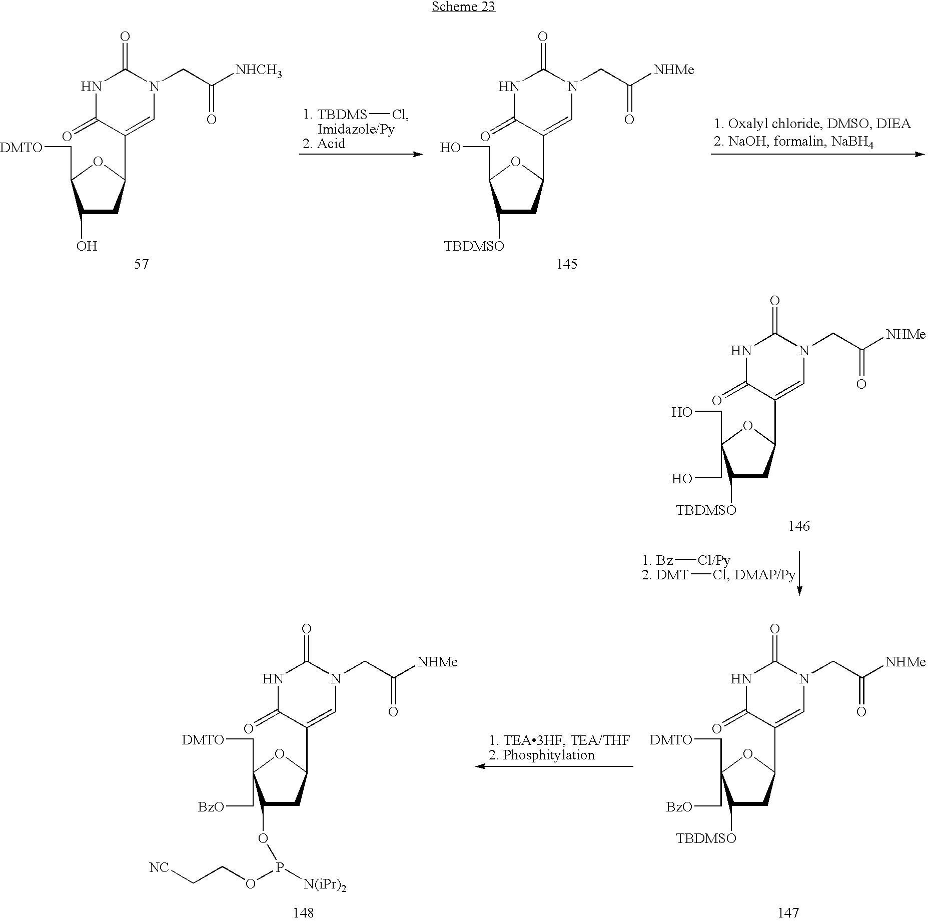

Compound 145 (Scheme 23): Compound 57 is stirred with 1.2 equivalent of TBDMS-Cl and 4 equivalent of imidazole in anhydrous pyridine for 6 h. The compound thus obtained is treated with acetic acid to obtain compound 145.

### EXAMPLE 138

Compound 147 (Scheme 23): Compound 146 is obtained from compound 145 by following a literature procedure (Thrane et. al., Tetrahedron, 1995, 51, 10389-10402).

### EXAMPLE 139

Compound 148 (Scheme 23): Treatment of compound 147with triethylamine trihydrofluoride in the presence of triethylamine in THF and subsequent phosphitylation as described in Example 8 yields compound 148.

### EXAMPLE 140

Scheme 24 is the synthetic scheme for monomers and intermediates described in Examples 140-144.

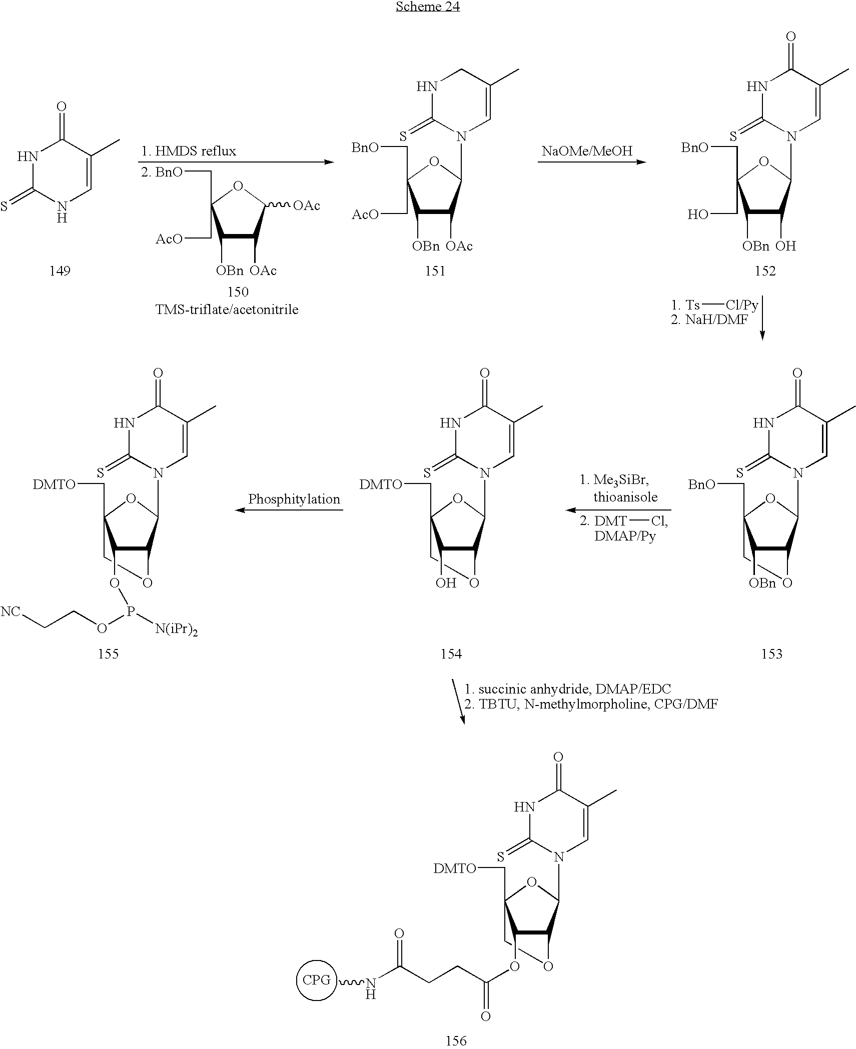

Compound 151 (Scheme 24): Compound 150 is prepared as reported in the literature (Koshkin et. al., Tetrahedron, 1998, 54, 3607-3630). 2-Thio-5-methyluracil (149) is refluxed in HMDS to obtain its corresponding dimethylsilylated derivative. The silylated derivative thus obtained is reacted with compound 150 according to a literature procedure (Koshkin et. al., Tetrahedron, 1998, 54, 3607-3630) to obtain compound 151.

### EXAMPLE 141

Compound 153 (Scheme 24): The desired compound 153 is prepared from compound 151 as reported by Koshikin et. al. (Tetrahedron, 1998, 54, 3607-3630).

### EXAMPLE 142

Compound 154 (Scheme 24): Treatment of compound 153 with trimethylsilylbromide in the presence of thioanisole (Fujii et. al., Chem. Pharm. Bull., 1987, 35, 3880) removes the benzyl protection from the sugar moiety. The unprotected nucleoside thus obtained is reacted with DMT-Cl in the presence of DMAP as described in Example 2 yields compound 154.

### EXAMPLE 143

Compound 155 (Scheme 24): Phsophitylation of compound 154 as described in Example 8 yields compound 155.

### EXAMPLE 144

Compound 156 (Scheme 24): Controlled pore glass support is conjugated to 3′-hydroxyl function of compound 154 as described in Examples 91 and 92 gives the desired solid support 156.

### EXAMPLE 145

Scheme 25 is the synthetic scheme for monomers and intermediates described in Examples 145-147, 165, and 166.

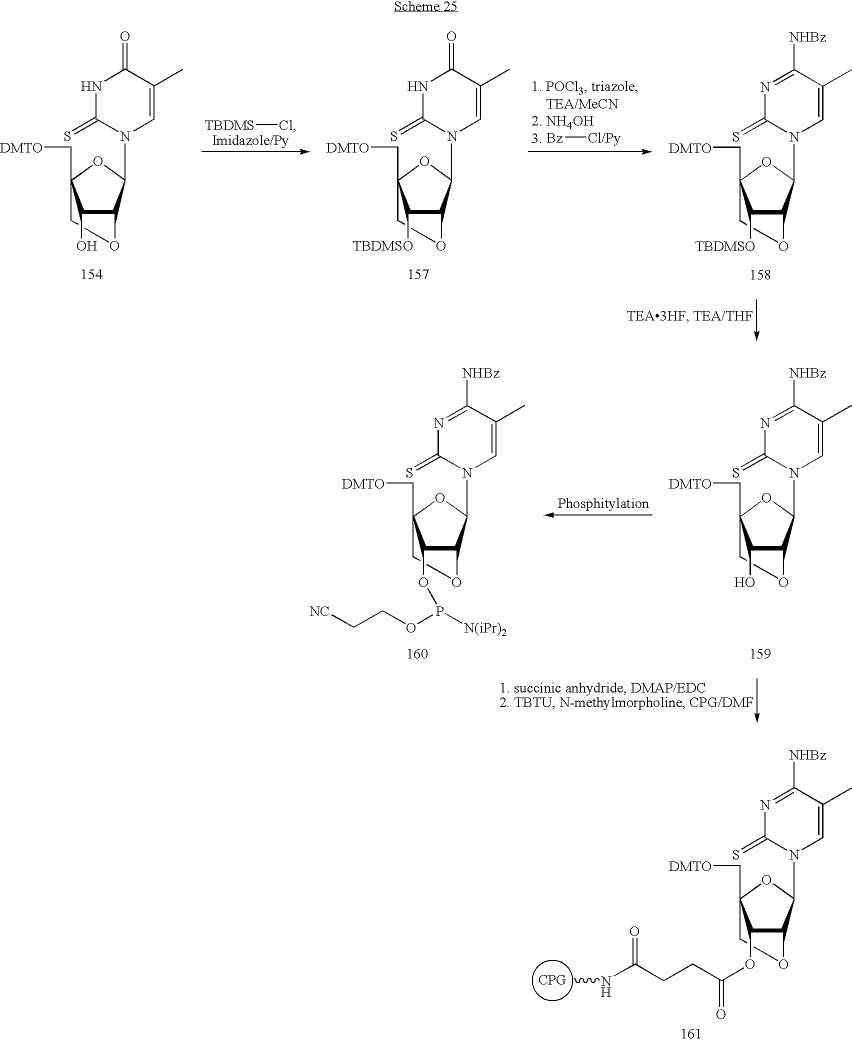

Compound 157 (Scheme 25): Treatment of compound 154 with TBDMS-Cl in the presence of imidazole in anhydrous pyridine as described in Example 15 gives compound 157.

### EXAMPLE 146

Compound 160 (Scheme 25): Compound 160 is prepared from compound 157 as described in Examples 79 (appropriate parts of the experimental procedure), 80 and 81.

### EXAMPLE 147

Compound 161 (Scheme 25): The desired solid support is obtained from compound 159 as described in Examples 91 and 92. Compound 159 is prepared from compound 157 as described in Examples 79 (appropriate parts of the experimental procedure) and 80.

### EXAMPLE 148

Scheme 26 is the synthetic scheme for monomers and intermediates described in Examples 148-152.

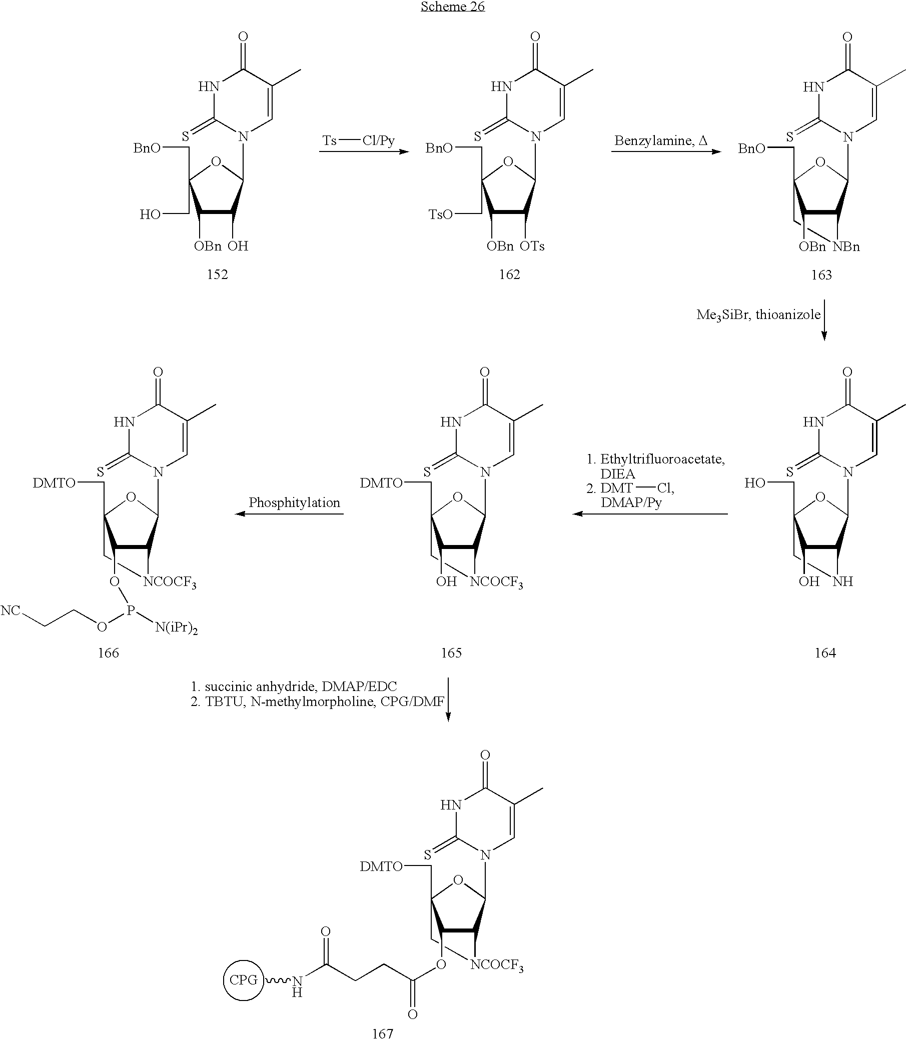

Compound 163 (Scheme 26): Compound 152 is prepared from compound 151 (Scheme 24) as reported in the literature (Koshkin et. al., Tetrahedron, 1998, 54, 3607-3630). The desired nucleoside 163 is prepared from compound 152 as reported in the literature (Singh et. al., J. Org. Chem., 1998, 63, 10035-10039).

### EXAMPLE 149

Compound 164 (Scheme 26): Treatment of compound 163 with trimethylsilylbromide in the presence of thioanisole (Fujii et. al., Chem. Pharm. Bull., 1987, 35, 3880) yields compound 164.

### EXAMPLE 150

Compound 165 (Scheme 26): Compound 165 is prepared from compound 164 as reported in the literature (Singh et. al., J. Org. Chem., 1998, 63, 10035-10039).

### EXAMPLE 151

Compound 166 (Scheme 26): The phosphoramidite 166 is obtained from compound 165 according to the literature procedure (Singh et. al., J. Org. Chem., 1998, 63, 10035-10039).

### EXAMPLE 152

Compound 167 (Scheme 26): Compound 167 is prepared from compound 165 as described in Examples 91 and 92.

### EXAMPLE 153

Scheme 27 is the synthetic scheme for monomers and intermediates described in Examples 153-155.

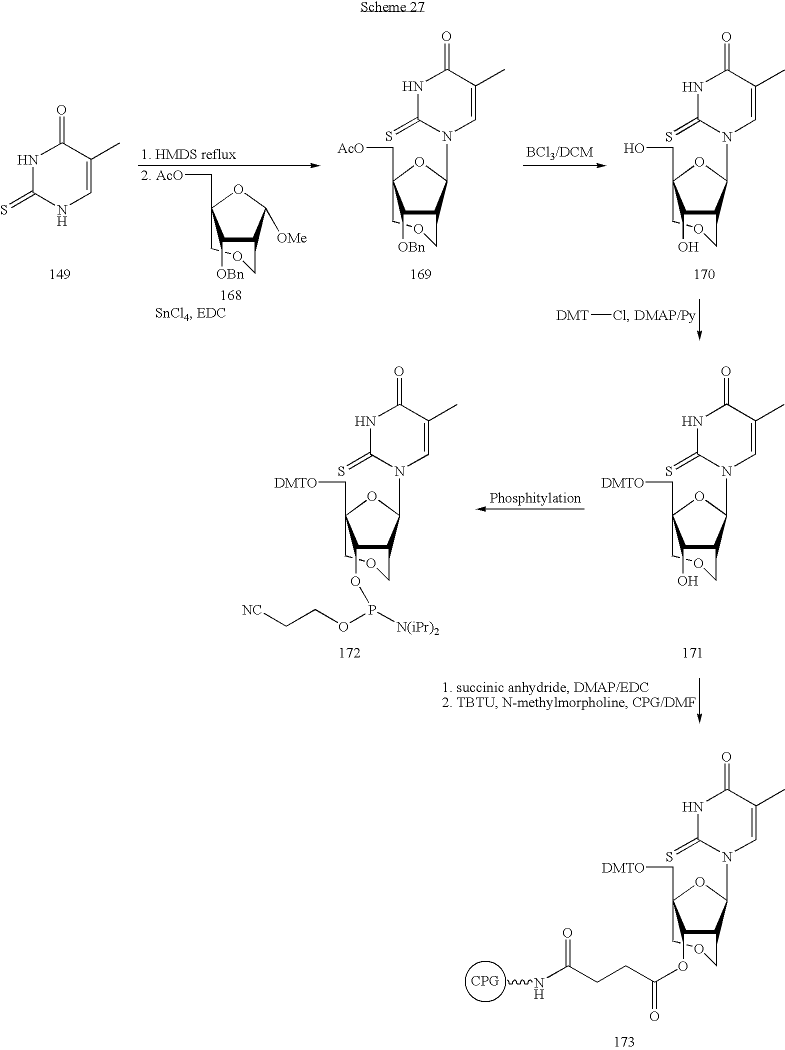

Compound 171 (Scheme 27): Compound 168 is prepared as reported in the literature (Wang et. al., Tetrahedron, 1999, 55, 7707-7724). The desired compound 171 is prepared from compound 168 and compound 149 according to the procedures reported by Wang et. al., (Tetrahedron, 1999, 55, 7707-7724).

### EXAMPLE 154

Compound 172 (Scheme 27): Phosphitylation of compound 171 as described in Example 8 yields compound 172.

### EXAMPLE 155

Compound 173 (Scheme 27): Controlled pore glass support is conjugated to 3′-hydroxyl function of compound 171 as described in Examples 91 and 92 gives the desired solid support 173.

### EXAMPLE 156

Scheme 28 is the synthetic scheme for monomers and intermediates described in Examples 156-158.

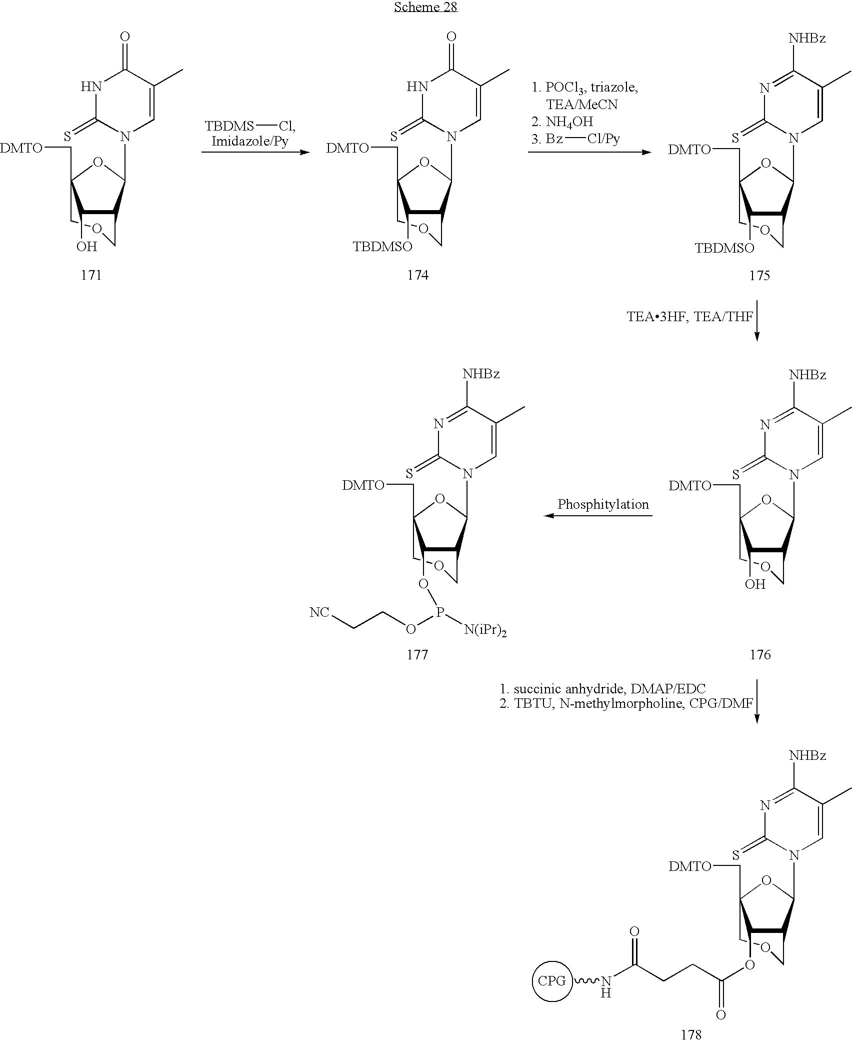

Compound 176 (Scheme 28): The desired compound 176 is prepared from compound 171 (obtained from Example 152) as described in Examples 79 (appropriate parts of the experimental procedure) and 80.

### EXAMPLE 157

Compound 177 (Scheme 28): Phosphitylation of compound 176 as described in Example 8 yields compound 177.

### EXAMPLE 158

Compound 178 (Scheme 28): Controlled pore glass support is conjugated to 3′-hydroxyl function of compound 176 as described in Examples 91 and 92 gives the desired solid support 178.

### EXAMPLE 159

Scheme 29 is the synthetic scheme for monomers and intermediates described in Examples 159-163 and 186.

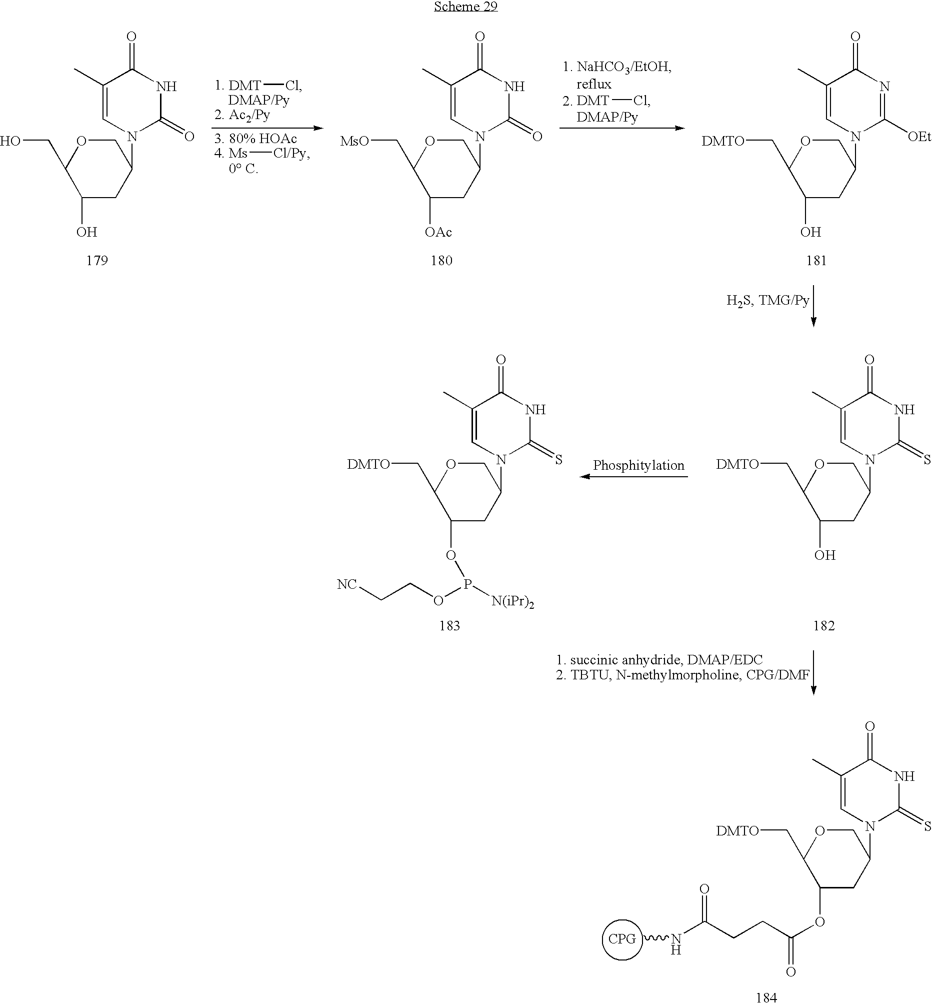

Compound 180 (Scheme 29): Compound 179 is prepared as reported in the literature (Wouters and Herdewijn, Bioorg Med. Chem. Lett., 1999, 9, 1563-1566). Compound 179 is reacted with DMT-Cl in the presence of DMAP as described in Example 2 to obtain DMT derivative. Treatment of the DMT derivative compound 179 with acetic anhydride in anhydrous pyridine in the presence of DAMP gives acetylation at the secondary hydroxyl function. After acetylation, the DMT group is removed from the primary hydroxyl group by stirring in 80% aqueous acetic acid. Treatment of the product obtained with methanesulfonyl chloride in anhydrous pyridine at 0° C. yields the desired compound 180.

### EXAMPLE 160

Compound 181 (Scheme 29): Compound 180 is refluxed in absolute ethanol in the presence of anhydrous NaHCO₃ as described in Example 1 (appropriate parts of the experimental procedure). The 2-ethoxy derivative thus forms is reacted with DMT-Cl in the presence of DMAP as described in Example 2 to yield compound 181.

### EXAMPLE 161

Compound 182 (Scheme 29): Compound 181 is treated with H₂S in the presence of TMG in pyridine as described in Example 3 yields compound-182.

### EXAMPLE 162

Compound 183 (Scheme 29): Phosphitylation of compound 182 as described in Example 8 yields the desired phosphoramidite 183.

### EXAMPLE 163

Compound 184 (Scheme 29): Controlled pore glass (CPG) support is conjugated to 3′-hydroxyl function of compound 182 as described in Examples 91 and 92 gives the desired solid support 184.

### EXAMPLE 164

Scheme 30 is the synthetic scheme for monomers and intermediates described in Example 164.

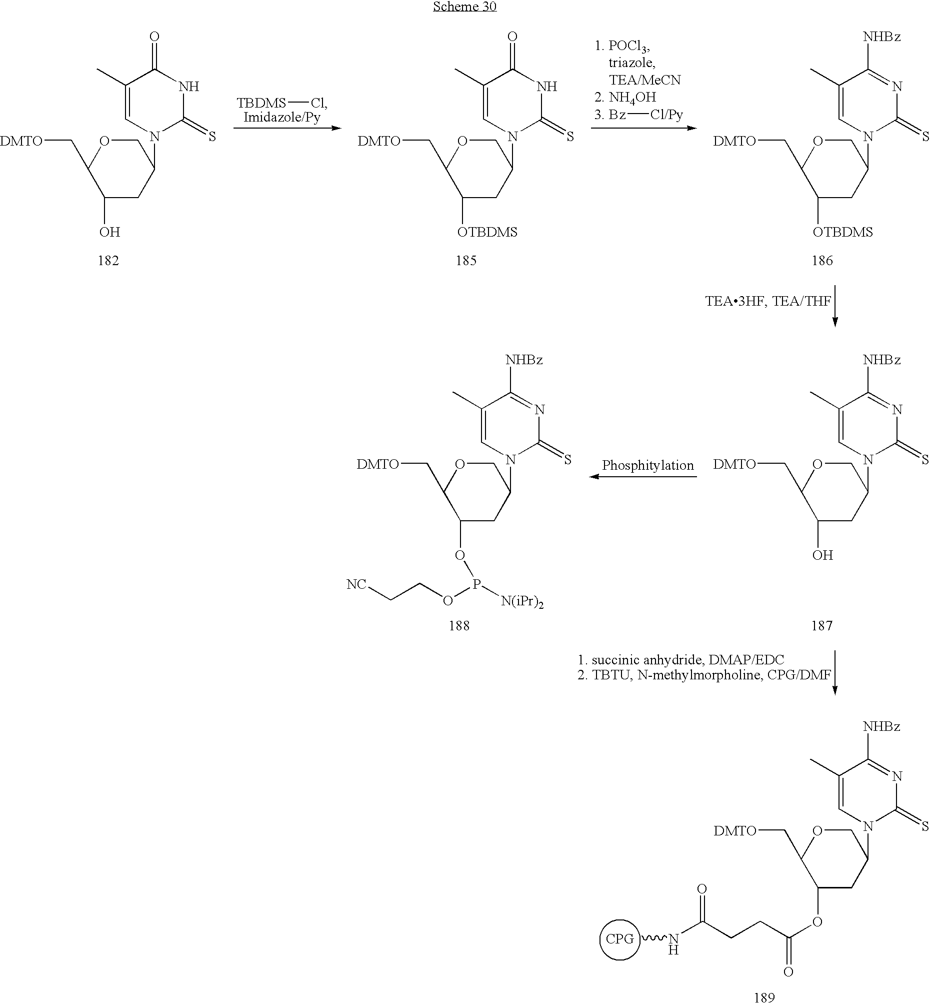

Compound 185 (Scheme 30): Treatment of compound 182 with TBDMS-Cl in the presence of imidazole in anhydrous pyridine as described in Example 15 gives compound 185.

### EXAMPLE 165

Compound 188 (Scheme 25): Compound 188 is prepared from compound 185 as described in Examples 79 (appropriate parts of the experimental procedure), 80 and 81.

### EXAMPLE 166

Compound 189 (Scheme 25): The desired solid support 189 is obtained from compound 187 as described in Examples 91 and 92. Compound 187 is prepared from compound 185 as described in Examples 79 (appropriate parts of the experimental procedure) and 80.

### EXAMPLE 167

Schemes 31a and 31b are the synthetic scheme for monomers and intermediates described in Examples 167-169.

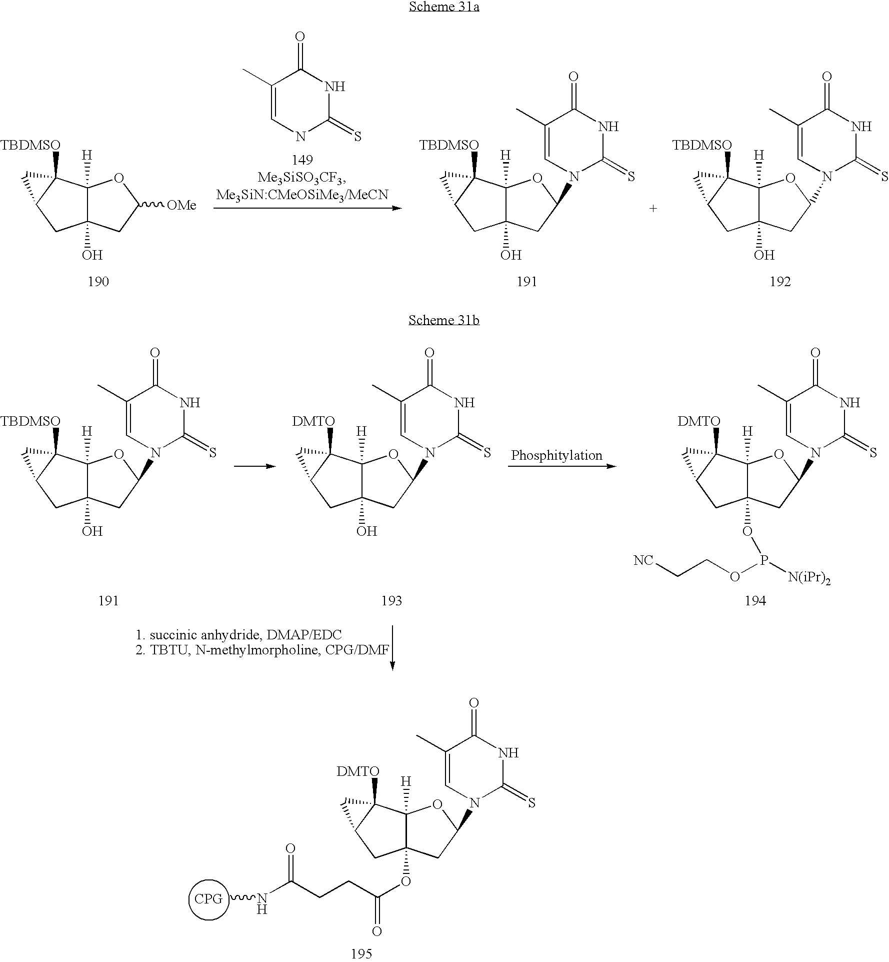

Compound 191 (Scheme 31A): Compound 190 is prepared as reported in the literature (Steffens and Leumann, Helv. Chim. Acta, 1997, 80, 2426-2439). Compound 191 is prepared from compounds 190 and 149 according to the reported procedure by Steffens and Leumann (Helv. Chim. Acta, 1997, 80, 2426-2439). The two stereo isomers formed are separated by flash column chromotography.

### EXAMPLE 168

Compound 194 (Scheme 31b): Compound 194 is prepared from compound 191 as reported by by Steffens and Leumann (Helv. Chim. Acta, 1997, 80, 2426-2439).

### EXAMPLE 169

Compound 195 (Scheme 31b): The desired solid support 195 is obtained from compound 193 as described in Examples 91 and 92. Compound 193 is prepared from compound 191 according to the literature procedure (Steffens and Leumann, Helv. Chim. Acta, 1997, 80, 2426-2439).

### EXAMPLE 170

Scheme 32 is the synthetic scheme for monomers and intermediates described in Examples 170-173.

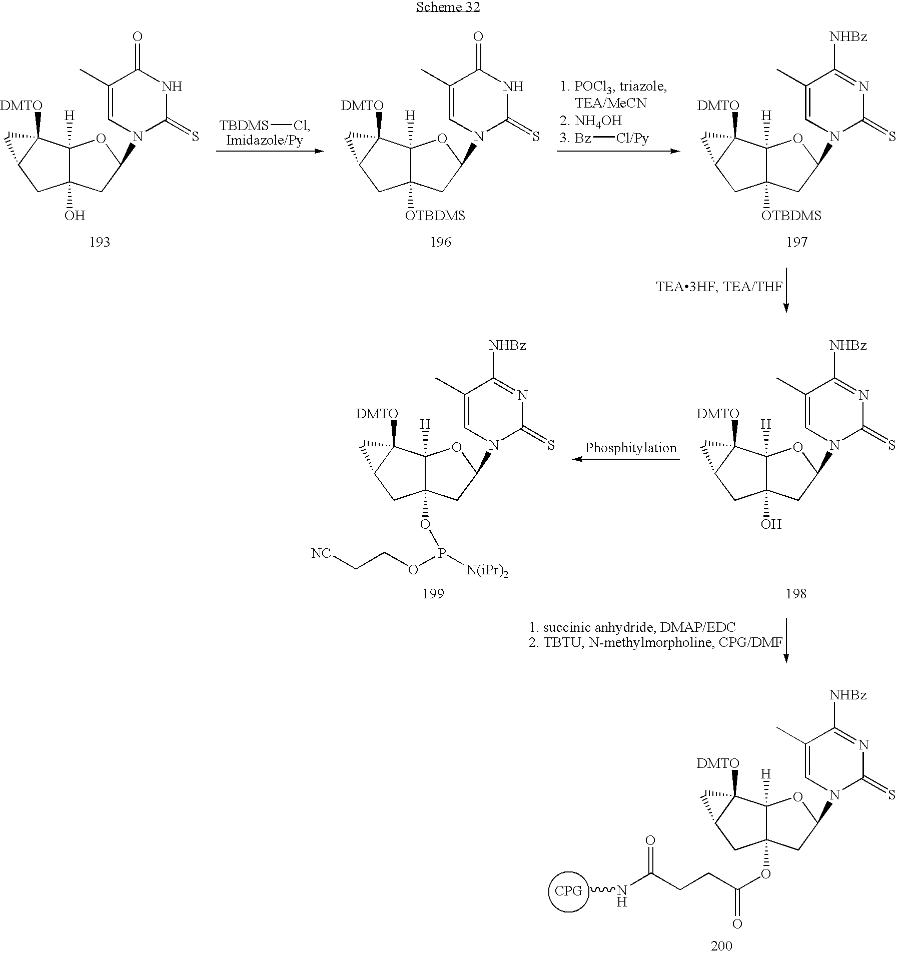

Compound 196 (Scheme 32): Treatment of compound 193 with TBDMS-Cl in the presence of imidazole in anhydrous pyridine as described in Example 15 gives compound 196.

### EXAMPLE 171

Compound 198 (Scheme 32): Compound 198 is prepared from compound 196 as described in Examples 79 (appropriate parts of the experimental procedure) and 80.

### EXAMPLE 172

Compound-199 (Scheme 32): Phosphitylation of compound 198 yields the desired phosphoramidite 199.

### EXAMPLE 173

Compound 200 (Scheme 32): The desired solid support 200 is prepared from compound 198 in two steps as described in Examples 91 and 92.

### EXAMPLE 174

Schemes 33a and 33b is the synthetic scheme for monomers and intermediates described in Examples 174-176.

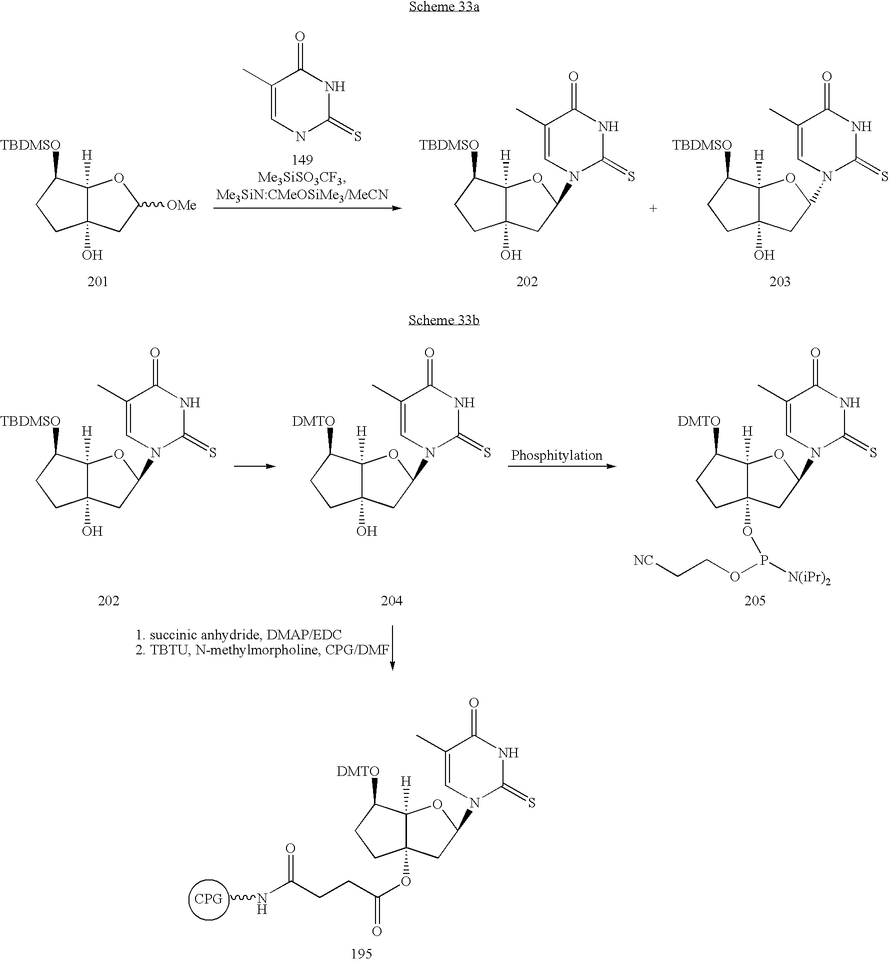

Compound 202 (Scheme 33A): Compound 201 is prepared as reported in the literature (Steffens and Leumann, Helv. Chim. Acta, 1997, 80, 2426-2439). Compound 202 is prepared from compounds 201 and 149 according to the reported procedure by Steffens and Leumann (Helv. Chim. Acta, 1997, 80, 2426-2439). The two stereo isomers formed are separated by flash column chromotography.

### EXAMPLE 175

Compound 205 (Scheme 33b): Compound 205 is prepared from compound 202 as reported by by Steffens and Leumann (Helv. Chim. Acta, 1997, 80, 2426-2439).

### EXAMPLE 176

Compound 206 (Scheme 33b): The desired solid support 206 is obtained from compound 204 as described in Examples 91 and 92. Compound 204 is prepared from compound 202 according to the literature procedure (Steffens and Leumann, Helv. Chim. Acta, 1997, 80, 2426-2439).

### EXAMPLE 177

Scheme 34 is the synthetic scheme for monomers and intermediates described in Examples 177-180.

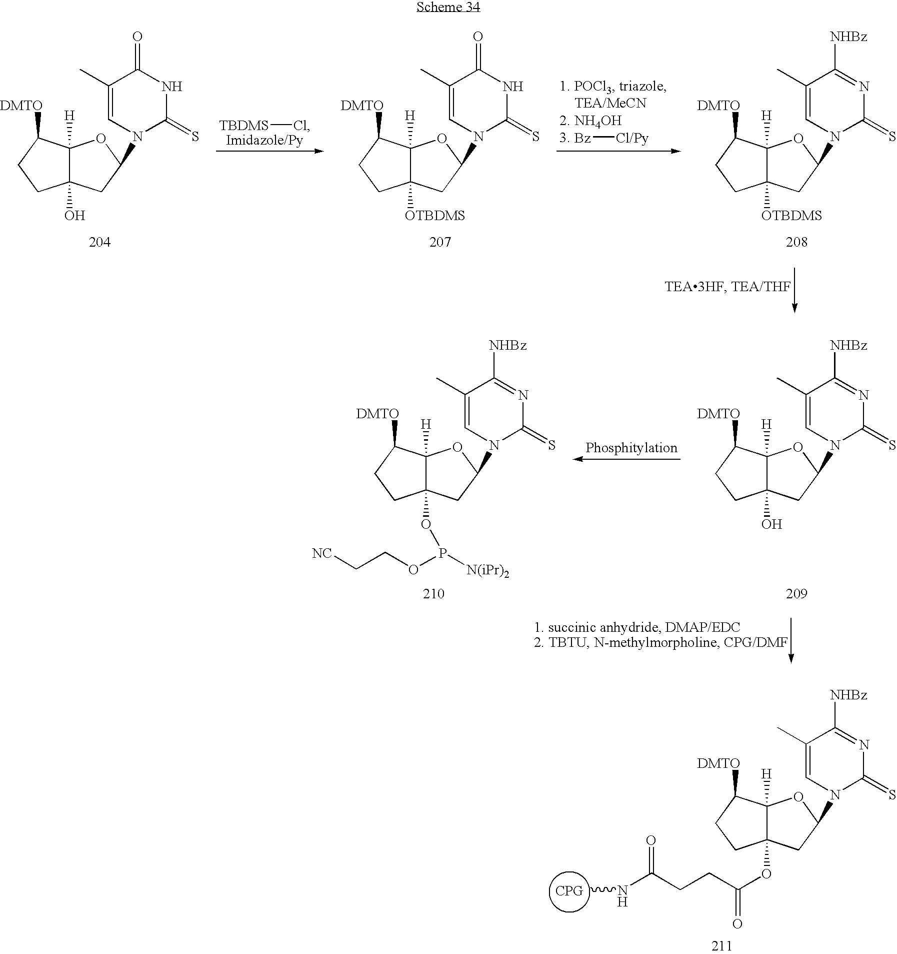

Compound 207 (Scheme 34): Treatment of compound 204 with TBDMS-Cl in the presence of imidazole in anhydrous pyridine as described in Example 15 gives compound 207.

### EXAMPLE 178

Compound 209 (Scheme 34): Compound 209 is prepared from compound 207 as described in Examples 79 (appropriate parts of the experimental procedure) and 80.

### EXAMPLE 179

Compound 210 (Scheme 34): Phosphitylation of compound 209 yields the desired phosphoramidite 210.

### EXAMPLE 180

Compound 211 (Scheme 34): The desired solid support 211 is prepared from compound 209 in two steps as described in Examples 91 and 92.

### EXAMPLE 181

Scheme 35 is the synthetic scheme for monomers and intermediates described in Examples 181-185 and 187.

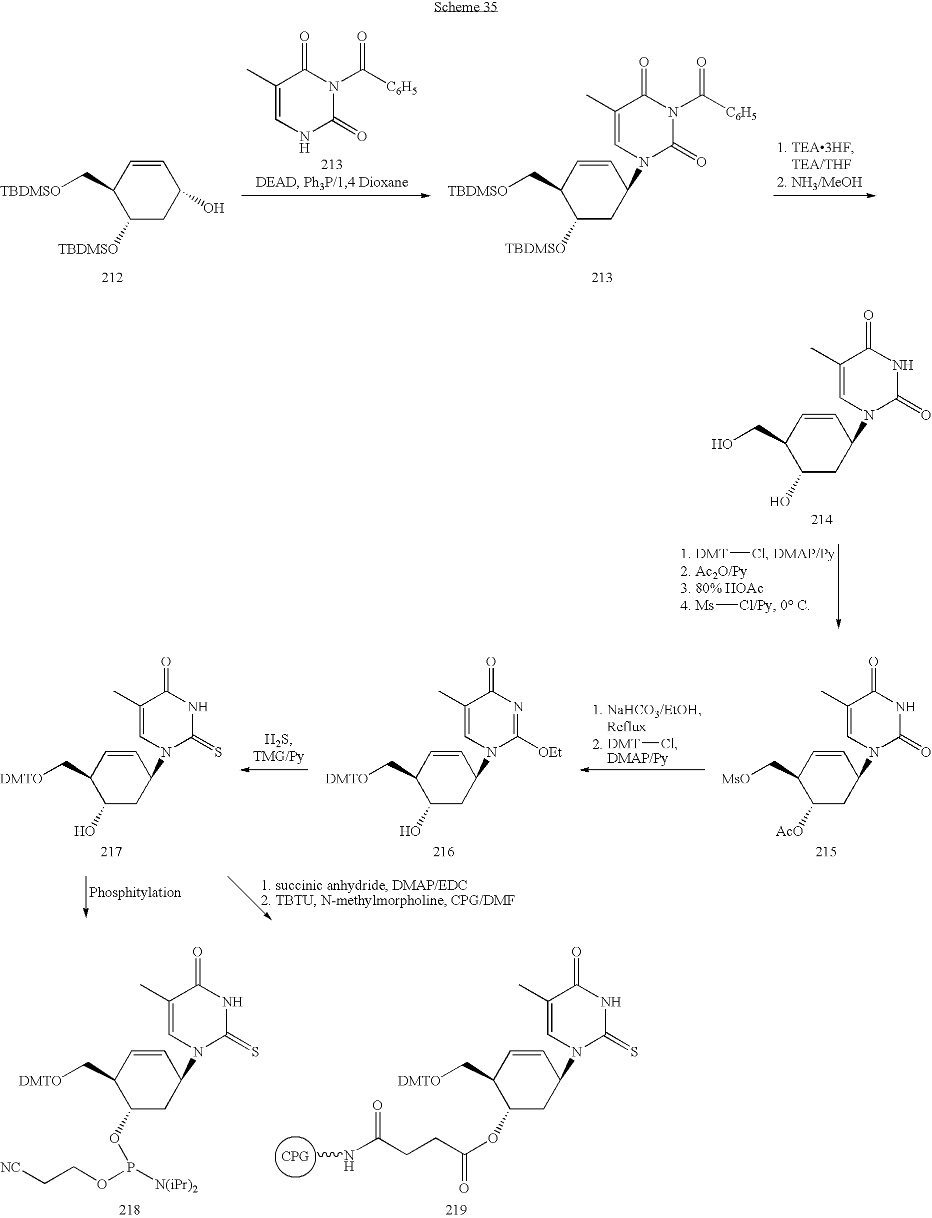

Compound 213 (Scheme 35): Compound 212 is prepared as reported in the literature (Wang and Herdewijn, J. Org. Chem., 1999, 64, 7820-7827). N3-Benzoylthymine is prepared as reported in the literature (Song, et. al., J. Med. Chem., 2001, 44, 3985-3993). Reaction of compound 212 with compound 213 in the presence of DEAD and Ph₃P as reported in the literature (Song, et. al., J. Med. Chem., 2001, 44, 3985-3993) yields compound 213.

### EXAMPLE 182

Compound 214 (Scheme 35): Desilylation of compound 213 as described in Example 80 (appropriate parts of the experimental procedure). The desilylated product thus obtained is treated with methanolic ammonia to obtain the desired compound 214.

### EXAMPLE 183

Compound 215 (Scheme 35): The desired compound 215 is prepared from compound 214 in 4 steps as described in Example 155 for the synthesis of compound 180.

### EXAMPLE 184

Compound 216 (Scheme 35): Compound 215 is refluxed in absolute ethanol in the presence of anhydrous NaHCO₃ as described in Example 1 (appropriate parts of the experimental procedure). The 2-ethoxy derivative thus forms is reacted with DMT-Cl in the presence of DMAP as described in Example 2 to yield compound 216.

### EXAMPLE 185

Compound 217 (Scheme 35): Compound 216 is treated with H₂S in the presence of TMG in pyridine as described in Example 3 yields compound 217.

### EXAMPLE 186

Compound 218 (Scheme 29): Phosphitylation of compound 217 as described in Example 8 yields the desired phosphoramidite 218.

### EXAMPLE 187

Compound 219 (Scheme 35): Controlled pore glass (CPG) support is conjugated to 3′-hydroxyl function of compound 217 as described in Examples 91 and 92 gives the desired solid support 219.

### EXAMPLE 188

Scheme 36 is the synthetic scheme for monomers and intermediates described in Examples 188-190.

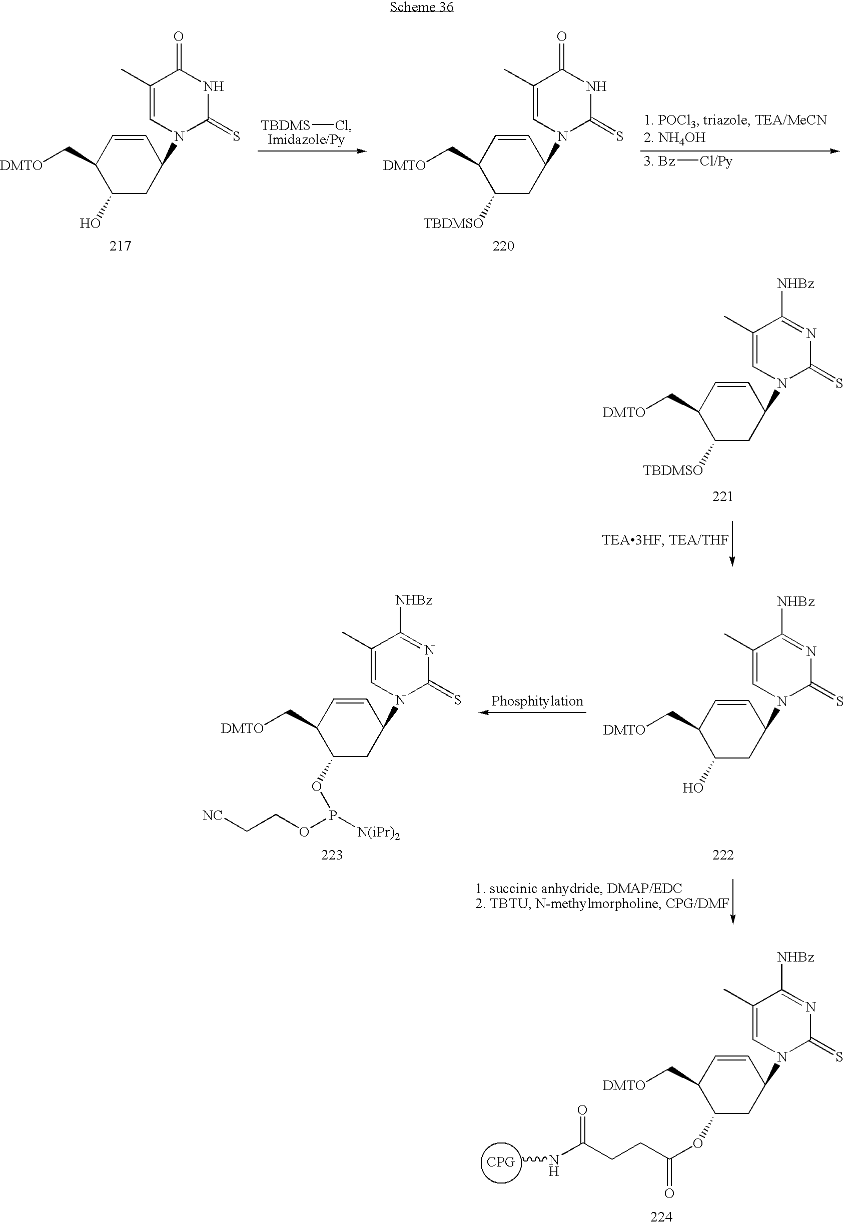

Compound 220 (Scheme 36): Treatment of compound 217 with TBDMS-Cl in the presence of imidazole in anhydrous pyridine as described in Example 15 gives compound 220.

### EXAMPLE 189

Compound 223 (Scheme 36): Compound 223 is prepared from compound 220 as described in Examples 79 (appropriate parts of the experimental procedure), 80 and 81.

### EXAMPLE 190

Compound 224 (Scheme 36): The desired solid support 224 is obtained from compound 222 as described in Examples 91 and 92. Compound 222 is prepared from compound 220 as described in Examples 79 (appropriate parts of the experimental procedure) and 80.

### EXAMPLE 191

Scheme 37 is the synthetic scheme for monomers and intermediates described in Examples 191-195.

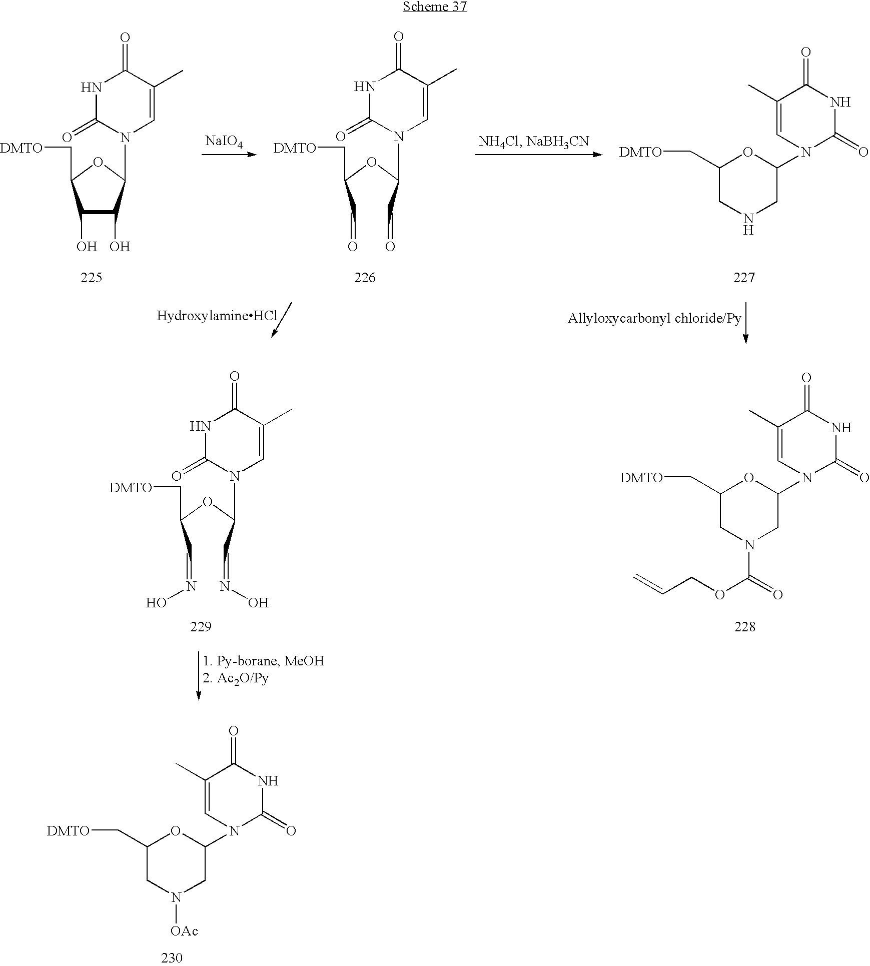

Compound 226 (Scheme 37): NaIO₄ oxidation of 5′-O-DMT-5-methylurdine yields the desired dialdehyde 226.

### EXAMPLE 192

Compound 227 (Scheme 37): Compound 226 is treated with one molar equivalent of ammonium chloride in the presence of excess NaBH₃CN in methanol to obtain compound 226.

### EXAMPLE 193

Compound 228 (Scheme 37): Compound 227 upon treatment with allylchloroformate in anhydrous pyridine at 0° C. (Corey and Suggs, J. Org. Chem., 1973, 38, 3223) yields the desired compound 228.

### EXAMPLE 194

Compound 229 (Scheme 37): Compound 229 is obtained from compound 226 according to the reported procedure (Tronchet, et. al., Tetrahedron Lett., 1991, 32, 4129-32).

### EXAMPLE 195

Compound 230 (Scheme 37): Reduction of compound 229 as reported in the literature (Tronchet, et. al., Nucleosides Nucleotides, 1993, 12, 615-629) and subsequent treatment with acetic anhydride in anhydrous pyridine yields the desired compound 230.

### EXAMPLE 196

Scheme 38 is the synthetic scheme for monomers and intermediates described in Examples 196-201.

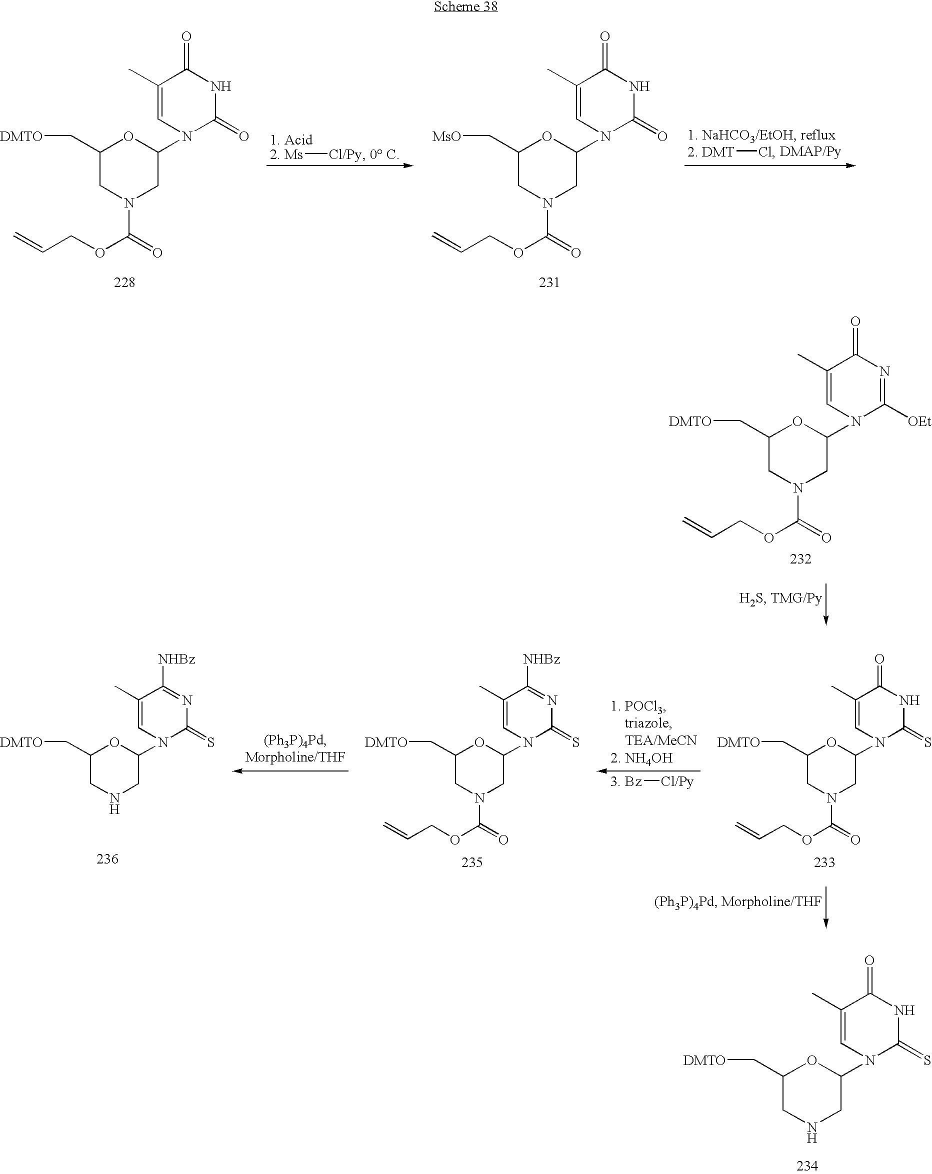

Compound 231 (Scheme 38): Acid treatment of compound 228 gives the corresponding hydroxy compound. The free hydroxyl thus formed is converted into its methane sulfonate 231 by reacting with Ms-Cl in pyridine at 0° C.

### EXAMPLE 197

Compound 232 (Scheme 38): Compound 231 is refluxed in absolute ethanol in the presence of anhydrous NAHCO₃ as described in Example 1 to obtain the corresponding 2-ethoxy derivative. The ethoxy derivative formed is treated with DMT-Cl in the presence of DMAP as described in Example 2 to obtain compound 232.

### EXAMPLE 198

Compound 233 (Scheme 38): Compound 232 is converted to the desired 2-thio analogue 233 by reacting with H₂S in the presence of TMG in anhydrous pyridine as described in Example 3.

### EXAMPLE 199

Compound 234 (Scheme 38): Compound 233 is treated with 10 molar excess of morpholine and catalytic amount of tetrakistriphenylphosphine palladium(0) in anhydrous THF (Kunz and Waldmann, Angew. Chem. Int. Ed. Engl., 1984, 23, 71-72) to obtain the desired compound 234.

### EXAMPLE 200

Compound 235 (Scheme 38): Compound 235 is prepared from compound 233 as described in Example 79 (second part of the procedure) and Example 80 (first part of the procedure).

### EXAMPLE 201

Compound 236 (Scheme 38): The allylcarbamate protection of compound 235 is removed as described in Example 193 to obtain the desired compound 236.

### EXAMPLE 202

Scheme 39 is the synthetic scheme for monomers and intermediates described in Examples 202-205.

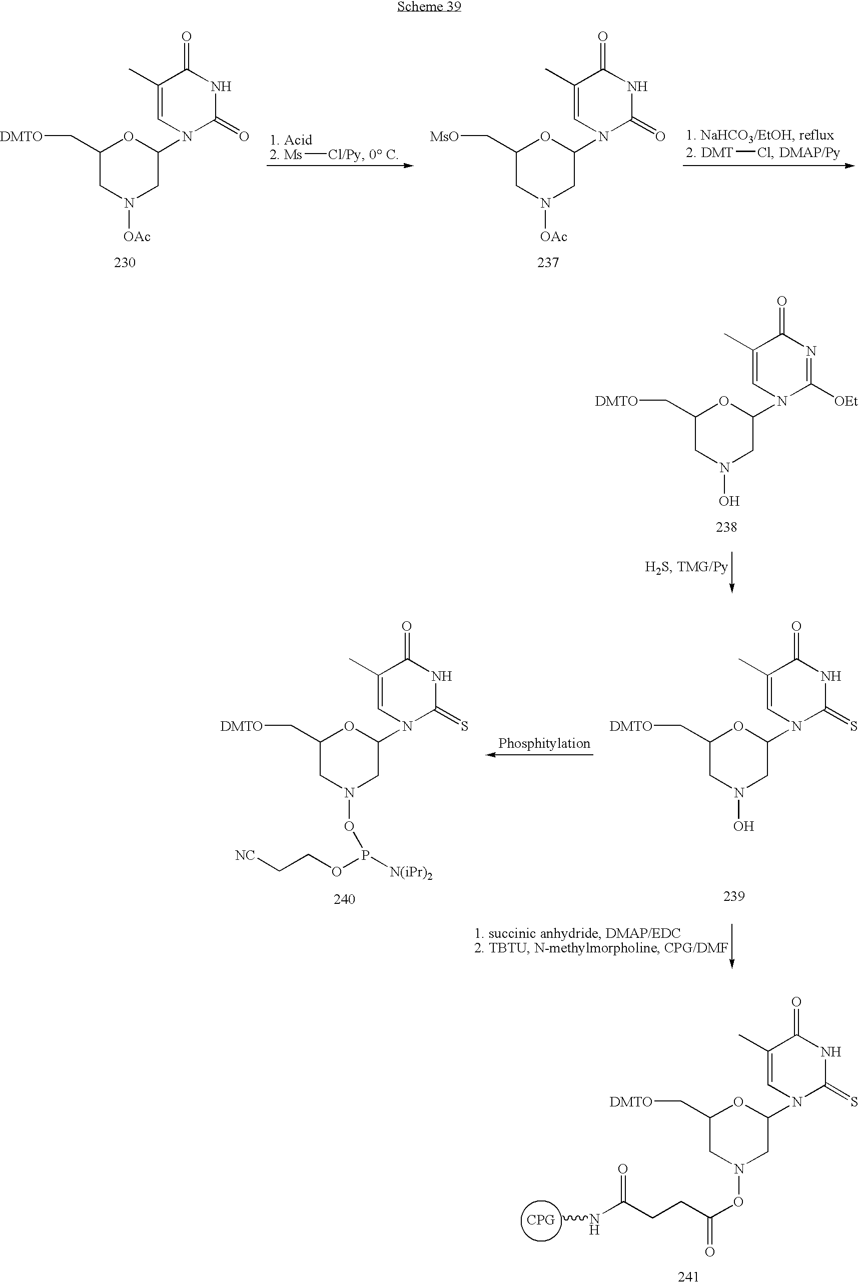

Compound 237 (Scheme 39): Compound 237 is prepared from compound 230 as described in Example 196.

### EXAMPLE 203

Compound 239 (Scheme 39): Compound 239 is prepared from compound 237 according to the procedure described in Examples 197 and 198

### EXAMPLE 204

Compound 240 (Scheme 39): Phosphitylation of compound 239 as described in Example 8 yields the desired phosphoramidite 240.

### EXAMPLE 205

Compound 241 (Scheme 39): Conjugation of compound 139 to control pore glass (CPG) support as described in Examples 91 and 92 yields the desired solid support 241.

### EXAMPLE 206

Scheme 40 is the synthetic scheme for monomers and intermediates described in Examples 206-208.

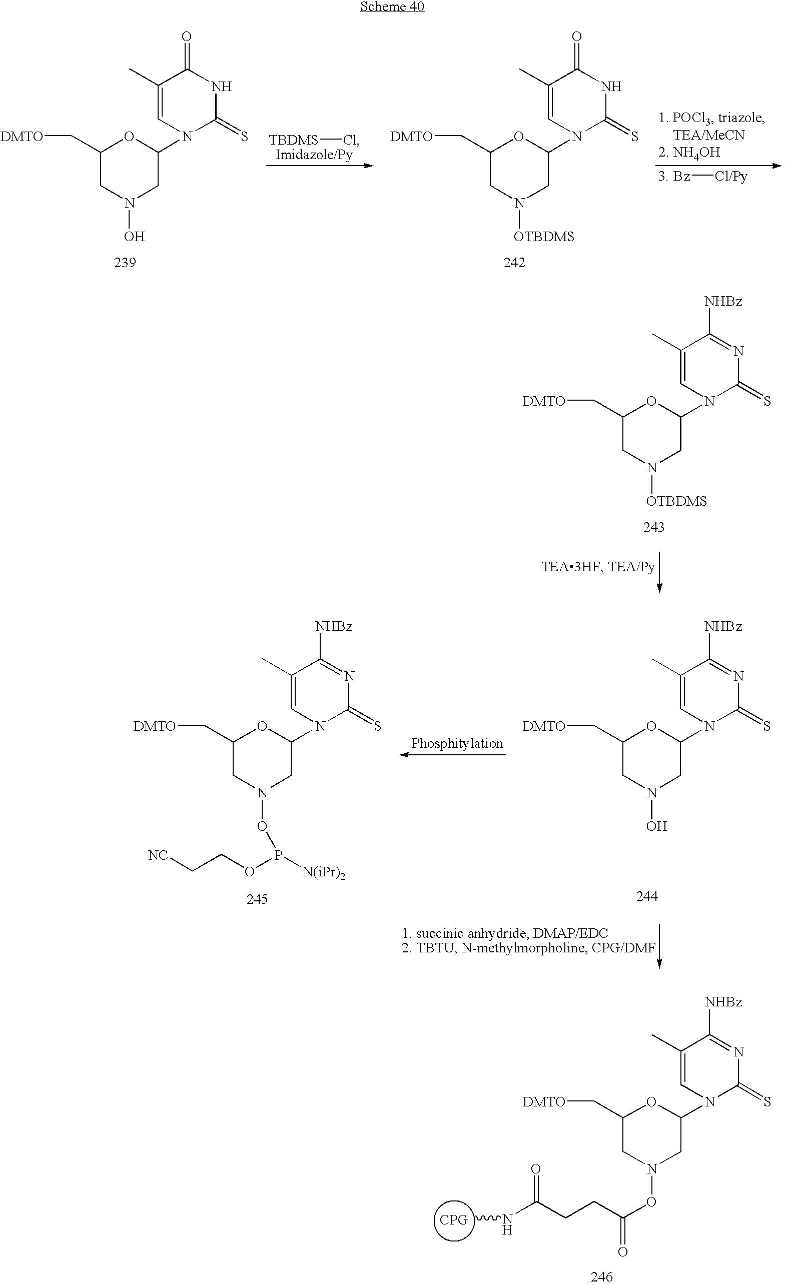

Compound 242 (Scheme 40): Treatment of compound 239 with TBDMS-Cl in the presence of imidazole in anhydrous pyridine as described in Example 15 gives compound 242.

### EXAMPLE 207

Compound 245 (Scheme 40): Compound 245 is prepared from compound 241 as described in Examples 79 (appropriate parts of the experimental procedure), 80 and 81.

### EXAMPLE 208

Compound 246 (Scheme 40): The desired solid support 246 is obtained from compound 244 as described in Examples 91 and 92. Compound 244 is prepared from compound 242 as described in Examples 79 (appropriate parts of the experimental procedure) and 80.

### EXAMPLE 209

Synthesis of 2′-O-MOE-2-thio modified Oligonucleotides. A 0.1 M solution of the amidite 6 (R=OCH₂CH₂OCH₃, X=CH₃) in anhydrous acetonitrile was used for the synthesis of modified oligonucleotides. The oligonucleotides were synthesized on functionalized controlled pore glass (CPG) on an automated solid phase DNA synthesizer. CPG functionalized with 2′-O-MOE-2-thio modified nucleosides were used wherever necessary. For incorporation of 2′-O-MOE-2-thio phosphoramidite solutions were delivered in two portions, each followed by a 5 min coupling wait time. All other steps in the protocol supplied by the manufacturer were used without modification. Oxidation of the internucleotide phosphite to the phosphate was carried out using 10% tert-butylhydroperoxide in acetonitrile with 10 min waiting time. The Beaucage reagent (0.1 M in acetonitrile) was used as a sulfurizing agent. Oligonucleotides were synthesized DMT on mode. The coupling efficiencies were more than 97%. After completion of the synthesis, the solid support was suspended in aqueous ammonium hydroxide (30 wt %, 2 mL for 2 micromole synthesis) and kept at room temperature for 2 h. The supernatant was decanted, the CPG was washed with additional 1 mL of aqueous ammonia. Combined ammonia solution was heated at 55° C. for 6 h. Concentrated the solution to half of the volume. Adjusted the pH of the solution to 8 and the crude oligonucleotides were purified by high performance liquid chromatography (HPLC, C-4 column, Waters, 7.8×300 mm, A=100 mM ammonium acetate, B=acetonitrile, 5-60% of B in 55 min, flow 2.5 mL min-1, X 260 nm). Fractions containing the full length oligonucleotides were pooled together and pH of the solution was adjusted to 4.2 with acetic acid and kept at room temperature for 24 h. An aliquot was withdrawn and analyzed by HPLC on C-4 column (condition same as above) to asses the completion of the detritylation reaction. Neutralized the solution with ammonia and desalted by HPLC on a C-4 column to yield 2′-modified oligonucleotides in 30-40% isolated yield. The oligonucleotides were characterized by ESMS and HPLC and Capillary Gel Electrophoresis assessed their purity.

TABLE 1

HPLC and Mass Spectral Analysis of the 2′-O-
MOE-2-thio oligonucleotides used for Tm analysis

| | | |
|---|---|---|
| | | HPLC |
| Seq. | Mass | Retention |

| | | | | |
|---|---|---|---|---|
| ID No. | Sequences | Calcd | Found | Time, min.^{a} |

| | | | | |
|---|---|---|---|---|
| 1 | 5′ T*oCoCoAoGoGo | 5194.1 | 5193.2 | 24.30 |
| | T*oGoT*oCoCoGoCo | | | |
| | AoT*oC 3′ | | | |
| 2 | 5′GoCoGoT*oT*oT* | 5776.6 | 5775.98 | 32.04 |
| | oT*oT*oT*oT*oT*o | | | |
| | T*oT*oGoCoG 3′ | | | |

^{a}Water C-4, 3.9 × 300 mm,
A = 50 mM triethylammonium acetate, pH 7,
B = acetonitrile, 5 to 60% B in 55 min, flow 1.5 mL min⁻¹,
λ = 260 nm,
T* = 2′-O-[2-(methoxy)ethyl}-2-thio-5-methyluridine

### EXAMPLE 210

Evaluation of Hybridization of 2′-O-MOE-2-thiopyrimidine Modified Oligonucleotides to Complementary RNA and DNA by Thermal Denaturation Studies:.

Thermal denaturation studies of duplex of oligonucleotides containing 2′-OMOE-2-thio moiety and complementary RNA have shown 3.2° C. per modification (Table 2) Tm enhancement compared to 2′-deoxy oligonucleotide phosphodiesters. This translates into 4° C. per modification increase in Tm per modification compared to 2′-deoxy oligonucleotide phosphorothioates. The 2′-O-MOE-2-thio modified oligonucleotides showed 2° C. per modification higher Tm compared to 2′-O-MOE modified oligonucleotides. Table 3 shows Tm value of modified olgonucleotide 2 against complementary DNA. This data suggest that 2′-O-MOE-2-thio modified oligonucleotide form less stable duplex with DNA than RNA.

TABLE 2

Effect of the 2′-O-MOE-2-thio
and 2′-O-MOE modification on duplex
stability against complementary RNA targets

| Seq. | | Tm | ΔTm/modi- |
|---|---|---|---|
| ID No. | sequence | ° C. | fication ° C. |
| 1 | 5′ T*oCoCoAoGoGoT*oGoT*o | 74.1 | 2.92 |
| | CoCoGoCoAoT*oC 3′ | | |
| 2 | 5′GoCoGoT*oT*oT*oT*oT*oT | 82.90 | 3.43 |
| | *oT*oT*oT*oT*oGoCoG 3′ | | |
| 3 | 5′ ToCoCoAoGoGoToGoToCoC | 62.4 | |
| | oGoCoAoToC 3′ | | |
| 4 | 5′ T^{&}oCoCoAoGoGoT^{&}oGoT^{&}o | 65.9 | 0.88 |
| | CoCoGoCoAoT^{&}oC 3′ | | |
| 5 | 5′ GoCoGoToToToToToToToT | 48.50 | |
| | oToToGoCoG 3′ | | |
| 6 | 5′ GoCoGoT^{&}oT^{&}oT^{&}oT^{&}oT^{&}o | 60.0 | 1.15 |
| | T^{&}oT^{&}oT^{&}oT^{&}oT^{&}oG^{&}oCoG 3′ | | |

T* = 2′-O-[2-(methoxy)ethyl}-2-thio-5-methyluridine,
T^{&} = 2′-O-[2-(methoxy)ethyl}-5-methyluridine,
o = P = O

TABLE 3

Effect of the 2′-O-MOE-2-thio
and 2′-O-MOE modifications on duplex
stability against complementary DNA targets

| Seq. | | Tm | ΔTm/unit |
|---|---|---|---|
| ID No. | sequence | ° C. | ° C. |
| 2 | 5′GoCoGoT*oT*oT*oT*oT*oT*oT*o | 73.5 | 1.93 |
| | T*oT*oT*oGoCoG 3′ | | |
| 5 | 5′ GoCoGoToToToToToToToToToToG | 54.2 | |
| | oCoG 3′ | | |
| 6 | 5′ GoCoGoT^{&}oT^{&}oT^{&}oT^{&}oT^{&}oT^{&}oT^{&}o | 42.4 | −1.12 |
| | T^{&}oT^{&}oT^{&}oG^{&}oCoG 3′ | | |

T* = 2′-O-[2-(methoxy)ethyl]-2-thio-5-methyluridine,
T^{&} = 2′-O-[2-(methoxy)ethyl]-5-methyluridine
o = P = O

### EXAMPLE 211

2′-O-MOE-2-thio Modified Antisense Oligonucleotides for In Vitro and In Vivo Evaluation:

Oligonucleotide Gapmers targeted to Mouse p38 alpha, PTEN and Mouse TRADD and hemimer targeted to m-A-raf with 2′-O-MOE-2-thio modifications are synthesized (Table 4). Fully modified oligonucleotides with 2′-O-MOE-2-thio modifications (Table 4) are also synthesized for evaluating their efficacy in non RNase H mediated antisense applications. The efficacy of these antisense oligonucleotides to reduce the messages is evaluated in vitro and in vivo.

TABLE 4

Oligonucleotides with 2′-O-MOE-2-thio and 2′-O-MOE
modifications for in vitro and in vivo evaluation

| Seq. | | |
|---|---|---|
| ID No. | sequence | Target |
| 7 | 5′ A^{&}sG^{&}sG^{&}sT*sGsCsTsCsAsGsGsAsCs | p38 alpha |
| | TsCsC*sA&sT*sT*sT* 3′ | |
| 8 | 5′ A^{&}oG^{&}oG^{&}oT*oG^{&}sCsTsCsAsGsGsAsCs | p38 alpha |
| | TsCsC*oA^{&}oT*oT*oT* 3′ | |
| 9 | 5′C*sT*sC*sC*sA&sGsCsGsCsCsTsCsCs | TRADD |
| | AsCsC*sA^{&}sG^{&}sG^{&}sC*3′ | |
| 10 | 5′C*oT*oC*oC*oA^{&}sGsCsGsCsCsTsCsCs | TRADD |
| | AsCsC*oA^{&}oG^{&}oG^{&}oC*3′ | |
| 11 | 5′ C*sT*sG&s C*sT*sAs GsCsCs TsCs | PTEN |
| | Ts GsGsAs T*sT*sT*s G^{&}sA^{&} 3′ | |
| 12 | 5′ C*oT*oG^{&}o C*oT*sAs GsCsCs TsCs | PTEN |
| | Ts GsGsAs T*oT*oT*o G^{&}oA^{&} 3′ | |
| 13 | 5′ CsCsGs GsTsAs CsCsCs C*sA^{&}sG^{&}s | m-Aaf |
| | G^{&}sT*sT*s C*sT*sT*s C*sA^{&} 3′ | |
| 14 | 5′ CsCsGs GsTsAs CsCsCs C*oA^{&}oG^{&}o | m-Aaf |
| | G^{&}oT*oT*o C*oT*oT*oC*oA^{&} 3′ | |
| 15 | 5′ A^{&}sT*sA^{&}sG^{&}sT*sT*sT*sC*sA^{&}sC*s | PTEN |
| | C*sT*sA^{&}sG^{&}sA^{&}sG^{&}s A^{&}sA^{&}sA^{&}sG^{&} 3′ | |
| 16 | 5′ A^{&}oT*oA^{&}oG^{&}oT*oT*OT*oC*oA^{&}oC*o | PTEN |
| | C*oT*oA^{&}oG^{&}oA^{&}oG^{&}oA^{&}oA^{&}oA^{&}oG^{&} 3′ | |
| 17 | 5′ TTT TTT TTT TTT TTT T*T*T*T* 3′ | Nuclease |
| | | Stability |

T* = 2′-O-[2-(methoxy)ethyl]-2-thio-5-methyluridine,
C* = 2′-O-[2-(methoxy)ethyl]-2-thio-5-methylcytidine,
T^{&} = 2′-O-[2-(methoxy)ethyl]-5-methyluridine,
A^{&} = 2′-O-[2-(methoxy)ethyl]-adenosine,
G^{&} = 2′-O-[2-(methoxy)ethyl]guanosine,
C^{&} = 2′-O-[2-(methoxy)ethyl]-5-methylcytidine,
o = P═ O,
s = P═ S

### EXAMPLE 212

### Synthesis of Oligonucleotides Containing Boronated Pyrimidine Bases

Oligonucltodies containing boronated pyrimidine bases are synthesized as described in U.S. Pat. No. 5,130,302.

### EXAMPLE 213

### Synthesis of Oligonucleotides Containing C-2 and C-4 Modified A and G Modified Binding Bases

Oligonucltodies containing C-2 and C-4 modified A and G modified binding bases are synthesized as described in U.S. Pat. No. 6,060,592.

### EXAMPLE 214

### Synthesis of Oligonucleotides Containing 1,2,6 Optionally Modified Pyrimidine Bases

Oligonucleotdies containing 1,2,6 optionally modified pyrimidine bases are synthesized as described in U.S. Pat. Nos. 6,174,998 and 6,320,035.

### EXAMPLE 215

### Synthesis of Oligonucleotides Containing C2 Modified Pyrimidine Bases

Oligonucleotdies containing C2 modified pyrimidine bases are synthesized as described in U.S. Pat. No. 6,248,878.

### EXAMPLE 216

### Synthesis of Oligonucleotides Containing 3-Deazauracil Bases

Oligonucleotdies containing 3-deazauracil bases are synthesized as described in U.S. Pat. No. 5,134,066.

### EXAMPLE 217

### Synthesis of Oligonucleotides Containing A and G Modified Binding Bases Containing a C4 Substituted with a Reactive Group Derivatizable with a Detectable Label

Oligonucleotides containing A and G modified binding bases containing a C4 substituted with a reactive group derivatizable with a detectable label are synthesized as described in U.S. Pat. No. 6,268,132.

### EXAMPLE 218

### Synthesis of Oligonucleotides Containing 5-Substituted Cytosine or Uracil

Oligonucleotides containing 5-substituted cytosine or uracil are synthesized as described in U.S. Pat. No. 5,484,908.

### EXAMPLE 219

### Synthesis of Oligonucleotides Containing 5-Substituted Cytosine or Uracil Optionally Modified at C2 and C4

Oligonucleotides containing 5-substituted cytosine or uracil optionally modified at C2 and C4 are synthesized as described in U.S. Pat. Nos. 5,645,985 and 6,380,368.

### EXAMPLE 220

### Synthesis of Oligonucleotides Containing C5 or C6 Modified Pyrimidine Bases

Oligonucleotides containing C5 or C6 modified pyrimidine bases are synthesized as described in U.S. Pat. No. 5,614,617.

### EXAMPLE 221

### Synthesis of Oligonucleotides Containing C5 and C6 Alkyl-, Aza-, or Halo-Modified Pyrimidine Bases

Oligonucleotides containing C5 and C6 alkyl-, aza-, or halo-modified pyrimidine bases are synthesized as described in U.S. Pat. No. 5,672,511.

### EXAMPLE 222

### Synthesis of Oligonucleotides Containing 5-Fluorouracil

Oligonucleotides containing 5-fluorouracil are synthesized as described in U.S. Pat. No. 5,457,187.

### EXAMPLE 223

### Synthesis of Oligonucleotides Containing C5 Halo- or Alkyl-Substituted Pyrimidine Bases

Oligonucleotides containing C5 halo- or alkyl-substituted pyrimidine bases are synthesized as described in U.S. Pat. No. 6,166,197.

### EXAMPLE 224

### Synthesis of Oligonucleotides Containing C5-Amino Modified Pyrimidine Bases

Oligonucleotides containing C5-amino modified pyrimidine bases are synthesized as described in U.S. Pat. No. 5,552,540.

### EXAMPLE 225

### Synthesis of Oligonucleotides Containing Pyrimidine Bases Containing C5 Substituted with a Cationic Moiety

Oligonucleotides containing pyrimidine bases containing C5 substituted with a cationic moiety are synthesized as described in U.S. Pat. No. 5,596,091.

### EXAMPLE 226

### Synthesis of Oligonucleotides Containing A and G Modified Binding Bases for Forming Non-Standard Base Pairs

Oligonucleotides containing A and G modified binding bases for forming non-standard base pairs are synthesized as described in U.S. Pat. Nos. 5,432,272, 6,001,983 and 6,037,120.

### EXAMPLE 227

### Synthesis of Oligonucleotides Containing A and G Modified Binding Universal Bases

Oligonucleotides containing A and G modified binding universal bases are synthesized as described in U.S. Pat. No. 5,681,947.

### EXAMPLE 228

### Synthesis of Oligonucleotides Containing A and G Modified Binding Bases Containing a Polycyclic Aromatic Group

Oligonucleotides containing A and G modified binding bases containing a polycyclic aromatic group are synthesized as described in U.S. Pat. No. 5,175,273.

### EXAMPLE 229

### Synthesis of Oligonucleotides Containing Tricyclic A and G Modified Binding Bases Optionally Containing a Detectable Label

Oligonucleotides containing tricyclic A and G modified binding bases optionally containing a detectable label are synthesized as described in U.S. Pat. Nos. 6,007,992; 6,028,183; and 6,414,127.

### EXAMPLE 230

### Synthesis of Oligonucleotides Containing Tricyclic Modified Pyrimidine Bases

Oligonucleotides containing tricyclic modified pyrimidine bases are synthesized as described in U.S. Pat. Nos. 5,502,177; 5,763,588; and 6,005,096.

### EXAMPLE 231

### Synthesis of Oligonucleotides Containing Non-Heterocyclic A and G Modified Binding Bases

Oligonucleotides containing non-heterocyclic A and G modified binding bases are synthesized as described in U.S. Pat. No. 5,367,066.

### EXAMPLE 232

### Synthesis of Nucleoside Phosphoramidites

The following compounds, including amidites and their intermediates were prepared as described in U.S. Pat. No. 6,426,220 and published PCT WO 02/36743; 5′-O-Dimethoxytrityl-thymidine intermediate for 5-methyl dC amidite, 5′-O-Dimethoxytrityl-2′-deoxy-5-methylcytidine intermediate for 5-methyl-dC amidite, 5′-O-Dimethoxytrityl-2′-deoxy-N4-benzoyl-5-methylcytidine penultimate intermediate for 5-methyl dC amidite, [5′-O-(4,4′-Dimethoxytriphenylmethyl)-2′-deoxy-N-4-benzoyl-5-methylcytidin-3′-O-yl]-2-cyanoethyl-N,N-diisopropylphosphoramidite (5-methyl dC amidite), 2′-Fluorodeoxyadenosine, 2′-Fluorodeoxyguanosine, 2′-Fluorouridine, 2′-Fluorodeoxycytidine, 2′-O-(2-Methoxyethyl) modified amidites, 2′-O-(2-methoxyethyl)-5-methyluridine intermediate, 5′-O-DMT-2′-O-(2-methoxyethyl)-5-methyluridine penultimate intermediate, [5′-O-(4,4′-Dimethoxytriphenylmethyl)-2′-O-(2-methoxyethyl)-5-methyluridin-3′-O-yl]-2-cyanoethyl-N,N-diisopropylphosphoramidite (MOE T amidite), 5′-O-Dimethoxytrityl-2′-O-(2-methoxyethyl)-5-methylcytidine intermediate, 5′-O-dimethoxytrityl-2′-O-(2-methoxyethyl)-N-4-benzoyl-5-methyl-cytidine penultimate intermediate, [5′-O-(4,4′-Dimethoxytriphenylmethyl)-2′-O-(2-methoxyethyl)-N-4-benzoyl-5-methylcytidin-3′-O-yl]-2-cyanoethyl-N,N-diisopropylphosphoramidite (MOE 5-Me-C amidite), [5′-O-(4,4′-Dimethoxytriphenyhnethyl)-2′-O-(2-methoxyethyl)-N-6-benzoyladenosin-3′-O-yl]-2-cyanoethyl-N,N-diisopropylphosphoramidite (MOE A amdite), [5′-O-(4,4′-Dimethoxytriphenylmethyl)-2′-O-(2-methoxyethyl)-N-4-isobutyrylguanosin-3′-O-yl]-2-cyanoethyl-N,N-diisopropylphosphoramidite (MOE G amidite), 2′-O-(Aminooxyethyl) nucleoside amidites and 2′-O-(dimethylaminooxy-ethyl) nucleoside amidites, 2′-(Dimethylaminooxyethoxy) nucleoside amidites, 5′-O-tert-Butyldiphenylsilyl-O₂-2′-anhydro-5-methyluridine, 5′-O-tert-Butyldiphenylsilyl-2′-O-(2-hydroxyethyl)-5-methyluridine, 2′-O-([2-phthalimidoxy)ethyl]-5′-t-butyldiphenylsilyl-5-methyluridine, 5′-O-tert-butyldiphenylsilyl-2′-O-[(2-formadoximinooxy)ethyl]-5-methyluridine, 5′-O-tert-Butyldiphenylsilyl-2′-O-[N,N dimethylaminooxyethyl]-5-methyluridine, 2′-O-(dimethylaminooxyethyl)-5-methyluridine, 5′-O-DMT-2′-O-(dimethylaminooxyethyl)-5-methyluridine, 5′-O-DMT-2′-O-(2-N,N-dimethylaminooxyethyl)-5-methyluridine-3′-[(2-cyanoethyl)-N,N-diisopropylphosphoramidite], 2′-(Aminooxyethoxy) nucleoside amidites, N2-isobutyryl-6-O-diphenylcarbamoyl-2′-O-(2-ethylacetyl)-5′-O-(4,4′-dimethoxytrityl)guanosine-3′-[(2-cyanoethyl)-N,N-diisopropylphosphoramidite], 2′-dimethylaminoethoxyethoxy(2′-DMAEOE) nucleoside amidites, 2′-O-[2(2-N,N-dimethylaminoethoxy)ethyl]-5-methyl uridine, 5′-O-dimethoxytrityl-2′-O-[2(2-N,N-dimethylaminoethoxy)-ethyl)]-5-methyl uridine and 5′-O-Dimethoxytrityl-2′-O-[2(2-N,N-dimethylaminoethoxy)-ethyl)]-5-methyl uridine-3′-O-(cyanoethyl-N,N-diisopropyl)phosphoramidite.

### EXAMPLE 233

### Oligonucleotide and Oligonucleoside Synthesis

Oligonucleotides: Unsubstituted and substituted phosphodiester (P═O) oligonucleotides are synthesized on an automated DNA synthesizer (Applied Biosystems model 394) using standard phosphoramidite chemistry with oxidation by iodine.

Phosphorothioates (P═S) are synthesized similar to phosphodiester oligonucleotides with the following exceptions: thiation was effected by utilizing a 10% w/v solution of 3,H-1,2-benzodithiole-3-one 1,1-dioxide in acetonitrile for the oxidation of the phosphite linkages. The thiation reaction step time was increased to 180 sec and preceded by the normal capping step. After cleavage from the CPG column and deblocking in concentrated ammonium hydroxide at 55° C. (12-16 hr), the oligonucleotides were recovered by precipitating with >3 volumes of ethanol from a 1 M NH₄OAc solution. Phosphinate oligonucleotides are prepared as described in U.S. Pat. No. 5,508,270, herein incorporated by reference.

Alkyl phosphonate oligonucleotides are prepared as described in U.S. Pat. No. 4,469,863, herein incorporated by reference.

3′-Deoxy-3′-methylene phosphonate oligonucleotides are prepared as described in U.S. Pat. No. 5,610,289 or 5,625,050, herein incorporated by reference.

Phosphoramidite oligonucleotides are prepared as described in U.S. Patent, 5,256,775 or U.S. Pat. No. 5,366,878, herein incorporated by reference.

Alkylphosphonothioate oligonucleotides are prepared as described in published PCT applications PCT/US94/00902 and PCT/US93/06976 (published as WO 94/17093 and WO 94/02499, respectively), herein incorporated by reference.

3′-Deoxy-3′-amino phosphoramidate oligonucleotides are prepared as described in U.S. Pat. No. 5,476,925, herein incorporated by reference.

Phosphotriester oligonucleotides are prepared as described in U.S. Pat. No. 5,023,243, herein incorporated by reference.

Borano phosphate oligonucleotides are prepared as described in U.S. Pat. Nos. 5,130,302 and 5,177,198, both herein incorporated by reference.

Oligonucleosides: Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleo-sides, as well as mixed backbone oligomeric compounds having, for instance, alternating MMI and P═O or P═S linkages are prepared as described in U.S. Pat. Nos. 5,378,825, 5,386,023, 5,489,677, 5,602,240 and 5,610,289, all of which are herein incorporated by reference.

Formacetal and thioformacetal linked oligonucleosides are prepared as described in U.S. Pat. Nos. 5,264,562 and 5,264,564, herein incorporated by reference.

Ethylene oxide linked oligonucleosides are prepared as described in U.S. Pat. No. 5,223,618, herein incorporated by reference.

### EXAMPLE 234

### RNA Synthesis

In general, RNA synthesis chemistry is based on the selective incorporation of various protecting groups at strategic intermediary reactions. Although one of ordinary skill in the art will understand the use of protecting groups in organic synthesis, a useful class of protecting groups includes silyl ethers. In particular bulky silyl ethers are used to protect the 5′-hydroxyl in combination with an acid-labile orthoester protecting group on the 2′-hydroxyl. This set of protecting groups is then used with standard solid-phase synthesis technology. It is important to lastly remove the acid labile orthoester protecting group after all other synthetic steps. Moreover, the early use of the silyl protecting groups during synthesis ensures facile removal when desired, without undesired deprotection of 2′ hydroxyl.

Following this procedure for the sequential protection of the 5′-hydroxyl in combination with protection of the 2′-hydroxyl by protecting groups that are differentially removed and are differentially chemically labile, RNA oligonucleotides were synthesized.

RNA oligonucleotides are synthesized in a stepwise fashion. Each nucleotide is added sequentially (3′- to 5′-direction) to a solid support-bound oligonucleotide. The first nucleoside at the 3′-end of the chain is covalently attached to a solid support. The nucleotide precursor, a ribonucleoside phosphoramidite, and activator are added, coupling the second base onto the 5′-end of the first nucleoside. The support is washed and any unreacted 5′-hydroxyl groups are capped with acetic anhydride to yield 5′-acetyl moieties. The linkage is then oxidized to the more stable and ultimately desired P(V) linkage. At the end of the nucleotide addition cycle, the 5′-silyl group is cleaved with fluoride. The cycle is repeated for each subsequent nucleotide.

Following synthesis, the methyl protecting groups on the phosphates are cleaved in 30 minutes utilizing 1 M disodium-2-carbamoyl-2-cyanoethylene-1,1-dithiolate trihydrate (S₂Na₂) in DMF. The deprotection solution is washed from the solid support-bound oligonucleotide using water. The support is then treated with 40% methylamine in water for 10 minutes at 55° C. This releases the RNA oligonucleotides into solution, deprotects the exocyclic amines, and modifies the 2′-groups. The oligonucleotides can be analyzed by anion exchange HPLC at this stage.

The 2′-orthoester groups are the last protecting groups to be removed. The ethylene glycol monoacetate orthoester protecting group developed by Dharmacon Research, Inc. (Lafayette, Colo.), is one example of a useful orthoester protecting group which, has the following important properties. It is stable to the conditions of nucleoside phosphoramidite synthesis and oligonucleotide synthesis. However, after oligonucleotide synthesis the oligonucleotide is treated with methylamine which not only cleaves the oligonucleotide from the solid support but also removes the acetyl groups from the orthoesters. The resulting 2-ethyl-hydroxyl substituents on the orthoester are less electron withdrawing than the acetylated precursor. As a result, the modified orthoester becomes more labile to acid-catalyzed hydrolysis. Specifically, the rate of cleavage is approximately 10 times faster after the acetyl groups are removed. Therefore, this orthoester possesses sufficient stability in order to be compatible with oligonucleotide synthesis and yet, when subsequently modified, permits deprotection to be carried out under relatively mild aqueous conditions compatible with the final RNA oligonucleotide product.

Additionally, methods of RNA synthesis are well known in the art (Scaringe, S. A. Ph.D. Thesis, University of Colorado, 1996; Scaringe, S. A., et al., J. Am. Chem. Soc., 1998, 120, 11820-11821; Matteucci, M. D. and Caruthers, M. H. J. Am. Chem. Soc., 1981, 103, 3185-3191; Beaucage, S. L. and Caruthers, M. H. Tetrahedron Lett., 1981, 22, 1859-1862; Dahl, B. J., et al., Acta Chem. Scand,. 1990, 44, 639-641; Reddy, M. P., et al., Tetrahedrom Lett., 1994, 25, 4311-4314; Wincott, F. et al., Nucleic Acids Res., 1995, 23, 2677-2684; Griffin, B. E., et al., Tetrahedron, 1967, 23, 2301-2313; Griffin, B. E., et al., Tetrahedron, 1967, 23, 2315-2331).

### EXAMPLE 235

### Synthesis of Chimeric Oligonucleotides

Chimeric oligonucleotides, oligonucleosides or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”.

[2′-O-Me]-[2′-deoxy]-[2′-O-Me] Chimeric Phosphorothioate Oligonucleotides

Chimeric oligonucleotides having 2′-O-alkyl phosphorothioate and 2′-deoxy phosphorothioate oligonucleotide segments are synthesized using an Applied Biosystems automated DNA synthesizer Model 394, as above. Oligonucleotides are synthesized using the automated synthesizer and 2′-deoxy-5′-dimethoxytrityl-3′-O-phosphoramidite for the DNA portion and 5′-dimethoxytrityl-2′-O-methyl-3′-O-phosphoramidite for 5′ and 3′ wings. The standard synthesis cycle is modified by incorporating coupling steps with increased reaction times for the 5′-dimethoxytrityl-2′-O-methyl-3′-O-phosphoramidite. The fully protected oligonucleotide is cleaved from the support and deprotected in concentrated ammonia (NH₄OH) for 12-16 hr at 55° C. The deprotected oligo is then recovered by an appropriate method (precipitation, column chromatography, volume reduced in vacuo and analyzed spetrophotometrically for yield and for purity by capillary electrophoresis and by mass spectrometry.

[2′-O-(2-Methoxyethyl)]-[2′-deoxy]-[2′-O-(Methoxyethyl)] Chimeric Phosphorothioate Oligonucleotides

[2′-O-(2-methoxyethyl)]-[2′-deoxy]-[2′-O-(methoxyethyl)] chimeric phosphorothioate oligonucleotides were prepared as per the procedure above for the 2′-O-methyl chimeric oligonucleotide, with the substitution of 2′-O-(methoxyethyl)amidites for the 2′-O-methyl amidites.

[2′-O-(2-Methoxyethyl)Phosphodiester]-[2′-deoxy Phosphorothioate]-[2′-O-(2-Methoxyethyl) Phosphodiester] Chimeric Oligonucleotides

[2′-O-(2-methoxyethyl phosphodiester]-[2′-deoxy phosphorothioate]-[2′-O-(methoxyethyl) phosphodiester] chimeric oligonucleotides are prepared as per the above procedure for the 2′-O-methyl chimeric oligonucleotide with the substitution of 2′-O-(methoxyethyl) amidites for the 2′-O-methyl amidites, oxidation with iodine to generate the phosphodiester internucleotide linkages within the wing portions of the chimeric structures and sulfurization utilizing 3,H-1,2 benzodithiole-3-one 1,1 dioxide (Beaucage Reagent) to generate the phosphorothioate internucleotide linkages for the center gap.

Other chimeric oligonucleotides, chimeric oligonucleosides and mixed chimeric oligonucleotides/oligonucleosides are synthesized according to U.S. Pat. No. 5,623,065, herein incorporated by reference.

### EXAMPLE 236

### Design and Screening of Duplexed Oligomeric Compounds Targeting a Target

In accordance with the present invention, a series of nucleic acid duplexes comprising the antisense oligomeric compounds of the present invention and their complements can be designed to target a target. The ends of the strands may be modified by the addition of one or more natural or modified nucleobases to form an overhang. The sense strand of the dsRNA is then designed and synthesized as the complement of the antisense strand and may also contain modifications or additions to either terminus. For example, in one embodiment, both strands of the dsRNA duplex would be complementary over the central nucleobases, each having overhangs at one or both termini.

For example, a duplex comprising an antisense strand having the sequence CGAGAGGCGGACGGGACCG (SEQ ID NO:1) and having a two-nucleobase overhang of deoxythymidine(dT) would have the following structure:

| | | |
|---|---|---|
| 5′ cgagaggcggacgggaccgTT 3′ | Antisense Strand | (SEQ ID NO: 2) |
| \|\|\|\|\|\|\|\|\|\|\|\|\|\|\|\|\|\|\| | | |
| 3′ TTgctctccgcctgccctggc 5′ | Complement Strand | (SEQ ID NO: 3) |

RNA strands of the duplex can be synthesized by methods disclosed herein or purchased from Dharmacon Research Inc., (Lafayette, Colo.). Once synthesized, the complementary strands are annealed. The single strands are aliquoted and diluted to a concentration of 50 uM. Once diluted, 30 uL of each strand is combined with 15 uL of a 5×solution of annealing buffer. The final concentration of said buffer is 100 mM potassium acetate, 30 mM HEPES-KOH pH 7.4, and 2 mM magnesium acetate. The final volume is 75 uL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 uM. This solution can be stored frozen (−20° C.) and freeze-thawed up to 5 times.

Once prepared, the duplexed antisense oligomeric compounds are evaluated for their ability to modulate a target expression.

When cells reached 80% confluency, they are treated with duplexed antisense oligomeric compounds of the invention. For cells grown in 96-well plates, wells are washed once with 200 μL OPTI-MEM-1 reduced-serum medium (Gibco BRL) and then treated with 130 μL of OPTI-MEM-1 containing 12 μg/mL LIPOFECTIN (Gibco BRL) and the desired duplex antisense oligomeric compound at a final concentration of 200 nM. After 5 hours of treatment, the medium is replaced with fresh medium. Cells are harvested 16 hours after treatment, at which time RNA is isolated and target reduction measured by RT-PCR.

### EXAMPLE 237

### Oligonucleotide Isolation

After cleavage from the controlled pore glass solid support and deblocking in concentrated ammonium hydroxide at 55° C. for 12-16 hours, the oligonucleotides or oligonucleosides are recovered by precipitation out of 1 M NH₄OAc with >3 volumes of ethanol. Synthesized oligonucleotides were analyzed by electrospray mass spectroscopy (molecular weight determination) and by capillary gel electrophoresis and judged to be at least 70% full length material. The relative amounts of phosphorothioate and phosphodiester linkages obtained in the synthesis was determined by the ratio of correct molecular weight relative to the −16 amu product (+/−32+/−48). For some studies oligonucleotides were purified by HPLC, as described by Chiang et al., J. Biol. Chem. 1991, 266, 18162-18171. Results obtained with HPLC-purified material were similar to those obtained with non-HPLC purified material.

### EXAMPLE 23°

### Oligonucleotide Synthesis—96 Well Plate Format

Oligonucleotides were synthesized via solid phase P(III) phosphoramidite chemistry on an automated synthesizer capable of assembling 96 sequences simultaneously in a 96-well format. Phosphodiester internucleotide linkages were afforded by oxidation with aqueous iodine. Phosphorothioate internucleotide linkages were generated by sulfurization utilizing 3,H-1,2 benzodithiole-3-one 1,1 dioxide (Beaucage Reagent) in anhydrous acetonitrile. Standard base-protected beta-cyanoethyl-diiso-propyl phosphoramidites were purchased from commercial vendors (e.g. PE-Applied Biosystems, Foster City, Calif., or Pharmacia, Piscataway, N.J.). Non-standard nucleosides are synthesized as per standard or patented methods. They are utilized as base protected beta-cyanoethyldiisopropyl phosphoramidites.

Oligonucleotides were cleaved from support and deprotected with concentrated NH₄OH at elevated temperature (55-60° C.) for 12-16 hours and the released product then dried in vacuo. The dried product was then re-suspended in sterile water to afford a master plate from which all analytical and test plate samples are then diluted utilizing robotic pipettors.

### EXAMPLE 239

### Oligonucleotide Analysis—96-Well Plate Format

The concentration of oligonucleotide in each well was assessed by dilution of samples and UV absorption spectroscopy. The full-length integrity of the individual products was evaluated by capillary electrophoresis (CE) in either the 96-well format (Beckman P/ACE™ MDQ) or, for individually prepared samples, on a commercial CE apparatus (e.g., Beckman P/ACE™ 5000, ABI 270). Base and backbone composition was confirmed by mass analysis of the oligomeric compounds utilizing electrospray-mass spectroscopy. All assay test plates were diluted from the master plate using single and multi-channel robotic pipettors. Plates were judged to be acceptable if at least 85% of the oligomeric compounds on the plate were at least 85% full length.

### EXAMPLE 240

### Cell culture and Oligonucleotide Treatment

The effect of oligomeric compounds on target nucleic acid expression can be tested in any of a variety of cell types provided that the target nucleic acid is present at measurable levels. This can be routinely determined using, for example, PCR or Northern blot analysis. The following cell types are provided for illustrative purposes, but other cell types can be routinely used, provided that the target is expressed in the cell type chosen. This can be readily determined by methods routine in the art, for example Northern blot analysis, ribonuclease protection assays, or RT-PCR. T-24 cells:

The human transitional cell bladder carcinoma cell line T-24 was obtained from the American Type Culture Collection (ATCC) (Manassas, Va.). T-24 cells were routinely cultured in complete McCoy's 5A basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence. Cells were seeded into 96-well plates (Falcon-Primaria #353872) at a density of 7000 cells/well for use in RT-PCR analysis.

For Northern blotting or other analysis, cells may be seeded onto 100 mm or other standard tissue culture plates and treated similarly, using appropriate volumes of medium and oligonucleotide.

A549 Cells:

The human lung carcinoma cell line A549 was obtained from the American Type Culture Collection (ATCC) (Manassas, Va.). A549 cells were routinely cultured in DMEM basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence.

NHDF Cells:

Human neonatal dermal fibroblast (NHDF) were obtained from the Clonetics Corporation (Walkersville, Md.). NHDFs were routinely maintained in Fibroblast Growth Medium (Clonetics Corporation, Walkersville, Md.) supplemented as recommended by the supplier. Cells were maintained for up to 10 passages as recommended by the supplier.

HEK Cells:

Human embryonic keratinocytes (HEK) were obtained from the Clonetics Corporation (Walkersville, Md.). HEKs were routinely maintained in Keratinocyte Growth Medium (Clonetics Corporation, Walkersville, Md.) formulated as recommended by the supplier. Cells were routinely maintained for up to 10 passages as recommended by the supplier. Treatment with antisense oligomeric compounds:

When cells reached 65-75% confluency, they were treated with oligonucleotide. For cells grown in 96-well plates, wells were washed once with 100 μL OPTI-MEM™-1 reduced-serum medium (Invitrogen Corporation, Carlsbad, Calif.) and then treated with 130 μL of OPTI-MEM™-1 containing 3.75 μg/mL LIPOFECTIN™ (Invitrogen Corporation, Carlsbad, Calif.) and the desired concentration of oligonucleotide. Cells are treated and data are obtained in triplicate. After 4-7 hours of treatment at 37° C., the medium was replaced with fresh medium. Cells were harvested 16-24 hours after oligonucleotide treatment.

The concentration of oligonucleotide used varies from cell line to cell line. To determine the optimal oligonucleotide concentration for a particular cell line, the cells are treated with a positive control oligonucleotide at a range of concentrations. For human cells the positive control oligonucleotide is selected from either ISIS 13920 (TCCGTCATCGCTCCTCAGGG, SEQ ID NO: 4) which is targeted to human H-ras, or ISIS 18078, (GTGCGCGCGAGCCCGAAATC, SEQ ID NO: 5) which is targeted to human Jun-N-terminal kinase-2 (JNK2). Both controls are 2′-O-methoxyethyl gapmers (2′-O-methoxyethyls shown in bold) with a phosphorothioate backbone. For mouse or rat cells the positive control oligonucleotide is ISIS 15770, ATGCATTCTGCCCCCAAGGA (SEQ ID NO: 6) a 2′-O-methoxyethyl gapmer (2′-O-methoxyethyls shown in bold) with a phosphorothioate backbone which is targeted to both mouse and rat c-raf. The concentration of positive control oligonucleotide that results in 80% inhibition of c-H-ras (for ISIS 13920), JNK2 (for ISIS 18078) or c-raf (for ISIS 15770) mRNA is then utilized as the screening concentration for new oligonucleotides in subsequent experiments for that cell line. If 80% inhibition is not achieved, the lowest concentration of positive control oligonucleotide that results in 60% inhibition of c-H-ras, JNK2 or c-raf mRNA is then utilized as the oligonucleotide screening concentration in subsequent experiments for that cell line. If 60% inhibition is not achieved, that particular cell line is deemed as unsuitable for oligonucleotide transfection experiments. The concentrations of antisense oligonucleotides used herein are from 50 nM to 300 nM.

### EXAMPLE 241

### Analysis of Oligonucleotide Inhibition of a Target Expression

Modulation of a target expression can be assayed in a variety of ways known in the art. For example, a target mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+ mRNA. The preferred method of RNA analysis of the present invention is the use of total cellular RNA as described in other examples herein. Methods of RNA isolation are well known in the art. Northern blot analysis is also routine in the art. Real-time quantitative (PCR) can be conveniently accomplished using the commercially available ABI PRISM™ 7600, 7700, or 7900 Sequence Detection System, available from PE-Applied Biosystems, Foster City, Calif. and used according to manufacturer's instructions.

Protein levels of a target can be quantitated in a variety of ways well known in the art, such as immunoprecipitation, Western blot analysis (immunoblotting), enzyme-linked immunosorbent assay (ELISA) or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art.

### EXAMPLE 242

### Design of Phenotypic Assays and In Vivo Studies for the Use of a Target Inhibitors

Phenotypic Assays

Once a target inhibitors have been identified by the methods disclosed herein, the oligomeric compounds are further investigated in one or more phenotypic assays, each having measurable endpoints predictive of efficacy in the treatment of a particular disease state or condition.

Phenotypic assays, kits and reagents for their use are well known to those skilled in the art and are herein used to investigate the role and/or association of a target in health and disease. Representative phenotypic assays, which can be purchased from any one of several commercial vendors, include those for determining cell viability, cytotoxicity, proliferation or cell survival (Molecular Probes, Eugene, Oreg.; PerkinElmer, Boston, Mass.), protein-based assays including enzymatic assays (Panvera, LLC, Madison, Wis.; BD Biosciences, Franklin Lakes, N.J.; Oncogene Research Products, San Diego, Calif.), cell regulation, signal transduction, inflammation, oxidative processes and apoptosis (Assay Designs Inc., Ann Arbor, Mich.), triglyceride accumulation (Sigma-Aldrich, St. Louis, Mo.), angiogenesis assays, tube formation assays, cytokine and hormone assays and metabolic assays (Chemicon International Inc., Temecula, Calif.; Amersham Biosciences, Piscataway, N.J.).

In one non-limiting example, cells determined to be appropriate for a particular phenotypic assay (i.e., MCF-7 cells selected for breast cancer studies; adipocytes for obesity studies) are treated with a target inhibitors identified from the in vitro studies as well as control compounds at optimal concentrations which are determined by the methods described above. At the end of the treatment period, treated and untreated cells are analyzed by one or more methods specific for the assay to determine phenotypic outcomes and endpoints. Phenotypic endpoints include changes in cell morphology over time or treatment dose as well as changes in levels of cellular components such as proteins, lipids, nucleic acids, hormones, saccharides or metals. Measurements of cellular status which include pH, stage of the cell cycle, intake or excretion of biological indicators by the cell, are also endpoints of interest.

Analysis of the geneotype of the cell (measurement of the expression of one or more of the genes of the cell) after treatment is also used as an indicator of the efficacy or potency of the target inhibitors. Hallmark genes, or those genes suspected to be associated with a specific disease state, condition, or phenotype, are measured in both treated and untreated cells.

In Vivo Studies

The individual subjects of the in vivo studies described herein are warm-blooded vertebrate animals, which includes humans.

The clinical trial is subjected to rigorous controls to ensure that individuals are not unnecessarily put at risk and that they are fully informed about their role in the study.

To account for the psychological effects of receiving treatments, volunteers are randomly given placebo or a target inhibitor. Furthermore, to prevent the doctors from being biased in treatments, they are not informed as to whether the medication they are administering is a a target inhibitor or a placebo. Using this randomization approach, each volunteer has the same chance of being given either the new treatment or the placebo.

Volunteers receive either the a target inhibitor or placebo for eight week period with biological parameters associated with the indicated disease state or condition being measured at the beginning (baseline measurements before any treatment), end (after the final treatment), and at regular intervals during the study period. Such measurements include the levels of nucleic acid molecules encoding a target or a target protein levels in body fluids, tissues or organs compared to pre-treatment levels. Other measurements include, but are not limited to, indices of the disease state or condition being treated, body weight, blood pressure, serum titers of pharmacologic indicators of disease or toxicity as well as ADME (absorption, distribution, metabolism and excretion) measurements.

Information recorded for each patient includes age (years), gender, height (cm), family history of disease state or condition (yes/no), motivation rating (some/moderate/great) and number and type of previous treatment regimens for the indicated disease or condition.

Volunteers taking part in this study are healthy adults (age 18 to 65 years) and roughly an equal number of males and females participate in the study. Volunteers with certain characteristics are equally distributed for placebo and a target inhibitor treatment. In general, the volunteers treated with placebo have little or no response to treatment, whereas the volunteers treated with the target inhibitor show positive trends in their disease state or condition index at the conclusion of the study.

### EXAMPLE 243

### RNA Isolation

Poly(A)+ mRNA isolation

Poly(A)+ mRNA was isolated according to Miura et al., (Clin. Chem., 1996, 42, 1758-1764). Other methods for poly(A)+ mRNA isolation are routine in the art. Briefly, for cells grown on 96-well plates, growth medium was removed from the cells and each well was washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) was added to each well, the plate was gently agitated and then incubated at room temperature for five minutes. 55 μL of lysate was transferred to Oligo d(T) coated 96-well plates (AGCT Inc., Irvine Calif.). Plates were incubated for 60 minutes at room temperature, washed 3 times with 200 μL of wash buffer (10 mM Tris-HCl pH 7.6, 1 mM EDTA, 0.3 M NaCl). After the final wash, the plate was blotted on paper towels to remove excess wash buffer and then air-dried for 5 minutes. 60 μL of elution buffer (5 mM Tris-HCl pH 7.6), preheated to 70° C., was added to each well, the plate was incubated on a 90° C. hot plate for 5 minutes, and the eluate was then transferred to a fresh 96-well plate.

Cells grown on 100 mm or other standard plates may be treated similarly, using appropriate volumes of all solutions.

Total RNA Isolation

Total RNA was isolated using an RNEASY 96™ kit and buffers purchased from Qiagen Inc. (Valencia, Calif.) following the manufacturer's recommended procedures. Briefly, for cells grown on 96-well plates, growth medium was removed from the cells and each well was washed with 200 μL cold PBS. 150 μL Buffer RLT was added to each well and the plate vigorously agitated for 20 seconds. 150 μL of 70% ethanol was then added to each well and the contents mixed by pipetting three times up and down. The samples were then transferred to the RNEASY 96™ well plate attached to a QIAVAC™ manifold fitted with a waste collection tray and attached to a vacuum source. Vacuum was applied for 1 minute. 500 μL of Buffer RW1 was added to each well of the RNEASY 96™ plate and incubated for 15 minutes and the vacuum was again applied for 1 minute. An additional 500 μL of Buffer RW1 was added to each well of the RNEASY 96™ plate and the vacuum was applied for 2 minutes. 1 mL of Buffer RPE was then added to each well of the RNEASY 96™ plate and the vacuum applied for a period of 90 seconds. The Buffer RPE wash was then repeated and the vacuum was applied for an additional 3 minutes. The plate was then removed from the QIAVAC™ manifold and blotted dry on paper towels. The plate was then re-attached to the QIAVAC™ manifold fitted with a collection tube rack containing 1.2 mL collection tubes. RNA was then eluted by pipetting 140 μL of RNAse free water into each well, incubating 1 minute, and then applying the vacuum for 3 minutes.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### EXAMPLE 244

### Real-time Quantitative PCR Analysis of a target mRNA Levels

Quantitation of a target mRNA levels was accomplished by real-time quantitative PCR using the ABI PRISM™ 7600, 7700, or 7900 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., FAM or JOE, obtained from either PE-Applied Biosystems, Foster City, Calif., Operon Technologies Inc., Alameda, Calif. or Integrated DNA Technologies Inc., Coralville, Iowa) is attached to the 5′ end of the probe and a quencher dye (e.g., TAMRA, obtained from either PE-Applied Biosystems, Foster City, Calif., Operon Technologies Inc., Alameda, Calif. or Integrated DNA Technologies Inc., Coralville, Iowa) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

PCR reagents were obtained from Invitrogen Corporation, (Carlsbad, Calif.). RT-PCR reactions were carried out by adding 20 μL PCR cocktail (2.5×PCR buffer minus MgCl₂, 6.6 mM MgCl₂, 375 μM each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5×ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by real time RT-PCR are normalized using either the expression level of GAPDH, a gene whose expression is constant, or by quantifying total RNA using RiboGreen™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent (Molecular Probes, Inc. Eugene, Oreg.). Methods of RNA quantification by RiboGreen™ are taught in Jones, L. J., et al, (Analytical Biochemistry, 1998, 265, 368-374).

In this assay, 170 μL of RiboGreen™ working reagent (RiboGreen™ reagent diluted 1:350 in 10 mM Tris-HCl, 1 mM EDTA, pH 7.5) is pipetted into a 96-well plate containing 30 μL purified, cellular RNA. The plate is read in a CytoFluor 4000 (PE Applied Biosystems) with excitation at 485 nm and emission at 530 nm.

Probes and primers are designed to hybridize to a human a target sequence, using published sequence information.

### EXAMPLE 245

### Northern Blot Analysis of a Target mRNA Levels

Eighteen hours after treatment, cell monolayers were washed twice with cold PBS and lysed in 1 mL RNAZOL™ (TEL-TEST “B” Inc., Friendswood, Tex.). Total RNA was prepared following manufacturer's recommended protocols. Twenty micrograms of total RNA was fractionated by electrophoresis through 1.2% agarose gels containing 1.1% formaldehyde using a MOPS buffer system (AMRESCO, Inc. Solon, Ohio). RNA was transferred from the gel to HYBOND™-N+nylon membranes (Amersham Pharmacia Biotech, Piscataway, N.J.) by overnight capillary transfer using a Northern/Southern Transfer buffer system (TEL-TEST “B” Inc., Friendswood, Tex.). RNA transfer was confirmed by UV visualization. Membranes were fixed by UV cross-linking using a STRATALNKER™ UV Crosslinker 2400 (Stratagene, Inc, La Jolla, Calif.) and then probed using QUICKHYB™ hybridization solution (Stratagene, La Jolla, Calif.) using manufacturer's recommendations for stringent conditions.

To detect human a target, a human a target specific primer probe set is prepared by PCR To normalize for variations in loading and transfer efficiency membranes are stripped and probed for human glyceraldehyde-3-phosphate dehydrogenase (GAPDH) RNA (Clontech, Palo Alto, Calif.).

Hybridized membranes were visualized and quantitated using a PHOSPHORIMAGER™ and IMAGEQUANT™ Software V3.3 (Molecular Dynamics, Sunnyvale, Calif.). Data was normalized to GAPDH levels in untreated controls.

### EXAMPLE 246

### Inhibition of human a target expression by oligonucleotides

In accordance with the present invention, a series of oligomeric compounds are designed to target different regions of the human target RNA. The oligomeric compounds are analyzed for their effect on human target mRNA levels by quantitative real-time PCR as described in other examples herein. Data are averages from three experiments. The target regions to which these preferred sequences are complementary are herein referred to as “preferred target segments” and are therefore preferred for targeting by oligomeric compounds of the present invention. The sequences represent the reverse complement of the preferred antisense oligomeric compounds.

As these “preferred target segments” have been found by experimentation to be open to, and accessible for, hybridization with the antisense oligomeric compounds of the present invention, one of skill in the art will recognize or be able to ascertain, using no more than routine experimentation, further embodiments of the invention that encompass other oligomeric compounds that specifically hybridize to these preferred target segments and consequently inhibit the expression of a target.

According to the present invention, antisense oligomeric compounds include antisense oligomeric compounds, antisense oligonucleotides, ribozymes, external guide sequence (EGS) oligonucleotides, alternate splicers, primers, probes, and other short oligomeric compounds that hybridize to at least a portion of the target nucleic acid.

### EXAMPLE 247

### Western Blot Analysis of a Target Protein Levels

Western blot analysis (immunoblot analysis) is carried out using standard methods. Cells are harvested 16-20 h after oligonucleotide treatment, washed once with PBS, suspended in Laemmli buffer (100 μl/well), boiled for 5 minutes and loaded on a 16% SDS-PAGE gel. Gels are run for 1.5 hours at 150 V, and transferred to membrane for western blotting. Appropriate primary antibody directed to a target is used, with a radiolabeled or fluorescently labeled secondary antibody directed against the primary antibody species. Bands are visualized using a PHOSPHORIMAGER™ (Molecular Dynamics, Sunnyvale Calif.).

1. A composition comprising a first oligomer and a second oligomer, wherein:
at least a portion of said first oligomer is capable of hybridizing with at least a portion of said second oligomer,
at least a portion of said first oligomer is complementary to and capable of hybridizing to a selected target nucleic acid, and
at least one of said first or said second oligomer includes at least one A and G modified binding base.

2. The composition of claim 1 wherein said first and said second oligomers are a complementary pair of siRNA oligomers.

3. The composition of claim 1 wherein said first and said second oligomers are an antisense/sense pair of oligomers.

4. The composition of claim 1 wherein each of said first and second oligomers has 12 to 50 nucleotides.

5. The composition of claim 1 wherein each of said first and second oligomers has 15 to 30 nucleotides.

6. The composition of claim 1 wherein each of said first and second oligomers has 21 to 24 nucleotides.

7. The composition of claim 1 wherein said first oligomer is an antisense oligomer.

8. The composition of claim 7 wherein said second oligomer is a sense oligomer.

9. The composition of claim 7 wherein said second oligomer has a plurality of ribose nucleotide units.

10. The composition of claim 1 wherein said first oligomer includes said nucleotide having an A and G modified binding base.

11. The composition of claim 1 wherein said A and G modified binding base is a boronated A and G modified binding base having a boron-containing substituent selected from the group consisting of —BH₂CN, —BH₃, and —BH₂COOR, wherein R is C1 to C18 alkyl.

12. The composition of claim 1 wherein said A and G modified binding base is an A and G modified binding base of one of the following structures: 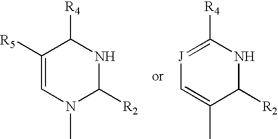
wherein:
J is N or CH;
R₅ is H or CH₃;
one of R₂ and R₄ is ═O, ═NH, or ═NH₂⁺ or the tautomeric form —OH, —NH₂, —NH₃⁺; and the other of R₂ and R₄ is Q, ═C(R_{A})-Q, C(R_{A})(R_{B})—C(R_{C})(R_{D})-Q, C(R_{A})═C(R_{C})-Q or C≡C-Q;
R_{A}, R_{B}, R_{C} and R_{D}, independently, are H, SH, OH, NH₂, or C₁-C₂₀ alkyl, or one of (R_{A})(R_{B}) or (R_{C})(R_{D}) is ═O;
Q is halogen, hydrogen, C₁-C₂₀ alkyl, C₁-C₂₀ alkylamine, C₁-C₂₀ alkyl-N-phthalimide, C₁-C₂₀ alkylimidazole, C₁-C₂₀ alkylbis-imidazole, imidazole, bis-imidazole, amine, N-phthalimide, C₂-C₂₀ alkenyl, C₂-C₂₀ alkynyl, hydroxyl, thiol, keto, carboxyl, nitrate, nitro, nitroso, nitrile, trifluoromethyl, trifluoromethoxy, O-alkyl, S-alkyl, NH-alkyl, N-dialkyl, O-aralkyl, S-aralkyl, NH-aralkyl, azido, hydrazino, hydroxylamino, isocyanato, sulfoxide, sulfone, sulfide, disulfide, or silyl; and
when R₂ is ═O, R₄ is other than hydroxyl or amine.

13. The composition of claim 1 wherein said A and G modified binding base is an A and G modified binding base of the following structure: 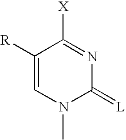
wherein
X is hydroxyl or amino;
R is halo or C₁-C₆ alkyl or substituted C₁-C₆ alkyl wherein said substitution is halo, amino, hydroxyl, thiol, ether or thioether;
L is oxygen or sulfur; and
when X is hydroxyl and L is oxygen, R is other than Cl alkyl.

14. The composition of claim 1 wherein said A and G modified binding base is an A and G modified binding base of the following structure: 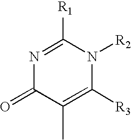
wherein
R₁, R₂, and R₃ can be same or different and are hydrogen, halogen, hydroxy, thio or substituted thio, amino or substituted amino, carboxy, lower alkyl, lower alkenyl, lower alkinyl, aryl, lower alkyloxy, aryloxy, aralkyl, aralkyloxy or a reporter group.

15. The composition of claim 1 wherein said A and G modified binding base is an A and G modified binding base selected from the group consisting of 2-fluoropyridine-3-yl, pyridin-2-one-3-yl, pyridin-2-(4-nitrophenylethyl)-one-3-yl, 2-bromopyridine-5-yl, pyridin-2-one-5-yl, 2-aminopyridine-5-yl, and pyridin-2-(4-nitrophenylethyl)-one-5-yl.

16. The composition of claim 1 wherein said A and G modified binding base is a 3-deazauracil or 3-deazacytosine analogue of one of the following structures: 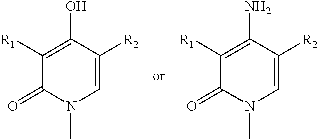
wherein R₁ and R₂, independently, are C₁-C₅ alkyl, C₂-C₅ alkenyl, halo or hydrogen.

17. The composition of claim 1 wherein said A and G modified binding base is a 5-substituted cytosine or uracil base of one of the following formulas: 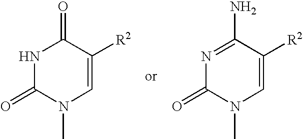
wherein
R₂ is selected from the group consisting of propynyl (—C≡C—CH₃), propenyl (—CH═CH—CH₃), 3-buten-1-ynyl (—C≡C—CH═CH₂), 3-methyl-1-butynyl (—C≡C—CH(CH₃)₂), 3,3-dimethyl-1-butynyl (—C≡C—C(CH₃)₃), phenyl, m-pyridinyl, p-pyridinyl and o-pyridinyl.

18. The composition of claim 1 wherein said A and G modified binding base is a 5-substituted cytosine or uracil base of one of the following formulas: 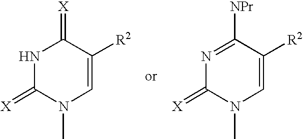
wherein
each X is independently O or S;
R₂ is selected from the group consisting of vinyl, 1-butenyl, 1-pentenyl, 1-hexenyl, 1-heptenyl, 1-octenyl, 1,3-pentadiynyl, 1-propynyl, 1-butynyl, 1-pentynyl, 3-methyl-1-butynyl, 3,3-dimethyl-1-butynyl, 3-buten-1-ynyl, bromovinyl, 1-hexynyl, 1-heptynyl, 1-octynyl, —C≡C-Z wherein Z is C₁₋₁₀ alkyl or C₁₋₁₀ haloalkyl, a 5-heteroaromatic group, or a 5-1-alkynyl)-heteroaromatic group; wherein the 5-heteroaromatic group and the 5-(1-alkynyl)-heteroaromatic group are optionally substituted on a ring carbon by oxygen or C₁₋₄ alkyl or are substituted on a ring nitrogen by C₁₋₄ alkyl; and
Pr is (H)₂ or a protecting group.

19. The composition of claim 1 wherein said A and G modified binding base is an A and G modified binding base having the following structure: 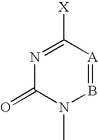
wherein
X is OH or NH₂, and
A and B may be the same or different and are C-lower alkyl, N, C—CF₃, C—F, C—Cl, C—Br, C—I, C-halocarbon, C—NO₂, C—OCF₃, C—SH, C—SCH₃, C—OH, C—O-lower alkyl, C—CH₂OH, C—CH₂SH, C—CH₂SCH₃, C—CH₂OCH₃, C—NH₂, C—CH₂ NH₂, C-alkyl-NH₂, C-benzyl, C-aryl, C-substituted aryl, C-substituted benzyl; or one of A and B are as defined above and the other is C—H; or together A and B are part of a carbocyclic or heterocyclic ring fused to the pyrimidine ring through A and B.

20. The composition of claim 1 wherein said A and G modified binding base is 5-alkylcytidine, 5-alkyluridine, 5-halouridine, 6-azapyrimidine, or 6-alkyluridine.

21. The composition of claim 1 wherein said A and G modified binding base is 5-fluorouracil.

22. The composition of claim 1 wherein said A and G modified binding base is an A and G modified binding base of the following structure: 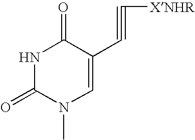
wherein
X′ is a branched or unbranched C₁₋₁₅ alkyl group;
R is an amino protecting group, a fluorophore, a non-radioactive detectable marker, or the group Y′NHA, where Y′ is a branched or unbranched C₁₋₄₀ alkyl carbonyl group and A is an amino protecting group, a fluorophore, or a non-radioactive detectable marker.

23. The composition of claim 1 wherein said A and G modified binding base is an A and G modified binding base of one of the following structures: 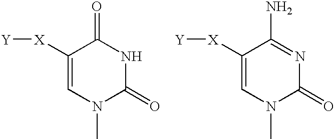
wherein
X is C₁-C₁₀ alkyl, C₁-C₁₀ unsaturated alkyl, dialkyl ether or dialkylthioether;
Y is —(NH₃)⁺, —(NH₂R¹)⁺, —(NHR¹R²)⁺, —(NR¹R²R³)⁺, dialkylsulfonium or trialkylphosphonium; and
R¹, R², and R³ are each independently lower alkyl having from one to ten carbon atoms.

24. The composition of claim 1 wherein said A and G modified binding base is an A and G modified binding base of the following structure: 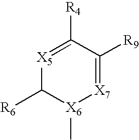
wherein
X₅ is N, O, C, S, or Si;
X₆ is CH or N, and at least one of X₅ and X₆ is N;
X₇ is —CH—;
R₄ is a reactive group derivatizable with a detectable label wherein said reactive group is selected from the group consisting of NH₂, SH, ═O, and a linking moiety selected from the group consisting of an amide, a thioether, a disulfide, a combination of an amide a thioether or a disulfide, R₁-(CH₂), —R₂ and R₁—R₂—(CH₂), —R₃ wherein x is an integer from 1 to 25 inclusive, and R₁, R₂, and R₃ are H, OH, alkyl, acyl, amide, thioether, or disulfide, and said detectable label is selected from the group consisting of radioisotopes, fluorescent or chemiluminescent reporter molecules, antibodies, haptens, biotin, photobiotin, digoxigenin, fluorescent aliphatic amino groups, avidin, enzymes, and acridinium;
R₆ is H, NH₂, SH, or ═O;
R₉ is hydrogen, methyl, bromine, fluorine, or iodine, alkyl or aromatic substituents, or an optional linking moiety selected from the group consisting of an amide, a thioether, a disulfide linkage, and a combination thereof.

25. The composition of claim 1 wherein said A and G modified binding base is an A and G modified binding base of one the following structures: 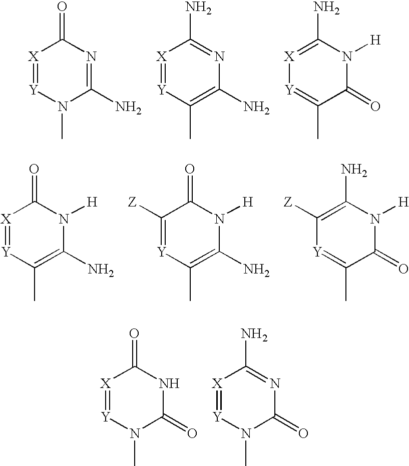
wherein
X is selected from the group consisting of a nitrogen atom and a carbon atom bearing a substituent Z;
Z is either a hydrogen, an unfunctionalized lower alkyl chain, or a lower alkyl chain bearing an amino, carboxyl, hydroxy, thiol, aryl, indole, or imidazoyl group; and
Y is selected from the group consisting of N and CH.

26. The composition of claim 1 wherein said A and G modified binding base is an A and G modified binding universal base of the following structure: 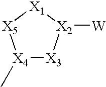
wherein
the foregoing structure has at least two double bonds in one of its possible tautomeric forms;
X₁, X₃ and X₅ are each members of the group consisting of N, O, C, S and Se;
X₂ and X₄ are each members of the group consisting of N and C; and
W is a member of the group consisting of F, Cl, Br, I, O, S, OH, SH, NH₂, NO₂, C(O)H, C(O)NHOH, C(S)NHOH, NO, C(NOCH₃)NH₂, OCH₃, SCH₃, SeCH₃, ONH₂, NHOCH₃, N₃, CN, C(O)NH₂, C(NOH)NH₂, CSNH₂ and CO₂H.

27. The composition of claim 1 wherein said A and G modified binding base is an A and G modified binding base of the following structure: 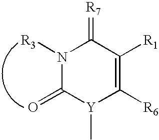
wherein
R₃ is a polycyclic aromatic group;
Y is C or N; R₇ is N or ═C(R₁)—; and
R₁ and Rr are independently selected from the group consisting of H, halogen, C₁-C₁₀-alkyl, saturated or unsaturated cycloalkyl, C₁-C₁₀-alkylcarbonyloxy, hydroxy-C₁-C₁₀-alkyl, heterocycle (N, O, or S), and nitro.

28. The composition of claim 1 wherein said A and G modified binding base is an A and G modified binding base analogue of the following structure: 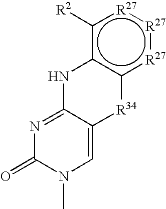
wherein
R² is A(Z)_{X1}, wherein A is a spacer and Z independently is a label bonding group optionally bonded to a detectable label;
R²⁷ is independently —CH═, —N═, —C(C₁₋₈ alkyl)=or —C(halogen)=, but no adjacent R²⁷ are both —N═, or two adjacent R²⁷ are taken together to form a ring having the structure, 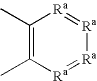
where each R^{a} is, independently, —CH═, —N═, —C(C₁₋₈ alkyl)=or —C(halogen)=, but no adjacent R^{a} are both —N═;
R³⁴ is —O—, —S— or —N(CH₃)—; and
X1 is 1, 2 or 3.

29. The composition of claim 1 wherein said A and G modified binding base is an A and G modified binding base of the following structure: 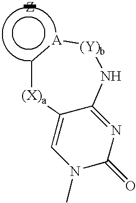
wherein
a and b are 0 or 1, and the total of a and b is 0 or 1;
A is N or C;
X is S, O, —C(O)—, NH or NCH₂Rr;
Y is —C(O)—;
Z is taken together with A to form an aryl or heteroaryl ring structure comprising 5 or 6 ring atoms wherein the heteroaryl ring comprises a single O ring heteroatom, a single N ring heteroatom, a single S ring heteroatom, a single 0 and a single N ring heteroatom separated by a carbon atom, a single S and a single N ring heteroatom separated by a carbon atom, 2 N ring heteroatoms separated by a carbon atom, or 3 N ring heteroatoms at least two of which are separated by a carbon atom, and wherein at least 1 nonbridging ring carbon atom is substituted with R₆ or ═O;
R₃ is a protecting group or H;
R₆ is independently H, C₁-C₆ alkyl, C₂-C₆ alkenyl, C₂-C₆ alkynyl, NO₂, N(R₃)₂, C≡N or halo, or R₆ is taken together with an adjacent R₆ to complete a ring containing 5 or 6 ring atoms.

30. The composition of claim 1 wherein said A and G modified binding base is a non-heterocyclic A and G modified binding base of the following structure:
—O—Rₘ—O—Rₙ
wherein
Rₘ is C₁ to C₁₆ alkylene or an oxyethylene oligomer —(CH₂CH₂O)_{z}— where z is an integer in the range of 1 to 16 inclusive, and
Rₙ is selected from the group consisting of:

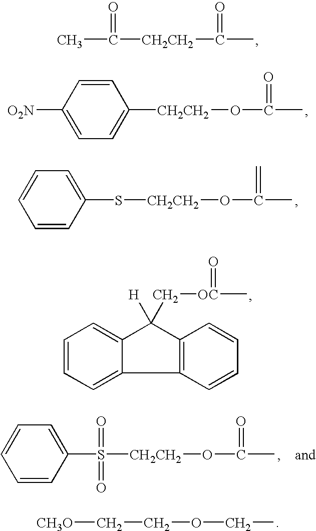

31. A pharmaceutical composition comprising the composition of claim 1 and a pharmaceutically acceptable carrier.

32. A method of modulating the expression of a target nucleic acid in a cell comprising contacting said cell with a composition of claim 1.

33. A method of treating or preventing a disease or disorder associated with a target nucleic acid comprising administering to an animal having or predisposed to said disease or disorder a therapeutically effective amount of a composition of claim 1.

34. A composition comprising an oligomer complementary to and capable of hybridizing to a selected target nucleic acid and at least one protein, said protein comprising at least a portion of a RNA-induced silencing complex (RISC), wherein:
said oligomer includes at least one nucleotide having an A and G modified binding base.

35. The composition of claim 34 wherein said oligomer is an antisense oligomer.

36. The composition of claim 34 wherein said oligomer has 12 to 50 nucleotides.

37. The composition of claim 34 wherein said oligomer has 15 to 30 nucleotides.

38. The composition of claim 34 wherein said oligomer has 21 to 24 nucleotides.

39. The composition of claim 34 including a further oligomer, wherein said further oligomer is complementary to and hydrizable to said oligomer.

40. The composition of claim 39 wherein said further oligomer is a sense oligomer.

41. The composition of claim 39 wherein said further oligomer is an oligomer having a plurality of ribose nucleotide units.

42. The composition of claim 34 wherein said A and G modified binding base is a boronated A and G modified binding base having a boron-containing substituent selected from the group consisting of —BH₂CN, —BH₃, and —BH₂COOR, wherein R is C1 to C18 alkyl.

43. The composition of claim 34 wherein said A and G modified binding base is an A and G modified binding base of one of the following structures: 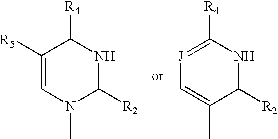
wherein:
J is N or CH;
R₅ is H or CH₃;
one of R₂ and R₄ is ═O, ═NH, or ═NH₂⁺ or the tautomeric form —OH, —NH₂, —NH₃⁺; and
the other of R₂ and R₄ is Q, ═C(R_{A})-Q, C(R_{A})(R_{B})—C(R_{C})(R_{D})-Q, C(R_{A})═C(R_{C})-Q or C—C-Q;
R_{A}, R_{B}, R_{C} and R_{D}, independently, are H, SH, OH, NH₂, or C₁-C₂₀ alkyl, or one of (R_{A})(R_{B}) or (R_{C})(R_{D}) is ═O;
Q is halogen, hydrogen, C₁-C₂₀ alkyl, C₁-C₂₀ alkylamine, C₁-C₂₀ alkyl-N-phthalimide, C₁-C₂₀ alkylimidazole, C₁-C₂₀ alkylbis-imidazole, imidazole, bis-imidazole, amine, N-phthalimide, C₂-C₂₀ alkenyl, C₂-C₂₀ alkynyl, hydroxyl, thiol, keto, carboxyl, nitrate, nitro, nitroso, nitrile, trifluoromethyl, trifluoromethoxy, O-alkyl, S-alkyl, NH-alkyl, N-dialkyl, O-aralkyl, S-aralkyl, NH-aralkyl, azido, hydrazino, hydroxylamino, isocyanato, sulfoxide, sulfone, sulfide, disulfide, or silyl; and
when R₂ is ═O, R₄ is other than hydroxyl or amine.

44. The composition of claim 34 wherein said A and G modified binding base is an A and G modified binding base of the following structure: 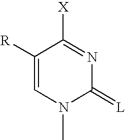
wherein
X is hydroxyl or amino;
R is halo or C₁-C₆ alkyl or substituted C₁-C₆ alkyl wherein said substitution is halo, amino, hydroxyl, thiol, ether or thioether;
L is oxygen or sulfur; and
when X is hydroxyl and L is oxygen, R is other than C₁ alkyl

45. The composition of claim 34 wherein said A and G modified binding base is an A and G modified binding base of the following structure: 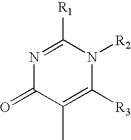
wherein
R₁, R₂, and R₃ can be same or different and are hydrogen, halogen, hydroxy, thio or substituted thio, amino or substituted amino, carboxy, lower alkyl, lower alkenyl, lower alkinyl, aryl, lower alkyloxy, aryloxy, aralkyl, aralkyloxy or a reporter group.

46. The composition of claim 34 wherein said A and G modified binding base is an A and G modified binding base selected from the group consisting of 2-fluoropyridine-3-yl, pyridin-2-one-3-yl, pyridin-2-(4-nitrophenylethyl)-one-3-yl, 2-bromopyridine-5-yl, pyridin-2-one-5-yl, 2-aminopyridine-5-yl, and pyridin-2-(4-nitrophenylethyl)-one-5-yl.

47. The composition of claim 34 wherein said A and G modified binding base is a 3-deazauracil or 3-deazacytosine analogue of one of the following structures: 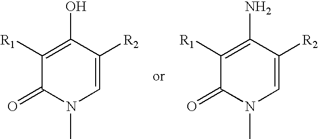
wherein R₁ and R₂, independently, are C₁-C₅ alkyl, C₂-C₅ alkenyl, halo or hydrogen.

48. The composition of claim 34 wherein said A and G modified binding base is a 5-substituted cytosine or uracil base of one of the following formulas: 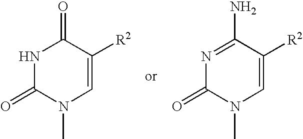
wherein
R₂ is selected from the group consisting of propynyl (—C≡C—CH₃), propenyl (—CH═CH—CH₃), 3-buten-1-ynyl (—C≡C—CH═CH₂), 3-methyl-1-butynyl (—C≡C—CH(CH₃)₂), 3,3-dimethyl-1-butynyl (—C≡C—C(CH₃)₃), phenyl, m-pyridinyl, p-pyridinyl and o-pyridinyl.

49. The composition of claim 34 wherein said A and G modified binding base is a 5-substituted cytosine or uracil base of one of the following formulas: 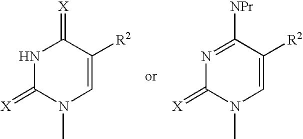
wherein
each X is independently O or S;
R² is selected from the group consisting of vinyl, 1-butenyl, 1-pentenyl, 1-hexenyl, 1-heptenyl, 1-octenyl, 1,3-pentadiynyl, 1-propynyl, 1-butynyl, 1-pentynyl, 3-methyl-1-butynyl, 3,3-dimethyl-1-butynyl, 3-buten-1-ynyl, bromovinyl, 1-hexynyl, 1-heptynyl, 1-octynyl, —C≡C-Z wherein Z is C₁₋₁₀ alkyl or C₁₋₁₀ haloalkyl, a 5-heteroaromatic group, or a 541-alkynyl)-heteroaromatic group; wherein the 5-heteroaromatic group and the 5-(1-alkynyl)-heteroaromatic group are optionally substituted on a ring carbon by oxygen or C₁₋₄ alkyl or are substituted on a ring nitrogen by C₁₋₄ alkyl; and
Pr is (H)₂ or a protecting group.

50. The composition of claim 34 wherein said A and G modified binding base is an A and G modified binding base having the following structure: 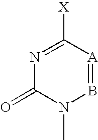
wherein
X is OH or NH₂, and
A and B may be the same or different and are C-lower alkyl, N, C—CF₃, C—F, C—Cl, C—Br, C—I, C-halocarbon, C—NO₂, C—OCF₃, C—SH, C—SCH₃, C—OH, C—O-lower alkyl, C—CH₂OH, C—CH₂SH, C—CH₂SCH₃, C—CH₂OCH₃, C—NH₂, C—CH₂ NH₂, C-alkyl-NH₂, C-benzyl, C-aryl, C-substituted aryl, C-substituted benzyl; or one of A and B are as defined above and the other is C—H; or together A and B are part of a carbocyclic or heterocyclic ring fused to the pyrimidine ring through A and B.

51. The composition of claim 34 wherein said A and G modified binding base is 5-alkylcytidine, 5-alkyluridine, 5-halouridine, 6-azapyrimidine, or 6-alkyluridine.

52. The composition of claim 34 wherein said A and G modified binding base is 5-fluorouracil.

53. The composition of claim 34 wherein said A and G modified binding base is an A and G modified binding base of the following structure: 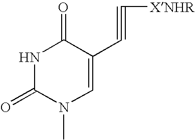
wherein
X′ is a branched or unbranched C₁₋₁₅ alkyl group;
R is an amino protecting group, a fluorophore, a non-radioactive detectable marker, or the group Y′NHA, where Y′ is a branched or unbranched C₁₋₄₀ alkyl carbonyl group and A is an amino protecting group, a fluorophore, or a non-radioactive detectable marker.

54. The composition of claim 34 wherein said A and G modified binding base is a pyrimidine base of one of the following structures: 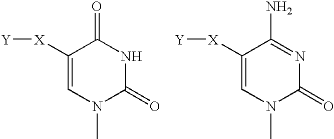
wherein
X is C₁-C₁₀ alkyl, C₁-C₁₀ unsaturated alkyl, dialkyl ether or dialkylthioether;
Y is —(NH₃)⁺, —(NH₂R¹)⁺, —(NHR¹R²)⁺, —(NR¹R²R³)⁺, dialkylsulfonium or trialkylphosphonium; and
R¹, R², and R³ are each independently lower alkyl having from one to ten carbon atoms.

55. The composition of claim 34 wherein said A and G modified binding base is an A and G modified binding base of the following structure: 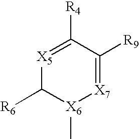
wherein
X₅ is N, O, C, S, or Si;
X₆ is CH or N, and at least one of X₅ and X₆ is N;
X₇ is —CH—;
R₄ is a reactive group derivatizable with a detectable label wherein said reactive group is selected from the group consisting of NH₂, SH, ═O, and a linking moiety selected from the group consisting of an amide, a thioether, a disulfide, a combination of an amide a thioether or a disulfide, R₁—(CH₂), —R₂ and R₁—R₂—(CH₂), —R₃ wherein x is an integer from 1 to 25 inclusive, and R₁, R₂, and R₃ are H, OH, alkyl, acyl, amide, thioether, or disulfide, and said detectable label is selected from the group consisting of radioisotopes, fluorescent or chemiluminescent reporter molecules, antibodies, haptens, biotin, photobiotin, digoxigenin, fluorescent aliphatic amino groups, avidin, enzymes, and acridinium;
R₆ is H, NH₂, SH, or ═O;
R₉ is hydrogen, methyl, bromine, fluorine, or iodine, alkyl or aromatic substituents, or an optional linking moiety selected from the group consisting of an amide, a thioether, a disulfide linkage, and a combination thereof.

56. The composition of claim 34 wherein said A and G modified binding base is an A and G modified binding base of one the following structures: 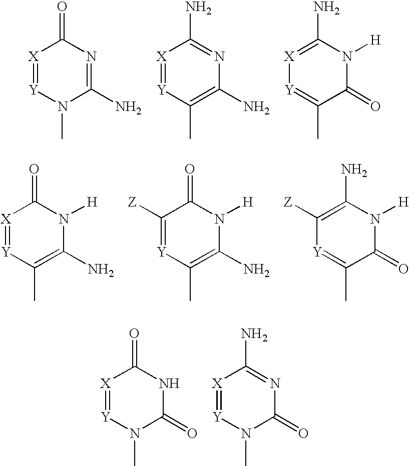
wherein
X is selected from the group consisting of a nitrogen atom and a carbon atom bearing a substituent Z;
Z is either a hydrogen, an unfunctionalized lower alkyl chain, or a lower alkyl chain bearing an amino, carboxyl, hydroxy, thiol, aryl, indole, or imidazoyl group; and
Y is selected from the group consisting of N and CH.

57. The composition of claim 34 wherein said A and G modified binding base is an A and G modified binding universal base of the following structure: 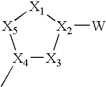
wherein
the foregoing structure has at least two double bonds in one of its possible tautomeric forms;
X₁, X₃ and X₅ are each members of the group consisting of N, O, C, S and Se;
X₂ and X₄ are each members of the group consisting of N and C; and
W is a member of the group consisting of F, Cl, Br, I, O, S, OH, SH, NH₂, NO₂, C(O)H, C(O)NHOH, C(S)NHOH, NO, C(NOCH₃)NH₂, OCH₃, SCH₃, SeCH₃, ONH₂, NHOCH₃, N₃, CN, C(O)NH₂, C(NOH)NH₂, CSNH₂ and CO₂H.

58. The composition of claim 34 wherein said A and G modified binding base is an A and G modified binding base of the following structure: 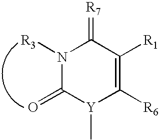
wherein
R₃ is a polycyclic aromatic group;
Y is C or N; R₇ is N or ═C(R₁)—; and
R₁ and R₆ are independently selected from the group consisting of H, halogen, C₁-C₁₀-alkyl, saturated or unsaturated cycloalkyl, C₁-C₁₀-alkylcarbonyloxy, hydroxy-C₁-C₁₀-alkyl, heterocycle (N, O, or S), and nitro.

59. The composition of claim 34 wherein said A and G modified binding base is an A and G modified binding base of the following structure: 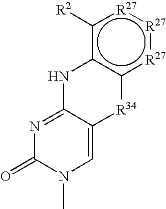
wherein
R² is A(Z)_{X1}, wherein A is a spacer and Z independently is a label bonding group optionally bonded to a detectable label;
R²⁷ is independently —CH═, —N═, —C(C_{1-8 alkyl})═ or —C(halogen)═, but no adjacent R²⁷ are both —N═, or two adjacent R²⁷ are taken together to form a ring having the structure, 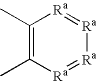
where each R^{a} is, independently, —CH═, —N═, —C(C_{1-8 alkyl})═ or —C(halogen)═, but no adjacent R^{a} are both —N═;
R³⁴ is —O—, —S— or —N(CH₃)—; and
X1 is 1, 2 or 3.

60. The composition of claim 34 wherein said A and G modified binding base is an A and G modified binding base of the following structure: 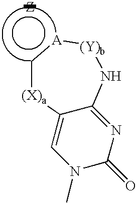
wherein
a and b are 0 or 1, and the total of a and b is 0 or 1;
A is N or C;
X is S, O, —C(O)—, NH or NCH₂R₆;
Y is —C(O)—;
Z is taken together with A to form an aryl or heteroaryl ring structure comprising 5 or 6 ring atoms wherein the heteroaryl ring comprises a single O ring heteroatom, a single N ring heteroatom, a single S ring heteroatom, a single 0 and a single N ring heteroatom separated by a carbon atom, a single S and a single N ring heteroatom separated by a carbon atom, 2 N ring heteroatoms separated by a carbon atom, or 3 N ring heteroatoms at least two of which are separated by a carbon atom, and wherein at least 1 nonbridging ring carbon atom is substituted with R₆ or ═O;
R₃ is a protecting group or H;
R₆ is independently H, C₁-C₆ alkyl, C₂-C₆ alkenyl, C₂-C₆ alkynyl, NO₂, N(R₃)₂, AN or halo, or R₆ is taken together with an adjacent R₆ to complete a ring containing 5 or 6 ring atoms.

61. The composition of claim 34 wherein said A and G modified binding base is a non-heterocyclic A and G modified binding base of the following structure:
—O—Rₘ—O—Rₙ
wherein
Rₘ is C₁ to C₁₆ alkylene or an oxyethylene oligomer —(CH₂CH₂O)_{z}— where z is an integer in the range of 1 to 16 inclusive, and
Rₙ is selected from the group consisting of:

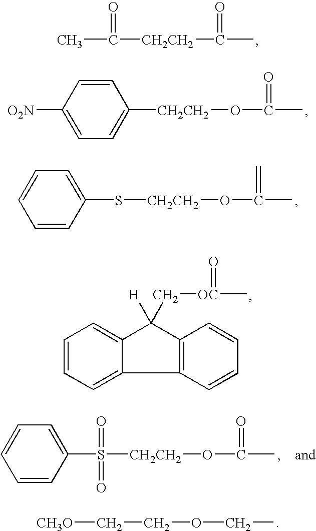

62. A pharmaceutical composition comprising the composition of claim 34 and a pharmaceutically acceptable carrier.

63. A method of modulating the expression of a target nucleic acid in a cell comprising contacting said cell with a composition of claim 34.

64. A method of treating or preventing a disease or disorder associated with a target nucleic acid comprising administering to an animal having or predisposed to said disease or disorder a therapeutically effective amount of a composition of claim 34.


SEQUENCE LISTING (23 sequences)

SEQ ID NO: 1 - RNA, 19 nt, Artificial
  Synthetic Construct
cgagaggcggacgggaccg

SEQ ID NO: 2 - DNA, 21 bp, Artificial
  Synthetic Construct
cgagaggcggacgggaccgtt

SEQ ID NO: 3 - DNA, 21 bp, Artificial
  Synthetic Construct
cggtcccgtccgcctctcgtt

SEQ ID NO: 4 - DNA, 20 bp, Artificial
  Synthetic Construct
tccgtcatcgctcctcaggg

SEQ ID NO: 5 - DNA, 20 bp, Artificial
  Synthetic Construct
gtgcgcgcgagcccgaaatc

SEQ ID NO: 6 - DNA, 20 bp, Artificial
  Synthetic Construct
atgcattctgcccccaagga

SEQ ID NO: 7 - DNA, 16 bp, Artificial
  Synthetic Construct
tccaggtgtccgcatc

SEQ ID NO: 8 - DNA, 16 bp, Artificial
  Synthetic Construct
gcgttttttttttgcg

SEQ ID NO: 9 - DNA, 16 bp, Artificial
  Synthetic Construct
tccaggtgtccgcatc

SEQ ID NO: 10 - DNA, 16 bp, Artificial
  Synthetic Construct
tccaggtgtccgcatc

SEQ ID NO: 11 - DNA, 16 bp, Artificial
  Synthetic Construct
gcgttttttttttgcg

SEQ ID NO: 12 - DNA, 16 bp, Artificial
  Synthetic Construct
gcgttttttttttgcg

SEQ ID NO: 13 - DNA, 20 bp, Artificial
  Synthetic Construct
aggtgctcaggactccattt

SEQ ID NO: 14 - DNA, 20 bp, Artificial
  Synthetic Construct
aggtgctcaggactccattt

SEQ ID NO: 15 - DNA, 20 bp, Artificial
  Synthetic Construct
ctccagcgcctccaccaggc

SEQ ID NO: 16 - DNA, 20 bp, Artificial
  Synthetic Construct
ctccagcgcctccaccaggc

SEQ ID NO: 17 - DNA, 20 bp, Artificial
  Synthetic Construct
ctgctagcctctggatttga

SEQ ID NO: 18 - DNA, 20 bp, Artificial
  Synthetic Construct
ctgctagcctctggatttga

SEQ ID NO: 19 - DNA, 20 bp, Artificial
  Synthetic Construct
ccggtaccccaggttcttca

SEQ ID NO: 20 - DNA, 20 bp, Artificial
  Synthetic Construct
ccggtaccccaggttcttca

SEQ ID NO: 21 - DNA, 20 bp, Artificial
  Synthetic Construct
atagtttcacctagagaaag

SEQ ID NO: 22 - DNA, 20 bp, Artificial
  Synthetic Construct
atagtttcacctagagaaag

SEQ ID NO: 23 - DNA, 19 bp, Artificial
  Synthetic Construct
ttttttttttttttttttt